(19) United States
(12) Patent Application Publication
(10) Pub. No.: US 2024/0115705 A1
(43) Pub. Date: Apr. 11, 2024

(54) Regulation of Butyrophilin subfamily 3 member A1 (BTN3A1, CD277)

(71) Applicants: The J. Gladstone Institutes, a testamentary trust established under the will of J. David Gladstone, San Francisco, CA (US); The Regents of the University of California, Oakland, CA (US)

(72) Inventors: Alexander Marson, San Francisco, CA (US); Murad Mamedov, San Francisco, CA (US)

(21) Appl. No.: 18/274,307

(22) PCT Filed: Feb. 4, 2022
(86) PCT No.: PCT/US2022/070520

§ 371 Date: Jul. 26, 2023

Related U.S. Application Data
(60) Provisional application No. 63/147,050, filed on Feb. 8, 2021.

(51) Int. Cl. A61K 39/00; A61K 45/06; A61P 35/00; C12N 15/86; G01N 33/50

(52) U.S. Cl. CPC A61K 39/464429; A61K 39/4611; A61K 45/06; A61P 35/00; C12N 15/86; G01N 33/5011; C12N 2740/15043

(57) ABSTRACT

Described herein are positive and negative regulators of BTN3A, as well as methods for identifying subjects who can benefit from T cell therapies and/or various chemotherapies. The subjects can for example be suffering from immune disorders, cancer and other diseases and conditions.

### PRIORITY APPLICATIONS

This application claims the benefit of priority of U.S. Provisional Application Ser. No. 63/147,050, filed Feb. 8, 2021, the content of which is specifically incorporated herein by reference in its entirety.

### INCORPORATION BY REFERENCE OF SEQUENCE LISTING

### Provided as a Text File

A Sequence Listing is provided herewith as a text file, “2213184.txt”, created on Feb. 3, 2022 and having a size of 475,136 bytes. The contents of the text file are incorporated by reference herein in their entirety.

### BACKGROUND

Examples of cellular therapeutic agents that can be useful as anticancer therapeutics include CD8+ T cells, CD4+ T cells, natural killer (NK) cells, natural killer T (NKT) cells, γδ T cells, dendritic cells, and CAR T cells. Use of patient-derived immune cells can also be an effective cancer treatment that has little or no side effects. NK cells have cell-killing efficacy but can have negative effects (Bolourian & Mojtahedi, Immunotherapy 9(3):281-288 (2017)). Dendritic cells are therapeutic agents belonging to the vaccine concept in that they have no function of directly killing cells but they are capable of delivering antigen specificity to T cells in the patient's body so that cancer cell specificity is imparted to T cells with high efficiency. In addition, CD4+ T cells play a role in helping other cells through antigen specificity, and CD8+ T cells are known to have the best antigen specificity and cell-killing effect. γδ T cells can be used both as autologous and allogeneic therapies, which do not cause graft-versus-host disease (GvHD).

However, most cell therapeutic agents that have been used or developed to date have limited clinical effect for most cancers. For example, cancer cells, on their own, secrete substances that suppress immune responses in the human body, or do not present antigens necessary for adaptive immune recognition of such cancer cells, thereby preventing an appropriate immune response from occurring.

### SUMMARY

Compositions and methods of modulating butyrophilin subfamily 3 member A1 (BTN3A1, CD277) expression and function are described herein. Such composition and methods can modulate T cell responses. The T cells can be modulated in vivo or ex vivo. T cells modulated ex vivo using the methods described herein can be administered to a subject who may benefit from such administration. Methods are also described herein for evaluating test agents and identifying agents that are useful for modulating T cells.

BTN3A1 can inhibit alpha-beta T cell activity in specific contexts, including cancer-related contexts (Payne et al., Science, 2020). Therefore, compositions and methods that silence or inhibit BTN3A1, or the positive regulators of BTN3A1; or compositions and methods that enhance the activities of negative regulators of BTN3A1 can reduce BTN3A1 levels in various cancer and infectious disease applications to achieve stronger alpha-beta CD4 or CD8 T cell responses.

However, BTN3A1 can also activate a subset of human gamma-delta T cells called Vgamma9Vdelta2 (Vγ9Vδ2) T cells, which can for example participate in the anti-tumor immune surveillance. Such Vγ9Vδ2 T cells can recognize phosphoantigen accumulation in target cells and molecules expressed on cells undergoing neoplastic transformation. Such Vγ9Vδ2 T cells can also recognize the presence of pathogen-derived phosphoantigens and target the infected cells. Therefore, compositions and methods that upregulate or enhance BTN3A1, or the positive regulators of BTN3A1; or compositions and methods that silence or inhibit the activities of negative regulators of BTN3A1 could upregulate BTN3A1 levels in various cancer and infectious disease applications to achieve stronger Vγ9Vδ2 T cell responses.

Experiments described herein reveal a multilayered regulatory framework exists that modulates interactions between γδ T cells and BTN3A1. For example, as shown herein, BTN3A1 abundance and/or accessibility is transcriptionally regulated by IRF1, IRF8, IRF9, NLRC5, SPI1, SPIB, ZNF217, RUNX1, AMPK, or a combination thereof. Also as shown herein, increased BTN3A surface abundance was also observed after disruption of the sialylation machinery (CMAS), after disruption of the retention in endoplasmic reticulum sorting receptor 1 (RER1), and after disruption of the iron-sulfur cluster formation (FAM96B). However, CtBP1 (a metabolic sensor whose transcriptional and trafficking regulation depends on the cellular NAD+/NADH ratio) negatively regulates BTN3A abundance. Knockout of PPAT (purine biosynthesis), GALE (galactose catabolism), NDUFA2 (OXPHOS), and TIMMDC1 (OXPHOS) led to upregulation of BTN3A1/2 transcription. Also as shown herein, AMPK is a regulator of BTN3A1 expression in cells undergoing an energy crisis. Hence, the experimental results shown herein illuminate a mechanism of stress-regulation of a key γδ T cell-cancer cell interaction.

Methods for identifying and/or treating candidates who can benefit from T cell therapies are described herein. For example, as illustrated herein, if a sample exhibits increased expression levels of any of the BTN3A positive regulators described herein (relative to a reference value or negative control), the subject from whom the sample was obtained is a good candidate for T cell therapy. However, if a sample exhibits increased expression levels of any of the BTN3A negative regulators described herein (relative to a reference value or negative control), the subject from whom the sample was obtained is likely not a good candidate for T cell therapy.

### DESCRIPTION OF THE FIGURES

FIG. 1A-1E illustrate that Vγ9Vδ2 T cell co-cultures with a genome-wide knockout library of Daudi cells reveal which genetic knockouts lead to Daudi cancer cell killing-evasion and which lead to Daudi cancer cell killing-enhancement by the T cells. FIG. 1A is a schematic of the screen of Vγ9Vδ2 T cells co-cultured with genome-wide knockout (KO) library of Daudi-Cas9 cells (ZOL=zoledronate, which enhances phosphoantigens). The Vγ9Vδ2 T cells kill some Daudi cell knockout mutants, which are detected by comparing gRNA abundance to that in the input population. FIG. 1B is a schematic diagram of the mevalonate pathway. Phosphoantigens are indicated by a crosshatched background, and the locus of zoledronate (ZOL) effects on phosphoantigen enhancement is shown. FIG. 1C graphically illustrates a ranking of all 18,010 genes from negative enrichment (left) to positive enrichment (right) of Daudi-Cas9 KO cells that enhance killing or evade killing, respectively. Genes identified to the left (circular symbols) enhance cancer cell killing, while those identified to the right (square symbols; right box) help cancer cells evade killing. Vertical lines on the x-axis identify the rank positions of OXPHOS Complex I-V subunits listed in the left box. The OXPHOS system comprises five multi-subunit protein complexes, of which NADH-ubiquinone oxidoreductase (complex 1, CI) is a major electron entry point into the electron transport chain (ETC) that is central to mitochondrial ATP synthesis. Boxes show only a subset of significant hits. All non-significant gene points are shown as diamond symbols. False-discovery rate (FDR)<0.05, except ^{#}FDR<0.1 for ICAM1 and SLC37A3. FIG. 1D shows a schematic of the enrichment or depletion of cells with specific genetic KOs within the mevalonate pathway and their statistical significance (fold change [FC]). Cross-hatching indicating log 2(fold change) is shown only for significant hits (FDR<0.05). As illustrated, knockouts of certain mevalonate pathway enzymes (HMGCS1, MVD, FDPS, GGPS1) within cancer cells significantly enhanced T cell-mediated killing of those cancer cells. However, knockouts of some mevalonate pathway enzymes (ACAT2, HMGCR, SQLE), two of which are upstream of FDPS phosphoantigen synthesis, did not enhance cancer cell killing. FIG. 1E graphically illustrates enrichment or depletion of individual single guide RNAs (sgRNA) for a selection of significant hits, overlaid on a gradient showing distribution of all sgRNAs. As illustrated, cells with knockout of some genes (e.g., FDPS, PPAT, NDUFA3, NDUFA2, NDUFB7, NDUFA6) were frequently killed by the T cells, so the sgRNAs for these genes were detected in only small numbers of cells. However, cells with knockout of other genes (BTN3A1, ACAT2, BTN2A1, IRF1) were not killed so frequently by the T cells, so the sgRNAs for these genes were detected in significantly greater numbers of cells. For FIG. 1B-1E, n=3 PBMC donors; enrichment and statistics calculated by the MAGeCK algorithm.

FIG. 2A-2L illustrate that regulation of BTN3A surface expression overlaps with enhancement and evasion of T cell killing. FIG. 2A is a schematic illustrating the genome-wide knockout (KO) screen for surface expression of BTN3A (CD277). A library of Daudi-Cas9 knockout mutant cells were generated and screened for expression of BTN3A (CD277). The top and bottom 25% BTN3A⁺ cells were sorted for downstream next generation sequencing (NGS) analysis. FIG. 2B is a schematic illustrating screen concordance. As illustrated, knockout of some genes (e.g., endoplasmic reticulum sorting receptor 1, RER1) can increase BTN3A surface expression and also increase cancer cell killing—such genes are negative regulators of BTN3A (when not mutated). However, loss of other genes (e.g., Interferon regulatory factor 1 (IRF1), IRF8, IRF9, NLRC5, SPIB, SPI1, TIMDC1) can decrease BTN3A surface expression and also decrease cancer cell killing—such genes are positive regulators of BTN3A (when not mutated). FIG. 2C graphically illustrates ranking of all 18,010 genes by their negative to positive cellular enrichment in Daudi-Cas9 KO cells that express low levels of BTN3A (BTN3A^{high}) relative to Daudi-Cas9 cells that express high levels of BTN3A (BTN3A^{high}). Concordant hits (BTN3A screen FDR<0.01, co-culture screen FDR<0.05) and non-concordant hits (BTN3A screen FDR<0.01) are highlighted. The distribution of KEGG gene sets is shown below the graph (see genome.jp/kegg/genes.html for KEGG genes). FIG. 2D graphically illustrates correlation of screen effect sizes (LFC) among concordant hits separated into positive regulators (circles) and negative regulators (triangles) of BTN3A surface expression. FIG. 2E is a schematic diagram illustrating which of the purine biosynthesis pathway genes are depleted in the KO cells across both screens. Crosshatched backgrounds of the gene names indicate the log 2(fold change), but only for significant hits (FDR<0.05). FIG. 2F shows representative histograms of surface BTN3A fluorescence for a subset of single gene KOs compared to an AAVS1 control. FIG. 2G graphically illustrates surface BTN3A median fluorescence intensity (MFI) at 13 days post-transduction for two distinct KOs per gene deletion identified on the y-axis, except for BTN3A1 where the data are shown for one KO. The results were normalized to BTN3A MFI in AAVS1 controls and log 2-transformed. Two distinct KOs were analyzed per gene deletion, except for BTN3A1 (one KO). Combined data from three separate experiments are shown. AAVS1 n=36, BTN3A1 n=9, n=18 all other deletions. FIG. 2H graphically illustrates TCR tetramer staining fluorescence (MFI) of the G115 Vγ9Vδ2 clone at 13 days post-transduction for cells with the different genetic KOs listed on the y-axis. Representative data from one experiment are shown. AAVS1 n=12, BTN3A1 n=3, n=6 all other deletions. FIG. 2I graphically illustrates qPCR data for BTN3A1 transcripts normalized to ACTB transcripts for cells with different types of gene KOs. Combined data from two independent experiments. n=5-6, AAVS1 n=12. FIG. 2J graphically illustrates qPCR data for BTN3A2 transcripts normalized to ACTR transcripts for cells with different types of gene KOs. Combined data from two independent experiments. n=5-6, AAVS1 n=12. One-way ANOVA with Dunnett's multiple comparisons test for FIG. 2G-2J. Mean±SD. p<0.0001 (****), p<0.001 (***), p<0.01 (**), p<0.05 (*). FIG. 2K graphically illustrates BTN3A expression on live Daudi-Cas9 cells treated with varying amounts of zoledronate for 72 hours. Representative data from one of three independent experiments. n=3 per ZOL dose. Mean±SD. FIG. 2L graphically illustrates BTN2A1 levels in cell lines, each with a knockout gene identified along the x-axis. The BTN2A1 levels were measured by qPCR. The type of gene is indicated by crosshatching as shown in the key to the right.

FIG. 3A-3M illustrate transcriptional and metabolic regulation of BTN3A. FIG. 3A is a schematic of the oxidative phosphorylation/electron transport-linked phosphorylation pathway (OXPHOS) with relevant inhibitors and genetic knockouts identified. FIG. 3B graphically illustrates surface BTN3A median fluorescence intensity (MFI) in Daudi-Cas9 knockout cells cultured in various glucose concentrations for 3 days in RPMI (+glutamine, +fetal calf serum, +penicillin/streptomycin, −glucose, −pyruvate). The fluorescence data were normalized to fluorescence data of cells grown without glucose (0 g/L). n=4 per condition, data combined from two independent experiments. One-way ANOVA with Dunnett's multiple comparisons test. FIG. 3C graphically illustrates surface BTN3A MFI in wildtype (WT) Daudi-Cas9 cells cultured with OXPHOS inhibitors of complex I (rotenone, circles), complex V (oligomycin A, triangles A), and mitochondrial membrane potential (carbonyl cyanide-4 (trifluoromethoxy) phenylhydrazone, FCCP, upside-down triangles) for 72 hours in complete RPMI. n=4 per condition, two independent experiments combined. One-way ANOVA with Dunnett's multiple comparisons test. FIG. 3D graphically illustrates surface BTN3A MFI in wildtype (WT) Daudi-Cas9 cells cultured with an OXPHOS inhibitor of complex III (antimycin A, circles), compared to control (squares), for 72 hours in complete RPMI. n=3 per condition, representative data from one of two experiments. Two-tailed unpaired Student's t test. FIG. 3E graphically illustrates surface BTN3A MFI in WT Daudi-Cas9 cells cultured with glycolysis-blocking 2-deoxy-D-glucose (2-DG), or equivalent amount of DMSO (vehicle) for 72 hours in complete RPMI. n=3 per condition. Representative data from one of three independent experiments. FIG. 3F graphically illustrates surface BTN3A MFI in WT Daudi-Cas9 cells cultured with AICAR (N¹-(β-D-Ribofuranosyl)-5-aminoimidazole-4-carboxamide); an allosteric activator of AMP-activated protein kinase (AMPK)), or equivalent amount of DMSO (vehicle) for 72 hours in complete RPMI. n=3 per condition. Representative data from one of three independent experiments. FIG. 3G graphically illustrates surface BTN3A MFI in WT Daudi-Cas9 cells cultured with Compound 991 or equivalent amount of DMSO (vehicle) for 72 hours in complete RPMI. n=3 per condition. Representative data from one of two independent experiments. Two-tailed unpaired Student's t test. FIG. 3H graphically illustrates fluorescence (MFI) of Vγ9Vδ2 G115 clone tetramers with WT Daudi-Cas9 cells treated with 80 μM Compound 991 (DMSO), DMSO (vehicle), 0.5 mM AICAR (aqueous), or nothing for 72 hours. n=4 per condition. Representative Data from one of two independent experiments. Two-tailed unpaired Student's t test. FIG. 3I graphically illustrates expression levels of BTN2A1, BTN3A1, and BTN3A2 transcripts as detected by qPCR in Daudi-Cas9 cells treated with Compound 991, internally normalized to ACTB transcripts and normalized to DMSO (vehicle)-treated cells. n=4 per condition. Representative from one of three independent experiments. Two-tailed unpaired Student's t test. FIG. 3J graphically illustrates surface BTN3A MFI in WT Daudi-Cas9 cells co-treated with increasing amounts of Compound C and the AMPK activator, AICAR. n=3 per conditions compared to DMSO-treated controls. Representative data from one of two independent experiments. FIG. 3K graphically illustrates surface BTN3A MFI in WT Daudi-Cas9 cells co-treated with increasing amounts of Compound C and one of the indicated OXPHOS/glycolysis inhibitors (Oligomycin, FCCP, 2-DG, Rotenone). n=3 per condition. Representative data from one of three independent experiments. Mean±SD. p<0.0001 (****), p<0.001 (***), p<0.01 (**), p<0.05 (*). FIG. 3L graphically illustrates surface BTN3A MFI in Daudi-Cas9 cells treated for 72 hours with the compounds identified along the X-axis in PPAT KO cells or in AAVS1 KO cells. As a control, aliquots of the KO cells were also treated with an equivalent amount of DMSO (vehicle). FIG. 3M graphically illustrates surface BTN3A MFI in Daudi-Cas9 cells treated for 72 hours with the AMPK agonist A-769662, or equivalent amount of DMSO (vehicle).

FIG. 4A-4F illustrate that the co-culture screen and BTN3A screen described herein correlate with patient survival, especially in cancers involving Vγ9Vδ2 T cell infiltration. FIG. 4A graphically illustrates survival of low grade-glioma (LGG) patients (n=529) exhibiting either high expression levels or low expression levels of the co-culture screen gene signature (HIT). FIG. 4B graphically illustrates survival of LGG patients expressing high levels of T Cell Receptor Gamma Variable 9 (TRGV9)/T Cell Receptor Gamma Variable (TRDV2) (i.e., TRGV9-TRDV2-high) or low levels of TRGV9/TRDV2 (TRGV9/TRDV2-low) while exhibiting either high or low expression of the co-culture screen gene signature (HIT). FIG. 4C graphically illustrates survival of bladder urothelial carcinoma (BLCA) patients (n=433) exhibiting either high expression levels or low expression levels of the co-culture screen gene signature (HIT). FIG. 4D graphically illustrates survival of TRGV9/TRDV2-high or TRGV9/TRDV2-low BLCA patients split by high and low expression of the co-culture screen gene signature (HIT). For FIG. 4A-4D, log-rank test (Kaplan-Meier survival analysis) was used. For FIGS. 4A and 4C Wald test (Cox regression), adjusted (p_{adj}) with Benjamini-Hochberg multiple comparisons correction. FIG. 4E graphically illustrates the survival of total LGG patients split by high and low expression of the BTN3A expression screen gene signature (HIT). Log-rank test (Kaplan-Meier survival analysis) and Wald test (Cox regression) were used, adjusted (p_{adj}) with Benjamini-Hochberg multiple comparisons correction. FIG. 4F graphically illustrates the survival of TRGV9/TRDV2-high/low LGG patients split by high and low expression of the BTN3A expression screen gene signature (HIT). Log-rank test (Kaplan-Meier survival analysis) and Wald test (Cox regression) were used, adjusted (p_{adj}) with Benjamini-Hochberg multiple comparisons correction.

### DETAILED DESCRIPTION

Methods are described herein for identifying and treating subjects who can benefit from T cell therapies. Methods and compositions are also described herein for detecting and modulating BTN3A expression and/or activity that are useful for modulating T cell responses.

Methods are described herein that can involve obtaining a sample from a subject and comparing gene expression levels in the sample with one or more reference values, where the expression levels of the following genes are compared: genes involved in oxidative phosphorylation (OXPHOS genes), genes involved in the mevalonate pathway, genes involved in metabolic sensing, genes involved in purine biosynthesis (PPAT genes), transcription factor genes, BTN3A genes, or a combination of those genes. The method can also include classifying the subject from whom the sample was obtained as having cancer (i.e., being a cancer patient) or not having cancer. The methods can also include classifying a cancer patient as being a candidate for T cell therapy based on the expression of those genes in the patient's sample. The methods can also involve administering T cells to cancer patients identified as candidates for T cell therapy.

For example, a method is described herein for treating or identifying a cancer patient who can benefit from administration of T cells, including Vγ9Vδ2 T cells. The method can include: (a) comparing the respective levels of expression of genes involved in oxidative phosphorylation (OXPHOS genes), genes involved in the mevalonate pathway, genes involved in metabolic sensing, genes involved in purine biosynthesis (PPAT genes), transcription factor genes, BTN3A genes, or a combination of those genes in one or more samples taken from one or more subjects suspected of having cancer to respective reference values of expression of the genes; and (b) obtaining T cells from one or more subjects (treatable subjects) exhibiting altered expression levels of the genes involved in oxidative phosphorylation (OXPHOS genes), genes involved in the mevalonate pathway, genes involved in metabolic sensing, genes involved in purine biosynthesis (PPAT genes), transcription factor genes, BTN3A genes, or a combination of those genes. The methods can also involve expanding the T cells obtained from one or more of the treatable subjects to provide one or more populations of T cells. The methods can also involve administering one or more populations of T cells to one or more of the treatable subjects. In some cases, the T cells that are expanded and/or administered are Vγ9Vδ2 T cells.

Hence, changes in BTN3A and/or the BTN3A regulators described herein can be used to detected cancer, infections, or a combination thereof. Detection of BTN3A1 on cancer cells in an assay mixture and/or quantification thereof can be used to determine whether the cancer cells can be treated by T cells or by any of the regulators or modulators described herein.

### Samples

Subjects with cancer who can benefit from T cell therapies or by modulating the expression or activity of BTN3A or any of its regulators can be assessed through the evaluation of expression patterns, or profiles, of genes described herein. For example, the expression levels of BTN3A and/or any of its regulators can be evaluated to identify candidates who can benefit from T cell therapies and/or by administration of agents that can modulate BTN3A or any of its regulators. Genes whose expression is particularly informative include, for example, the BTN3A regulator genes involved in oxidative phosphorylation (OXPHOS genes), genes involved in the mevalonate pathway, genes involved in metabolic sensing, genes involved in purine biosynthesis (PPAT genes), transcription factor genes, BTN3A genes, or a combination of those genes in one or more subject samples. The term subject, or subject sample, refers to an individual regardless of health and/or disease status. A subject can be a patient, a study participant, a control subject, a screening subject, or any other class of individual from whom a sample is obtained and who is to be assessed using the markers and/or methods described herein. Accordingly, a subject can be diagnosed with cancer, can present with one or more symptoms of cancer, can have a predisposing factor, such as a family (genetic) or medical history (medical) factor, can be undergoing treatment or therapy for cancer, or the like. Alternatively, a subject can be healthy with respect to any of the aforementioned factors or criteria. It will be appreciated that the term “healthy” as used herein, is relative to cancer status, as the term “healthy” cannot be defined to correspond to any absolute evaluation or status. Thus, an individual defined as healthy with reference to any specified disease or disease criterion, can in fact be diagnosed with any one or more other diseases, or exhibit any of one or more other disease criterion, including one or more infections or conditions other than cancer. Healthy controls are preferably free of any cancer.

In some cases, the methods for detecting, predicting, assessing the prognosis of cancer, and/or assessing the benefits of T cell therapy for a subject can include collecting a biological sample comprising a cell or tissue, such as a bodily fluid sample, tissue sample, or a primary tumor tissue sample. By “biological sample” is intended any sampling of cells, tissues, or bodily fluids in which expression of genes can be detected. Examples of such biological samples include, but are not limited to, biopsies and smears. Bodily fluids useful in the present invention include blood, lymph, urine, saliva, nipple aspirates, gynecological fluids, hematopoietic cells, semen, or any other bodily secretion or derivative thereof. Blood can include whole blood, plasma, serum, or any derivative of blood. In some embodiments, the biological sample includes cells, particularly hematopoietic cells. Biological samples may be obtained from a subject by a variety of techniques including, for example, by using a needle to withdraw or aspirate cells or bodily fluids, by scraping or swabbing an area, or by removing a tissue sample (i.e., biopsy). In some embodiments, a sample includes hematopoietic cells, immune cells, B cells, or combinations thereof.

The samples can be stabilized for evaluating and/or quantifying expression levels of the oxidative phosphorylation (OXPHOS) genes, genes involved in the mevalonate pathway, genes involved in metabolic sensing, genes involved in purine biosynthesis (PPAT genes), transcription factor genes, BTN3A genes, or a combination of those genes in one or more subject samples.

In some cases, fixative and staining solutions may be applied to some of the cells or tissues for preserving the specimen and for facilitating examination. Biological samples may be transferred to a glass slide for viewing under magnification. The biological sample can be formalin-fixed, and/or paraffin-embedded breast tissue samples. However, in some cases the sample is immediately treated to preserve RNA, for example, by disruption of cells, disruption of proteins, addition of RNase inhibitors, or a combination thereof.

Samples can have cancer cells but may also not have cancer cells. In some cases, the samples can include leukemia cells, lymphoma cells, Hodgkin's disease cells, sarcomas of the soft tissue and bone, lung cancer cells, mesothelioma, esophagus cancer cells, stomach cancer cells, pancreatic cancer cells, hepatobiliary cancer cells, small intestinal cancer cells, colon cancer cells, colorectal cancer cells, rectum cancer cells, kidney cancer cells, urethral cancer cells, bladder cancer cells, prostate cancer cells, testis cancer cells, cervical cancer cells, ovarian cancer cells, breast cancer cells, endocrine system cancer cells, skin cancer cells, central nervous system cancer cells, melanoma cells of cutaneous and/or intraocular origin, cancer cells associated with AIDS, or a combination thereof. In addition, metastatic cancer cells at any stage of progression can be tested in the assays, such as micrometastatic tumor cells, megametastatic tumor cells, and recurrent cancer cells. For example, as explained herein, malignancy associated response signature expression levels in a sample can be assessed relative to normal tissue from the same subject or from a sample from another subject or from a repository of normal subject samples.

### Gene Expression

Various methods can be used for evaluating and/or quantifying expression levels of genes involved in oxidative phosphorylation (OXPHOS genes), genes involved in the mevalonate pathway, genes involved in metabolic sensing, genes involved in purine biosynthesis (PPAT genes), transcription factor genes, BTN3A genes, or a combination of those genes in one or more subject samples. By “evaluating and/or quantifying” is intended determining the quantity or presence of an RNA transcript or its expression product (i.e., protein product).

Examples of BTN3A genes include BTN3A1, BTN3A2, BTN3A3, variants and isoforms thereof, or combinations thereof. Examples of one or more of the transcription factor genes include CTBP1, IRF1, IRF8, IRF9, NLRC5, RUNX1, ZNF217, or a combination thereof. Examples of one or more of the mevalonate pathway genes include FDPS, HMGCS1, MVD, FDPS, GGPS1, or a combination thereof. Examples of one or more of the purine biosynthesis (PPAT) genes include PPAT, GART, ADSL, PAICS, PFAS, ATIC, ADSS, GMPS, or a combination thereof. CtBP1 is an example of a metabolic sensing gene.

A number of OXPHOS genes exist and the expression of any of these OXPHOS genes can be evaluated/measured in the methods described herein. For example, one or more of the following genes are OXPHOS genes: ATP5A1, ATP5B, ATP5C1, ATP5D, ATP5E, ATP5F1, ATP5G1, ATP5G2, ATP5G3, ATP5H, ATP5I, ATP5J, ATP5J2, ATP5L, ATP5O, ATP5S, COX4I1, COX4I2, COX5A, COX5B, COX6A1, COX6A2, COX6B1, COX6B2, COX6C, COX7A1, COX7A2, COX7B, COX7B2, COX7C, COX8A, COX8C, CYC1, NDUFA1, NDUFA10, NDUFA11, NDUFA12, NDUFA13, NDUFA2, NDUFA3, NDUFA4, NDUFA5, NDUFA6, NDUFA7, NDUFA8, NDUFA9, NDUFAB1, NDUFB1, NDUFB10, NDUFB11, NDUFB2, NDUFB3, NDUFB4, NDUFB5, NDUFB6, NDUFB7, NDUFB8, NDUFB9, NDUFC1, NDUFC2, NDUFS1, NDUFS2, NDUFS3, NDUFS4, NDUFS5, NDUFS6, NDUFS7, NDUFS8, NDUFV1, NDUFV2, NDUFV3, SDHA, SDHB, SDHC, SDHD, UQCR10, UQCR11, UQCRC1, UQCRC2, UQCRFS1, UQCRH, UQCRQ, or a combination thereof. In some cases, one or more of the following OXPHOS genes can be evaluated/measured in the methods described herein. ATP5, ATP5A1, ATP5B, ATP5D, ATP5J2, COX (e.g., COX4I1, COX5A, COX6B1, COX6C, COX7B, COX8A), GALE, NDUFA (e.g., NDUFA2, NDUFA3, NDUFA6, and/or NDUFB7), NDUFB, NDUFC2, NDUFS, NDUFV1, SDHC, TIMMDC1, UQCRC1, UQCRC2, or a combination thereof.

Methods for detecting expression of the genes, including gene expression profiling, can involve methods based on hybridization analysis of polynucleotides, methods based on sequencing of polynucleotides, immunohistochemistry methods, and proteomics-based methods. The methods generally involve detect expression products (e.g., mRNA or proteins) encoding by the genes.

In some cases, RNA transcripts are reverse transcribed and sequenced. For example, quantitative polymerase chain reaction (qPCR) can be used to evaluate expression levels of genes. In some cases, next generation sequencing (NGS) can be used to evaluate expression levels. For example, RNA sequencing (RNA-Seq) using NGS can detect both known and novel transcripts. Because RNA-Seq does not require predesigned probes, the data sets are unbiased, allowing for hypothesis-free experimental design.

In some cases, PCR-based methods, which can include reverse transcription PCR (RT-PCR) (Weis et al., TIG 8:263-64, 1992), array-based methods such as microarray (Schena et al., Science 270:467-70, 1995), or combinations thereof are used. By “microarray” is intended an ordered arrangement of hybridizable array elements, such as, for example, polynucleotide probes, on a substrate. The term “probe” refers to any molecule that is capable of selectively binding to a specifically intended target biomolecule, for example, a nucleotide transcript or a protein encoded by or corresponding to one or genes involved in oxidative phosphorylation (OXPHOS genes), genes involved in the mevalonate pathway, genes involved in metabolic sensing, genes involved in purine biosynthesis (PPAT genes), transcription factor genes, BTN3A genes, or a combination of those genes. Probes may be specifically designed to be labeled. Examples of molecules that can be utilized as probes include, but are not limited to, RNA, DNA, proteins, antibodies, and organic molecules.

Many expression detection methods use isolated RNA. The starting material is typically total RNA isolated from a biological sample, such as one or more types of cell or tissue sample, one or more types of hematopoietic cells, one or more types of tumor or tumor cell line, one or more types of corresponding normal tissue or cell line, or a combination thereof. If the source of RNA is a sample from a subject, RNA (e.g., mRNA) can be extracted, for example, from stabilized, frozen or archived paraffin-embedded, or fixed (e.g., formalin-fixed) tissue or cell samples (e.g., pathologist-guided tissue core samples).

General methods for RNA extraction are available and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In some cases, RNA isolation can be performed using a purification kit, a buffer set and protease from commercial manufacturers, such as Qiagen (Valencia, Calif.), according to the manufacturer's instructions. For example, total RNA from cells can be isolated using Qiagen RNeasy mini-columns. Other commercially available RNA isolation kits include MASTERPURE™ Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from tissue or cell samples (e.g. tumors) can be isolated, for example, by cesium chloride density gradient centrifugation. Additionally, large numbers of tissue samples can readily be processed using available techniques, such as, for example, the single-step RNA isolation process of Chomczynski (U.S. Pat. No. 4,843,155).

Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 60, 100, 250, or 500 nucleotides in length and sufficient to specifically hybridize under stringent conditions to any of genes of RNA transcripts involved in oxidative phosphorylation (OXPHOS genes), genes involved in the mevalonate pathway, genes involved in metabolic sensing, genes involved in purine biosynthesis (PPAT genes), transcription factor genes, or a combination of those genes, BTN3A genes, or any DNA or RNA fragment thereof. Hybridization of an mRNA with the probe indicates that the genes involved in oxidative phosphorylation (OXPHOS genes), genes involved in the mevalonate pathway, genes involved in metabolic sensing, genes involved in purine biosynthesis (PPAT genes), transcription factor genes, BTN3A genes, or a combination of those genes in question are being expressed.

In some cases, the mRNA from the sample is immobilized on a solid surface and contacted with a probe, for example by running the isolated mRNA on an agarose gel and transferring the mRNA from the gel to a membrane, such as nitrocellulose. In other cases, the probes are immobilized on a solid surface and the mRNA is contacted with the probes, for example, in an Agilent gene chip array. A skilled artisan can readily adapt available mRNA detection methods for use in detecting the level of expression of the genes involved in oxidative phosphorylation (OXPHOS genes), genes involved in the mevalonate pathway, genes involved in metabolic sensing, genes involved in purine biosynthesis (PPAT genes), transcription factor genes, BTN3A genes, or a combination of those genes.

Another method for determining the level of gene expression in a sample can involve nucleic acid amplification of one or more mRNAs (or cDNAs thereof), for example, by RT-PCR (U.S. Pat. No. 4,683,202), ligase chain reaction (Barany, Proc. Natl. Acad. Sci. USA 88:189-93, 1991), self-sustained sequence replication (Guatelli et al., Proc. Natl. Acad. Sci. USA 87:1874-78, 1990), transcriptional amplification system (Kwoh et al., Proc. Natl. Acad. Sci. USA 86:1173-77, 1989), Q-Beta Replicase (Lizardi et al., Bio/Technology 6:1197, 1988), rolling circle replication (U.S. Pat. No. 5,854,033), or any other nucleic acid amplification method, followed by the detection of the amplified molecules using available techniques. These detection schemes are especially useful for the detection of nucleic acid molecules if such molecules are present in very low numbers.

In some cases, gene expression is assessed by quantitative RT-PCR. Numerous different PCR or QPCR protocols are available and can be directly applied or adapted for use for the genes involved in oxidative phosphorylation (OXPHOS genes), genes involved in the mevalonate pathway, genes involved in metabolic sensing, genes involved in purine biosynthesis (PPAT genes), transcription factor genes, BTN3A genes, or a combination of those genes. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR. However, preferred are cyclers with real-time fluorescence measurement capabilities, for example, SMARTCYCLER® (Cepheid, Sunnyvale, Calif.), ABI PRISM 7700® (Applied Biosystems, Foster City, Calif.), ROTOR-GENE® (Corbett Research, Sydney, Australia), LIGHTCYCLER® (Roche Diagnostics Corp, Indianapolis, Ind.), ICYCLER® (Biorad Laboratories, Hercules, Calif.) and MX4000® (Stratagene, La Jolla, Calif.).

Quantitative PCR (QPCR) (also referred as real-time PCR) is preferred under some circumstances because it provides not only a quantitative measurement, but also reduced time and contamination. In some instances, the availability of full gene expression profiling techniques is limited due to requirements for fresh frozen tissue and specialized laboratory equipment, making the routine use of such technologies difficult in a clinical setting. However, QPCR gene measurement can be applied to standard formalin-fixed paraffin-embedded clinical tumor blocks, such as those used in archival tissue banks and routine surgical pathology specimens (Cronin et al. (2007) Clin Chem 53:1084-91)[Mullins 2007] [Paik 2004]. As used herein, “quantitative PCR (or “real time QPCR”) refers to the direct monitoring of the progress of PCR amplification as it is occurring without the need for repeated sampling of the reaction products. In quantitative PCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time.

In some cases, microarrays are used for expression profiling. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. High-density oligonucleotide arrays are particularly useful for determining the gene expression profile for a large number of RNAs in a sample. Techniques for the synthesis of these arrays using mechanical synthesis methods are described in, for example, U.S. Pat. No. 5,384,261. Although a planar array surface can be used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992. Arrays can be packaged in such a manner as to allow for diagnostics or other manipulation of an all-inclusive device. See, for example, U.S. Pat. Nos. 5,856,174 and 5,922,591.

When using microarray techniques, PCR amplified inserts of cDNA clones can be applied to a substrate in a dense array. The microarrayed genes, immobilized on the microchip, are suitable for hybridization under stringent conditions. Fluorescently labeled cDNA probes can be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance.

With dual color fluorescence, separately labeled cDNA probes generated from two sources of RNA can be hybridized pairwise to the array. The relative abundance of the transcripts from the two sources corresponding to each specified gene is thus determined simultaneously. A miniaturized scale can be used for the hybridization, which provides convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93:106-49, 1996). Microarray analysis can be performed by commercially available equipment, following manufacturer's protocols, such as by using the Affymetrix GenChip technology, or Agilent ink jet microarray technology. The development of microarray methods for large-scale analysis of gene expression makes it possible to search systematically for molecular markers of cancer classification and outcome prediction in a variety of tumor types.

As used herein “level”, refers to a measure of the amount of, or a concentration of a transcription product, for instance an mRNA, or a translation product, for instance a protein or polypeptide.

As used herein “activity” refers to a measure of the ability of a transcription product or a translation product to produce a biological effect or to a measure of a level of biologically active molecules.

As used herein “expression level” further refer to gene expression levels or gene activity. Gene expression can be defined as the utilization of the information contained in a gene by transcription and translation leading to the production of a gene product.

The terms “increased,” or “increase” in connection with expression of the genes or biomarkers described herein generally means an increase by a statically significant amount. For the avoidance of any doubt, the terms “increased” or “increase” means an increase of at least 10% as compared to a reference value, for example an increase of at least about 20%, or at least about 30%, or at least about 40%, or at least about 50%, or at least about 60%. or at least about 70%, or at least about 80%, or at least about 90% or up to and including a 100% increase or any increase between 10-100% as compared to a reference value or level, or at least about a 1.5-fold, at least about a 1.6-fold, at least about a 0.7-fold, at least about a 1.8-fold, at least about a 1.9-fold, at least about a 2-fold, at least about a 3-fold, or at least about a 4-fold, or at least about a 5-fold, at least about a 10-fold increase, any increase between 2-fold and 10-fold, at least about a 25-fold increase, or greater as compared to a reference level. In some embodiments, an increase is at least about 1.8-fold increase over a reference value.

Similarly, the terms “decrease,” or “reduced,” or “reduction,” or “inhibit” in connection with expression of the genes or biomarkers described herein generally to refer to a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level or non-detectable level as compared to a reference sample), or any decrease between 10-100% as compared to a reference level.

A “reference value” is a predetermined reference level, such as an average or median of expression levels of each of genes involved in oxidative phosphorylation (OXPHOS genes), genes involved in the mevalonate pathway, genes involved in metabolic sensing, genes involved in purine biosynthesis (PPAT genes), transcription factor genes, BTN3A genes, or a combination of those genes in, for example, biological samples from a population of healthy subjects. The reference value can be an average or median of expression levels of each of genes or biomarkers in a chronological age group matched with the chronological age of the tested subject. In some embodiments, the reference biological samples can also be gender matched. In some embodiments, a positive reference biological sample can be cancer-containing tissue from a specific subgroup of patients, such as stage 1, stage 2, stage 3, or grade 1, grade 2, grade 3 cancers, non-metastatic cancers, untreated cancers, hormone treatment resistant cancers, HER2 amplified cancers, triple negative cancers, estrogen negative cancers, or other relevant biological or prognostic subsets.

If the expression level of a gene or biomarker is greater or less than that of the reference or the average expression level, the expression level of the gene or biomarker is said to be “increased” or “decreased,” respectively, as those terms are defined herein. Exemplary analytical methods for classifying expression of a gene or biomarker, determining a malignancy associated response signature status, and scoring of a sample for expression of a malignancy associated response signature biomarker are explained herein.

### BTN3A

The BTN2A1-3A1-3A2 cell surface complex can be activated by phosphoantigens of the mevalonate pathway through intracellular binding to BTN3A1, allowing BTN2A1 to engage Vγ9Vδ2 T cell receptors (TCRs). Previous models of Vγ9Vδ2 T cell-target cell interactions have relied on static abundance of the surface butyrophilin complex, with phosphoantigen abundance being the main relevant variable.

As confirmed herein, BTN3A1 abundance is an important variable. However, the application also shows that BTN3A1 abundance is regulated by a variety of pathways, transcriptional switches, and by the cellular metabolic state. BTN3A1 levels and the cellular metabolic state can signal to surveilling γδ T cells that a target cell could be transformed or could be stressed.

Experiments described herein reveal a multilayered regulatory framework exists that modulates this interaction by regulating BTN3A1 abundance and/or accessibility through transcriptional regulators (e.g., IRF1, NLRC5, ZNF217, RUNX1), glycosylation and sialylation (CMAS), iron-sulfur cluster formation (FAM96B), trafficking (RER1), metabolic sensing (CtBP1), and various metabolic pathways (PPAT of purine biosynthesis; NDUFA2 and TIMMDC1 of OXPHOS; GALE of galactose metabolism). Also as shown herein, AMPK is a regulator of BTN3A1 expression in cells undergoing an energy crisis. Hence, the experimental results shown herein illuminate a mechanism of stress-regulation of a key γδ T cell-cancer cell interaction.

The butyrophilin (BTN) genes are a group of major histocompatibility complex (MHC)-associated genes that encode type I membrane proteins with 2 extracellular immunoglobulin (Ig) domains and an intracellular B30.2 (PRYSPRY) domain. Three subfamilies of human BTN genes are located in the MHC class I region: the single-copy BTN1A1 gene (MIM 601610) and the BTN2 (e.g., BTN2A1; MIM 613590) and BTN (e.g., BNT3A1) genes, which have undergone tandem duplication, resulting in three copies of each.

At least three BTN3A genes have therefore been characterized in humans, BTN3A1, BTN3A2, and BTN3A3, which are members of a large family of butyrophilin genes located in the telomeric end of the major histocompatibility complex class I region and encode cell surface-expressed proteins that have high similarity in their extracellular domains yet differ in the domain structure of their intracellular domains. BTN3A1 and BTN3A3 both contain an intracellular B30.2 domain, whereas BTN3A2 does not. The B30.2 domain was first identified as a protein domain encoded by an exon (named B30-2) in the human class I major histocompatibility complex region (chromosome 6p21.3).

For example, a Homo sapiens butyrophilin subfamily 3 member A1 (BTN3A1) isoform a precursor can be a 513 amino acid protein with NCBI accession no. NP 008979.3 (GI: 37595558) (SEQ ID NO:1)

| | |
|---|---|
| 1 | MKMASFLAFL LLNFRVCLLL LQLLMPHSAQ FSVLGPSGPI |
| 41 | LAMVGEDADL PCHLFPTMSA ETMELKWVSS SLRQVVNVYA |
| 81 | DGKEVEDRQS APYRGRTSIL RDGITAGKAA LRIHNVTASD |
| 121 | SGKYLCYFQD GDFYEKALVE LKVAALGSDL HVDVKGYKDG |
| 161 | GIHLECRSTG WYPQPQIQWS NNKGENIPTV EAPVVADGVG |
| 201 | LYAVAASVIM RGSSGEGVSC TIRSSLLGLE KTASISIADP |
| 241 | FFRSAQRWIA ALAGTLPVLL LLLGGAGYFL WQQQEEKKTQ |
| 281 | FRKKKREQEL REMAWSTMKQ EQSTRVKLLE ELRWRSIQYA |
| 321 | SRGERHSAYN EWKKALFKPA DVILDPKTAN PILLVSEDQR |
| 361 | SVQRAKEPQD LPDNPERFNW HYCVLGCESF ISGRHYWEVE |
| 401 | VGDRKEWHIG VCSKNVQRKG WVKMTPENGF WIMGLTDGNK |
| 441 | YRTLTEPRTN LKLPKPPKKV GVFLDYETGD ISFYNAVDGS |
| 481 | HIHTFLDVSF SEALYPVFRI LTLEPTALTI CPA |

A Homo sapiens butyrophilin subfamily 3 member A1 isoform b precursor can be a 352 amino acid protein with NCBI accession no. NP_919423.1 (GI: 37221189) (SEQ ID NO:2).

| | |
|---|---|
| 1 | MKMASFLAFL LLNFRVCLLL LQLLMPHSAQ FSVLGPSGPI |
| 41 | LAMVGEDADL PCHLFPTMSA ETMELKWVSS SLRQVVNVYA |
| 81 | DGKEVEDRQS APYRGRTSIL RDGITAGKAA LRIHNVTASD |
| 121 | SGKYLCYFQD GDFYEKALVE LKVAALGSDL HVDVKGYKDG |
| 161 | GIHLECRSTG WYPQPQIQWS NNKGENIPTV EAPVVADGVG |
| 201 | LYAVAASVIM RGSSGEGVSC TIRSSLLGLE KTASISIADP |
| 241 | FFRSAQRWIA ALAGTLPVLL LLLGGAGYFL WQQQEEKKTQ |
| 281 | FRKKKREQEL REMAWSTMKQ EQSTRVKLLE ELRWRSIQYA |
| 321 | SRGERHSAYN EWKKALFKPG EEMLQMRLHF VK |

A Homo sapiens butyrophilin subfamily 3 member A1 isoform c precursor can be a 461 amino acid protein with NCBI accession no. NP_001138480.1 (GI: 222418658) (SEQ ID NO:3).

| | |
|---|---|
| 1 | MKMASFLAFL LLNFRVCLLL LQLLMPHSAQ FSVLGPSGPI |
| 41 | LAMVGEDADL PCHLFPTMSA ETMELKWVSS SLRQVVNVYA |
| 81 | DGKEVEDRQS APYRGRTSIL RDGITAGKAA LRIHNVTASD |
| 121 | SGKYLCYFQD GDFYEKALVE LKVADGVGLY AVAASVIMRG |
| 161 | SSGEGVSCTI RSSLLGLEKT ASISIADPFF RSAQRWIAAL |
| 201 | AGTLPVLLLL LGGAGYFLWQ QQEEKKTQFR KKKREQELRE |
| 241 | MAWSTMKQEQ STRVKLLEEL RWRSIQYASR GERHSAYNEW |
| 281 | KKALFKPADV ILDPKTANPI LLVSEDQRSV QRAKEPQDLP |
| 321 | DNPERFNWHY CVLGCESFIS GRHYWEVEVG DRKEWHIGVC |
| 361 | SKNVQRKGWV KMTPENGFWT MGLTDGNKYR TLTEPRTNLK |
| 401 | LPKPPKKVGV FLDYETGDIS FYNAVDGSHI HTFLDVSFSE |
| 441 | ALYPVFRILT LEPTALTICP A |

A Homo sapiens butyrophilin subfamily 3 member A1 isoform d precursor [Homo sapiens] a 378 amino acid protein with NCBI accession no. NP_00113848.1 (GI: 222418660) (SEQ ID NO: 4).

| | |
|---|---|
| 1 | MKMASFLAFL LLNFRVCLLL LQLLMPHSAQ FSVLGPSGPI |
| 41 | LAMVGEDADL PCHLFPTMSA ETMELKWVSS SLRQVVNVYA |
| 81 | DGKEVEDRQS APYRGRTSIL RDGITAGKAA LRIHNVTASD |
| 121 | SGKYLCYFQD GDFYEKALVE LKVAALGSDL HVDVKGYKDG |
| 161 | GIHLECRSTG WYPQPQIQWS NNKGENIPTV EAPVVADGVG |
| 201 | LYAVAASVIM RGSSGEGVSC TIRSSLLGLE KTASISIADP |
| 241 | FFRSAQRWIA ALAGTLPVLL LLLGGAGYFL WQQQEEKKTQ |
| 281 | FRKKKREQEL REMAWSTMKQ EQSTRVKLLE ELRWRSIQYA |
| 321 | SRGERHSAYN EWKKALFKPG PPIGQTQQQT RGQGSPVALS |
| 361 | QESAQRTDSW GPEEGGES |

A Homo sapiens butyrophilin subfamily 3 member A1 isoform X1 can be a 506 amino acid protein with NCBI accession no. XP_005248890.1 (GI: 530381430) (SEQ ID NO: 5).

| | |
|---|---|
| 1 | MKMASFLAFL LLNFRVCLLL LQLLMPHSAQ FSVLGPSGPI |
| 41 | LAMVGEDADL PCHLFPTMSA ETMELKWVSS SLRQVVNVYA |
| 81 | DGKEVEDRQS APYRGRISIL RDGITAGKAA LRIHNVTASD |
| 121 | SGKYLCYFQD GDFYEKALVE LKVAALGSDL HVDVKGYKDG |
| 161 | GIHLECRSTG WYPQPQIQWS NNKGENIPTV EAPVVADGVG |
| 201 | LYAVAASVIM RGSSGEGVSC TIRSSLLGLE KTASISIADP |
| 241 | FFRSAQRWIA ALAGTLPVLL LLLGGAGYFL WQQQEEKKTQ |
| 281 | FRKKKREQEL REMAWSTMKQ EQSTRGWRSI QYASRGERHS |
| 321 | AYNEWKKALF KPADVILDPK TANPILLVSE DQRSVQRAKE |
| 361 | PQDLPDNPER FNWHYCVLGC ESFISGRHYW EVEVGDRKEW |
| 401 | HIGVCSKNVQ RKGWVKMTPE NGFWTMGLTD GNKYRTLTEP |
| 441 | RTNLKLPKPP KKVGVELDYE TGDISFYNAV DGSHIHTFLD |
| 481 | VSFSEALYPV FRILTLEPTA LTICPA |

A Homo sapiens butyrophilin subfamily 3 member A11 isoform X3 can be a 352 amino acid protein with NCBI accession no. XP_005248891.1 (GI: 530381432) (SEQ ID NO:6).

| | |
|---|---|
| 1 | MKMASFLAFL LLNFRVCLLL LQLLMPHSAQ FSVLGPSGPI |
| 41 | LAMVGEDADL PCHLFPTMSA ETMELKWVSS SLRQVVNVYA |
| 81 | DGKEVEDRQS APYRGRTSIL RDGITAGKAA LRIHNVTASD |
| 121 | SGKYLCYFQD GDFYEKALVE LKVAALGSDL HVDVKGYKDG |
| 161 | GIHLECRSTG WYPQPQIQWS NNKGENIPTV EAPVVADGVG |
| 201 | LYAVAASVIM RGSSGEGVSC TIRSSLIGLE KTASISIADP |
| 241 | FFRSAQRWIA ALAGTLPVLL LLLGGAGYFL WQQQEEKKTQ |
| 281 | FRKKKREQEL REMAWSTMKQ EQSTRVKLLE ELRWRSIQYA |
| 321 | SRGERHSAYN EWKKALFKPG EEMLQMRLHF VK |

A Homo sapiens butyrophilin subfamily 3 member A11 isoform X2 can be a 419 amino acid protein with NCBI accession no. XP_006715046.1 (GI: 578811397) (SEQ ID NO: 7).

| | |
|---|---|
| 1 | MKMASFLAFL LLNFRVCLLL LQLLMPHSAQ FSVLGPSGPI |
| 41 | LAMVGEDADL PCHLFPTMSA ETMELKWVSS SLRQVVNVYA |
| 81 | DGKEVEDRQS APYRGRISIL RDGITAGKAA LRIHNVTASD |
| 121 | SGKYLCYFQD GDFYEKALVE LKVADPFFRS AQRWIAALAG |
| 161 | TLPVLLLLLG GAGYFLWQQQ EEKKTQFRKK KREQELREMA |
| 201 | WSTMKQEQST RVKLLEELRW RSIQYASRGE RHSAYNEWKK |
| 241 | ALFKPADVIL DPKTANPILL VSEDQRSVQR AKEPQDLPDN |
| 281 | PERFNWHYCV LGCESFISGR HYWEVEVGDR KEWHIGVCSK |
| 321 | NVQRKGWVKM TPENGFWTMG LTDGNKYRTL TEPRTNLKLP |
| 361 | KPPKKVGVFL DYETGDISFY NAVDGSHIHT FLDVSFSEAL |
| 401 | YPVFRILTLE PTALTICPA |

The sequences provided herein are exemplary. Isoforms and variants of the BTN3A sequences described herein can also be used in the methods described herein.

For example, isoforms and variants of the BTN3A proteins and nucleic acids can be used in the methods described herein when they are substantially identical to the ‘reference’ BTN3A sequences described herein. The terms “substantially identity” indicates that a polypeptide or nucleic acid comprises a sequence with between 55-100% sequence identity to a reference sequence, for example with at least 55% sequence identity, preferably 60%, preferably 70%, preferably 80%, preferably at least 90%, preferably at least 95%, preferably at least 96%, preferably at least 97% sequence, preferably at least 98%, preferably at least 99% identity to a reference sequence over a specified comparison window. Optimal alignment may be ascertained or conducted using the homology alignment algorithm of Needleman and Wunsch, J. Mol. Biol. 48:443-53 (1970).

### Negative BTN3A Regulators

The negative BTN3A regulators include any of those listed in Table 1. Human sequences for any of these negative regulator protein and nucleic acids are available, for example in the NCBI database (ncbi.nlm.nih.gov) or the Uniprot database (uniprot.org). Negative regulators of BTN3A can be used to reduce or inhibit the expression or function of BTN3A.

However, increased expression of a negative regulator of BTN3A by cancer cells can be an indication that the cancer cells may not be effectively treated by T cell therapies. Alternatively, reduced expression of a negative regulator of BTN3A by cancer cells can be an indication that the cancer cells may be effectively treated by T cell therapies. For example, if cancer cells in a sample express increased levels of ZNF217 (negative regulator) compared to a reference value or control, the subject providing the sample can be a poor candidate for γδ T cell treatment in the form of cell transfer, antibodies targeting or enhancing γδ T cell-cancer interactions, or drugs similarly enhancing such interactions. However, if cancer cells in a sample express ZNF217 (negative regulator) at a low levels, the patient is a good candidate for γδ T cell treatment in the form of cell transfer, antibodies targeting or enhancing γδ T cell-cancer interactions, or drugs similarly enhancing such interactions.”

The negative regulators of BTN3A can include any of those listed in Table 1. In some cases, the methods and compositions described herein utilize the first fifty of the negative BTN3A1 regulators listed in Table 1. The first fifty negative BTN3A regulators are CTBP1, UBE2E1, RING1, ZNF217, HDAC8, RUNX1, RBM38, CBFB, RER1, IKZF1, KCTD5, ST6GAL1, ZNF296, NFKBIA, ATIC, TIAL1, CMAS, CSRNP1, GADD45A, EDEM3, AGO2, RNASEH2A, SRD5A3, ZNF281, MAP2K3, SUPT7L, SLC19A1, CCNL1, AUP1, ZRSR2, CDK13, RASA2, ERF, EIF4ENIF1, PRMT7, MOCS3, HSCB, EDC4, CD79A, SLC16A1, RBM10, GALE, MEF2B, FAM96B, ATXN7, COG8, DERL1, TGFBR2, CHTF8, and AHCYL1. In some cases, the methods and compositions focus on using the following negative regulators of BTN3A: ZNF217, CTBP1, RUNX1, GALE, TIMMDC1, NDUFA2, PPAT, CMAS, RER1, FAM96B, or a combination thereof.

An example of a human negative BTN3A1 regulator sequence for a CTBP1 protein is shown below (Uniprot Q13363; SEQ ID NO:8).

```
                10 20 30 40
MGSSHLLNKG LPLGVRPPIM NGPLHPRPLV ALLDGRDCTV
                50 60 70 80
EMPILKDVAT VAFCDAQSTQ EIHEKVLNEA VGALMYHTIT
                90 100 110 120
LTREDLEKFK ALRIIVRIGS GFDNIDIKSA GDLGIAVCNV
              130 140 150 160
PAASVEETAD STLCHILNLY RRATWLHQAL REGTRVQSVE
              170 180 190 200
QIREVASGAA RIRGETLGII GLGRVGQAVA LRAKAFGFNV
              210 220 230 240
LFYDPYLSDG VERALGLQRV STLQDLLFHS DCVTLHCGLN
              250 260 270 280
EHNHHLINDF TVKQMRQGAF LVNTARGGLV DEKALAQALK
              290 300 310 320
EGRIRGAALD VHESEPFSFS QGPLKDAPNL ICTPHAAWYS
              330 340 350 360
EQASIEMREE AAREIRRAIT GRIPDSLKNC VNKDHLTAAT
              370 380 390 400
HWASMDPAVV HPELNGAAYR YPPGVVGVAP TGIPAAVEGI
              410 420 430 440
VPSAMSLSHG LPPVAHPPHA PSPGQTVKPE ADRDHASDQL
```

This CTBP1 protein is encoded by a cDNA sequence with accession number U37408.1 in the NCBI database.

An example of a human negative BTN3A1 regulator sequence for a UBE2E1 protein is shown below (Uniprot P51965; SEQ ID NO:9).

```
                10 20 30 40
MSDDDSRAST SSSSSSSSNQ QTEKETNTPK KKESKVSMSK
                50 60 70 80
NSKLLSTSAK RIQKELADIT LDPPPNCSAG PKGDNIYEWR
                90 100 110 120
STILGPPGSV YEGGVFFLDI TFTPEYPFKP PKVTFRTRIY
              130 140 150 160
HCNINSQGVI CLDILKDNWS PALTISKVLL SICSLLTDCN
              170 180 190
PADPLVGSIA TQYMTNRAEH DRMARQWTKR YAT
```

This UBE2E1 protein is encoded by a cDNA sequence with accession number X92963 in the NCBI database.

An example of a human negative BTN3A1 regulator sequence for a RING1 protein is shown below (Uniprot Q06587; SEQ ID NO.-10).

```
                10 20 30 40
MTTPANAQNA SKTWELSLYE LHRTPQEAIM DGTEIAVSPR
                50 60 70 80
SLHSELMCPI CLDMLKNTMT TKECLHRFCS DCIVTALRSG
                90 100 110 120
NKECPTCRKK LVSKRSLRPD PNFDALISKI YPSREEYEAH
              130 140 150 160
QDRVLIRLSR LHNQQALSSS IEEGLRMQAM HRAQRVRRPI
              170 180 190 200
PGSDQTTTMS GGEGEPGEGE GDGEDVSSDS APDSAPGPAP
              210 220 230 240
KRPRGGGAGG SSVGTGGGGT GGVGGGAGSE DSGDRGGTLG
              250 260 270 280
GGTLGPPSPP GAPSPPEPGG EIELVFRPHP LLVEKGEYCQ
              290 300 310 320
TRYVKTTGNA TVDHLSKYLA LRIALERRQQ QEAGEPGGPG
              330 340 350 360
GGASDTGGPD GCGGEGGGAG GGDGPEEPAL PSLEGVSEKQ
              370 380 390 400
YTIYIAPGGG AFTTLNGSLT LELVNEKFWK VSRPLELCYA
PTKDPK
```

This RING1 protein is encoded by a cDNA sequence with accession number Z14000 in the NCBI database.

An example of human negative BTN3A1 regulator sequence for a ZNF217 protein is shown below (Uniprot O75362; SEQ ID NO:11).

```
                10 20 30 40
MQSKVTGNMP TQSLLMYMDG PEVIGSSLGS PMEMEDALSM
                50 60 70 80
KGTAVVPFRA TQEKNVIQIE GYMPLDCMFC SQTFTHSEDL
                90 100 110 120
NKHVLMQHRP TLCEPAVLRV EAEYLSPLDK SQVRTEPPKE
              130 140 150 160
KNCKENEFSC EVCGQTFRVA FDVEIHMRTH KDSFTYGCNM
              170 180 190 200
CGRRFKEPWF LKNHMRTHNG KSGARSKLQQ GLESSPATIN
              210 220 230 240
EVVQVHAAES ISSPYKICMV CGFLFPNKES LIEHRKVHTK
              250 260 270 280
KTAFGTSSAQ TDSPQGGMPS SREDFLQLFN LRPKSHPETG
              290 300 310 320
KKPVRCIPQL DPFTTFQAWQ LATKGKVAIC QEVKESGQEG
              330 340 350 360
STDNDDSSSE KELGETNKGS CAGLSQEKEK CKHSHGEAPS
              370 380 390 400
VDADPKLPSS KEKPTHCSEC GKAFRTYHQL VLHSRVHKKD
              410 420 430 440
RRAGAESPTM SVDGRQPGTC SPDLAAPLDE NGAVDRGEGG
              450 460 470 480
SEDGSEDGLP EGIHLDKNDD GGKIKHLTSS RECSYCGKFF
              490 500 510 520
RSNYYLNIHL RTHTGEKPYK CEFCEYAAAQ KTSLRYHLER
              530 540 550 560
HHKEKQTDVA AEVKNDGKNQ DTEDALLTAD SAQTKNLKRF
              570 580 590 600
FDGAKDVTGS PPAKQLKEMP SVFQNVLGSA VLSPAHKDTQ
              610 620 630 640
DFHKNAADDS ADKVNKNPTP AYLDLLKKRS AVETQANNLI
              650 660 670 680
CRTKADVTPP PDGSTTHNLE VSPKEKQTET AADCRYRPSV
              690 700 710 720
DCHEKPLNLS VGALHNCPAI SLSKSLIPSI TCPFCTFKTF
              730 740 750 760
YPEVLMMHQR LEHKYNPDVH KNCRNKSLLR SRRTGCPPAL
              770 780 790 800
LGKDVPPLSS FCKPKPKSAF PAQSKSLPSA KGKQSPPGPG
              810 820 830 840
KAPLTSGIDS STLAPSNLKS HRPQQNVGVQ GAATRQQQSE
              850 860 870 880
MFPKTSVSPA PDKTKRPETK LKPLPVAPSQ PTLGSSNING
              890 900 910 920
SIDYPAKNDS PWAPPGRDYF CNRSASNTAA EFGEPLPKRL
              930 940 950 960
KSSVVALDVD QPGANYRRGY DLPKYHMVRG ITSLLPQDCV
              970 980 990 1000
YPSQALPPKP RFLSSSEVDS PNVLTVQKPY GGSGPLYTCV
            1010 1020 1030 1040
PAGSPASSST LEGKRPVSYQ HLSNSMAQKR NYENFIGNAH
YRPNDKKT
```

This ZNF217 protein is encoded by a cDNA sequence with accession number AF041259 in the NCBI database.

An example of a human negative BTN3A1 regulator sequence for a HDAC8 protein is shown below (Uniprot Q9BY41; SEQ ID NO: 12).

```
                10 20 30 40
MEEPEEPADS GQSLVPVYIY SPEYVSMCDS LAKIPKRASM
                50 60 70 80
VHSLIEAYAL HKQMRIVKPK VASMEEMATF HTDAYLQHLQ
                90 100 110 120
KVSQEGDDDH PDSIEYGLGY DCPATEGIFD YAAAIGGATI
              130 140 150 160
TAAQCLIDGM CKVAINWSGG WHHAKKDEAS GFCYLNDAVL
              170 180 190 200
GILRLRRKFE RILYVDLDLH HGDGVEDAFS FTSKVMTVSL
              210 220 230 240
HKFSPGFFPG TGDVSDVGLG KGRYYSVNVP IQDGIQDEKY
              250 260 270 280
YQICESVLKE VYQAFNPKAV VLQLGADTIA GDPMCSFNMT
              290 300 310 320
PVGIGKCLKY ILQWQLATLI LGGGGYNLAN TARCWTYLTG
              330 340 350 360
VILGKTLSSE IPDHEFFTAY GPDYVLEITP SCRPDRNEPH
              370
RIQQILNYIK GNLKHVV
```

This H-DAC8 protein is encoded by a cDNA sequence with accession number AF230097 in the NCBI database.

An example of a human negative BTN3A1 regulator sequence for a RUNX1 protein is shown below (Uniprot Q011196; SEQ ID NO: 13).

```
                10 20 30 40
MRIPVDASTS RRFTPPSTAL SPGKMSEALP LGAPDAGAAL
                50 60 70 80
AGKLRSGDRS MVEVLADHPG ELVRTDSPNF LCSVLPTHWR
                90 100 110 120
CNKTLPIAFK VVALGDVPDG TLVTVMAGND ENYSAELRNA
              130 140 150 160
TAAMKNQVAR FNDLRFVGRS GRGKSFTLTI TVFTNPPQVA
              170 180 190 200
TYHRAIKITV DGPREPRRHR QKLDDQTKPG SLSFSERLSE
              210 220 230 240
LEQLRRTAMR VSPHHPAPTP NPRASLNHST AFNPQPQSQM
              250 260 270 280
QDTRQIQPSP PWSYDQSYQY LGSIASPSVH PATPISPGRA
              290 300 310 320
SGMTTLSAEL SSRLSTAPDL TAFSDPRQFP ALPSISDPRM
              330 340 350 360
HYPGAFTYSP TPVTSGIGIG MSAMGSATRY HTYLPPPYPG
              370 380 390 400
SSQAQGGPFQ ASSPSYHLYY GASAGSYQFS MVGGERSPPR
              410 420 430 440
ILPPCTNAST GSALLNPSLP NQSDVVEAEG SHSNSPTNMA
              450
PSARLEEAVW RPY
```

This protein is encoded by a cDNA sequence with accession number L34598 in the NCBI database.

An example of a human negative BTN3A1 regulator sequence for a RBM38 protein is shown below (Uniprot Q9H0Z9; SEQ ID NO: 14).

```
                10 20 30 40
MLLQPAPCAP SAGFPRPLAA PGAMHGSQKD TTFTKIFVGG
                50 60 70 80
LPYHTTDASL RKYFEGFGDI EEAVVITDRQ TGKSRGYGFV
                90 100 110 120
TMADRAAAER ACKDPNPIID GRKANVNLAY LGAKPRSLQT
              130 140 150 160
GFAIGVQQLH PTLIQRTYGL TPHYIYPPAI VQPSVVIPAA
              170 180 190 200
PVPSLSSPYI EYTPASPAYA QYPPATYDQY PYAASPATAA
              210 220 230
SEVGYSYPAA VPQALSAAAP AGTTFVQYQA PQLQPDRMQ
```

This protein is encoded by a cDNA sequence with accession number AF432218 in the NCBI database.

An example of a human negative BTN3A1 regulator sequence for a CBFB protein is shown below (Uniprot Q13951; SEQ ID NO-15).

```
                10 20 30 40
MPRVVPDQRS KFENEEFFRK LSRECEIKYT GFRDRPHEER
                50 60 70 80
QARFQNACRD GRSEIAFVAT GTNLSLQFFP ASWQGEQRQT
                90 100 110 120
PSREYVDLER EAGKVYLKAP MILNGVCVIW KGWIDLQRLD
              130 140 150 160
GMGCLEFDEE RAQQEDALAQ QAFEEARRRT REFEDRDRSH
              170 180
REEMEVRVSQ LLAVTGKKTT RP
```

This protein is encoded by a cDNA sequence with accession number AF294326 in the NCBI database.

An example of a human negative BTN3A1 regulator sequence for a RER1 protein is shown below (Uniprot O15258; SEQ ID NO:16).

```
                10 20 30 40
MSEGDSVGES VHGKPSVVYR FFTRLGQIYQ SWLDKSTPYT
                50 60 70 80
AVRWVVTLGL SFVYMIRVYL LQGWYIVTYA LGIYHLNLFI
                90 100 110 120
AFLSPKVDPS LMEDSDDGPS LPTKQNEEFR PFIRRLPEFK
              130 140 150 160
FWHAATKGIL VAMVCTFFDA FNVPVFWPIL VMYFIMLFCI
              170 180 190
TMKRQIKHMI KYRYIPFTHG KRRYRGKEDA GKAFAS
```

This protein is encoded by a cDNA sequence with accession number AJ001421 in the NCBI database.

An example of a human negative BTN3A1 regulator sequence for an IKZF1 protein is shown below (Uniprot Q13422; SEQ ID NO: 17).

```
                10 20 30 40
MDADEGQDMS QVSGKESPPV SDTPDEGDEP MPIPEDLSTT
                50 60 70 80
SGGQQSSKSD RVVASNVKVE TQSDEENGRA CEMNGEECAE
                90 100 110 120
DLRMLDASGE KMNGSHRDQG SSALSGVGGI RLPNGKLKCD
              130 140 150 160
ICGIICIGPN VLMVHKRSHT GERPFQCNQC GASFTQKGNL
              170 180 190 200
LRHIKLHSGE KPFKCHLCNY ACRRRDALTG HLRTHSVGKP
              210 220 230 240
HKCGYCGRSY KQRSSLEEHK ERCHNYLESM GLPGTLYPVI
              250 260 270 280
KEETNHSEMA EDLCKIGSER SLVLDRLASN VAKRKSSMPQ
              290 300 310 320
KFLGDKGLSD TPYDSSASYE KENEMMKSHV MDQAINNAIN
              330 340 350 360
YLGAESLRPL VQTPPGGSEV VPVISPMYQL HKPLAEGTPR
              370 380 390 400
SNHSAQDSAV ENLLLLSKAK LVPSEREASP SNSCQDSTDT
              410 420 430 440
ESNNEEQRSG LIYLTNHIAP HARNGLSLKE EHRAYDLLRA
              450 460 470 480
ASENSQDALR VVSTSGEQMK VYKCEHCRVL FLDHVMYTIH
              490 500 510
MGCHGFRDPF ECNMCGYHSQ DRYEFSSHIT RGEHRFHMS
```

This protein is encoded by a cDNA sequence with accession number U40462 in the NCBI database.

An example of a human negative BTN3A1 regulator sequence for a KCTD5 protein is shown below (Uniprot Q9NXV2; SEQ ID NO:18).

```
                10 20 30 40
MAENHCELLS PARGGIGAGL GGGLCRRCSA GLGALAQRPG
                50 60 70 80
SVSKWVRLNV GGTYFLTTRQ TLCRDPKSFL YRLCQADPDL
                90 100 110 120
DSDKDETGAY LIDRDPTYFG PVLNYLRHGK LVINKDLAEE
              130 140 150 160
GVLEEAEFYN ITSLIKLVKD KIRERDSKTS QVPVKHVYRV
              170 180 190 200
LQCQEEELTQ MVSTMSDGWK FEQLVSIGSS YNYGNEDQAE
              210 220 230
FLCVVSKELH NTPYGTASEP SEKAKILQER GSRM
```

This protein is encoded by a cDNA sequence with accession number AK000047 in the NCBI database.

An example of a human negative BTN3A1 regulator sequence for a ST6GAL1 protein is shown below (Uniprot P15907; SEQ ID NO: 19).

```
                10 20 30 40
MIHTNLKKKF SCCVLVFLLF AVICVWKEKK KGSYYDSFKL
                50 60 70 80
QTKEFQVLKS LGKLAMGSDS QSVSSSSTQD PHRGRQTLGS
                90 100 110 120
LRGLAKAKPE ASFQVWNKDS SSKNLIPRLQ KIWKNYLSMN
              130 140 150 160
KYKVSYKGPG PGIKFSAEAL RCHLRDHVNV SMVEVTDFPF
              170 180 190 200
NTSEWEGYLP KESIRTKAGP WGRCAVVSSA GSLKSSQLGR
              210 220 230 240
EIDDHDAVLR FNGAPTANFQ QDVGTKTTIR LMNSQLVTTE
              250 260 270 280
KRFLKDSLYN EGILIVWDPS VYHSDIPKWY QNPDYNFFNN
              290 300 310 320
YKTYRKLHPN QPFYILKPQM PWELWDILQE ISPEEIQPNP
              330 340 350 360
PSSGMLGIII MMTLCDQVDI YEFLPSKRKT DVCYYYQKFF
              370 380 390 400
DSACTMGAYH PLLYEKNLVK HLNQGTDEDI YLLGKATLPG
FRTIHC
```

This protein is encoded by a cDNA sequence with accession number X17247 in the NCBI database.

An example of a human negative BTN3A1 regulator sequence for a ZNF296 protein is shown below (Uniprot Q8WUU4; SEQ ID NO:20).

```
                10 20 30 40
MSRRKAGSAP RRVEPAPAAN PDDEMEMQDL VIELKPEPDA
                50 60 70 80
QPQQAPRLGP FSPKEVSSAG RFGGEPHHSP GPMPAGAALL
                90 100 110 120
ALGPRNPWTL WTPLTPNYPD RQPWTDKHPD LLTCGRCLQT
              130 140 150 160
FPLEAITAFM DHKKLGCQLF RGPSRGQGSE REELKALSCL
              170 180 190 200
RCGKQFTVAW KLLRHAQWDH GLSIYQTESE APEAPLLGLA
              210 220 230 240
EVAAAVSAVV GPAAEAKSPR ASGSGLTRRS PTCPVCKKTL
              250 260 270 280
SSFSNLKVHM RSHTGERPYA CDQCPYACAQ SSKLNRHKKT
              290 300 310 320
HRQVPPQSPL MADTSQEQAS AAPPEPAVHA AAPTSTLPCS
              330 340 350 360
GGEGAGAAAT AGVQEPGAPG SGAQAGPGGD TWGAITTEQR
              370 380 390 400
TDPANSQKAS PKKMPKSGGK SRGPGGSCEF CGKHFTNSSN
              410 420 430 440
LTVHRRSHTG ERPYTCEFCN YACAQSSKLN RHRRMHGMTP
              450 460 470
GSTRFECPHC HVPFGLRATL DKHLRQKHPE AAGEA
```

This protein is encoded by a cDNA sequence with accession number BC019352 in the NCBI database.

An example of a human negative BTN3A1 regulator sequence for a NFKBIA protein is shown below (Uniprot P25963; SEQ ID NO:21).

```
                10 20 30 40
MFQAAERPQE WAMEGPRDGL KKERLLDDRH DSGLDSMKDE
                50 60 70 80
EYEQMVKELQ EIRLEPQEVP RGSEPWKQQL TEDGDSFLHL
                90 100 110 120
AIIHEEKALT MEVIRQVKGD LAFLNFQNNL QQTPLHLAVI
              130 140 150 160
TNQPEIAEAL LGAGCDPELR DFRGNTPLHL ACEQGCLASV
              170 180 190 200
GVLTQSCTTP HLHSILKATN YNGHTCLHLA SIHGYLGIVE
              210 220 230 240
LLVSLGADVN AQEPCNGRTA LHLAVDLQNP DLVSLLLKCG
              250 260 270 280
ADVNRVTYQG YSPYQLTWGR PSTRIQQQLG QLTLENLQML
              290 300 310
PESEDEESYD TESEFTEFTE DELPYDDCVF GGQRLTL
```

This protein is encoded by a cDNA sequence with accession number M69043 in the NCBI database.

An example of a human negative BTN3A1 regulator sequence for an ATIC protein is shown below (Uniprot P31939; SEQ ID NO:22).

```
                10 20 30 40
MAPGQLALFS VSDKTGLVEF ARNLTALGLN LVASGGTAKA
                50 60 70 80
LRDAGLAVRD VSELTGFPEM LGGRVKTLHP AVHAGILARN
                90 100 110 120
IPEDNADMAR LDFNLIRVVA CNLYPFVKTV ASPGVTVEEA
              130 140 150 160
VEQIDIGGVT LLRAAAKNHA RVTVVCEPED YVVVSTEMQS
              170 180 190 200
SESKDTSLET RRQLALKAFT HTAQYDEAIS DYFRKQYSKG
              210 220 230 240
VSQMPLRYGM NPHQTPAQLY TLQPKLPITV LNGAPGFINL
              250 260 270 280
CDALNAWQLV KELKEALGIP AAASFKHVSP AGAAVGIPLS
              290 300 310 320
EDEAKVCMVY DLYKTLTPIS AAYARARGAD RMSSFGDFVA
              330 340 350 360
LSDVCDVPTA KIISREVSDG IIAPGYEEEA LTILSKKKNG
              370 380 390 400
NYCVLQMDQS YKPDENEVRT LFGLHLSQKR NNGVVDKSLF
              410 420 430 440
SNVVTKNKDL PESALRDLIV ATIAVKYTQS NSVCYAKNGQ
              450 460 470 480
VIGIGAGQQS RIHCTRLAGD KANYWWLRHH PQVLSMKFKT
              490 500 510 520
GVKRAEISNA IDQYVTGTIG EDEDLIKWKA LFEEVPELLT
              530 540 550 560
EAEKKEWVEK LTEVSISSDA FFPFRDNVDR AKRSGVAYIA
              570 580 590
APSGSAADKV VIEACDELGI ILAHTNLRLF HH
```

This protein is encoded by a cDNA sequence with accession number U37436 in the NCBI database.

An example of a human negative BTN3A1 regulator sequence for a TIAL1 protein is shown below (Uniprot Q01085; SEQ ID NO:23).

```
                10 20 30 40 50
MMEDDGQPRT LYVGNLSRDV TEVLILQLFS QIGPCKSCKM ITEHTSNDPY
                60 70 80 90 100
CFVEFYEHRD AAAALAAMNG RKILGKEVKV NWATTPSSQK KDTSNHFHVF
              110 120 130 140 150
VGDLSPEITT EDIKSAFAPF GKISDARVVK DMATGKSKGY GFVSFYNKLD
              160 170 180 190 200
AENAIVHMGG QWLGGRQIRT NWATRKPPAP KSTQENNTKQ LRFEDVVNQS
              210 220 230 240 250
SPKNCTVYCG GIASGLTDQL MRQTFSPFGQ IMEIRVEPEK GYSFVRFSTH
              260 270 280 290 300
ESAAHAIVSV NGTTIEGHVV KCYWGKESPD MTKNFQQVDY SQWGQWSQVY
              310 320 330 340 350
GNPQQYGQYM ANGWQVPPYG VYGQPWNQQG FGVDQSPSAA WMGGFGAQPP
              360 370
QGQAPPPVIP PPNQAGYGMA SYQTO
```

This protein is encoded by a cDNA sequence with accession number M96954 in the NCBI database.

An example of a sequence for a human negative BTN3A1 regulator is shown below as the sequence for a CMAS protein (Uniprot Q8NFW8; SEQ ID NO:24).

```
                10 20 30 40 50
MDSVEKGAAT SVSNPRGRPS RGRPPKLQRN SRGGQGRGVE KPPHLAALIL
                60 70 80 90 100
ARGGSKGIPL KNIKHLAGVP LIGWVLRAAL DSGAFQSVWV STDHDEIENV
              110 120 130 140 150
AKQFGAQVHR RSSEVSKDSS TSLDAIIEFL NYHNEVDIVG NIQATSPCLH
              160 170 180 190 200
PTDLQKVAEM IREEGYDSVF SVVRRHQFRW SEIQKGVREV TEPLNINPAK
              210 220 230 240 250
RPRRQDWDGE LYENGSFYFA KRHLIEMGYL QGGKMAYYEM RAEHSVDIDV
              260 270 280 290 300
DIDWPIAEQR VLRYGYFGKE KLKEIKLLVC NIDGCLTNGH IYVSGDQKEI
              310 320 330 340 350
ISYDVKDAIG ISLLKKSGIE VRLISERACS KQTLSSLKLD CKMEVSVSDK
              360 370 380 390 400
LAVVDEWRKE MGLCWKEVAY LGNEVSDEEC LKRVGLSGAP ADACSTAQKA
              410 420 430
VGYICKCNGG RGAIREFAEH ICLLMEKVNN SCQK
```

This protein is encoded by a cDNA sequence with accession number AF397212 in the NCBI database.

An example of a human negative BTN3A1 regulator sequence for a CSRNP1 protein is shown below (Uniprot Q96S65; SEQ ID NO:25).

```
                10 20 30 40 50
MTGLLKRKFD QLDEDNSSVS SSSSSSGCQS RSCSPSSSVS RAWDSEEEGP
                60 70 80 90 100
WDQMPLPDRD FCGPRSFTPL SILKRARRER PGRVAFDGIT VFYFPRCQGF
              110 120 130 140 150
TSVPSRGGCT LGMALRHSAC RRFSLAEFAQ EQARARHEKL RQRLKEEKLE
              160 170 180 190 200
MLQWKLSAAG VPQAEAGLPP VVDAIDDASV EEDLAVAVAG GRLEEVSFLQ
              210 220 230 240 250
PYPARRRRAL LRASGVRRID REEKRELQAL RQSREDCGCH CDRICDPETC
              260 270 280 290 300
SCSLAGIKCQ MDHTAFPCGC CREGCENPMG RVEFNQARVQ THFIHTLTRL
              310 320 330 340 350
QLEQEAESER ELEAPAQGSP PSPGEEALVP TFPLAKPPMN NELGDNSCSS
              360 370 380 390 400
DMTDSSTASS SASGTSEAPD CPTHPGLPGP GFQPGVDDDS LARILSFSDS
              410 420 430 440 450
DFGGEEEEEE EGSVGNLDNL SCFHPADIFG TSDPGGLASW THSYSGCSFT
              460 470 480 490 500
SGVLDENANL DASCFLNGGL EGSREGSLPG TSVPPSMDAG RSSSVDLSLS
              510 520 530 540 550
SCDSFELLQA LPDYSLGPHY TSQKVSDSLD NIEAPHFPLP GLSPPGDASS
              560 570 580
CFLESLMGES EPAAEALDPF IDSQFEDTVP ASLMEPVPV
```

This protein is encoded by a cDNA sequence with accession number AB053121 in the NCBI database.

An example of a human negative BTN3A1 regulator sequence for a GADD45A protein is shown below (Uniprot P24522; SEQ ID NO:26).

```
                10 20 30 40 50
MTLEEFSAGE QKTERMDKVG DALEEVLSKA LSQRTITVGV YEAAKLLNVD
                60 70 80 90 100
PDNVVLCLLA ADEDDDRDVA LQIHFTLIQA FCCENDINIL RVSNPGRLAE
              110 120 130 140 150
LLLLETDAGP AASEGAEQPP DLHCVLVINP HSSQWKDPAL SQLICECRES
              160
RYMDQWVPVI NLPER
```

This protein is encoded by a cDNA sequence with accession number M60974 in the NCBI database.

An example of a human negative BTN3A1 regulator sequence for an EDEM3 protein is shown below (Uniprot Q9BZQ6; SEQ ID NO:27).

```
                10 20 30 40 50
MSEAGGRGCG SPVPQRARWR LVAATAAFCL VSATSVWTAG AEPMSREEKQ
                60 70 80 90 100
KLGNQVLEMF DHAYGNYMEH AYPADELMPL TCRGRVRGQE PSRGDVDDAL
              110 120 130 140 150
GKFSLTLIDS LDTLVVLNKT KEFEDAVRKV LRDVNLDNDV VVSVFETNIR
              160 170 180 190 200
VLGGLLGGHS LAIMLKEKGE YMQWYNDELL QMAKQLGYKL LPAFNTTSGL
              210 220 230 240 250
PYPRINLKFG IRKPEARTGT ETDTCTACAG TLILEFAALS RFTGATIFEE
              260 270 280 290 300
YARKALDFLW EKRQRSSNLV GVTINIHTGD WVRKDSGVGA GIDSYYEYLL
              310 320 330 340 350
KAYVLIGDDS FLERFNTHYD AIMRYISQPP LLLDVHIHKP MLNARTWMDA
              360 370 380 390 400
LLAFFPGLQV LKGDIRPAIE THEMLYQVIK KHNFLPEAFT TDFRVHWAQH
              410 420 430 440 450
PLRPEFAEST YFLYKATGDP YYLEVGKTLI ENLNKYARVP CGFAAMKDVR
              460 470 480 490 500
TGSHEDRMDS FFLAEMFKYL YLLFADKEDI IFDIEDYIFT TEAHLLPLWL
              510 520 530 540 550
STTNQSISKK NTTSEYTELD DSNEDWTCPN TQILFPNDPL YAQSIREPLK
              560 570 580 590 600
NVVDKSCPRG IIRVEESFRS GAKPPLRARD FMATNPEHLE ILKKMGVSLI
              610 620 630 640 650
HLKDGRVQLV QHAIQAASSI DAEDGLRFMQ EMIELSSQQQ KEQQLPPRAV
              660 670 680 690 700
QIVSHPFFGR VVLTAGPAQF GLDLSKHKET RGFVASSKPS NGCSELTNPE
              710 720 730 740 750
AVMGKIALIQ RGQCMFAEKA RNIQNAGAIG GIVIDDNEGS SSDTAPLFQM
              760 770 780 790 800
AGDGKDTDDI KIPMLFLFSK EGSIILDAIR EYEEVEVLLS DKAKDRDPEM
              810 820 830 840 850
ENEEQPSSEN DSQNQSGEQI SSSSQEVDLV DQESSEENSL NSHPESLSLA
              860 870 880 890 900
DMDNAASISP SEQTSNPTEN HETTNLNGEC TDLDNQLQEQ SETEEDSNPN
              910 920 930
VSWGKKVQPI DSILADWNED IEAFEMMEKD EL
```

This protein is encoded by a cDNA sequence with accession number AK315118 in the NCBI database.

An example of a human negative BTN3A1 regulator sequence for an AGO2 protein is shown below (Uniprot Q9UKV8; SEQ ID NO:28).

```
                10 20 30 40 50
MYSGAGPALA PPAPPPPIQG YAFKPPPRPD FGTSGRTIKL QANFFEMDIP
                60 70 80 90 100
KIDIYHYELD IKPEKCPRRV NREIVEHMVQ HFKTQIFGDR KPVFDGRKNL
              110 120 130 140 150
YTAMPLPIGR DKVELEVTLP GEGKDRIFKV SIKWVSCVSL QALHDALSGR
              160 170 180 190 200
LPSVPFETIQ ALDVVMRHLP SMRYTPVGRS FFTASEGCSN PLGGGREVWF
              210 220 230 240 250
GFHQSVRPSL WKMMLNIDVS ATAFYKAQPV IEFVCEVLDF KSIEEQQKPL
              260 270 280 290 300
TDSQRVKFTK EIKGLKVEIT HCGQMKRKYR VCNVTRRPAS HQTFPLQQES
              310 320 330 340 350
GQTVECTVAQ YFKDRHKLVL RYPHLPCLQV GQEQKHTYLP LEVCNIVAGQ
              360 370 380 390 400
RCIKKLTDNQ TSTMIRATAR SAPDRQEEIS KLMRSASFNT DPYVREFGIM
              410 420 430 440 450
VKDEMTDVTG RVLQPPSILY GGRNKAIATP VQGVWDMRNK QFHTGIEIKV
              460 470 480 490 500
WAIACFAPQR QCTEVHLKSF TEQLRKISRD AGMPIQGQPC FCKYAQGADS
              510 520 530 540 550
VEPMFRHLKN TYAGLQLVVV ILPGKTPVYA EVKRVGDTVL GMATQCVQMK
              560 570 580 590 600
NVQRTTPQTL SNLCLKINVK LGGVNNILLP QGRPPVEQQP VIFLGADVTH
              610 620 630 640 650
PPAGDGKKPS IAAVVGSMDA HPNRYCATVR VQQHRQEIIQ DLAAMVRELL
              660 670 680 690 700
IQFYKSTRFK PTRIIFYRDG VSEGQFQQVL HHELLAIREA CIKLEKDYQP
              710 720 730 740 750
GITFIVVQKR HHTRLFCTDK NERVGKSGNI PAGTTVDTKI THPTEFDFYL
              760 770 780 790 800
CSHAGIQGTS RPSHYHVLWD DNRFSSDELQ ILTYQLCHTY VRCTRSVSIP
              810 820 830 840 850
APAYYAHLVA FRARYHLVDK EHDSAEGSHT SGQSNGRDHQ ALAKAVQVHQ
DTLRTMYFA
```

This protein is encoded by a cDNA sequence with accession number AC067931 in the NCBI database.

An example of a human negative BTN3A1 regulator sequence for a RNASEH2A protein is shown below (Uniprot O75792; SEQ MD NO:29).

```
                10 20 30 40 50
MDLSELERDN TGRCRLSSPV PAVCRKEPCV LGVDEAGRGP VLGPMVYAIC
                60 70 80 90 100
YCPLPRLADL EALKVADSKT LLESERERLF AKMEDTDFVG WALDVLSPNL
              110 120 130 140 150
ISTSMLGRVK YNLNSLSHDT ATGLIQYALD QGVNVTQVFV DTVGMPETYQ
              160 170 180 190 200
ARLQQSFPGI EVTVKAKADA LYPVVSAASI CAKVARDQAV KKWQFVEKLQ
              210 220 230 240 250
DLDTDYGSGY PNDPKTKAWL KEHVEPVFGF PQFVRFSWRT AQTILEKEAE
              260 270 280 290
DVIWEDSASE NQEGLRKITS YFLNEGSQAR PRSSHRYFLE RGLESATSL
```

This protein is encoded by a cDNA sequence with accession number Z97029 in the NCBI database.

An example of a human negative BTN3A1 regulator sequence for a SRD5A3 protein is shown below (Uniprot Q9H8P0; SEQ ID NO:30).

```
                10 20 30 40 50
MAPWAEAEHS ALNPLRAVWL TLTAAFLLTL LLQLLPPGLL PGCAIFQDLI
                60 70 80 90 100
RYGKTKCGEP SRPAACRAFD VPKRYFSHFY IISVLWNGFL LWCLTQSLFL
              110 120 130 140 150
GAPFPSWLHG LLRILGAAQF QGGELALSAF LVLVFLWLHS LRRLFECLYV
              160 170 180 190 200
SVFSNVMIHV VQYCFGLVYY VLVGLTVLSQ VPMDGRNAYI TGKNLLMQAR
              210 220 230 240 250
WFHILGMMMF IWSSAHQYKC HVILGNLRKN KAGVVIHCNH RIPFGDWFEY
              260 270 280 290 300
VSSPNYLAEL MIYVSMAVTF GFHNLTWWLV VTNVFFNQAL SAFLSHQFYK
              310
SKFVSYPKHR KAFLPFLF
```

This protein is encoded by a cDNA sequence with accession number AK023414 in the NCBI database.

An example of a human negative BTN3A1 regulator sequence for a ZNF281 protein is shown below (Uniprot Q9Y2X9; SEQ ID NO:31).

```
                10 20 30 40 50
MKIGSGFLSG GGGTGSSGGS GSGGGGSGGG GGGGSSGRRA EMEPTFPQGM
                60 70 80 90 100
VMFNHRLPPV TSFTRPAGSA APPPQCVLSS STSAAPAAEP PPPPAPDMTF
              110 120 130 140 150
KKEPAASAAA FPSQRTSWGF LQSLVSIKQE KPADPEEQQS HHHHHHHHYG
              160 170 180 190 200
GLFAGAEERS PGLGGGEGGS HGVIQDLSIL HQHVQQQPAQ HHRDVLLSSS
              210 220 230 240 250
SRTDDHHGTE EPKQDTNVKK AKRPKPESQG IKAKRKPSAS SKPSLVGDGE
              260 270 280 290 300
GAILSPSQKP HICDHCSAAF RSSYHLRRHV LIHTGERPFQ CSQCSMGFIQ
              310 320 330 340 350
KYLLQRHEKI HSREKPFGCD QCSMKFIQKY HMERHKRTHS GEKPYKCDTC
              360 370 380 390 400
QQYFSRTDRL LKHRRTCGEV IVKGATSAEP GSSNHTNMGN LAVLSQGNTS
              410 420 430 440 450
SSRRKTKSKS IAIENKEQKT GKTNESQISN NINMQSYSVE MPTVSSSGGI
              460 470 480 490 500
IGTGIDELOK RVPKLIFKKG SRKNTDKNYL NFVSPLPDIV GQKSLSGKPS
              510 520 530 540 550
GSLGIVSNNS VETIGLLQST SGKQGQISSN YDDAMQFSKK RRYLPTASSN
              560 570 580 590 600
SAFSINVGHM VSQQSVIQSA GVSVLDNEAP LSLIDSSALN AEIKSCHDKS
              610 620 630 640 650
GIPDEVLQSI LDQYSNKSES QKEDPFNIAE PRVDLHTSGE HSELVQEENL
              660 670 680 690 700
SPGTQTPSND KASMLQEYSK YLQQAFEKST NASFTLGHGF QFVSLSSPLH
              710 720 730 740 750
NHTLFPEKQI YTTSPLECGF GQSVTSVLPS SLPKPPFGML FGSQPGLYLS
              760 770 780 790 800
ALDATHQQLT PSQELDDLID SQKNLETSSA FQSSSQKLTS QKEQKNLESS
              810 820 830 840 850
TGFQIPSQEL ASQIDPQKDI EPRTTYQIEN FAQAFGSQFK SGSRVPMTFI
              860 870 880 890
TNSNGEVDHR VRTSVSDFSG YTNMMSDVSE PCSTRVKTPT SQSYR
```

This protein is encoded by a cDNA sequence with accession number AF125158 in the NCBI database.

An example of a human negative BTN3A1 regulator sequence for a MAP2K3 protein is shown below (Uniprot P46734; SEQ ID NO:32).

```
                10 20 30 40 50
MESPASSQPA SMPQSKGKSK RKKDLRISCM SKPPAPNPTP PRNLDSRTFI
                60 70 80 90 100
TIGDRNFEVE ADDLVTISEL GRGAYGVVEK VRHAQSGTIM AVKRIRATVN
              110 120 130 140 150
SQEQKRLLMD LDINMRTVDC FYTVTFYGAL FREGDVWICM ELMDTSLDKE
              160 170 180 190 200
YRKVLDKNMT IPEDILGEIA VSIVRALEHL HSKLSVIHRD VKPSNVLINK
              210 220 230 240 250
EGHVKMCDFG ISGYLVDSVA KTMDAGCKPY MAPERINPEL NQKGYNVKSD
              260 270 280 290 300
VWSLGITMIE MAILRFPYES WGTPFQQLKQ VVEEPSPQLP ADRFSPEFVD
              310 320 330 340
FTAQCLRKNP AERMSYLELM EHPFFTLHKT KKTDIAAFVK EILGEDS
```

This protein is encoded by a cDNA sequence with accession number L36719 in the NCBI database.

An example of a human negative BTN3A1 regulator sequence for a SUPT7L protein is shown below (Uniprot O94864; SEQ ID NO:33).

```
                10 20 30 40 50
MNLQRYWGEI PISSSQTNRS SFDLLPREFR LVEVHDPPLH QPSANKPKPP
                60 70 80 90 100
TMLDIPSEPC SLTIHTIQLI QHNRRLRNLI ATAQAQNQQQ TEGVKTEESE
              110 120 130 140 150
PLPSCPGSPP LPDDLLPLDC KNPNAPFQIR HSDPESDFYR GKGEPVTELS
              160 170 180 190 200
WHSCRQLLYQ AVATILAHAG FDCANESVLE TLTDVAHEYC LKFTKLLRFA
              210 220 230 240 250
VDREARLGQT PFPDVMEQVF HEVGIGSVLS LQKFWQHRIK DYHSYMLQIS
              260 270 280 290 300
KQLSEEYERI VNPEKATEDA KPVKIKEEPV SDITFPVSEE LEADLASGDQ
              310 320 330 340 350
SLPMGVLGAQ SERFPSNLEV EASPQASSAE VNASPLWNLA HVKMEPQESE
              360 370 380 390 400
EGNVSGHGVL GSDVFEEPMS GMSEAGIPQS PDDSDSSYGS HSTDSLMGSS
              410
PVFNQRCKKR MRKI
```

This protein is encoded by a cDNA sequence with accession number AF197954 in the NCBI database.

An example of a human negative BTN3A1 regulator sequence for a SLC19A1 protein is shown below (Uniprot P41440; SEQ ID NO:34).

```
                10 20 30 40 50
MVPSSPAVEK QVPVEPGPDP ELRSWRHLVC YLCFYGFMAQ IRPGESFITP
                60 70 80 90 100
YLLGPDKNFT REQVTNEITP VLSYSYLAVL VPVFLLTDYL RYTPVLLLQG
              110 120 130 140 150
LSFVSVWLLL LLGHSVAHMQ LMELFYSVTM AARIAYSSYI FSLVRPARYQ
              160 170 180 190 200
RVAGYSRAAV LLGVFTSSVL GQLLVTVGRV SFSTLNYISL AFLTFSVVLA
              210 220 230 240 250
LFLKRPKRSL FFNRDDRGRC ETSASELERM NPGPGGKLGH ALRVACGDSV
              260 270 280 290 300
LARMLRELGD SLRRPQLRLW SLWWVFNSAG YYLVVYYVHI LWNEVDPTTN
              310 320 330 340 350
SARVYNGAAD AASTLLGAIT SFAAGFVKIR WARWSKLLIA GVTATQAGLV
              360 370 380 390 400
FLLAHTRHPS SIWLCYAAFV LFRGSYQFLV PIATFQIASS LSKELCALVF
              410 420 430 440 450
GVNTFFATIV KTIITFIVSD VRGLGLPVRK QFQLYSVYFL ILSIIYFLGA
              460 470 480 490 500
MLDGLRHCQR GHHPRQPPAQ GLRSAAEEKA AQALSVQDKG LGGLQPAQSP
              510 520 530 540 550
PLSPEDSLGA VGPASLEQRQ SDPYLAQAPA PQAAEFLSPV TTPSPCTLCS
              560 570 580 590
AQASGPEAAD ETCPQLAVHP PGVSKLGLQC LPSDGVONVN Q
```

This protein is encoded by a cDNA sequence with accession number U15939 in the NCBI database.

An example of a human negative BTN3A1 regulator sequence for a CCNL1 protein is shown below (Uniprot Q9UK58; SEQ ID NO:35).

```
                10 20 30 40 50
MASGPHSTAT AAAAASSAAP SAGGSSSGTT TTTTTTTGGI LIGDRLYSEV
                60 70 80 90 100
SLTIDHSLIP EERLSPTPSM QDGLDLPSET DLRILGCELI QAAGILLRLP
              110 120 130 140 150
QVAMATGQVL FHRFFYSKSF VKHSFEIVAM ACINLASKIE EAPRRIRDVI
              160 170 180 190 200
NVFHHLRQLR GKRTPSPLIL DQNYINTKNQ VIKAERRVLK ELGFCVHVKH
              210 220 230 240 250
PHKIIVMYLQ VLECERNQTL VQTAWNYMND SLRTNVFVRF QPETIACACI
              260 270 280 290 300
YLAARALQIP LPTRPHWFLL FGTTEEEIQE ICIETLRLYT RKKPNYELLE
              310 320 330 340 350
KEVEKRKVAL QEAKLKAKGL NPDGTPALST LGGFSPASKP SSPREVKAEE
              360 370 380 390 400
KSPISINVKT VKKEPEDRQQ ASKSPYNGVR KDSKRSRNSR SASRSRSRTR
              410 420 430 440 450
SRSRSHTPRR HYNNRRSRSG TYSSRSRSRS RSHSESPRRH HNHGSPHLKA
              460 470 480 490 500
KHTRDDLKSS NRHGHKRKKS RSRSQSKSRD HSDAAKKHRH ERGHHRDRRE
              510 520
RSRSFERSHK SKHHGGSRSG HGRHRR
```

This protein is encoded by a cDNA sequence with accession number AF180920 in the NCBI database.

An example of a human negative BTN3A1 regulator sequence for an AUP1 protein is shown below (Uniprot Q9Y679; SEQ ID NO:36).

```
                10 20 30 40 50
MELPSGPGPE RLFDSHRLPG DCFLLLVLLL YAPVGFCLLV LRLFLGIHVF
                60 70 80 90 100
LVSCALPDSV LRRFVVRTMC AVLGLVARQE DSGLRDHSVR VLISNHVTPF
              110 120 130 140 150
DHNIVNLLTT CSTPLLNSPP SFVCWSRGFM EMNGRGELVE SLKRFCASTR
              160 170 180 190 200
LPPTPLLLFP EEEATNGREG LLRFSSWPFS IQDVVQPLTL QVQRPLVSVT
              210 220 230 240 250
VSDASWVSEL LWSLFVPFTV YQVRWLRPVH RQLGEANEEF ALRVQQLVAK
              260 270 280 290 300
ELGQTGTRLT PADKAEHMKR QRHPRIRPQS AQSSFPPSPG PSPDVQLATL
              310 320 330 340 350
AQRVKEVLPH VPLGVIQRDL AKTGCVDLTI TNLLEGAVAF MPEDITKGTQ
              360 370 380 390 400
SLPTASASKF PSSGPVTPQP TALTFAKSSW ARQESLQERK QALYEYARRR
FTERRAQEAD
```

This protein is encoded by a cDNA sequence with accession number AF100754 in the NCBI database.

An example of a human negative BTN3A1 regulator sequence for a ZRSR2 protein is shown below (Uniprot Q15696; SEQ ID NO:37).

```
                10 20 30 40 50
MAAPEKMTFP EKPSHKKYRA ALKKEKRKKR RQELARLRDS GLSQKEEEED
                60 70 80 90 100
TFIEEQQLEE EKLLERERQR LHEEWLLREQ KAQEEFRIKK EKEEAAKKRQ
              110 120 130 140 150
EEQERKLKEQ WEEQQRKERE EEEQKRQEKK EKEEALQKML DQAENELENG
              160 170 180 190 200
TTWQNPEPPV DFRVMEKDRA NCPFYSKTGA CRFGDRCSRK HNFPTSSPTL
              210 220 230 240 250
LIKSMFTTFG MEQCRRDDYD PDASLEYSEE ETYQQFLDEY EDVLPEFKNV
              260 270 280 290 300
GKVIQFKVSC NLEPHLRGNV YVQYQSEEEC QAALSLFNGR WYAGRQLQCE
              310 320 330 340 350
FCPVTRWKMA ICGLFEIQQC PRGKHCNFLH VFRNPNNEFW EANRDIYLSP
              360 370 380 390 400
DRTGSSFGKN SERRERMGHH DDYYSRLRGR RNPSPDHSYK RNGESERKSS
              410 420 430 440 450
RHRGKKSHKR TSKSRERHNS RSRGRNRDRS RDRSRGRGSR SRSRSRSRRS
              460 470 480
RRSRSQSSSR SRSRGRRRSG NRDRTVQSPK SK
```

This protein is encoded by a cDNA sequence with accession number D49677 in the NCBI database.

An example of a human negative BTN3A1 regulator sequence for a CDK13 protein is shown below (Uniprot Q14004; SEQ ID NO:38).

```
                10 20 30 40
MPSSSDTALG GGGGLSWAEK KLEERRKRRR FLSPQQPPLL
                50 60 70 80
LPLLQPQLLQ PPPPPPPLLF LAAPGTAAAA AAAAAASSSC
                90 100 110 120
FSPGPPLEVK RLARGKRRAG GRQKRRRGPR AGQEAEKRRV
              130 140 150 160
FSLPQPQQDG GGGASSGGGV TPLVEYEDVS SQSEQGLLLG
              170 180 190 200
GASAATAATA AGGTGGSGGS PASSSGTQRR GEGSERRPRR
              210 220 230 240
DRRSSSGRSK ERHREHRRRD GQRGGSEASK SRSRHSHSGE
              250 260 270 280
ERAEVAKSGS SSSSGGRRKS ASATSSSSSS RKDRDSKAHR
              290 300 310 320
SRTKSSKEPP SAYKEPPKAY REDKTEPKAY RRRRSLSPLG
              330 340 350 360
GRDDSPVSHR ASQSLRSRKS PSPAGGGSSP YSRRLPRSPS
              370 380 390 400
PYSRRRSPSY SRHSSYERGG DVSPSPYSSS SWRRSRSPYS
              410 420 430 440
PVLRRSGKSR SRSPYSSRHS RSRSRHRLSR SRSRHSSISP
              450 460 470 480
STLTLKSSLA AELNKNKKAR AAEAARAAEA AKAAEATKAA
              490 500 510 520
EAAAKAAKAS NTSTPTKGNT ETSASASQTN HVKDVKKIKI
              530 540 550 560
EHAPSPSSGG TLKNDKAKTK PPLQVTKVEN NLIVDKATKK
              570 580 590 600
AVIVGKESKS AATKEESVSL KEKTKPLTPS IGAKEKEQHV
              610 620 630 640
ALVTSTLPPL PLPPMLPEDK EADSLRGNIS VKAVKKEVEK
              650 660 670 680
KLRCLLADLP LPPELPGGDD LSKSPEEKKT ATQLHSKRRP
              690 700 710 720
KICGPRYGET KEKDIDWGKR CVDKFDIIGI IGEGTYGQVY
              730 740 750 760
KARDKDTGEM VALKKVRLDN EKEGFPITAI REIKILRQLT
              770 780 790 800
HQSIINMKEI VTDKEDALDF KKDKGAFYLV FEYMDHDLMG
              810 820 830 840
LLESGLVHFN ENHIKSFMRQ LMEGLDYCHK KNFLHRDIKC
              850 860 870 880
SNILLNNRGQ IKLADFGLAR LYSSEESRPY TNKVITLWYR
              890 900 910 920
PPELLLGEER YTPAIDVWSC GCILGELFTK KPIFQANQEL
              930 940 950 960
AQLELISRIC GSPCPAVWPD VIKLPYFNTM KPKKQYRRKL
              970 980 990 1000
REEFVFIPAA ALDLFDYMLA LDPSKRCTAE QALQCEFLRD
            1010 1020 1030 1040
VEPSKMPPPD LPLWQDCHEL WSKKRRRQKQ MGMTDDVSTI
            1050 1060 1070 1080
KAPRKDLSLG LDDSRTNTPQ GVLPSSQLKS QGSSNVAPVK
            1090 1100 1110 1120
TGPGQHLNHS ELAILLNLLQ SKTSVNMADF VQVLNIKVNS
            1130 1140 1150 1160
ETQQQLNKIN LPAGILATGE KQTDPSTPQQ ESSKPLGGIQ
            1170 1180 1190 1200
PSSQTIQPKV ETDAAQAAVQ SAFAVLLTQL IKAQQSKQKD
            1210 1220 1230 1240
VLLEERENGS GHEASLQLRP PPEPSTPVSG QDDLIQHQDM
            1250 1260 1270 1280
RILELTPEPD RPRILPPDQR PPEPPEPPPV TEEDLDYRTE
            1290 1300 1310 1320
NQHVPTTSSS LTDPHAGVKA ALLQLLAQHQ PQDDPKREGG
            1330 1340 1350 1360
IDYQAGDTYV STSDYKDNFG SSSFSSAPYV SNDGLGSSSA
            1370 1380 1390 1400
PPLERRSFIG NSDIQSLDNY STASSHSGGP PQPSAFSESF
            1410 1420 1430 1440
PSSVAGYGDI YLNAGPMLFS GDKDHRFEYS HGPIAVLANS
            1450 1460 1470 1480
SDPSTGPEST HPLPAKMHNY NYGGNLQENP SGPSLMHGQT
            1490 1500 1510
WTSPAQGPGY SQGYRGHIST STGRGRGRGL PY
```

This protein is encoded by a cDNA sequence with accession number AJ297709 in the NCBI database.

An example of a human negative BTN3A1 regulator sequence for a RASA2 protein is shown below (Uniprot Q15283; SEQ ID NO:39).

```
                10 20 30 40
MAAAAPAAAA ASSEAPAASA TAEPEAGDQD SREVRVLQSL
                50 60 70 80
RGKICEAKNL LPYLGPHKMR DCFCTINLDQ EEVYRTQVVE
                90 100 110 120
KSLSPFFSEE FYFEIPRTFQ YLSFYVYDKN VLQRDLRIGK
              130 140 150 160
VAIKKEDLCN HSGKETWFSL QPVDSNSEVQ GKVHLELKLN
              170 180 190 200
ELITENGTVC QQLVVHIKAC HGLPLINGQS CDPYATVSLV
              210 220 230 240
GPSRNDQKKT KVKKKTSNPQ FNEIFYFEVT RSSSYTRKSQ
              250 260 270 280
FQVEEEDIEK LEIRIDLWNN GNLVQDVFLG EIKVPVNVLR
              290 300 310 320
TDSSHQAWYL LQPRDNGNKS SKTDDLGSLR LNICYTEDYV
              330 340 350 360
LPSEYYGPLK TLLLKSPDVQ PISASAAYIL SEICRDKNDA
              370 380 390 400
VLPLVRLLLH HDKLVPFATA VAELDLKDTQ DANTIFRGNS
              410 420 430 440
LATRCLDEMM KIVGGHYLKV TLKPILDEIC DSSKSCEIDP
              450 460 470 480
IKLKEGDNVE NNKENLRYYV DKLFNTIVKS SMSCPTVMCD
              490 500 510 520
IFYSLRQMAT QRFPNDPHVQ YSAVSSFVFL RFFAVAVVSP
              530 540 550 560
HTFHLRPHHP DAQTIRTLTL ISKTIQTLGS WGSLSKSKSS
              570 580 590 600
FKETFMCEFF KMFQEEGYII AVKKELDEIS STETKESSGT
              610 620 630 640
SEPVHLKEGE MYKRAQGRTR IGKKNFKKRW FCLTSRELTY
              650 660 670 680
HKQPGSKDAI YTIPVKNILA VEKLEESSFN KKNMFQVIHT
              690 700 710 720
EKPLYVQANN CVEANEWIDV LCRVSRCNQN RLSFYHPSVY
              730 740 750 760
LNGNWLCCQE TGENTLGCKP CTAGVPADIQ IDIDEDRETE
              770 780 790 800
RIYSLFTLSL LKLQKMEEAC GTIAVYQGPQ KEPDDYSNFV
              810 820 830 840
IEDSVTTFKT IQQIKSIIEK LDEPHEKYRK KRSSSAKYGS
              850
KENPIVGKAS
```

This protein is encoded by a cDNA sequence with accession number D78155 in the NCBI database.

An example of a human negative BTN3A1 regulator sequence for an ERF protein is shown below (Uniprot P50548; SEQ ID NO:40).

```
                10 20 30 40
MKTPADTGFA FPDWAYKPES SPGSRQIQLW HFILELLRKE
                50 60 70 80
EYQGVIAWQG DYGEFVIKDP DEVARLWGVR KCKPQMNYDK
                90 100 110 120
LSRALRYYYN KRILHKTKGK RFTYKFNFNK LVLVNYPFID
              130 140 150 160
VGLAGGAVPQ SAPPVPSGGS HFRFPPSTPS EVLSPTEDPR
              170 180 190 200
SPPACSSSSS SLFSAVVARR LGRGSVSDCS DGTSELEEPL
              210 220 230 240
GEDPRARPPG PPDLGAFRGP PLARLPHDPG VFRVYPRPRG
              250 260 270 280
GPEPLSPFPV SPLAGPGSLL PPQLSPALPM TPTHLAYTPS
              290 300 310 320
PTLSPMYPSG GGGPSGSGGG SHFSFSPEDM KRYLQAHTQS
              330 340 350 360
VYNYHLSPRA FLHYPGLVVP QPQRPDKCPL PPMAPETPPV
              370 380 390 400
PSSASSSSSS SSSPFKFKLQ PPPLGRRQRA AGEKAVAGAD
              410 420 430 440
KSGGSAGGLA EGAGALAPPP PPPQIKVEPI SEGESEEVEV
              450 460 470 480
TDISDEDEED GEVFKTPRAP PAPPKPEPGE APGASQCMPL
              490 500 510 520
KLRFKRRWSE DCRLEGGGGP AGGFEDEGED KKVRGEGPGE
              530 540
AGGPLTPRRV SSDLQHATAQ LSLEHRDS
```

This protein is encoded by a cDNA sequence with accession number U15655 in the NCBI database.

An example of a human negative BTN3A1 regulator sequence for an EIF4ENIF1 protein is shown below (Uniprot Q9NRA8; SEQ ID NO:41).

```
                10 20 30 40
MDRRSMGETE SGDAFLDLKK PPASKCPHRY TKEELLDIKE
                50 60 70 80
LPHSKQRPSC LSEKYDSDGV WDPEKWHASL YPASGRSSPV
                90 100 110 120
ESLKKELDTD RPSLVRRIVD PRERVKEDDL DVVLSPQRRS
              130 140 150 160
FGGGCHVTAA VSSRRSGSPL EKDSDGLRLL GGRRIGSGRI
              170 180 190 200
ISARTFEKDH RLSDKDLRDL RDRDRERDFK DKRFRREFGD
              210 220 230 240
SKRVFGERRR NDSYTEEEPE WFSAGPTSQS ETIELTGFDD
              250 260 270 280
KILEEDHKGR KRTRRRTASV KEGIVECNGG VAEEDEVEVI
              290 300 310 320
LAQEPAADQE VPRDAVLPEQ SPGDFDFNEF FNLDKVPCLA
              330 340 350 360
SMIEDVLGEG SVSASRFSRW FSNPSRSGSR SSSLGSTPHE
              370 380 390 400
ELERLAGLEQ AILSPGQNSG NYFAPIPLED HAENKVDILE
              410 420 430 440
MLQKAKVDLK PLLSSLSANK EKLKESSHSG VVLSVEEVEA
              450 460 470 480
GLKGLKVDQQ VKNSTPFMAE HLEETLSAVT NNRQLKKDGD
              490 500 510 520
MTAFNKLVST MKASGTLPSQ PKVSRNLESH LMSPAEIPGQ
              530 540 550 560
PVPKNILQEL LGQPVQRPAS SNLLSGLMGS LEPTTSLLGQ
              570 580 590 600
RAPSPPLSQV FQTRAASADY LRPRIPSPIG FTPGPQQLLG
              610 620 630 640
DPFQGMRKPM SPITAQMSQL ELQQAALEGL ALPHDLAVQA
              650 660 670 680
ANFYQPGFGK PQVDRTRDGF RNRQQRVTKS PAPVHRGNSS
              690 700 710 720
SPAPAASITS MLSPSFTPTS VIRKMYESKE KSKEEPASGK
              730 740 750 760
AALGDSKEDT QKASEENLLS SSSVPSADRD SSPTTNSKLS
              770 780 790 800
ALQRSSCSTP LSQANRYTKE QDYRPKATGR KTPTLASPVP
              810 820 830 840
TTPFLRPVHQ VPLVPHVPMV RPAHQLHPGL VQRMLAQGVH
              850 860 870 880
PQHLPSLLQT GVLPPGMDLS HLQGISGPIL GQPFYPLPAA
              890 900 910 920
SHPLLNPRPG TPLHLAMVQQ QLQRSVLHPP GSGSHAAAVS
              930 940 950 960
VQTTPQNVPS RSGLPHMHSQ LEHRPSQRSS SPVGLAKWFG
              970 980
SDVLQQPLPS MPAKVISVDE LEYRQ
```

This protein is encoded by a cDNA sequence with accession number AF240775 in the NCBI database.

An example of a human negative BTN3A1 regulator sequence for a PRMT7 protein is shown below (Uniprot Q9NVM4; SEQ ID NO:42).

```
                10 20 30 40
MKIFCSRANP TTGSVEWLEE DEHYDYHQEI ARSSYADMLH
                50 60 70 80
DKDRNVKYYQ GIRAAVSRVK DRGQKALVLD IGTGTGLLSM
                90 100 110 120
MAVTAGADFC YAIEVFKPMA DAAVKIVEKN GFSDKIKVIN
              130 140 150 160
KHSTEVTVGP EGDMPCRANI LVTELFDTEL IGEGALPSYE
              170 180 190 200
HAHRHLVEEN CEAVPHRATV YAQLVESGRM WSWNKLFPIH
              210 220 230 240
VQTSLGEQVI VPPVDVESCP GAPSVCDIQL NQVSPADFTV
              250 260 270 280
LSDVLPMFSI DFSKQVSSSA ACHSRRFEPL TSGRAQVVLS
              290 300 310 320
WWDIEMDPEG KIKCTMAPFW AHSDPEEMQW RDHWMQCVYF
              330 340 350 360
LPQEEPVVQG SALYLVAHHD DYCVWYSLQR TSPEKNERVR
              370 380 390 400
QMRPVCDCQA HLLWNRPRFG EINDQDRTDR YVQALRTVLK
              410 420 430 440
PDSVCLCVSD GSLLSVLAHH LGVEQVFTVE SSAASHKLLR
              450 460 470 480
KIFKANHLED KINIIEKRPE LLTNEDLQGR KVSLLLGEPF
              490 500 510 520
FTTSLLPWHN LYFWYVRTAV DQHLGPGAMV MPQAASLHAV
              530 540 550 560
VVEFRDLWRI RSPCGDCEGF DVHIMDDMIK RALDFRESRE
              570 580 590 600
AEPHPLWEYP CRSLSEPWQI LTFDFQQPVP LQPLCAEGTV
              610 620 630 640
ELRRPGQSHA AVLWMEYHLT PECTLSTGLL EPADPEGGCC
              650 660 670 680
WNPHCKQAVY FFSPAPDPRA LLGGPRTVSY AVEFHPDTGD
              690
IIMEFRHADT PD
```

This protein is encoded by a cDNA sequence with accession number AK001502 in the NCBI database.

An example of a human negative BTN3A1 regulator sequence for a MOCS3 protein is shown below (Uniprot Q9NVM4; SEQ ID NO:43).

```
                10 20 30 40
MKIFCSRANP TTGSVEWLEE DEHYDYHQEI ARSSYADMLH
                50 60 70 80
DKDRNVKYYQ GIRAAVSRVK DRGQKALVLD IGTGTGLLSM
                90 100 110 120
MAVTAGADFC YAIEVFKPMA DAAVKIVEKN GFSDKIKVIN
              130 140 150 160
KHSTEVTVGP EGDMPCRANI LVTELFDTEL IGEGALPSYE
              170 180 190 200
HAHRHLVEEN CEAVPHRATV YAQLVESGRM WSWNKLFPIH
              210 220 230 240
VQTSLGEQVI VPPVDVESCP GAPSVCDIQL NQVSPADFTV
              250 260 270 280
LSDVLPMFSI DFSKQVSSSA ACHSRRFEPL TSGRAQVVLS
              290 300 310 320
WWDIEMDPEG KIKCTMAPFW AHSDPEEMQW RDHWMQCVYF
              330 340 350 360
LPQEEPVVQG SALYLVAHHD DYCVWYSLQR TSPEKNERVR
              370 380 390 400
QMRPVCDCQA HLLWNRPRFG EINDQDRTDR YVQALRTVLK
              410 420 430 440
PDSVCLCVSD GSLLSVLAHH LGVEQVFTVE SSAASHKLLR
              450 460 470 480
KIFKANHLED KINIIEKRPE LLTNEDLQGR KVSLLLGEPF
              490 500 510 520
FTTSLLPWHN LYFWYVRTAV DQHLGPGAMV MPQAASLHAV
              530 540 550 560
VVEFRDLWRI RSPCGDCEGF DVHIMDDMIK RALDFRESRE
              570 580 590 600
AEPHPLWEYP CRSLSEPWQI LTFDFQQPVP LQPLCAEGTV
              610 620 630 640
ELRRPGQSHA AVLWMEYHLT PECTLSTGLL EPADPEGGCC
              650 660 670 680
WNPHCKQAVY FFSPAPDPRA LLGGPRTVSY AVEFHPDTGD
              690
IIMEFRHADT PD
```

This protein is encoded by a cDNA sequence with accession number AK001502 in the NCBI database.

An example of a human negative BTN3A1 regulator sequence for an HSCB protein is shown below (Uniprot Q8IWL3; SEQ ID NO 44).

```
                10 20 30 40
MWRGRAGALL RVWGFWPTGV PRRRPLSCDA ASQAGSNYPR
                50 60 70 80
CWNCGGPWGP GREDRFFCPQ CRALQAPDPT RDYFSLMDCN
                90 100 110 120
RSFRVDTAKL QHRYQQLQRL VHPDFFSQRS QTEKDFSEKH
              130 140 150 160
STLVNDAYKT LLAPLSRGLY LLKLHGIEIP ERTDYEMDRQ
              170 180 190 200
FLIEIMEINE KLAEAESEAA MKEIESIVKA KQKEFTDNVS
              210 220 230
SAFEQDDFEE AKEILTKMRY FSNIEEKIKL KKIPL
```

This protein is encoded by a cDNA sequence with accession number AY191719 in the NCBI database.

An example of a human negative BTN3A1 regulator sequence for an EDC4 protein is shown below (Uniprot Q6P2E9; SEQ ID NO:45).

```
                10 20 30 40
MASCASIDIE DATQHLRDIL KLDRPAGGPS AESPRPSSAY
                50 60 70 80
NGDLNGLLVP DPLCSGDSTS ANKTGLRTMP PINLQEKQVI
                90 100 110 120
CLSGDDSSTC IGILAKEVEI VASSDSSISS KARGSNKVKI
              130 140 150 160
QPVAKYDWEQ KYYYGNLIAV SNSFLAYAIR AANNGSAMVR
              170 180 190 200
VISVSTSERT LLKGFTGSVA DLAFAHLNSP QLACLDEAGN
              210 220 230 240
LFVWRLALVN GKIQEEILVH IRQPEGTPLN HFRRIIWCPF
              250 260 270 280
IPEESEDCCE ESSPTVALLH EDRAEVWDLD MLRSSHSTWP
              290 300 310 320
VDVSQIKQGF IVVKGHSTCL SEGALSPDGT VLATASHDGY
              330 340 350 360
VKFWQIYIEG QDEPRCLHEW KPHDGRPLSC LLFCDNHKKQ
              370 380 390 400
DPDVPFWREL ITGADQNREL KMWCTVSWTC LQTIRFSPDI
              410 420 430 440
FSSVSVPPSL KVCLDLSAEY LILSDVQRKV LYVMELLQNQ
              450 460 470 480
EEGHACFSSI SEFLLTHPVL SFGIQVVSRC RLRHTEVLPA
              490 500 510 520
EEENDSLGAD GTHGAGAMES AAGVLIKLFC VHTKALQDVQ
              530 540 550 560
IRFQPQLNPD VVAPLPTHTA HEDFTFGESR PELGSEGLGS
              570 580 590 600
AAHGSQPDLR RIVELPAPAD FLSLSSETKP KLMTPDAFMT
              610 620 630 640
PSASLQQITA SPSSSSSGSS SSSSSSSSSL TAVSAMSSTS
              650 660 670 680
AVDPSLTRPP EELTLSPKLQ LDGSLTMSSS GSLQASPRGL
              690 700 710 720
LPGLLPAPAD KLTPKGPGQV PTATSALSLE LQEVEPLGLP
              730 740 750 760
QASPSRTRSP DVISSASTAL SQDIPEIASE ALSRGFGSSA
              770 780 790 800
PEGLEPDSMA SAASALHLLS PRPRPGPELG PQLGLDGGPG
              810 820 830 840
DGDRHNTPSL LEAALTQEAS TPDSQVWPTA PDITRETCST
              850 860 870 880
LAESPRNGLQ EKHKSLAFHR PPYHLLQQRD SQDASAEQSD
              890 900 910 920
HDDEVASLAS ASGGFGTKVP APRLPAKDWK TKGSPRTSPK
              930 940 950 960
LKRKSKKDDG DAAMGSRLTE HQVAEPPEDW PALIWQQQRE
              970 980 990 1000
LAELRHSQEE LLQRLCTQLE GLQSTVTGHV ERALETRHEQ
            1010 1020 1030 1040
EQRRLERALA EGQQRGGQLQ EQLTQQLSQA LSSAVAGRLE
            1050 1060 1070 1080
RSIRDEIKKT VPPCVSRSLE PMAGQLSNSV ATKLTAVEGS
            1090 1100 1110 1120
MKENISKLLK SKNLTDAIAR AAADTLQGPM QAAYREAFQS
            1130 1140 1150 1160
VVLPAFEKSC QAMFQQINDS FRLGTQEYLQ QLESHMKSRK
            1170 1180 1190 1200
AREQEAREPV LAQLRGLVST LQSATEQMAA TVAGSVRAEV
            1210 1220 1230 1240
QHQLHVAVGS LQESILAQVQ RIVKGEVSVA LKEQQAAVTS
            1250 1260 1270 1280
SIMQAMRSAA GTPVPSAHLD CQAQQAHILQ LLQQGHLNQA
            1290 1300 1310 1320
FQQALTAADL NLVLYVCETV DPAQVFGQPP CPLSQPVLLS
            1330 1340 1350 1360
LIQQLASDLG TRTDLKLSYL EEAVMHLDHS DPITRDHMGS
            1370 1380 1390 1400
VMAQVRQKLF QFLQAEPHNS LGKAARRLSL MLHGLVTPSL
P
```

This protein is encoded by a cDNA sequence with accession number L26339 in the NCBI database.

An example of a human negative BTN3A1 regulator sequence for a CD79A protein is shown below (Uniprot P11912; SEQ ID NO:46).

```
                10 20 30 40
MPGGPGVLQA LPATIFLLFL LSAVYLGPGC QALWMHKVPA
                50 60 70 80
SLMVSLGEDA HFQCPHNSSN NANVTWWRVL HGNYTWPPEF
                90 100 110 120
LGPGEDPNGT LIIQNVNKSH GGIYVCRVQE GNESYQQSCG
              130 140 150 160
TYLRVRQPPP RPFLDMGEGT KNRIITAEGI ILLFCAVVPG
              170 180 190 200
TLLLFRKRWQ NEKLGLDAGD EYEDENLYEG LNLDDCSMYE
              210 220
DISRGLQGTY QDVGSLNIGD VQLEKP
```

This protein is encoded by a cDNA sequence with accession number S46706 in the NCBI database.

An example of a human negative BTN3A1 regulator sequence for a SLC16A1 protein is shown below (Uniprot P53985; SEQ ID NO:47).

```
                10 20 30 40
MPPAVGGPVG YTPPDGGWGW AVVIGAFISI GFSYAFPKSI
                50 60 70 80
TVFFKEIEGI FHATTSEVSW ISSIMLAVMY GGGPISSILV
                90 100 110 120
NKYGSRIVMI VGGCLSGCGL IAASFCNTVQ QLYVCIGVIG
              130 140 150 160
GLGLAFNLNP ALTMIGKYFY KRRPLANGLA MAGSPVFLCT
              170 180 190 200
LAPLNQVFFG IFGWRGSFLI LGGLLLNCCV AGALMRPIGP
              210 220 230 240
KPTKAGKDKS KASLEKAGKS GVKKDLHDAN TDLIGRHPKQ
              250 260 270 280
EKRSVFQTIN QFLDLTLFTH RGFLLYLSGN VIMFFGLFAP
              290 300 310 320
LVFLSSYGKS QHYSSEKSAF LLSILAFVDM VARPSMGLVA
              330 340 350 360
NTKPIRPRIQ YFFAASVVAN GVCHMLAPLS TTYVGFCVYA
              370 380 390 400
GFFGFAFGWL SSVLFETLMD LVGPQRFSSA VGLVTIVECC
              410 420 430 440
PVLLGPPLLG RLNDMYGDYK YTYWACGVVL IISGIYLFIG
              450 460 470 480
MGINYRLLAK EQKANEQKKE SKEEETSIDV AGKPNEVTKA
              490 500
AESPDQKDTD GGPKEEESPV
```

This protein is encoded by a cDNA sequence with accession number L31801 in the NCBI database.

An example of a human negative BTN3A1 regulator sequence for a RBM10 protein is shown below (Uniprot P98175; SEQ ID NO:48).

```
                10 20 30 40
MEYERRGGRG DRTGRYGATD RSQDDGGENR SRDHDYRDMD
                50 60 70 80
YRSYPREYGS QEGKHDYDDS SEEQSAEDSY EASPGSETQR
                90 100 110 120
RRRRRHRHSP TGPPGFPRDG DYRDQDYRTE QGEEEEEEED
              130 140 150 160
EEEEEKASNI VMLRMLPQAA TEDDIRGQLQ SHGVQAREVR
              170 180 190 200
LMRNKSSGQS RGFAFVEFSH LQDATRWMEA NQHSLNILGQ
              210 220 230 240
KVSMHYSDPK PKINEDWLCN KCGVQNFKRR EKCFKCGVPK
              250 260 270 280
SEAEQKLPLG TRLDQQTLPL GGRELSQGLL PLPQPYQAQG
              290 300 310 320
VLASQALSQG SEPSSENAND TIILRNLNPH STMDSILGAL
              330 340 350 360
APYAVLSSSN VRVIKDKQTQ LNRGFAFIQL STIVEAAQLL
              370 380 390 400
QILQALHPPL TIDGKTINVE FAKGSKRDMA SNEGSRISAA
              410 420 430 440
SVASTAIAAA QWAISQASQG GEGTWATSEE PPVDYSYYQQ
              450 460 470 480
DEGYGNSQGT ESSLYAHGYL KGTKGPGITG TKGDPTGAGP
              490 500 510 520
EASLEPGADS VSMQAFSRAQ PGAAPGIYQQ SAEASSSQGT
              530 540 550 560
AANSQSYTIM SPAVLKSELQ SPTHPSSALP PATSPTAQES
              570 580 590 600
YSQYPVPDVS TYQYDETSGY YYDPQTGLYY DPNSQYYYNA
              610 620 630 640
QSQQYLYWDG ERRTYVPALE QSADGHKETG APSKEGKEKK
              650 660 670 680
EKHKTKTAQQ IAKDMERWAR SLNKQKENFK NSFQPISSLR
              690 700 710 720
DDERRESATA DAGYAILEKK GALAERQHTS MDLPKLASDD
              730 740 750 760
RPSPPRGLVA AYSGESDSEE EQERGGPERE EKLTDWQKLA
              770 780 790 800
CLLCRRQFPS KEALIRHQQL SGLHKQNLEI HRRAHLSENE
              810 820 830 840
LEALEKNDME QMKYRDRAAE RREKYGIPEP PEPKRRKYGG
              850 860 870 880
ISTASVDFEQ PTRDGLGSDN IGSRMLQAMG WKEGSGLGRK
              890 900 910 920
KQGIVTPIEA QTRVRGSGLG ARGSSYGVTS TESYKETLHK
              930
TMVTRFNEAQ
```

This protein is encoded by a cDNA sequence with accession number D50912 in the NCBI database.

An example of a human negative BTN3A1 regulator sequence for a GALE protein is shown below (Uniprot Q14376; SEQ ID NO:49).

```
                10 20 30 40
MAEKVLVTGG AGYIGSHTVL ELLEAGYLPV VIDNFHNAFR
                50 60 70 80
GGGSLPESLR RVQELTGRSV EFEEMDILDQ GALQRLFKKY
                90 100 110 120
SFMAVIHFAG LKAVGESVQK PLDYYRVNLT GTIQLLEIMK
              130 140 150 160
AHGVKNLVFS SSATVYGNPQ YLPLDEAHPT GGCTNPYGKS
              170 180 190 200
KFFIEEMIRD LCQADKTWNA VLLRYFNPTG AHASGCIGED
              210 220 230 240
PQGIPNNLMP YVSQVAIGRR EALNVFGNDY DTEDGTGVRD
              250 260 270 280
YIHVVDLAKG HIAALRKLKE QCGCRIYNLG TGTGYSVLQM
              290 300 310 320
VQAMEKASGK KIPYKVVARR EGDVAACYAN PSLAQEELGW
              330 340
TAALGLDRMC EDLWRWQKQN PSGFGTQA
```

This protein is encoded by a cDNA sequence with accession number L41668 in the NCBI database.

An example of a human negative BTN3A1 regulator sequence for a MEF2B protein is shown below (Uniprot Q02080; SEQ ID NO:50).

```
                10 20 30 40
MGRKKIQISR ILDQRNRQVT FTKRKFGLMK KAYELSVLCD
                50 60 70 80
CEIALIIFNS ANRLFQYAST DMDRVLLKYT EYSEPHESRT
                90 100 110 120
NTDILETLKR RGIGLDGPEL EPDEGPEEPG EKFRRLAGEG
              130 140 150 160
GDPALPRPRL YPAAPAMPSP DVVYGALPPP GCDPSGLGEA
              170 180 190 200
LPAQSRPSPF RPAAPKAGPP GLVHPLFSPS HLTSKTPPPL
              210 220 230 240
YLPTEGRRSD LPGGLAGPRG GLNTSRSLYS GLQNPCSTAT
              250 260 270 280
PGPPLGSFPF LPGGPPVGAE AWARRVPQPA APPRRPPQSA
              290 300 310 320
SSLSASLRPP GAPATFLRPS PIPCSSPGPW QSLCGLGPPC
              330 340 350 360
AGCPWPTAGP GRRSPGGTSP ERSPGTARAR GDPTSLQASS
EKTQQ
```

This protein is encoded by a cDNA sequence with accession number X68502 in the NCBI database.

An example of a human negative BTN3A1 regulator sequence for a FAM96B protein is shown below (Uniprot Q9Y3D0; SEQ ID NO:51).

```
                10 20 30 40
MVGGGGVGGG LLENANPLIY QRSGERPVTA GEEDEQVPDS
                50 60 70 80
IDAREIFDLI RSINDPEHPL TLEELNVVEQ VRVQVSDPES
                90 100 110 120
TVAVAFTPTI PHCSMATLIG LSIKVKLLRS LPQRFKMDVH
              130 140 150 160
ITPGTHASEH AVNKQLADKE RVAAALENTH LLEVVNQCLS
ARS
```

This protein is encoded by a cDNA sequence with accession number AF151886 in the NCBI database.

An example of a human negative BTN3A1 regulator sequence for an ATXN7 protein is shown below (Uniprot O15265; SEQ ID NO: 52).

```
                10 20 30 40
MSERAADDVR GEPRRAAAAA GGAAAAAARQ QQQQQQQQQP
                50 60 70 80
PPPQPQRQQH PPPPPRRTRP EDGGPGAAST SAAAMATVGE
                90 100 110 120
RRPLPSPEVM LGQSWNLWVE ASKLPGKDGT ELDESFKEFG
              130 140 150 160
KNREVMGLCR EDMPIFGFCP AHDDFYLVVC NDCNQVVKPQ
              170 180 190 200
AFQSHYERRH SSSSKPPLAV PPTSVFSFFP SLSKSKGGSA
              210 220 230 240
SGSNRSSSGG VLSASSSSSK LLKSPKEKLQ LRGNTRPMHP
              250 260 270 280
IQQSRVPHGR IMTPSVKVEK IHPKMDGTLL KSAVGPTCPA
              290 300 310 320
TVSSLVKPGL NCPSIPKPTL PSPGQILNGK GLPAPPTLEK
              330 340 350 360
KPEDNSNNRK FLNKRLSERE FDPDIHCGVI DLDTKKPCTR
              370 380 390 400
SLTCKTHSLT QRRAVQGRRK RFDVLLAEHK NKTREKELIR
              410 420 430 440
HPDSQQPPQP LRDPHPAPPR TSQEPHQNPH GVIPSESKPF
              450 460 470 480
VASKPKPHTP SLPRPPGCPA QQGGSAPIDP PPVHESPHPP
              490 500 510 520
LPATEPASRL SSEEGEGDDK EESVEKLDCH YSGHHPQPAS
              530 540 550 560
FCTFGSRQIG RGYYVFDSRW NRLRCALNLM VEKHLNAQLW
              570 580 590 600
KKIPPVPSTT SPISTRIPHR TNSVPTSQCG VSYLAAATVS
              610 620 630 640
TSPVLLSSTC ISPNSKSVPA HGTTLNAQPA ASGAMDPVCS
              650 660 670 680
MQSRQVSSSS SSPSTPSGLS SVPSSPMSRK PQKLKSSKSL
              690 700 710 720
RPKESSGNST NCQNASSSTS GGSGKKRKNS SPLLVHSSSS
              730 740 750 760
SSSSSSSSHS MESFRKNCVA HSGPPYPSTV TSSHSIGLNC
              770 780 790 800
VTNKANAVNV RHDQSGRGPP TGSPAESIKR MSVMVNSSDS
              810 820 830 840
TLSLGPFIHQ SNELPVNSHG SFSHSHTPLD KLIGKKRKCS
              850 860 870 880
PSSSSINNSS SKPTKVAKVP AVNNVHMKHT GTIPGAQGLM
              890
NSSLLHQPKA RP
```

This protein is encoded by a cDNA sequence with accession number AJ000517 in the NCBI database.

An example of a human negative BTN3A1 regulator sequence for a COG8 protein is shown below (Uniprot Q96MW5; SEQ ID NO:53).

```
                10 20 30 40 50
MATAATIPSV ATATAAALGE VEDEGLLASL FRDRFPEAQW RERPDVGRYL
                60 70 80 90 100
RELSGSGLER LRREPERLAE ERAQLLQQTR DLAFANYKTF IRGAECTERI
              110 120 130 140 150
HRLFGDVEAS LGRLLDRLPS FQQSCRNFVK EAEEISSNRR MNSLTLNRHT
              160 170 180 190 200
EILEILEIPQ LMDTCVRNSY YEEALELAAY VRRLERKYSS IPVIQGIVNE
              210 220 230 240 250
VRQSMQLMLS QLIQQLRTNI QLPACLRVIG YLRRMDVFTE AELRVKFLQA
              260 270 280 290 300
RDAWLRSILT AIPNDDPYFH ITKTIEASRV HLFDIITQYR AIFSDEDPLL
              310 320 330 340 350
PPAMGEHTVN ESAIFHGWVL QKVSQFLQVL ETDLYRGIGG HLDSLLGQCM
              360 370 380 390 400
YFGLSFSRVG ADFRGQLAPV FQRVAISTFQ KAIQETVEKF QEEMNSYMLI
              410 420 430 440 450
SAPAILGTSN MPAAVPATQP GTLQPPMVLL DFPPLACFLN NILVAFNDLR
              460 470 480 490 500
LCCPVALAQD VTGALEDALA KVTKIILAFH RAEEAAFSSG EQELFVQFCT
              510 520 530 540 550
VFLEDLVPYL NRCLQVLFPP AQIAQTLGIP PTQLSKYGNL GHVNIGAIQE
              560 570 580 590 600
PLAFILPKRE TLFTLDDQAL GPELTAPAPE PPAEEPRLEP AGPACPEGGR
              610
AETQAEPPSV GP
```

This protein is encoded by a cDNA sequence with accession number AK056344 in the NCBI database.

An example of a human negative BTN3A1 regulator sequence for a DERL1 protein is shown below (Uniprot Q9BUN8; SEQ ID NO:54).

```
                10 20 30 40 50
MSDIGDWFRS IPAITRYWFA ATVAVPLVGK LGLISPAYLF LWPEAFLYRF
                60 70 80 90 100
QIWRPITATF YFPVGPGTGF LYLVNLYFLY QYSTRLETGA FDGRPADYLF
              110 120 130 140 150
QIWRPOTATF YFPVGPGTGF LYLVNLYFLY QYSTRLETGA FDGRPADLYF
              160 170 180 190 200
MLLFNWICIV ITGLAMDMQL LMIPLIMSVL YVWAQLNRDM IVSFWFGTRF
              210 220 230 240 250
KACYLPWVIL GFNYIIGGSV INELIGNLVG HLYFFLMFRY PMDLGGRNFL
              260 270 280 290 300
STPQFLYRWL PSRRGGVSGF GVPPASMRRA ADQNGGGGRH NWGQGFRLGD
Q
```

This protein is encoded by a cDNA sequence with accession number AY358818 in the NCBI database.

An example of a human negative BTN3A1 regulator sequence for a TGFBR2 protein is shown below (Uniprot P37173; SEQ ID NO:55).

```
                10 20 30 40 50
MGRGLIRGLW PLHIVLWTRI ASTIPPHVOK SVNNDMIVTD NNGAVKFPQL
                60 70 80 90 100
CKFCDVRFST CDNQKSCMSN CSITSICEKP QEVCVAVWRK NDENITLETV
              110 120 130 140 150
CHDPKLPYHD FILEDAASPK CIMKEKKKPG ETFFMCSCSS DECNDNIIFS
              160 170 180 190 200
EEYNTSNPDL LLVIFQVTGI SLLPPLGVAI SVIIIFYCYR VNRQQKLSST
              210 220 230 240 250
WETGKTRKLM EFSEHCAIIL EDDRSDISST CANNINHNTE LLPIELDTLV
              260 270 280 290 300
GKGRFAEVYK AKLKQNTSEQ FETVAVKIFP YEEYASWKTE KDIFSDINLK
              310 320 330 340 350
HENILQFLTA EERKTELGKQ YWLITAFHAK GNLQEYLTRH VISWEDLRKL
              360 370 380 390 400
GSSLARGIAH LHSDHTPCGR PKMPIVHRDL KSSNILVKND LTCCLCDFGL
              410 420 430 440 450
SLRLDPTLSV DDLANSGQVG TARYMAPEVL ESRMNLENVE SFKQTDVYSM
              460 470 480 490 500
ALVIWEMTSR CNAVGEVKDY EPPFGSKVRE HPCVESMKDN VLRDRGRPEI
              510 520 530 540 550
PSFWLNHQGI QMVCETLTEC WDHDPEARLT AQCVAERFSE LEHLDRLSGR
              560
SCSEEKIPED GSLNTTK
```

This protein is encoded by a cDNA sequence with accession number M85079 in the NCBI database.

An example of a human negative BTN3A1 regulator sequence for a CHTF8 protein is shown below (Uniprot P0CG13; SEQ ID NO:56).

```
                10 20 30 40 50
MVQIVISSAR AGGLAEWVLM ELQGEIEARY STGLAGNLLG DLHYTTEGIP
                60 70 80 90 100
VLIVGHHILY GKIIHLEKPF AVLVKHTPGD QDCDELGRET GTRYLVTALI
              110 120
KDKILFKTRP KPIITSVPKK V
```

This protein is encoded by a cDNA sequence with accession number BC018700 in the NCBI database.

An example of a human negative BTN3A1 regulator sequence for an AHCYL1 protein is shown below (Uniprot O43865; SEQ ID NO:57).

```
                10 20 30 40 50
MSMPDAMPLP GVGEELKQAK EIEDAEKYSF MATVTKAPKK QIQFADDMQE
                60 70 80 90 100
FTKFPTKTGR RSLSRSISQS STDSYSSAAS YTDSSDDEVS PREKQQTNSK
              110 120 130 140 150
GSSNFCVKNI KQAEFGRREI EIAEQDMSAL ISLRKRAQGE KPLAGAKIVG
              160 170 180 190 200
CTHITAQTAV LIETLCALGA QCRWSACNIY STQNEVAAAL AEAGVAVFAW
              210 220 230 240 250
KGESEDDFWW CIDRCVNMDG WQANMILDDG GDLTHWVYKK YPNVFKKIRG
              260 270 280 290 300
IVEESVTGVH RLYQLSKAGK LCVPAMNVND SVTKQKFDNL YCCRESILDG
              310 320 330 340 350
LKRTIDVMFG GKQVVVCGYG EVGKGCCAAL KALGAIVYIT EIDPICALQA
              360 370 380 390 400
CMDGFRVVKL NEVIRQVDVV ITCTGNKNVV TREHLDRMKN SCIVCNMGHS
              410 420 430 440 450
NTEIDVTSLR TPELTWERVR SQVDHVIWPD GKRVVLLAEG RLLNLSCSTV
              460 470 480 490 500
PTFVLSITAT TQALALIELY NAPEGRYKQD VYLLPKKMDE YVASLHLPSF
              510 520 530
DAHLTELTDD QAKYLGLNKN GPFKPNYYRY
```

This protein is encoded by a cDNA sequence with accession number AF315687 in the NCBI database.

The sequences provided herein are exemplary. Isoforms and variants of the sequences described herein and of any of regulators listed in Tables 1 and 2 can also be used in the methods and compositions described herein.

For example, isoforms and variants of the proteins and nucleic acids can be used in the methods and compositions described herein when they are substantially identical to the ‘reference’ sequences described herein and/or substantially identical to the any of the genes listed in Tables 1 or 2. The terms “substantially identity” indicates that a polypeptide or nucleic acid comprises a sequence with between 55-100% sequence identity to a reference sequence, for example with at least 55% sequence identity, preferably 60%, preferably 70%, preferably 80%, preferably at least 90%, preferably at least 95%, preferably at least 96%, preferably at least 97% sequence, preferably at least 98%, preferably at least 99% identity to a reference sequence over a specified comparison window. Optimal alignment may be ascertained or conducted using the homology alignment algorithm of Needleman and Wunsch, J. Mol. Biol. 48:443-53 (1970).

### Positive BTN3A1 Regulators

The positive BTN3A1 regulators can be used as markers that identify cancer cell types that can be killed by T cells such as γδ T cells, or Vγ9Vδ2 T cells. Hence, methods are described herein for identifying and/or treating subjects who can benefit from T cell therapies that can involve detection and/or quantification of positive BTN3A1 regulator expression levels in samples suspected of containing cancer cells. For example, if a sample exhibits increased expression levels of any of BTN3A or any of the BTN3A positive regulators described herein (relative to a reference value or negative control), the subject from whom the sample was obtained is a good candidate for T cell therapy. However, if a sample exhibits increased expression levels of any of the BTN3A negative regulators described herein (relative to a reference value or negative control), the subject from whom the sample was obtained is likely not a good candidate for T cell therapy.

Lists of negative and positive regulators of BTN3A1 are provided in Table 1 and 2. In some cases, the expression of one or more genes involved in oxidative phosphorylation (OXPHOS genes), genes involved in the mevalonate pathway, genes involved in metabolic sensing, genes involved in purine biosynthesis (PPAT genes), transcription factor genes, BTN3A genes, or a combination of those genes is evaluated. For example, positive regulators of BTN3A that may be markers indicating that T cell therapy is useful can, for example, include the first fifty genes listed in Table 2. The first fifty of the positive BTN3A1 regulators listed in Table 2 are ECSIT, FBXW7, SPIB, IRF1, NLRC5, IRF8, NDUFA2, NDUFV1, NDUFA13, USP7, C17orf89, RFXAP, UBE2A, SRPK1, NDUFS7, PDS5B, CNOT11, NDUFB7, BTN3A2, FOXRED1, NDUFS8, JMJD6, NDUFS2, NDUFC2, HSF1, ACAD9, NDUFAF5, TIMMDC1, HSD17B10, BRD2, NDUFA6, CNOT4, SPI1, MDH2, DARS2, TMEM261, STIP1, FIBP, FXR1, NFU1, GGNBP2, STAT2, TRUB2, BIRC6, MARS2, NDUFA9, USP19, UBA6, MTG1, AMPK, and KIAA0391.

In some cases, positive regulators of BTN3A that may be good markers indicating that T cell therapy is useful include IRF1, IRF8, IRF9, NLRC5, SPI1, SPIB, AMP-activated protein kinase (AMPK), or a combination thereof. Note that AMPK is made up of the following three subunits, each encoded by 2 or 3 different genes: α—PRKAA1, PRKAA2; β—PRKAB1, PRKAB2; and γ—PRKAG1, PRKAG2, PRKAG3. Hence, levels of AMPK can be measured by measuring any one (or more) of these three AMPK subunits. When measuring BTN3A positive regulator expression levels, it can also be useful to measure BTN3A expression levels.

The positive BTN3A1 regulators include any of those listed in Table 2. Human sequences for any of these positive regulator protein and nucleic acids are available, for example in the NCBI database (ncbi.nlm.nih.gov) or the Uniprot database (uniprot.org).

For example, the first fifty of the positive BTN3A1 regulators listed in Table 2 are ECSIT, FBXW7, SPIB, IRF1, NLRC5, IRF8, NDUFA2, NDUFV1, NDUFA13, USP7, C17orf89, RFXAP, UBE2A, SRPK1, NDUFS7, PDS5B, CNOT11, NDUFB7, BTN3A2, FOXRED1, NDUFS8, JMJD6, NDUFS2, NDUFC2, HSF1, ACAD9, NDUFAF5, TIMMDC1, HSD17B10, BRD2, NDUFA6, CNOT4, SPI1, MDH2, DARS2, TMEM261, STIP1, FIBP, FXR1, NFU1, GGNBP2, STAT2, TRUB2, BIRC6, MARS2, NDUFA9, USP19, UBA6, MTG1, AMPK, KIAA0391, and IRF9.

An example of a human positive BTN3A1 regulator sequence for an ECSIT protein is shown below (Uniprot Q9BQ95; SEQ ID NO:58).

```
                10 20 30 40 50
MSWVQATLLA RGLCRAWGGT CGAALTGTSI SQVPRRLPRG LHCSAAAHSS
                60 70 80 90 100
EQSLVPSPPE PRQRPTKALV PFEDLFGQAP GGERDKASFL QTVQKFAEHS
              110 120 130 140 150
VRKRGHIDFI YLALRKMREY GVERDLAVYN QLLNIFPKEV FRPRNIIQRI
              160 170 180 190 200
FVHYPRQQEC GIAVLEQMEN HGVMPNKETE FLLIQIFGRK SYPMLKLVRL
              210 220 230 240 250
KLWFPRFMNV NPFPVPRDLP QDPVELAMFG LRHMEPDLSA RVTIYQVPLP
              260 270 280 290 300
KDSTGAADPP QPHIVGIQSP DQQAALARHN PARPVFVEGP FSLWIRNKCV
              310 320 330 340 350
YYHILRADLL PPEEREVEET PEEWNLYYPM QLDLEYVRSG WDNYEFDINE
              360 370 380 390 400
VEEGPVFAMC MAGAHDQATM AKWIQGLQET NPTLAQIPVV FRLAGSTREL
              410 420 430
QTSSAGLEEP PLPEDHQEED DNLQRQQQGQ S
```

This ECSIT protein is encoded by a cDNA sequence with accession number AF243044 in the NCBI database.

An example of a human positive BTN3A1 regulator sequence for an FBXW7 protein is shown below (Uniprot Q969H0; SEQ ID NO:59).

```
                10 20 30 40 50
MNQELLSVGS KRRRTGGSLR GNPSSSQVDE EQMNRVVEEE QQQQLRQQEE
                60 70 80 90 100
EHTARNGEVV GVEPRPGGQN DSQQGQLEEN NNRFISVDED SSGNQEEQEE
              110 120 130 140 150
DEEHAGEQDE EDEEEEEMDQ ESDDFDQSDD SSREDEHTHT NSVTNSSSIV
              160 170 180 190 200
DLPVHQLSSP FYTKTTKMKR KLDHGSEVRS FSLGKKPCKV SEYTSTTGLV
              210 220 230 240 250
PCSATPTTFG DLRAANGQGQ QRRRITSVQP PTGLQEWLKM FQSWSGPEKL
              260 270 280 290 300
LALDELIDSC EPTQVKHMMQ VIEPQFQRDF ISLLPKELAL YVLSFLEPKD
              310 320 330 340 350
LLQAAQTCRY WRILAEDNLL WREKCKEEGI DEPLHIKRRK VIKPGFIHSP
              360 370 380 390 400
WKSAYIRQHR IDTNWRRGEL KSPKVLKGHD DHVITCLQFC GNRIVSGSDD
              410 420 430 440 450
NTLKVWSAVT GKCLRTLVGH TGGVWSSQMR DNIIISGSTD RTLKVWNAET
              460 470 480 490 500
GECIHTLYGH TSTVRCMHLH EKRVVSGSRD ATLRVWDIET GQCLHVLMGH
              510 520 530 540 550
VAAVRCVQYD GRRVVSGAYD FMVKVWDPET ETCLHTLQGH TNRVYSLQFD
              560 570 580 590 600
GIHVVSGSLD TSIRVWDVET GNCIHTLTGH QSLTSGMELK DNILVSGNAD
              610 620 630 640 650
STVKIWDIKT GQCLQTLQGP NKHQSAVTCL QFNKNFVITS SDDGTVKLWD
              660 670 680 690 7 00
LKTGEFIRNL VILESGGSGG VVWRIRASNT KLVCAVGSRN GTEETKLLVL
DFDVDMK
```

This protein is encoded by a cDNA sequence with accession number AY033553 in the NCBI database.

An example of a human positive BTN3A1 regulator sequence for a SPIB protein is shown below (Uniprot Q01892; SEQ ID NO:60).

```
                10 20 30 40 50
MLALEAAQLD GPHFSCLYPD GVFYDLDSCK HSSYPDSEGA PDSLWDWTVA
                60 70 80 90 100
PPVPATPYEA FDPAAAAFSH PQAAQLCYEP PTYSPAGNLE LAPSLEAPGP
              110 120 130 140 150
GLPAYPTENF ASQTLVPPAY APYPSPVLSE EEDLPLDSPA LEVSDSESDE
              160 170 180 190 200
ALVAGPEGKG SEAGTRKKLR LYQFLLGLLT RGDMRECVWW VEPGAGVFQF
              210 220 230 240 250
SSKHKELLAR RWGQQKGNRK RMTYQKLARA LRNYAKTGEI RKVKRKLTYQ
              260
FDSALLPAVR RA
```

This protein is encoded by a cDNA sequence with accession number X66079 in the NCBI database.

An example of a human positive BTN3A1 regulator sequence for an IRF1 protein is shown below (Uniprot P10914; SEQ ID NO:61).

```
                10 20 30 40 50
MPITRMRMRP WLEMQINSNQ IPGLIWINKE EMIFQIPWKH AAKHGWDINK
                60 70 80 90 100
DACLFRSWAI HTGRYKAGEK EPDPKTWKAN FRCAMNSLPD IEEVKDQSRN
              110 120 130 140 150
KGSSAVRVYR MLPPLTKNQR KERKSKSSRD AKSKAKRKSC GDSSPDTFSD
              160 170 180 190 200
GLSSSTLPDD HSSYTVPGYM QDLEVEQALT PALSPCAVSS TLPDWHIPVE
              210 220 230 240 250
VVPDSTSDLY NFQVSPMPST SEATTDEDEE GKLPEDIMKL LEQSEWQPTN
              260 270 280 290 300
VDGKGYLLNE PGVQPTSVYG DFSCKEEPEI DSPGGDIGLS LQRVFTDLKN
              310 320
MDATWLDSLL TPVRLPSIQA IPCAP
```

This protein is encoded by a cDNA sequence with accession number X14454.1 in the NCBI database.

An example of a human positive BTN3A1 regulator sequence for a NLRC5 protein is shown below (Uniprot 86W13” SEQ ID NO:62.

```
                10 20 30 40 50
MDPVGLQLGN KNLWSCLVRL LTKDPEWLNA KMKFFLPNTD LDSRNETLDP
                60 70 80 90 100
EQRVILQLNK LHVQGSDTWQ SFIHCVCMQL EVPLDLEVEL LSTFGYDDGF
              110 120 130 140 150
TSQLGAEGKS QPESQLHHGL KRPHQSCGSS PRRKQCKKQQ LELAKKYLQL
              160 170 180 190 200
LRTSAQQRYR SQIPGSGQPH AFHQVYVPPI LRRATASLDT PEGAIMGDVK
              210 220 230 240 250
VEDGADVSIS DLFNTRVNKG PRVTVLLGKA GMGKTTLAHR LCQKWAEGHL
              260 270 280 290 300
NCFQALFLFE FRQLNLITRF LTPSELLFDL YLSPESDHDT VFQYLEKNAD
              310 320 330 340 350
QVLLIFDGLD EALQPMGPDG PGPVLTLFSH LCNGTLLPGC RVMATSRPGK
              360 370 380 390 400
LPACLPAEAA MVHMLGFDGP RVEEYVNHFF SAQPSREGAL VELQTNGRLR
              410 420 430 440 450
SLCAVPALCQ VACLCLHHLL PDHAPGQSVA LLPNMTQLYM QMVLALSPPG
              460 470 480 490 500
HLPTSSLLDL GEVALRGLET GKVIFYAKDI APPLIAFGAT HSLLTSFCVC
              510 520 530 540 550
TGPGHQQTGY AFTHLSLQEF LAALHLMASP KVNKDTLTQY VTLHSRWVQR
              560 570 580 590 600
TKARLGLSDH LPTFLAGLAS CTCRPFLSHL AQGNEDCVGA KQAAVVQVLK
              610 620 630 640 650
KLATRKLTGP KVVELCHCVD ETQEPELASL TAQSLPYQLP FHNFPLTCTD
              660 670 680 690 700
LATLTNILEH REAPIHLDFD GCPLEPHCPE ALVGCGQIEN LSFKSRKCGD
              710 720 730 740 750
AFAEALSRSL PTMGRLQMLG LAGSKITARG ISHLVKALPL CPQLKEVSFR
              760 770 780 790 800
DNQLSDQVVL NIVEVLPHLP RLRKLDLSSN SICVSTLLCL ARVAVTCPTV
              810 820 830 840 850
RMLQAREADL IFLLSPPTET TAELQRAPDL QESDGQRKGA QSRSLTLRLQ
              860 870 880 890 900
KCQLQVHDAE ALIALLQEGP HLEEVDLSGN QLEDEGCRLM AEAASQLHIA
              910 920 930 940 950
RKLDLSNNGL SVAGVHCVLR AVSACWTLAE LHISLQHKTV IFMFAQEPEE
              960 970 980 990 1000
QKGPQERAAF LDSLMLQMPS ELPLSSRRMR LTHCGLQEKH LEQLCKALGG
            1010 1020 1030 1040 1050
SCHLGHLHLD FSGNALGDEG AARLAQLLPG LGALQSLNLS ENGLSLDAVL
            1060 1070 1080 1090 1100
GLVRCFSTLQ WLFRLDISFE SQHILLRGDK TSRDMWATGS LPDFPAAAKF
            1110 1120 1130 1140 1150
LGFRQRCIPR SLCLSECPLE PPSLTRLCAT LKDCPGPLEL QLSCEFLSDQ
            1160 1170 1180 1190 1200
SLETLLDCLP QLPQLSLLQL SQTGLSPKSP FLLANTLSLC PRVKKVDLRS
            1210 1220 1230 1240 1250
LHHATLHFRS NEEEEGVCCG RFTGCSLSQE HVESLCWLLS KCKDLSQVDL
            1260 1270 1280 1290 1300
SANLLGDSGL RCLLECLPQV PISGLLDISH NSISQESALY LLETLPSCPR
            1310 1320 1330 1340 1350
VREASVNLGS EQSFRIHFSR EDQAGKTLRL SECSFRPEHV SRLATGLSKS
            1360 1370 1380 1390 1400
LQLTELTLTQ CCLGQKQLAI LLSLVGRPAG LFSLRVQEPW ADRARVLSLL
            1410 1420 1430 1440 1450
EVCAQASGSV TEISISETQQ QLCVQLEFPR QEENPEAVAL RLAHCDLGAH
            1460 1470 1480 1490 1500
HSLLVGQLME TCARLQQLSL SQVNLCEDDD ASSLLLQSLL LSLSELKTFR
            1510 1520 1530 1540 1550
LTSSCVSTEG LAHLASGLGH CHHLEELDLS NNQFDEEGTK ALMRALEGKW
            1560 1570 1580 1590 1600
MLKRLDLSHL LLNSSTLALL THRLSQMTCL QSLRLNRNSI GDVGCCHLSE
            1610 1620 1630 1640 1650
ALRAATSLEE LDLSHNQIGD AGVQHLATIL PGLPELRKID LSGNSISSAG
            1660 1670 1680 1690 1700
GVQLAESLVL CRRLEELMLG CNALGDPTAL GLAQELPQHL RVLHLPFSHL
            1710 1720 1730 1740 1750
GPGGALSLAQ ALDGSPHLEE ISLAENNLAG GVLRFCMELP LLRQIDLVSC
            1760 1770 1780 1790 1800
KIDNQTAKLL TSSFTSCPAL EVILLSWNLL GDEAAAELAQ VLPQMGRLKR
            1810 1820 1830 1840 1850
VDLEKNQITA LGAWLLAEGL AQGSSIQVIR LWNNPIPCDM AQHLKSQEPR
            1860
LDFAFFDNQP QAPWGT
```

This protein is encoded by a cDNA sequence with accession number AF389420 in the NCBI database.

An example of a human positive BTN3A1 regulator sequence for an IRF8 protein is shown below (Uniprot Q02556; SEQ ID NO:63).

```
                10 20 30 40 50
MCDRNGGRRL RQWLIEQIDS SMYPGLIWEN EEKSMFRIPW KHAGKQDYNQ
                60 70 80 90 100
EVDASIFKAW AVFKGKFKEG DKAEPATWKT RLRCALNKSP DFEEVTDRSQ
              110 120 130 140 150
LDISEPYKVY RIVPEEEQKC KLGVATAGCV NEVTEMECGR SEIDELIKEP
              160 170 180 190 200
SVDDYMGMIK RSPSPPEACR SQLLPDWWAQ QPSTGVPLVT GYTTYDAHHS
              210 220 230 240 250
AFSQMVISFY YGGKLVGQAT TTCPEGCRLS LSQPGLPGTK LYGPEGLELV
              260 270 280 290 300
RFPPADAIPS ERQRQVTRKL FGHLERGVLL HSSRQGVEVK RLCQGRVFCS
              310 320 330 340 350
GNAVVCKGRP NKLERDEVVQ VFDTSQFFRE LQQFYNSQGR LPDGRVVLCF
              360 370 380 390 400
GEEFPDMAPL RSKLILVQIE QLYVRQLAEE AGKSCGAGSV MQAPEEPPPD
              410 420
QVFRMFPDIC ASHQRSFFRE NQQITV
```

This protein is encoded by a cDNA sequence with accession number M91196 in the NCBI database.

An example of a human positive BTN3A1 regulator sequence for a NDUFA2 protein is shown below (Uniprot O43678; SEQ ID NO:64).

```
                10 20 30 40 50
MAAAAASRGV GAKLGLREIR IHLCQRSPGS QGVRDFIEKR YVELKKANPD
                60 70 80 90
LPILIRECSD VQPKLWARYA FGQETNVPLN NFSADQVTRA LENVLSGKA
```

This protein is encoded by a cDNA sequence with accession number AF047185 in the NCBI database.

An example of a human positive BTN3A1 regulator sequence for an NDUFV1 protein is shown below (Uniprot P49821; SEQ ID NO:65).

```
                10 20 30 40 50
MLATRRLLGW SLPARVSVRF SGDTTAPKKT SFGSLKDEDR IFTNLYGRHD
                60 70 80 90 100
WRLKGSLSRG DWYKTKEILL KGPDWILGEI KTSGLRGRGG AGFPTGLKWS
              110 120 130 140 150
FMNKPSDGRP KYLVVNADEG EPGTCKDREI LRHDPHKLLE GCLVGGRAMG
              160 170 180 190 200
ARAAYIYIRG EFYNEASNLQ VAIREAYEAG LIGKNACGSG YDFDVFVVRG
              210 220 230 240 250
AGAYICGEET ALIESIEGKQ GKPRLKPPEP ADVGVEGCPT TVANVETVAV
              260 270 280 290 300
SPTICRRGGT WFAGFGRERN SGTKLFNISG HVNHPCTVEE EMSVPLKELI
              310 320 330 340 350
EKHAGGVTGG WDNLLAVIPG GSSTPLIPKS VCETVLMDFD ALVQAQTGLG
              360 370 380 390 400
TAAVIVMDRS TDIVKAIARL IEFYKHESCG QCTPCREGVD WMNKVMARFV
              410 420 430 440 450
RGDARPAEID SLWEISKQIE GHTICALGDG AAWPVQGLIR HERPELEERM
QRFAQQHQAR QAAS
```

This protein is encoded by a cDNA sequence with accession number AF053070 in the NCBI database.

An example of a human positive BTN3A1 regulator sequence for a NDUFA13 protein is shown below (Uniprot Q9P0J0; SEQ ID NO:66).

```
                10 20 30 40 50
MAASKVKQDM PPPGGYGPID YKRNLPRRGL SGYSMLAIGI GTLIYGHWSI
                60 70 80 90 100
MKWNRERRRL QIEDFEARIA LLPLLQAETD RRTLQMLREN LEEEAIIMKD
              110 120 130 140
VPDWKVGESV FHTTRWVPPL IGELYGLRTT EEALHASHGF MWYT
```

This protein is encoded by a cDNA sequence with accession number AF286697 in the NCBI database.

An example of a human positive BTN3A1 regulator sequence for a USP7 protein is shown below (Uniprot Q93009; SEQ ID NO:67).

```
                10 20 30 40 50
MNHQQQQQQQ KAGEQQLSEP EDMEMEAGDT DDPPRITQNP VINGNVALSD
                60 70 80 90 100
GHNTAEEDME DDTSWRSEAT FQFTVERFSR LSESVLSPPC FVRNLPWKIM
              110 120 130 140 150
VMPRFYPDRP HQKSVGFFLQ CNAESDSTSW SCHAQAVLKI INYRDDEKSF
              160 170 180 190 200
SRRISHLFFH KENDWGFSNF MAWSEVTDPE KGFIDDDKVT FEVFVQADAP
              210 220 230 240 250
HGVAWDSKKH TGYVGLKNQG ATCYMNSLLQ TLFFTNQLRK AVYMMPTEGD
              260 270 280 290 300
DSSKSVPLAL QRVFYELQHS DKPVGTKKLT KSFGWETLDS FMQHDVQELC
              310 320 330 340 350
RVLLDNVENK MKGTCVEGTI PKLFRGKMVS YIQCKEVDYR SDRREDYYDI
              360 370 380 390 400
QLSIKGKKNI FESFVDYVAV EQLDGDNKYD AGEHGLQEAE KGVKFLTLPP
              410 420 430 440 450
VLHLQLMRFM YDPQTDQNIK INDRFEFPEQ LPLDEFLQKT DPKDPANYIL
              460 470 480 490 500
HAVLVHSGDN HGGHYVVYLN PKGDGKWCKF DDDVVSRCTK EEAIEHNYGG
              510 520 530 540 550
HDDDLSVRHC TNAYMLVYIR ESKLSEVLQA VTDHDIPQQL VERLQEEKRI
              560 570 580 590 600
EAQKRKERQE AHLYMQVQIV AEDQFCGHQG NDMYDEEKVK YTVFKVLKNS
              610 620 630 640 650
SLAEFVQSLS QTMGFPQDQI RLWPMQARSN GTKRPAMLDN EADGNKTMIE
              660 670 680 690 700
LSDNENPWTI FLETVDPELA ASGATLPKFD KDHDVMLFLK MYDPKTRSLN
              710 720 730 740 750
YCGHIYTPIS CKIRDLLPVM CDRAGFIQDT SLILYEEVKP NLTERIQDYD
              760 770 780 790 800
VSLDKALDEL MDGDIIVFQK DDPENDNSEL PTAKEYFRDL YHRVDVIFCD
              810 820 830 840 850
KTIPNDPGFV VTLSNRMNYF QVAKTVAQRL NTDPMLLQFF KSQGYRDGPG
              860 870 880 890 900
NPLRHNYEGT LRDLLQFFKP RQPKKLYYQQ LKMKITDFEN RRSFKCIWLN
              910 920 930 940 950
SQFREEEITL YPDKHGCVRD LLEECKKAVE LGEKASGKLR LLEIVSYKII
              960 970 980 990 1000
GVHQEDELLE CLSPATSRTF RIEEIPLDQV DIDKENEMLV TVAHFHKEVF
            1010 1020 1030 1040 1050
GTFGIPFLLR IHQGEHFREV MKRIQSLLDI QEKEFEKFKF AIVMMGRHQY
            1060 1070 1080 1090 1100
INEDEYEVNL KDFEPQPGNM SHPRPWLGLD HFNKAPKRSR YTYLEKAIKI HN
```

This protein is encoded by a cDNA sequence with accession number Z72499 in the NCBI database.

An example of a human positive BTN3A1 regulator sequence for a C17orf89 protein is shown below (Uniprot A1L188; SEQ ID NO:68).

```
                10 20 30 40 50
MSANGAVWGR VRSRLRAFPE RLAACGAEAA AYGRCVQAST APGGRLSKDF
                60 70
CAREFEALRS CFAAAAKKTL EGGC
```

This protein is encoded by a cDNA sequence with accession number BC127837 in the NCBI database.

An example of a human positive BTN3A1 regulator sequence for a RFXAP protein is shown below (Uniprot O00287; SEQ ID NO:69).

```
                10 20 30 40 50
MEAQGVAEGA GPGAASGVPH PAALAPAAAP TLAPASVAAA ASQFTLLVMQ
                60 70 80 90 100
PCAGQDEAAA PGGSVGAGKP VRYLCEGAGD GEEEAGEDEA DLLDTSDPPG
              110 120 130 140 150
GGESAASLED LEDEETHSGG EGSSGGARRR GSGGGSMSKT CTYEGCSETT
              160 170 180 190 200
SQVAKQRKPW MCKKHRNKMY KDKYKKKKSD QALNCGGTAS TGSAGNVKLE
              210 220 230 240 250
ESADNILSIV KQRTGSFGDR PARPTLLEQV LNQKRISLLR SPEVVQFLQK
              260 270
QQQLLNQQVL EQRQQQFPGT SM
```

This protein is encoded by a cDNA sequence with accession number AK313912 in the NCBI database.

An example of a human positive BTN3A1 regulator sequence for a UBE2A protein is shown below (Uniprot P49459; SEQ ID NO:70).

```
                10 20 30 40 50
MSTPARRRLM RDFKRLQEDP PAGVSGAPSE NNIMVWNAVI FGPEGTPFED
                60 70 80 90 100
GTFKLTIEFT EEYPNKPPTV RFVSKMFHPN VYADGSICLD ILQNRWSPTY
              110 120 130 140 150
DVSSILTSIQ SLLDEPNPNS PANSQAAQLY QENKREYEKR VSAIVEQSWR DC
```

This protein is encoded by a cDNA sequence with accession number M74524 in the NCBI database.

An example of a human positive BTN3A1 regulator sequence for a SRPK1 protein is shown below (Uniprot Q96SB4; SEQ ID NO:71).

```
                10 20 30 40 50
MERKVLALQA RKKRTKAKKD KAQRKSETQH RGSAPHSESD LPEQEEEILG
                60 70 80 90 100
SDDDEQEDPN DYCKGGYHLV KIGDLFNGRY HVIRKLGWGH FSTVWLSWDI
              110 120 130 140 150
QGKKEVAMKV VKSAEHYTET ALDEIRLLKS VRNSDPNDPN REMVVQLLDD
              160 170 180 190 200
FKISGVNGTH ICMVFEVLGH HLLKWIIKSN YQGLPLPCVK KIIQQVLQGL
              210 220 230 240 250
DYLHTKCRII HTDIKPENIL LSVNEQYIRR LAAEATEWQR SGAPPPSGSA
              260 270 280 290 300
VSTAPQPKPA DKMSKNKKKK LKKKQKRQAE LLEKRMQEIE EMEKESGPGQ
              310 320 330 340 350
KRPNKQEESE SPVERPLKEN PPNKMTQEKL EESSTIGQDQ TLMERDTEGG
              360 370 380 390 400
AAEINCNGVI EVINYTQNSN NETLRHKEDL HNANDCDVQN LNQESSELSS
              410 420 430 440 450
QNGDSSTSQE TDSCTPITSE VSDTMVCQSS STVGQSFSEQ HISQLQESIR
              460 470 480 490 500
AEIPCEDEQE QEHNGPLDNK GKSTAGNFLV NPLEPKNAEK LKVKIADLGN
              510 520 530 540 550
ACWVHKHFTE DIQTRQYRSL EVLIGSGYNT PADIWSTACM AFELATGDYL
              560 570 580 590 600
FEPHSGEEYT RDEDHIALII ELLGKVPRKL IVAGKYSKEF FTKKGDLKHI
              610 620 630 640 650
TKLKPWGLFE VIVEKYEWSQ EEAAGFTDFL LPMLELIPEK RATAAECLRH PWINS
```

This protein is encoded by a cDNA sequence with accession number U09564 in the NCBI database.

An example of a human positive BTN3A1 regulator sequence for a NDUFS7 protein is shown below (Uniprot O75251; SEQ ID NO: 72).

```
                10 20 30 40 50
MAVLSAPGLR GFRILGLRSS VGPAVQARGV HQSVATDGPS STQPALPKAR
                60 70 80 90 100
AVAPKPSSRG EYVVAKLDDL VNWARRSSLW PMTFGLACCA VEMMHMAAPR
              110 120 130 140 150
YDMDRFGVVF RASPRQSDVM IVAGTLINKM APALRKVYDQ MPEPRYVVSM
              160 170 180 190 200
GSCANGGGYY HYSYSVVRGC DRIVPVDIYI PGCPPTAEAL LYGILQLQRK
              210
IKRERRLQIW YRR
```

This protein is encoded by a cDNA sequence with accession number AK091623 in the NCBI database.

An example of a human positive BTN3A1 regulator sequence for a PDS5B protein is shown below (Uniprot Q9NTI5; SEQ ID NO:73).

```
                10 20 30 40 50
MAHSKTRTND GKITYPPGVK EISDKISKEE MVRRLKMVVK TEMDMDQDSE
                60 70 80 90 100
EEKELYLNLA LHLASDFFLK HPDKDVRLLV ACCLADIFRI YAPEAPYTSP
              110 120 130 140 150
DKLKDIFMFI TRQLKGLEDT KSPQFNRYFY LLENIAWVKS YNICFELEDS
              160 170 180 190 200
NEIFTQLYRT LESVINNGHN QKVHMHMVDL MSSIICEGDT VSQELLDTVL
              210 220 230 240 250
VNLVPAHKNL NKQAYDLAKA LLKRTAQAIE PYITNFFNQV LMLGKTSISD
              260 270 280 290 300
LSEHVEDLIL ELYNIDSHEL LSVLPQLEFK LKSNDNEERL QVVKLLAKMF
              310 320 330 340 350
GAKDSELASQ NKPLWQCYLG RFNDIHVPIR LECVKFASHC LMNHPDLAKD
              360 370 380 390 400
LTEYLKVRSH DPEEAIRHDV IVSIVTAAKK DILLVNDHLL NFVRERTLDK
              410 420 430 440 450
RWRVRKEAMM GLAQIYKKYA LQSAAGKDAA KQIAWIKDKL LHIYYQNSID
              460 470 480 490 500
DRLLVERIFA QYMVPHNLET TERMKCLYYL YATLDLNAVK ALNEMWKCQN
              510 520 530 540 550
LLRHQVKDLL DLIKQPKTDA SVKAIFSKVM VITRNLPDPG KAQDEMKKFT
              560 570 580 590 600
QVLEDDEKIR KQLEVLVSPT CSCKQAEGCV REITKKLGNP KQPTNPFLEM
              610 620 630 640 650
IKFLLERIAP VHIDTESISA LIKQVNKSID GTADDEDEGV PTDQAIRAGL
              660 670 680 690 700
ELLKVLSFTH PISFHSAETF ESLLACLKMD DEKVAEAALQ IFKNTGSKIE
              710 720 730 740 750
EDFPHIRSAL LPVLHHKSKK GPPRQAKYAI HCIHAIFSSK ETQFAQIFEP
              760 770 780 790 800
LHKSLDPSNL EHLITPLVTI GHIALLAPDQ FAAPLKSLVA TFIVKDLLMN
              810 820 830 840 850
DRLPGKKTTK LWVPDEEVSP ETMVKIQAIK MMVRWLLGMK NNHSKSGTST
              860 870 880 890 900
LRLLTTILHS DGDLTEQGKI SKPDMSRLRL AAGSAIVKLA QEPCYHEIIT
              910 920 930 940 950
LEQYQLCALA INDECYQVRQ VFAQKLHKGL SRLRLPLEYM AICALCAKDP
              960 970 980 990 1000
VKERRAHARQ CLVKNINVRR EYLKQHAAVS EKLLSLLPEY VVPYTIHLLA
            1010 1020 1030 1040 1050
HDPDYVKVQD IEQLKDVKEC LWFVLEILMA KNENNSHAFI RKMVENIKQT
            1060 1070 1080 1090 1100
KDAQGPDDAK MNEKLYTVCD VAMNIIMSKS TTYSLESPKD PVLPARFFTQ
            1110 1120 1130 1140 1150
PDKNFSNTKN YLPPEMKSFF TPGKPKTTNV LGAVNKPLSS AGKQSQTKSS
            1160 1170 1180 1190 1200
RMETVSNASS SSNPSSPGRI KGRLDSSEMD HSENEDYTMS SPLPGKKSDK
            1210 1220 1230 1240 1250
RDDSDLVRSE LEKPRGRKKT PVTEQEEKLG MDDLTKLVQE QKPKGSQRSR
            1260 1270 1280 1290 1300
KRGHTASESD EQQWPEEKRL KEDILENEDE QNSPPKKGKR GRPPKPLGGG
            1310 1320 1330 1340 1350
TPKEEPTMKT SKKGSKKKSG PPAPEEEEEE ERQSGNTEQK SKSKQHRVSR
            1360 1370 1380 1390 1400
RAQQRAESPE SSAIESTQST PQKGRGRPSK TPSPSQPKKN VRVGRSKQAA
            1410 1420 1430 1440
TKENDSSEEV DVFQGSSPVD DIPQEETEEE EVSTVNVRRR SAKRERR
```

This protein is encoded by a cDNA sequence with accession number U95825 in the NCBI database.

An example of a human positive BTN3A1 regulator sequence for a CNOT11 protein is shown below (Uniprot Q9UKZ1; SEQ ID NO:74).

```
                10 20 30 40 50
MPGGGASAAS GRLLTAAEQR GSREAAGSAS RSGEGGSGGG RGGASGPGSG
                60 70 80 90 100
SGGPGGPAGR MSLTPKELSS LLSIISEEAG GGSTFEGLST AFHHYFSKAD
              110 120 130 140 150
HERLGSVLVM LLQQPDLLPS AAQRLTALYL LWEMYRTEPL AANPFAASFA
              160 170 180 190 200
HLINPAPPAR GGQEPDRPPL SGFLPPITPP EKFFLSQLML APPRELFKKT
              210 220 230 240 250
PROIALMDVG NMGQSVDISG LOLALAERQS ELPTOSKASE PSILSDPDPD
              260 270 280 290 300
SSNSGEDSSV ASQITEALVS GPKPPIESHF RPEFIRPPPP LHICEDELAW
              310 320 330 340 350
LNPTEPDHAI QWDKSMCVKN STGVEIKRIM AKAFKSPLSS PQQTOLLGEL
              360 370 380 390 400
EKDPKLVYHI GLTPAKLPDL VENNPLVAIE MLLKLMOSSQ ITEYFSVLVN
              410 420 430 440 450
MDMSLHSMEV VNRLTTAVDL PPEFIHLYIS NCISTCEQIK DKYMQNRLVR
              460 470 480 490 500
LVCVFLQSLI RNKIINVODL FIEVQAFCIE FSRIREAAGL FRLLKTLDTG
              510
ETPSETKMSK
```

This protein is encoded by a cDNA sequence with accession number AF103798 in the NCBI database.

An example of a human positive BTN3A1 regulator sequence for a NDUFB7 protein is shown below (Uniprot P17568; SEQ ID NO:75).

```
                10 20 30 40 50
MGAHLVRRYL GDASVEPDPL QMPTFPPDYG FPERKEREMV ATQQEMMDAQ
                60 70 80 90 100
LRLQLRDYCA HHLIRLIKCK RDSFPNFLAC KQERHDWDYC EHRDYVMRMK
              110 120 130
EFERERRLLQ RKKRREKKAA ELAKGQGPGE VDPKVAL
```

This protein is encoded by a cDNA sequence with accession number M33374 in the NCBI database.

An example of a human positive BTN3A1 regulator sequence for a BTN3A2 protein is shown below (Uniprot P78410; SEQ ID NO:76).

```
                10 20 30 40 50
MKMASSLAFL LLNFHVSLLL VQLLTPCSAQ FSVLGPSGPI LAMVGEDADL
                60 70 80 90 100
PCHLFPTMSA ETMELKWVSS SLRQVVNVYA DGKEVEDRQS APYRGRTSIL
              110 120 130 140 150
RDGITAGKAA LRIHNVTASD SGKYLCYFQD GDFYEKALVE LKVAALGSNL
              160 170 180 190 200
HVEVKGYEDG GIHLECRSTG WYPQPQIQWS NAKGENIPAV EAPVVADGVG
              210 220 230 240 250
LYEVAASVIM RGGSGEGVSC IIRNSLLGLE KTASISIADP FFRSAQPWIA
              260 270 280 290 300
ALAGTLPILL LLLAGASYFL WRQQKEITAL SSEIESEQEM KEMGYAATER
              310 320 330
EISLRESLQE ELKRKKIQYL TRGEESSSDT NKSA
```

This protein is encoded by a cDNA sequence with accession number U90546 in the NCBI database.

An example of a human positive BTN3A1 regulator sequence for a FOXRED1 protein is shown below (Uniprot Q96CU9; SEQ ID NO:77).

```
                10 20 30 40 50
MIRRVLPHGM GRGLLTRRPG TRRGGFSLDW DGKVSEIKKK IKSILPGRSC
                60 70 80 90 100
DLLQDTSHLP PEHSDVVIVG GGVLGLSVAY WLKKLESRRG AIRVLVVERD
              110 120 130 140 150
HTYSQASTGL SVGGICQQFS LPENIQLSLE SASFLRNINE YLAVVDAPPL
              160 170 180 190 200
DLRENPSGYL LLASEKDAAA MESNVKVQRQ EGAKVSLMSP DQLRNKFPWI
              210 220 230 240 250
NTEGVALASY GMEDEGWFDP WCLLQGLRRK VQSLGVLFCQ GEVTREVSSS
              260 270 280 290 300
QRMLTTDDKA VVLKRIHEVH VKMDRSLEYQ PVECAIVINA AGAWSAQIAA
              310 320 330 340 350
LAGVGEGPPG TLQGTKLPVE PRKRYVYVWH CPQGPGLETP LVADTSGAYF
              360 370 380 390 400
RREGLGSNYL GGRSPTEQEE PDPANLEVDH DEFQDKVWPH LALRVPAFET
              410 420 430 440 450
LKVQSAWAGY YDYNTFDQNG VVGPHPLVVN MYFATGFSGH GLQQAPGIGR
              460 470 480
AVAEMVLKGR FQTIDLSPFL FTRFYLGEKI QENNII
```

This protein is encoded by a cDNA sequence with accession number AF103801 in the NCBI database.

An example of a human positive BTN3A1 regulator sequence for a NDUFS8 protein is shown below (Uniprot O00217; SEQ ID NO:78).

```
                10 20 30 40 50
MRCLTTPMLL RALAQAARAG PPGGRSLHSS AVAATYKYVN MQDPEMDMKS
                60 70 80 90 100
VIDRAARTLL WTELFRGIGM TLSYLFREPA TINYPFEKGP LSPRERGEHA
              110 120 130 140 150
LRRYPSGEER CIACKLCEAI CPAQAITIEA EPRADGSRRT TRYDIDMTKC
              160 170 180 190 200
IYCGFCQEAC PVDAIVEGPN FEESTETHEE LLYNKEKLIN NGDKWEAEIA
              210
ANIQADYLYR
```

This protein is encoded by a cDNA sequence with accession number U65579 in the NCBI database.

An example of a human positive BTN3A1 regulator sequence for a JMJD6 protein is shown below (Uniprot Q6NYC1; SEQ ID NO: 79).

```
                10 20 30 40 50
MNHKSKKRIR EAKRSARPEL KDSLDWTRHN YYESFSLSPA AVADNVERAD
                60 70 80 90 100
ALQLSVEEFV ERYERPYKPV VLLNAQEGWS AQEKWTLERL KRKYRNQKFK
              110 120 130 140 150
CGEDNDGYSV KMKMKYYIEY MESTRDDSPL YIFDSSYGEH PKRRKLLEDY
              160 170 180 190 200
KVPKFFTDDL FQYAGEKRRP PYRWFVMGPP RSGTGIHIDP LGTSAWNALV
              210 220 230 240 250
QGHKRWCLFP TSTPRELIKV TRDEGGNQQD EAITWFNVIY PRTQLPTWPP
              260 270 280 290 300
EFKPLEILOK PGETVFVPGG WWHVVLNLDT TIAITQNFAS STNFPVVWHK
              310 320 330 340 350
TVRGRPKLSR KWYRILKQEH PELAVLADSV DLQESTGIAS DSSSDSSSSS
              360 370 380 390 400
SSSSSDSDSE CESGSEGDGT VHRRKKRRTC SMVGNGDTTS QDDCVSKERS SSR
```

This protein is encoded by a cDNA sequence with accession number AB073711 in the NCBI database.

An example of a human positive BTN3A1 regulator sequence for a NDUFS2 protein is shown below (Uniprot O75306; SEQ ID NO:80).

```
                10 20 30 40 50
MAALRALCGF RGVAAQVLRP GAGVRLPIQP SRGVRQWQPD VEWAQQEGGA
                60 70 80 90 100
VMYPSKETAH WKPPPWNDVD PPKDTIVKNI TLNFGPQHPA AHGVLRLVME
              110 120 130 140 150
LSGEMVRKCD PHIGLLHRGT EKLIEYKTYL QALPYFDRLD YVSMMCNEQA
              160 170 180 190 200
YSLAVEKLLN IRPPPRAQWI RVLFGEITRL LNHIMAVTTH ALDLGAMTPE
              210 220 230 240 250
FWLFEEREKM FEFYERVSGA RMHAAYIRPG GVHODLPLGL MDDIYQESKN
              260 270 280 290 300
FSLRLDELEE LLTNNRIWRN RTIDIGVVTA EEALNYGFSG VMLRGSGIQW
              310 320 330 340 350
DLRKTQPYDV YDQVEFDVPV GSRGDCYDRY LCRVEEMRQS LRIIAQCLNK
              360 370 380 390 400
MPPGEIKVDD AKVSPPKRAE MKTSMESLIH HEKLYTEGYQ VPPGATYTAI
              410 420 430 440 450
EAPKGEFGVY LVSDGSSRPY RCKIKAPGFA HLAGLDKMSK GHMLADVVAI
              460
IGTQDIVEGE VDR
```

This protein is encoded by a cDNA sequence with accession number AF050640 in the NCBI database.

An example of a human positive BTN3A1 regulator sequence for a NDUFC2 protein is shown below (Uniprot O95298; SEQ ID NO:81).

```
                10 20 30 40 50
MIARRNPEPL RFLPDEARSL PPPKLIDPRL LYIGELGYCS GLIDNLIRRR
                60 70 80 90 100
PIATAGLHRQ LLYITAFFFA GYYLVKREDY LYAVRDREMF GYMKLHPEDE
              110
PEEDKKTYGE IFEKFHPIR
```

This protein is encoded by a cDNA sequence with accession number AF087659 in the NCBI database.

An example of a human positive BTN3A1 regulator sequence for a HSF1 protein is shown below (Uniprot Q00613: SEQ ID NO:82).

```
                10 20 30 40
MDLPVGPGAA GPSNVPAFLT KLWTLVSDPD TDALICWSPS
                50 60 70 80
GNSFHVFDQG QFAKEVLPKY FKHNNMASFV RQLNMYGFRK
                90 100 110 120
VVHIEQGGLV KPERDDTEFQ HPCFLRGQEQ LLENIKRKVT
              130 140 150 160
SVSTLKSEDI KIRQDSVTKL LTDVQLMKGK QECMDSKLLA
              170 180 190 200
MKHENEALWR EVASLRQKHA QQQKVVNKLI QFLISLVQSN
              210 220 230 240
RILGVKRKIP LMLNDSGSAH SMPKYSRQFS LEHVHGSGPY
              250 260 270 280
SAPSPAYSSS SLYAPDAVAS SGPIISDITE LAPASPMASP
              290 300 310 320
GGSIDERPLS SSPLVRVKEE PPSPPQSPRV EEASPGRPSS
              330 340 350 360
VDTLLSPTAL IDSILRESEP APASVTALTD ARGHTDTEGR
              370 380 390 400
PPSPPPTSTP EKCLSVACLD KNELSDHLDA MDSNLDNLQT
              410 420 430 440
MLSSHGFSVD TSALLDLFSP SVTVPDMSLP DLDSSLASIQ
              450 460 470 480
ELLSPQEPPR PPEAENSSPD SGKQLVHYTA QPLFLLDPGS
              490 500 510 520
VDTGSNDLPV LFELGEGSYF SEGDGFAEDP TISLLTGSEP
PKAKDPTVS
```

This protein is encoded by a cDNA sequence with accession number M64673 in the NCBI database.

An example of a human positive BTN3A1 regulator sequence for an ACAD9 protein is shown below (Uniprot Q9H845; SEQ ID NO:83).

```
                10 20 30 40
MSGCGLFLRT TAAARACRGL VVSTANRRLL RTSPPVRAFA
                50 60 70 80
KELFLGKIKK KEVFPFPEVS QDELNEINQF LGPVEKFFTE
                90 100 110 120
EVDSRKIDQE GKIPDETLEK LKSLGLFGLQ VPEEYGGLGF
              130 140 150 160
SNTMYSRLGE IISMDGSITV TLAAHQAIGL KGIILAGTEE
              170 180 190 200
QKAKYLPKLA SGEHIAAFCL TEPASGSDAA SIRSRATLSE
              210 220 230 240
DKKHYILNGS KVWITNGGLA NIFTVFAKTE VVDSDGSVKD
              250 260 270 280
KITAFIVERD FGGVINGKPE DKLGIRGSNT CEVHFENTKI
              290 300 310 320
PVENILGEVG DGFKVAMNIL NSGRFSMGSV VAGLLKRLIE
              330 340 350 360
MTAEYACTRK QFNKRLSEFG LIQEKFALMA QKAYVMESMT
              370 380 390 400
YLTAGMLDQP GFPDCSIEAA MVKVFSSEAA WQCVSEALQI
              410 420 430 440
LGGLGYTRDY PYERILRDTR ILLIFEGTNE ILRMYIALTG
              450 460 470 480
LQHAGRILTT RIHELKQAKV STVMDTVGRR LRDSLGRTVD
              490 500 510 520
LGLTGNHGVV HPSLADSANK FEENTYCFGR TVETLLLRFG
              530 540 550 560
KTIMEEQLVL KRVANILINL YGMTAVLSRA SRSIRIGLRN
              570 580 590 600
HDHEVLLANT FCVEAYLQNL FSLSQLDKYA PENLDEQIKK
              610 620
VSQQILEKRA YICAHPLDRT C
```

This protein is encoded by a cDNA sequence with accession number AF327351 in the NCBI database.

An example of a human positive BTN3A1 regulator sequence for a NDUFAF5 protein is shown below (Uniprot Q5TEU4; SEQ ID NO:84).

```
                10 20 30 40
MLRPAGLWRL CRRPWAARVP AENLGRREVT SGVSPRGSTS
                50 60 70 80
PRTLNIFDRD LKRKQKNWAA RQPEPTKFDY LKEEVGSRIA
                90 100 110 120
DRVYDIPRNF PLALDLGCGR GYIAQYINKE TIGKFFQADI
              130 140 150 160
AENALKNSSE TEIPTVSVLA DEEFLPFKEN TFDLVVSSLS
              170 180 190 200
LHWVNDLPRA LEQIHYILKP DGVFIGAMFG GDTLYELRCS
              210 220 230 240
LQLAETEREG GFSPHISPFT AVNDLGHLLG RAGFNTLTVD
              250 260 270 280
TDEIQVNYPG MFELMEDLQG MGESNCAWNR KALLHRDTML
              290 300 310 320
AAAAVYREMY RNEDGSVPAT YQIYYMIGWK YHESQARPAE
              330 340
RGSATVSFGE LGKINNLMPP GKKSQ
```

This protein is encoded by a cDNA sequence with accession number AK025977 in the NCBI database.

An example of a human positive BTN3A1 regulator sequence for a TIMMDC1 protein is shown below (Uniprot Q9NPL8; SEQ ID NO:85).

```
                10 20 30 40
MEVPPPAPRS FLCRALCLFP RVFAAEAVTA DSEVLEERQK
                50 60 70 80
RLPYVPEPYY PESGWDRIRE LFGKDEQQRI SKDLANICKT
                90 100 110 120
AATAGIIGWV YGGIPAFIHA KQQYIEQSQA EIYHNRFDAV
              130 140 150 160
QSAHRAATRG FIRYGWRWGW RTAVFVTIFN TVNTSLNVYR
              170 180 190 200
NKDALSHFVI AGAVTGSLFR INVGLRGLVA GGIIGALLGT
              210 220 230 240
PVGGLIMAFQ KYSGETVQER KQKDRKALHE LKLEEWKGRL
              250 260 270 280
QVTEHLPEKI ESSLQEDEPE NDAKKIEALL NLPRNPSVID
KQDKD
```

This protein is encoded by a cDNA sequence with accession number AF210057 in the NCBI database.

An example of a human positive BTN3A1 regulator sequence for a HSD17B10 protein is shown below (Uniprot Q99714; SEQ ID NO:86).

```
                10 20 30 40
MAAACRSVKG LVAVITGGAS GLGLATAERL VGQGASAVLL
                50 60 70 80
DLPNSGGEAQ AKKLGNNCVF APADVTSEKD VQTALALAKG
                90 100 110 120
KFGRVDVAVN CAGIAVASKT YNLKKGQTHT LEDFQRVLDV
              130 140 150 160
NLMGTFNVIR LVAGEMGQNE PDQGGQRGVI INTASVAAFE
              170 180 190 200
GQVGQAAYSA SKGGIVGMTL PIARDLAPIG IRVMTIAPGL
              210 220 230 240
FGTPLLTSLP EKVQNFLASQ VPFPSRLGDP AEYAHLVQAI
              250 260
IENPFLNGEV IRLDGAIRMQ P
```

This protein is encoded by a cDNA sequence with accession number U96132 in the NCBI database.

An example of a human positive BTN3A1 regulator sequence for a BRD2 protein is shown below (Uniprot P25440; SEQ ID NO:87).

```
                10 20 30 40
MLQNVTPHNK LPGEGNAGLL GLGPEAAAPG KRIRKPSLLY
                50 60 70 80
EGFESPTMAS VPALQLTPAN PPPPEVSNPK KPGRVTNQLQ
                90 100 110 120
YLHKVVMKAL WKHQFAWPFR QPVDAVKLGL PDYHKIIKQP
              130 140 150 160
MDMGTIKRRL ENNYYWAASE CMQDFNTMFT NCYIYNKPTD
              170 180 190 200
DIVLMAQTLE KIFLQKVASM PQEEQELVVT IPKNSHKKGA
              210 220 230 240
KLAALQGSVT SAHQVPAVSS VSHTALYTPP PEIPTTVLNI
              250 260 270 280
PHPSVISSPL LKSLHSAGPP LLAVTAAPPA QPLAKKKGVK
              290 300 310 320
RKADTTTPTP TAILAPGSPA SPPGSLEPKA ARLPPMRRES
              330 340 350 360
GRPIKPPRKD LPDSQQQHQS SKKGKLSEQL KHQNGILKEL
              370 380 390 400
LSKKHAAYAW PFYKPVDASA LGLHDYHDII KHPMDLSTVK
              410 420 430 440
RKMENRDYRD AQEFAADVRL MFSNCYKYNP PDHDVVAMAR
              450 460 470 480
KLQDVFEFRY AKMPDEPLEP GPLPVSTAMP PGLAKSSSES
              490 500 510 520
SSEESSSESS SEEEEEEDEE DEEEEESESS DSEEERAHRL
              530 540 550 560
AELQEQLRAV HEQLAALSQG PISKPKRKRE KKEKKKKRKA
              570 580 590 600
EKHRGRAGAD EDDKGPRAPR PPQPKKSKKA SGSGGGSAAL
              610 620 630 640
GPSGFGPSGG SGTKLPKKAT KTAPPALPTG YDSEEEEESR
              650 660 670 680
PMSYDEKRQL SLDINKLPGE KLGRVVHIIQ AREPSLRDSN
              690 700 710 720
PEEIEIDFET LKPSTLRELE RYVLSCLRKK PRKPYTIKKP
              730 740 750 760
VGKTKEELAL EKKRELEKRL QDVSGQLNST KKPPKKANEK
              770 780 790 800
TESSSAQQVA VSRLSASSSS SDSSSSSSSS SSSDTSDSDS
```

This protein is encoded by a cDNA sequence with accession number X62083 in the NCBI database.

An example of a human positive BTN3A1 regulator sequence for a NDUFA6 protein is shown below (Uniprot P56556; SEQ ID NO:88).

```
                10 20 30 40
MAGSGVRQAT STASTFVKPI FSRDMNEAKR RVRELYRAWY
                50 60 70 80
REVPNTVHQF QLDITVKMGR DKVREMFMKN AHVTDPRVVD
                90 100 110 120
LIVIKGKIEL EETIKVWKQR THVMRFFHET EAPRPKDELS
KFYVGHDP
```

This protein is encoded by a cDNA sequence with accession number AF047182 in the NCBI database.

An example of a human positive BTN3A1 regulator sequence for a CNOT4 protein is shown below (Uniprot O95628; SEQ ID NO:89).

```
                10 20 30 40
MSRSPDAKED PVECPLCMEP LEIDDINFFP CTCGYQICRF
                50 60 70 80
CWHRIRTDEN GLCPACRKPY PEDPAVYKPL SQEELQRIKN
                90 100 110 120
EKKQKQNERK QKISENRKHL ASVRVVQKNL VFVVGLSQRL
              130 140 150 160
ADPEVLKRPE YFGKFGKIHK VVINNSTSYA GSQGPSASAY
              170 180 190 200
VTYIRSEDAL RAIQCVNNVV VDGRTLKASL GTTKYCSYFL
              210 220 230 240
KNMQCPKPDC MYLHELGDEA ASFTKEEMQA GKHQEYEQKL
              250 260 270 280
LQELYKLNPN FLQLSTGSVD KNKNKVTPLQ RYDTPIDKPS
              290 300 310 320
DSLSIGNGDN SQQISNSDTP SPPPGLSKSN PVIPISSSNH
              330 340 350 360
SARSPFEGAV TESQSLFSDN FRHPNPIPSG LPPFPSSPQT
              370 380 390 400
SSDWPTAPEP QSLFTSETIP VSSSTDWQAA FGFGSSKQPE
              410 420 430 440
DDLGFDPFDV TRKALADLIE KELSVQDQPS ISPTSLQNSS
              450 460 470 480
SHTTTAKGPG SGFLHPAAAT NANSLNSTFS VLPQRFPQFQ
              490 500 510 520
QHRAVYNSFS FPGQAARYPW MAFPRNSIMH LNHTANPTSN
              530 540 550 560
SNFLDLNLPP QHNTGLGGIP VAGEEEVKVS IMPLSTSSHS
              570
LQQGQQPTSL HTTVA
```

This protein is encoded by a cDNA sequence with accession number U71267 in the NCBI database.

An example of a human positive BTN3A1 regulator sequence for a SPI1 protein is shown below (Uniprot P17947; SEQ ID NO:90).

```
                10 20 30 40
MLQACKMEGF PLVPPPSEDL VPYDTDLYQR QTHEYYPYLS
                50 60 70 80
SDGESHSDHY WDFHPHHVHS EFESFAENNF TELQSVQPPQ
                90 100 110 120
LQQLYRHMEL EQMHVIDTPM VPPHPSIGHQ VSYLPRMCLQ
              130 140 150 160
YPSLSPAQPS SDEEEGERQS PPLEVSDGEA DGLEPGPGLL
              170 180 190 200
PGETGSKKKI RLYQFLLDLL RSGDMKDSIW WVDKDKGTFQ
              210 220 230 240
FSSKHKEALA HRWGIQKGNR KKMTYQKMAR ALRNYGKTGE
              250 260 270
VKKVKKKLTY QFSGEVIGRG GLAERRHPPH
```

This protein is encoded by a cDNA sequence with accession number X52056 in the NCBI database.

An example of a human positive BTN3A1 regulator sequence for a MDH2 protein is shown below (Uniprot P40926; SEQ ID NO:91).

```
                10 20 30 40
MLSALARPAS AALRRSFSTS AQNNAKVAVL GASGGIGQPL
                50 60 70 80
SLLLKNSPLV SRLTLYDIAH TPGVAADLSH IETKAAVKGY
                90 100 110 120
LGPEQLPDCL KGCDVVVIPA GVPRKPGMTR DDLFNTNATI
              130 140 150 160
VATLTAACAQ HCPEAMICVI ANPVNSTIPI TAEVFKKHGV
              170 180 190 200
YNPNKIFGVT TLDIVRANTF VAELKGLDPA RVNVPVIGGH
              210 220 230 240
AGKTIIPLIS QCTPKVDFPQ DQLTALTGRI QEAGTEVVKA
              250 260 270 280
KAGAGSATLS MAYAGARFVF SLVDAMNGKE GVVECSFVKS
              290 300 310 320
QETECTYFST PLLLGKKGIE KNIGIGKVSS FEEKMISDAI
              330
PELKASIKKG EDFVKTLK
```

This protein is encoded by a cDNA sequence with accession number AF047470 in the NCBI database.

An example of a human positive BTN3A1 regulator sequence for a DARS2 protein is shown below (Uniprot Q6PI48; SEQ ID NO:92).

```
                10 20 30 40
MYFPSWLSQL YRGLSRPIRR TTQPIWGSLY RSLLQSSQRR
                50 60 70 80
IPEFSSFVVR TNTCGELRSS HLGQEVTLCG WIQYRRQNTF
                90 100 110 120
LVLRDFDGLV QVIIPQDESA ASVKKILCEA PVESVVQVSG
              130 140 150 160
TVISRPAGQE NPKMPTGEIE IKVKTAELLN ACKKLPFEIK
              170 180 190 200
NFVKKTEALR LQYRYLDLRS FQMQYNLRLR SQMVMKMREY
              210 220 230 240
LCNLHGFVDI ETPTLFKRTP GGAKEFLVPS REPGKFYSLP
              250 260 270 280
QSPQQFKQLL MVGGLDRYFQ VARCYRDEGS RPDRQPEFTQ
              290 300 310 320
IDIEMSFVDQ TGIQSLIEGL LQYSWPNDKD PVVVPFPTMT
              330 340 350 360
FAEVLATYGT DKPDTRFGMK IIDISDVFRN TEIGFLQDAL
              370 380 390 400
SKPHGTVKAI CIPEGAKYLK RKDIESIRNF AADHFNQEIL
              410 420 430 440
PVFLNANRNW NSPVANFIME SQRLELIRLM ETQEEDVVLL
              450 460 470 480
TAGEHNKACS LLGKLRLECA DLLETRGVVL RDPTLFSFLW
              490 500 510 520
VVDFPLFLPK EENPRELESA HHPFTAPHPS DIHLLYTEPK
              530 540 550 560
KARSQHYDLV LNGNEIGGGS IRIHNAELQR YILATLLKED
              570 580 590 600
VKMISHLLQA LDYGAPPHGG IALGLDRLIC LVTGSPSIRD
              610 620 630 640
VIAFPKSFRG HDLMSNTPDS VPPEELKPYH IRVSKPTDSK
AERAH
```

This protein is encoded by a cDNA sequence with accession number BC045173 in the NCBI database.

An example of a human positive BTN3A1 regulator sequence for a TMEM261 protein is shown below (Uniprot Q96GE9; SEQ ID NO:93).

```
                10 20 30 40
MGSRLSQPFE SYITAPPGTA AAPAKPAPPA TPGAPTSPAE
                50 60 70 80
HRLLKTCWSC RVLSGLGLMG AGGYVYWVAR KPMKMGYPPS
                90 100 110
PWTITQMVIG LSENQGIATW GIVVMADPKG KAYRVV
```

This protein is encoded by a cDNA sequence with accession number AK292632 in the NCBI database.

An example of a human positive BTN3A1 regulator sequence for a STIP1 protein is shown below (Uniprot P31948; SEQ ID NO:94).

```
                10 20 30 40
MEQVNELKEK GNKALSVGNI DDALQCYSEA IKLDPHNHVL
                50 60 70 80
YSNRSAAYAK KGDYQKAYED GCKTVDLKPD WGKGYSRKAA
                90 100 110 120
ALEFLNRFEE AKRTYEEGLK HEANNPQLKE GLQNMEARLA
              130 140 150 160
ERKFMNPENM PNLYQKLESD PRTRILLSDP TYRELIEQLR
              170 180 190 200
NKPSDLGTKL QDPRIMTTLS VLLGVDLGSM DEEEEIATPP
              210 220 230 240
PPPPPKKETK PEPMEEDLPE NKKQALKEKE LGNDAYKKKD
              250 260 270 280
FDTALKHYDK AKELDPTNMT YITNQAAVYF EKGDYNKCRE
              290 300 310 320
LCEKAIEVGR ENREDYRQIA KAYARIGNSY FKEEKYKDAI
              330 340 350 360
HFYNKSLAEH RTPDVLKKCQ QAEKILKEQE RLAYINPDLA
              370 380 390 400
LEEKNKGNEC FQKGDYPQAM KHYTEAIKRN PKDAKLYSNR
              410 420 430 440
AACYTKLLEF QLALKDCEEC IQLEPTFIKG YTRKAAALEA
              450 460 470 480
MKDYTKAMDV YQKALDLDSS CKEAADGYQR CMMAQYNRHD
              490 500 510 520
SPEDVKRRAM ADPEVQQIMS DPAMRLILEQ MQKDPQALSE
              530 540
HLKNPVIAQK IQKLMDVGLI AIR
```

This protein is encoded by a cDNA sequence with accession number M86752 in the NCBI database.

An example of a human positive BTN3A1 regulator sequence for a FIBP protein is shown below (Uniprot O43427; SEQ ID NO:95).

```
                10 20 30 40
MTSELDIFVG NTTLIDEDVY RLWLDGYSVT DAVALRVRSG
                50 60 70 80
ILEQTGATAA VLQSDTMDHY RTFHMLERLL HAPPKLLHQL
                90 100 110 120
IFQIPPSRQA LLIERYYAFD EAFVREVLGK KLSKGTKKDL
              130 140 150 160
DDISTKTGIT LKSCRRQFDN FKRVFKVVEE MRGSLVDNIQ
              170 180 190 200
QHFLLSDRLA RDYAAIVFFA NNRFETGKKK LQYLSFGDFA
              210 220 230 240
FCAELMIQNW TLGAVGEAPT DPDSQMDDMD MDLDKEFLQD
              250 260 270 280
LKELKVLVAD KDLLDLHKSL VCTALRGKLG VFSEMEANFK
              290 300 310 320
NLSRGLVNVA AKLTHNKDVR DLFVDLVEKF VEPCRSDHWP
              330 340 350 360
LSDVRFFLNQ YSASVHSLDG FRHQALWDRY MGTLRGCLLR
LYHD
```

This protein is encoded by a cDNA sequence with accession number AF010187 in the NCBI database.

An example of a human positive BTN3A1 regulator sequence for a FXR1 protein is shown below (Uniprot P51114; SEQ ID NO:96).

```
                10 20 30 40
MAELTVEVRG SNGAFYKGFI KDVHEDSLIV VFENNWQPER
                50 60 70 80
QVPFNEVRLP PPPDIKKEIS EGDEVEVYSR ANDQEPCGWW
                90 100 110 120
LAKVRMMKGE FYVIEYAACD ATYNEIVTFE RLRPVNQNKT
              130 140 150 160
VKKNTFFKCT VDVPEDLREA CANENAHKDF KKAVGACRIF
              170 180 190 200
YHPETTQLMI LSASEATVKR VNILSDMHLR SIRTKLMLMS
              210 220 230 240
RNEEATKHLE CTKQLAAAFH EEFVVREDLM GLAIGTHGSN
              250 260 270 280
IQQARKVPGV TAIELDEDTG TFRIYGESAD AVKKARGFLE
              290 300 310 320
FVEDFIQVPR NLVGKVIGKN GKVIQEIVDK SGVVRVRIEG
              330 340 350 360
DNENKLPRED GMVPFVFVGT KESIGNVQVL LEYHIAYLKE
              370 380 390 400
VEQLRMERLQ IDEQLRQIGS RSYSGRGRGR RGPNYTSGYG
              410 420 430 440
TNSELSNPSE TESERKDELS DWSLAGEDDR DSRHQRDSRR
              450 460 470 480
RPGGRGRSVS GGRGRGGPRG GKSSISSVLK DPDSNPYSLL
              490 500 510 520
DNTESDQTAD TDASESHHST NRRRRSRRRR TDEDAVIMDG
              530 540 550 560
MTESDTASVN ENGLVIVADY ISRAESQSRQ RNLPRETLAK
              570 580 590 600
NKKEMAKDVI EEHGPSEKAI NGPTSASGDD ISKLQRTPGE
              610 620
EKINTLKEEN TQEAAVINGV S
```

This protein is encoded by a cDNA sequence with accession number U25165 in the NCBI database.

An example of a human positive BTN3A1 regulator sequence for a NFU1 protein is shown below (Uniprot Q9UMS0; SEQ ID NO:97).

```
                10 20 30 40
MAATARRGWG AAAVAAGLRR RFCHMLKNPY TIKKQPLHQF
                50 60 70 80
VQRPLFPLPA AFYHPVRYMF IQTQDTPNPN SLKFIPGKPV
                90 100 110 120
LETRIMDFPT PAAAFRSPLA RQLFRIEGVK SVFFGPDFIT
              130 140 150 160
VTKENEELDW NLLKPDIYAT IMDFFASGLP LVTEETPSGE
              170 180 190 200
AGSEEDDEVV AMIKELLDTR IRPTVQEDGG DVIYKGFEDG
              210 220 230 240
IVQLKLQGSC TSCPSSIITL KNGIQNMLQF YIPEVEGVEQ
              250
VMDDESDEKE ANSP
```

This protein is encoded by a cDNA sequence with accession number AJ132584 in the NCBI database.

An example of a human positive BTN3A1 regulator sequence for a GGNBP2 protein is shown below (Uniprot Q9H3C7; SEQ ID NO:98).

```
                10 20 30 40
MARLVAVCRD GEEEFPFERR QIPLYIDDTL TMVMEFPDNV
                50 60 70 80
LNLDGHQNNG AQLKQFIQRH GMLKQQDLSI AMVVTSREVL
                90 100 110 120
SALSQLVPCV GQRRSVERLF SQLVESGNPA LEPLTVGPKG
              130 140 150 160
VLSVIRSCMT DAKKLYTLFY VHGSKINDMI DAIPKSKKNK
              170 180 190 200
RCQLHSLDTH KPKPIGGCWM DVWELMSQEC RDEVVLIDSS
              210 220 230 240
CLLETLETYL RKHRFCTDCK NKVLRAYNIL IGELDCSKEK
              250 260 270 280
GYCAALYEGL RCCPHERHIH VCCETDFIAH LLGRAEPEFA
              290 300 310 320
GGRRERHAKT IDIAQEEVLT CLGIHLYERL HRIWQKLRAE
              330 340 350 360
EQTWQMLFYL GVDALRKSFE MTVEKVQGIS RLEQLCEEFS
              370 380 390 400
EEERVRELKQ EKKRQKRKNR RKNKCVCDIP TPLQTADEKE
              410 420 430 440
VSQEKETDFI ENSSCKACGS TEDGNTCVEV IVTNENTSCT
              450 460 470 480
CPSSGNLLGS PKIKKGLSPH CNGSDCGYSS SMEGSETGSR
              490 500 510 520
EGSDVACTEG ICNHDEHGDD SCVHHCEDKE DDGDSCVECW
              530 540 550 560
ANSEENDTKG KNKKKKKKSK ILKCDEHIQK LGSCITDPGN
              570 580 590 600
RETSGNTMHT VFHRDKTKDT HPESCCSSEK GGQPLPWFEH
              610 620 630 640
RKNVPQFAEP TETLEGPDSG KGAKSLVELL DESECTSDEE
              650 660 670 680
IFISQDEIQS FMANNQSFYS NREQYRQHLK EKFNKYCRLN
              690
DHKRPICSGW LTTAGAN
```

This protein is encoded by a cDNA sequence with accession number AF268387 in the NCBI database.

An example of a human positive BTN3A1 regulator sequence for a STAT2 protein is shown below (Uniprot P52630; SEQ ID NO:99).

```
                10 20 30 40
MAQWEMLQNL DSPFQDQLHQ LYSHSLLPVD IRQYLAVWIE
                50 60 70 80
DQNWQEAALG SDDSKATMLF FHFLDQLNYE CGRCSQDPES
                90 100 110 120
LLLQHNLRKF CRDIQPFSQD PTQLAEMIFN LLLEEKRILI
              130 140 150 160
QAQRAQLEQG EPVLETPVES QQHEIESRIL DLRAMMEKLV
              170 180 190 200
KSISQLKDQQ DVFCFRYKIQ AKGKTPSLDP HQTKEQKILQ
              210 220 230 240
ETLNELDKRR KEVLDASKAL LGRLTTLIEL LLPKLEEWKA
              250 260 270 280
QQQKACIRAP IDHGLEQLET WFTAGAKLLF HLRQLLKELK
              290 300 310 320
GLSCLVSYQD DPLTKGVDLR NAQVTELLQR LLHRAFVVET
              330 340 350 360
QPCMPQTPHR PLILKTGSKF TVRTRLLVRL QEGNESLTVE
              370 380 390 400
VSIDRNPPQL QGFRKFNILT SNQKTLTPEK GQSQGLIWDF
              410 420 430 440
GYLTLVEQRS GGSGKGSNKG PLGVTEELHI ISFTVKYTYQ
              450 460 470 480
GLKQELKTDT LPVVIISNMN QLSIAWASVL WFNLLSPNLQ
              490 500 510 520
NQQFFSNPPK APWSLLGPAL SWQFSSYVGR GLNSDQLSML
              530 540 550 560
RNKLFGQNCR TEDPLLSWAD FTKRESPPGK LPFWTWLDKI
              570 580 590 600
LELVHDHLKD LWNDGRIMGF VSRSQERRLL KKTMSGTFLL
              610 620 630 640
RFSESSEGGI TCSWVEHQDD DKVLIYSVQP YTKEVLQSLP
              650 660 670 680
LTEIIRHYQL LTEENIPENP IRFLYPRIPR DEAFGCYYQE
            690 700 710 720
KVNLQERRKY LKHRLIVVSN RQVDELQQPL ELKPEPELES
              730 740 750 760
LELELGLVPE PELSLDLEPL LKAGLDLGPE LESVLESTLE
              770 780 790 800
PVIEPTLCMV SQTVPEPDQG PVSQPVPEPD LPCDLRHLNT
              810 820 830 840
EPMEIFRNCV KIEEIMPNGD PLLAGQNTVD EVYVSRPSHF
              850
YTDGPLMPSD F
```

This protein is encoded by a cDNA sequence with accession number M97934 in the NCBI database.

An example of a human positive BTN3A1 regulator sequence for a TRUB2 protein is shown below (Uniprot O95900; SEQ ID NO: 100).

```
                10 20 30 40
MGSAGLSRLH GLFAVYKPPG LKWKHLRDTV ELQLLKGLNA
                50 60 70 80
RKPPAPKQRV RFLLGPMEGS EEKELTLTAT SVPSFINHPL
                90 100 110 120
VCGPAFAHLK VGVGHRLDAQ ASGVLVLGVG HGCRLLTDMY
              130 140 150 160
NAHLTKDYTV RGLLGKATDD FREDGRLVEK TTYDHVTREK
              170 180 190 200
LDRILAVIQG SHQKALVMYS NLDLKTQEAY EMAVRGLIRP
              210 220 230 240
MNKSPMLITG IRCLYFAPPE FLLEVQCMHE TQKELRKLVH
              250 260 270 280
EIGLELKTTA VCTQVRRTRD GFFTLDSALL RTQWDLTNIQ
              290 300 310 320
DAIRAATPQV AAELEKSLSP GLDTKQLPSP GWSWDSQGPS
              330
STLGLERGAG Q
```

This protein is encoded by a cDNA sequence with accession number AF131848 in the NCBI database.

An example of a human positive BTN3A1 regulator sequence for a BIRC6 protein is shown below (Uniprot Q9NR09; SEQ ID NO:101).

```
                10 20 30 40 50 60 70
MVTGGGAAPP GTVTEPLPSV IVLSAGRKMA AAAAAASGPG CSSAAGAGAA GVSEWLVLRD GCMHCDADGL
                80 90 100 110 120 130 140
HSLSYHPALN AILAVTSRGT IKVIDGTSGA TLQASALSAK PGGQVKCQYI SAVDKVIFVD DYAVGCRKDL
              150 160 170 180 190 200 210
NGILLIDTAL QTPVSKQDDV VQLELPVTEA QQLLSACLEK VDISSTEGYD LFITQLKDGL KNTSHETAAN
              220 230 240 250 260 270 280
HKVAKWATVT FHLPHHVLKS IASAIVNELK KINQNVAALP VASSVMDRLS YLLPSARPEL GVGPGRSVDR
              290 300 310 320 330 340 350
SLMYSEANRR ETFTSWPHVG YRWAQPDPMA QAGFYHQPAS SGDDRAMCFT CSVCLVCWEP TDEPWSEHER
              360 370 380 390 400 410 420
HSPNCPFVKG EHTQNVPLSV TLATSPAQFP CTDGTDRISC FGSGSCPHFL AAATKRGKIC IWDVSKLMKV
              430 440 450 460 470 480 490
HLKFEINAYD PAIVQQLILS GDPSSGVDSR RPTLAWLEDS SSCSDIPKLE GDSDDLLEDS DSEEHSRSDS
              500 510 520 530 540 550 560
VTGHTSQKEA MEVSLDITAL SILQQPEKLQ WEIVANVLED TVKDLEELGA NPCLTNSKSE KTKEKHQEQH
              570 580 590 600 610 620 630
NIPFPCLLAG GLLTYKSPAT SPISSNSHRS LDGLSRTQGE SISEQGSTDN ESCTNSELNS PLVRRTLPVL
              640 650 660 670 680 690 700
LLYSIKESDE KAGKIFSQMN NIMSKSLHDD GFTVPQIIEM ELDSQEQLLL QDPPVTYIQQ FADAAANLTS
              710 720 730 740 750 760 770
PDSEKWNSVF PKPGTLVQCL RLPKFAEEEN LCIDSITPCA DGIHLLVGLR TCPVESLSAI NQVEALNNLN
              780 790 800 810 820 830 840
KLNSALCNRR KGELESNLAV VNGANISVIQ HESPADVQTP LIIQPEQRNV SGGYLVLYKM NYATRIVTLE
              850 860 870 880 890 900 910
EEPIKIQHIK DPQDTITSLI LLPPDILDNR EDDCEEPIED MQLTSKNGFE REKTSDISTL GHLVITTQGG
              920 930 940 950 960 970 980
YVKILDLSNF EILAKVEPPK KEGTEEQDTF VSVIYCSGTD RLCACTKGGE LHFLQIGGTC DDIDEADILV
              990 1000 1010 1020 1030 1040 1050
DGSLSKGIEP SSEGSKPLSN PSSPGISGVD LLVDQPFTLE ILTSLVELTR FETLTPRFSA TVPPCWVEVQ
              1060 1070 1080 1090 1100 1100 1120
QEQQQRRHPQ HLHQQHHGDA AQHTRTWKLQ TDSNSWDEHV FELVLPKACM VGHVDFKFVL NSNITNIPQI
            1130 1140 1150 1160 1170 1180 1190
QVTLLKNKAP GLGKVNALNI EVEQNGKPSL VDLNEEMQHM DVEESQCLRL CPFLEDHKED ILCGPVWLAS
            1200 1210 1220 1230 1240 1250 1260
GLDLSGHAGM LTLTSPKLVK GMAGGKYRSF LIHVKAVNER GTEEICNGGM RPVVRLPSLK HQSNKGYSLA
            1270 1280 1290 1300 1310 1320 1330
SLLAKVAAGK EKSSNVKNEN TSGTRKSENL RGCDLLQEVS VTIRRFKKTS ISKERVQRCA MLQFSEFHEK
            1340 1350 1360 1370 1380 1390 1400
LVNTLCRKTD DGQITEHAQS LVLDTLCWLA GVHSNGPGSS KEGNENLLSK TRKFLSDIVR VCFFEAGRSI
            1410 1420 1430 1440 1450 1460 1470
AHKCARFLAL CISNGKCDPC QPAFGPVLLK ALLDNMSFLP AATTGGSVYW YFVLLNYVKD EDLAGCSTAC
            1480 1490 1500 1510 1520 1530 1540
ASLLTAVSRQ LQDRETPMEA LLQTRYGLYS SPFDPVLFDL EMSGSSCKNV YNSSIGVQSD EIDLSDVLSG
            1550 1560 1570 1580 1590 1600 1610
NGKVSSCTAA EGSFTSLTGL LEVEPLHFTC VSTSDGTRIE RDDAMSSFGV TPAVGGLSSG TVGEASTALS
            1620 1630 1640 1650 1660 1670 1680
SAAQVALQSL SHAMASAEQQ LQVLQEKQQQ LLKLQQQKAK LEAKLHQTTA AAAAAASAVG PVHNSVPSNP
            1690 1700 1710 1720 1730 1740 1750
VAAPGFFIHP SDVIPPTPKT TPLFMTPPLT PPNEAVSVVI NAELAQLFPG SVIDPPAVNL AAHNKNSNKS
            1760 1770 1780 1790 1800 1810 1820
RMNPIGSGLA LAISHASHFL QPPPHQSIII ERMHSGARRF VTLDFGRPIL LTDVLIPTCG DLASLSIDIW
            1830 1840 1850 1860 1870 1880 1890
TLGEEVDGRR LVVATDISTH SLILHDLIPP PVCRFMKITV IGRYGSTNAR AKIPLGFYYG HTYILPWESE
            1900 1910 1920 1930 1940 1950 1960
LKLMHDPLKG EGESANQPEI DQHLAMMVAL QEDIQCRYNL ACHRLETLLQ SIDLPPLNSA NNAQYFLRKP
            1970 1980 1990 2000 2010 2020 2030
DKAVEEDSRV FSAYQDCIQL QLQLNLAHNA VQRLKVALGA SRKMLSETSN PEDLIQTSST EQLRTIIRYL
            2040 2050 2060 2070 2080 2090 2100
LDTLLSLLHA SNGHSVPAVL QSTFHAQACE ELFKHLCISG TPKIRLHTGL LLVQLCGGER WWGQFLSNVL
            2110 2120 2130 2140 2150 2160 2170
QELYNSEQLL IFPQDRVFML LSCIGQRSLS NSGVLESLLN LLDNLLSPLQ PQLPMHRRTE GVLDIPMISW
            2180 2190 2200 2210 2220 2230 2240
VVMLVSRLLD YVATVEDEAA AAKKPLNGNQ WSFINNNLHT QSLNRSSKGS SSLDRLYSRK IRKQLVHHKQ
            2250 2260 2270 2280 2290 2300 2310
QLNLLKAKQK ALVEQMEKEK IQSNKGSSYK LIVEQAKLKQ ATSKHFKDLI RLRRTAEWSR SNLDTEVTTA
            2320 2330 2340 2350 2360 2370 2380
KESPEIEPLP FTLAHERCIS VVQKLVLFLL SMDFTCHADL LLFVCKVLAR IANATRPTIH LCEIVNEPQL
            2390 2400 2410 2420 2430 2440 2450
ERLLLLLVGT DFNRGDISWG GAWAQYSLTC MLQDILAGEL LAPVAAEAME EGTVGDDVGA TAGDSDDSLQ
            2460 2470 2480 2490 2500 2510 2520
QSSVQLLETI DEPLTHDITG APPLSSLEKD KEIDLELLQD LMEVDIDPLD IDLEKDPLAA KVFKPISSTW
            2530 2540 2550 2560 2570 2580 2590
YDYWGADYGT YNYNPYIGGL GIPVAKPPAN TEKNGSQTVS VSVSQALDAR LEVGLEQQAE LMLKMMSTLE
            2600 2610 2620 2630 2640 2650 2660
ADSILQALTN TSPTLSQSPT GTDDSLLGGL QAANQTSQLI IQLSSVPMLN VCFNKLFSML QVHHVQLESL
            2670 2680 2690 2700 2710 2720 2730
LQLWLTLSLN SSSTGNKENG ADIFLYNANR IPVISLNQAS ITSFLTVLAW YPNTLLRTWC LVLHSLTLMT
            2740 2750 2760 2770 2780 2790 2800
NMQLNSGSSS AIGTQESTAH LLVSDPNLIH VLVKFLSGTS PHGTNQHSPQ VGPTATQAMQ EFLTRLQVHL
            2810 2820 2830 2840 2850 2860 2870
SSTCPQIFSE FLLKLIHILS TERGAFQTGQ GPLDAQVKLL EFTLEQNFEV VSVSTISAVI ESVTFLVHHY
            2880 2890 2900 2910 2920 2930 2940
ITCSDKVMSR SGSDSSVGAR ACFGGLFANL IRPGDAKAVC GEMTRDQLMF DLLKLVNILV QLPLSGNREY
            2950 2960 2970 2980 2990 3000 3010
SARVSVTTNT TDSVSDEEKV SGGKDGNGSS TSVQGSPAYV ADLVLANQQI MSQILSALGL CNSSAMAMII
            3020 3030 3040 3050 3060 3070 3080
GASGLHLTKH ENFHGGLDAI SVGDGLFTIL TTLSKKASTV HMMLQPILTY MACGYMGRQG SLATCQLSEP
            3090 3100 3110 3120 3130 3140 3150
LLWFILRVLD TSDALKAFHD MGGVQLICNN MVTSTRAIVN TARSMVSTIM KFLDSGPNKA VDSTLKTRIL
            3160 3170 3180 3190 3200 3210 3220
ASEPDNAEGI HNFAPLGTIT SSSPTAQPAE VLLQATPPHR RARSAAWSYI FLPEEAWCDL TIHLPAAVLL
            3230 3240 3250 3260 3270 3280 3290
KEIHIQPHLA SLATCPSSVS VEVSADGVNM LPLSTPVVTS GLTYIKIQLV KAEVASAVCL RLHRPRDAST
            3300 3310 3320 3330 3340 3350 3360
LGLSQIKLLG LTAFGTTSSA TVNNPFLPSE DQVSKTSIGW LRLLHHCLTH ISDLEGMMAS AAAPTANLLQ
            3370 3380 3390 3400 3410 3420 3430
TCAALLMSPY CGMHSPNIEV VLVKIGLQST RIGLKLIDIL LRNCAASGSD PTDLNSPLLF GRLNGLSSDS
            3440 3450 3460 3470 3480 3490 3500
TIDILYQLGT TQDPGTKDRI QALLKWVSDS ARVAAMKRSG RMNYMCPNSS TVEYGLLMPS PSHLHCVAAI
            3510 3520 3530 3540 3550 3560 3570
LWHSYELLVE YDLPALLDQE LFELLFNWSM SLPCNMVLKK AVDSLLCSMC HVHPNYFSLL MGWMGITPPP
            3580 3590 3600 3610 3620 3630 3640
VQCHHRLSMT DDSKKQDLSS SLTDDSKNAQ APLALTESHL ATLASSSQSP EAIKQLLDSG LPSLLVRSLA
            3650 3660 3670 3680 3690 3700 3710
SFCFSHISSS ESIAQSIDIS QDKLRRHHVP QQCNKMPITA DLVAPILRFL TEVGNSHIMK DWLGGSEVNP
            3720 3730 3740 3750 3760 3770 3780
LWTALLFLLC HSGSTSGSHN LGAQQTSARS ASLSSAATTG LTTQQRTAIE NATVAFFLQC ISCHPNNQKL
            3790 3800 3810 3820 3830 3840 3850
MAQVLCELFQ TSPQRGNLPT SGNISGFIRR LFLQLMLEDE KVTMFLQSPC PLYKGRINAT SHVIQHPMYG
            3860 3870 3880 3890 3900 3910 3920
AGHKFRTLHL PVSTTLSDVL DRVSDTPSIT AKLISEQKDD KEKKNHEEKE KVKAENGFQD NYSVVVASGL
            3930 3940 3950 3960 3970 3980 3990
KSQSKRAVSA TPPRPPSRRG RTIPDKIGST SGAEAANKII TVPVFHLFHK LLAGQPLPAE MTLAQLLTLL
            4000 4010 4020 4030 4040 4050 4060
YDRKLPQGYR SIDLTVKLGS RVITDPSLSK TDSYKRLHPE KDHGDLLASC PEDEALTPGD ECMDGILDES
            4070 4080 4090 4100 4110 4120 4130
LLETCPIQSP LQVFAGMGGL ALIAERLPML YPEVIQQVSA PVVTSTTQEK PKDSDQFEWV TIEQSGELVY
            4140 4150 4160 4170 4180 4190 4200
EAPETVAAEP PPIKSAVQTM SPIPAHSLAA FGLFLRLPGY AEVLLKERKH AQCLLRLVLG VTDDGEGSHI
            4210 4220 4230 4240 4250 4260 4270
LQSPSANVLP TLPFHVLRSL FSTTPLTTDD GVLLRRMALE IGALHLILVC LSALSHHSPR VPNSSVNQTE
            4280 4290 4300 4310 4320 4330 4340
PQVSSSHNPT STEEQQLYWA KGTGFGTGST ASGWDVEQAL TKQRLEEEHV TCLLQVLASY INPVSSAVNG
            4350 4360 4370 4380 4390 4400 4410
EAQSSHETRG QNSNALPSVL LELLSQSCLI PAMSSYLRND SVLDMARHVP LYRALLELLR AIASCAAMVP
            4420 4430 4440 4450 4460 4470 4480
ILLPLSTENG EEEEEQSECQ TSVGTLLAKM KTCVDTYTNR LRSKRENVKT GVKPDASDQE PEGLTLLVPD
            4490 4500 4510 4520 4530 4540 4550
IQKTAEIVYA ATTSLRQANQ EKKLGEYSKK AAMKPKPLSV LKSLEEKYVA VMKKLQFDTF EMVSEDEDGK
            4560 4570 4580 4590 4600 4610 4620
LGFKVNYHYM SQVKNANDAN SAARARRLAQ EAVTLSTSLP LSSSSSVFVR CDEERLDIMK VLITGPADTP
            4630 4640 4650 4660 4670 4680 4690
YANGCFEFDV YFPQDYPSSP PLVNLETTGG HSVRFNPNLY NDGKVCLSIL NTWHGRPEEK WNPQTSSFLQ
            4700 4710 4720 4730 4740 4750 4760
VLVSVQSLIL VAEPYFNEPG YERSRGTPSG TQSSREYDGN IRQATVKWAM LEQIRNPSPC FKEVIHKHFY
            4770 4780 4790 4800 4810 4820 4830
LKRVEIMAQC EEWIADIQQY SSDKRVGRTM SHHAAALKRH TAQLREELLK LPCPEGLDPD TDDAPEVCRA
              4840 4850
TTGAEETLMH DQVKPSSSKE LPSDFQL
```

This protein is encoded by a cDNA sequence with accession number AF265555 in the NCBI database.

An example of a human positive BTN3A1 regulator sequence for a MARS2 protein is shown below (Uniprot Q96GW9; SEQ ID NO:102).

```
                10 20 30 40
MLRTSVLRLL GRTGASRLSL LEDFGPRYYS SGSLSAGDDA
                50 60 70 80
CDVRAYFTTP IFYVNAAPHI GHLYSALLAD ALCRHRRLRG
                90 100 110 120
PSTAATRFST GTDEHGLKIQ QAAATAGLAP TELCDRVSEQ
              130 140 150 160
FQQLFQEAGI SCTDFIRTTE ARHRVAVQHF WGVLKSRGLL
              170 180 190 200
YKGVYEGWYC ASDECFLPEA KVTQQPGPSG DSFPVSLESG
              210 220 230 240
HPVSWTKEEN YIFRLSQFRK PLQRWLRGNP QAITPEPFHH
              250 260 270 280
VVLQWLDEEL PDLSVSRRSS HLHWGIPVPG DDSQTIYVWL
              290 300 310 320
DALVNYLTVI GYPNAEFKSW WPATSHIIGK DILKFHAIYW
              330 340 350 360
PAFLLGAGMS PPQRICVHSH WTVCGQKMSK SLGNVVDPRT
              370 380 390 400
CLNRYTVDGF RYFLLRQGVP NWDCDYYDEK VVKLLNSELA
              410 420 430 440
DALGGLLNRC TAKRINPSET YPAFCTTCFP SEPGLVGPSV
              450 460 470 480
RAQAEDYALV SAVATLPKQV ADHYDNFRIY KALEAVSSCV
              490 500 510 520
RQTNGFVQRH APWKLNWESP VDAPWLGTVL HVALECLRVF
              530 540 550 560
GTLLQPVTPS LADKLLSRIG VSASERSLGE LYFLPRFYGH
              570 580 590
PCPFEGRRLG PETGLLFPRL DQSRTWLVKA HRT
```

This protein is encoded by a cDNA sequence with accession number AB107013 in the NCBI database.

An example of a human positive BTN3A1 regulator sequence for a NDUFA9 protein is shown below (Uniprot Q16795; SEQ ID NO: 103).

```
                10 20 30 40
MAAAAQSRVV RVLSMSRSAI TAIATSVCHG PPCRQLHHAL
                50 60 70 80
MPHGKGGRSS VSGIVATVFG ATGFLGRYVV NHLGRMGSQV
                90 100 110 120
IIPYRCDKYD IMHLRPMGDL GQLLFLEWDA RDKDSIRRVV
              130 140 150 160
QHSNVVINLI GRDWETKNFD FEDVFVKIPQ AIAQLSKEAG
              170 180 190 200
VEKFIHVSHL NANIKSSSRY LRNKAVGEKV VRDAFPEAII
              210 220 230 240
VKPSDIFGRE DRFLNSFASM HRFGPIPLGS LGWKTVKQPV
              250 260 270 280
YVVDVSKGIV NAVKDPDANG KSFAFVGPSR YLLFHLVKYI
              290 300 310 320
FAVAHRLFLP FPLPLFAYRW VARVFEISPF EPWITRDKVE
              330 340 350 360
RMHITDMKLP HLPGLEDLGI QATPLELKAI EVLRRHRTYR
WLSAEIEDVK PAKTVNI
```

This protein is encoded by a cDNA sequence with accession number AF050641 in the NCBI database.

An example of a human positive BTN3A1 regulator sequence for a USP19 protein is shown below (Uniprot O94966; SEQ ID NO: 104).

```
                10 20 30 40
MSGGASATGP RRGPPGLEDT TSKKKQKDRA NQESKDGDPR
                50 60 70 80
KETGSRYVAQ AGLEPLASGD PSASASHAAG ITGSRHRTRL
                90 100 110 120
FFPSSSGSAS TPQEEQTKEG ACEDPHDLLA TPTPELLLDW
              130 140 150 160
RQSAEEVIVK LRVGVGPLQL EDVDAAFTDT DCVVRFAGGQ
              170 180 190 200
QWGGVFYAEI KSSCAKVQTR KGSLLHLTLP KKVPMLTWPS
              210 220 230 240
LLVEADEQLC IPPLNSQTCL LGSEENLAPL AGEKAVPPGN
              250 260 270 280
DPVSPAMVRS RNPGKDDCAK EEMAVAADAA TLVDEPESMV
              290 300 310 320
NLAFVKNDSY EKGPDSVVVH VYVKEICRDT SRVLFREQDF
              330 340 350 360
TLIFQTRDGN FLRLHPGCGP HTTFRWQVKL RNLIEPEQCT
              370 380 390 400
FCFTASRIDI CLRKRQSQRW GGLEAPAARV GGAKVAVPTG
              410 420 430 440
PTPLDSTPPG GAPHPLTGQE EARAVEKDKS KARSEDTGLD
              450 460 470 480
SVATRTPMEH VTPKPETHLA SPKPTCMVPP MPHSPVSGDS
              490 500 510 520
VEEEEEEEKK VCLPGFTGLV NLGNTCFMNS VIQSLSNTRE
              530 540 550 560
LRDFFHDRSF EAEINYNNPL GTGGRLAIGF AVLLRALWKG
              570 580 590 600
THHAFQPSKL KAIVASKASQ FTGYAQHDAQ EFMAFLLDGL
              610 620 630 640
HEDINRIQNK PYTETVDSDG RPDEVVAEEA WQRHKMRNDS
              650 660 670 680
FIVDLFQGQY KSKLVCPVCA KVSITFDPFL YLPVPLPQKQ
              690 700 710 720
KVLPVFYFAR EPHSKPIKFL VSVSKENSTA SEVLDSLSQS
              730 740 750 760
VHVKPENLRL AEVIKNRFHR VFLPSHSLDT VSPSDTLLCF
              770 780 790 800
ELLSSELAKE RVVVLEVQQR PQVPSVPISK CAACQRKQQS
              810 820 830 840
EDEKLKRCTR CYRVGYCNQL CQKTHWPDHK GLCRPENIGY
              850 860 870 880
PFLVSVPASR LTYARLAQLL EGYARYSVSV FQPPFQPGRM
              890 900 910 920
ALESQSPGCT TLLSTGSLEA GDSERDPIQP PELQLVTPMA
              930 940 950 960
EGDTGLPRVW AAPDRGPVPS TSGISSEMLA SGPIEVGSLP
              970 980 990 1000
AGERVSRPEA AVPGYQHPSE AMNAHTPQFF IYKIDSSNRE
            1010 1020 1030 1040
QRLEDKGDTP LELGDDCSLA LVWRNNERLQ EFVLVASKEL
            1050 1060 1070 1080
ECAEDPGSAG EAARAGHFTL DQCLNLFTRP EVLAPEEAWY
            1090 1100 1110 1120
CPQCKQHREA SKQLLLWRLP NVLIVQLKRF SFRSFIWRDK
            1130 1140 1150 1160
INDLVEFPVR NLDLSKFCIG QKEEQLPSYD LYAVINHYGG
            1170 1180 1190 1200
MIGGHYTACA RLPNDRSSQR SDVGWRLFDD STVTTVDESQ
            1210 1220 1230 1240
VVTRYAYVLF YRRRNSPVER PPRAGHSEHH PDLGPAAEAA
            1250 1260 1270 1280
ASQASRIWQE LEAEEEPVPE GSGPLGPWGP QDWVGPLPRG
            1290 1300 1310
PTTPDEGCLR YFVLGTVAAL VALVLNVFYP LVSQSRWR
```

This protein is encoded by a cDNA sequence with accession number AB020698 in the NCBI database.

An example of a human positive BTN3A1 regulator sequence for a UBA6 protein is shown below (Uniprot A0AVT1; SEQ ID NO-105).

```
                10 20 30 40
MEGSEPVAAH QGEEASCSSW GTGSTNKNLP IMSTASVEID
                50 60 70 80
DALYSRQRYV LGDTAMQKMA KSHVFLSGMG GLGLEIAKNL
                90 100 110 120
VLAGIKAVTI HDTEKCQAWD LGTNFFLSED DVVNKRNRAE
              130 140 150 160
AVLKHIAELN PYVHVTSSSV PFNETTDLSF LDKYQCVVLT
              170 180 190 200
EMKLPLQKKI NDFCRSQCPP IKFISADVHG IWSRLFCDFG
              210 220 230 240
DEFEVLDTTG EEPKEIFISN ITQANPGIVT CLENHPHKLE
              250 260 270 280
TGQFLTFREI NGMTGLNGSI QQITVISPFS FSIGDTTELE
              290 300 310 320
PYLHGGIAVQ VKTPKTVFFE SLERQLKHPK CLIVDFSNPE
              330 340 350 360
APLEIHTAML ALDQFQEKYS RKPNVGCQQD SEELLKLATS
              370 380 390 400
ISETLEEKPD VNADIVHWLS WTAQGFLSPL AAAVGGVASQ
              410 420 430 440
EVLKAVTGKF SPLCQWLYLE AADIVESLGK PECEEFLPRG
              450 460 470 480
DRYDALRACI GDTLCQKLQN INIFLVGCGA IGCEMLKNFA
              490 500 510 520
LLGVGTSKEK GMITVTDPDL IEKSNLNRQF LFRPHHIQKP
              530 540 550 560
KSYTAADATL KINSQIKIDA HLNKVCPTTE TIYNDEFYTK
              570 580 590 600
QDVIITALDN VEARRYVDSR CLANLRPLLD SGTMGTKGHT
              610 620 630 640
EVIVPHLTES YNSHRDPPEE EIPFCTLKSF PAAIEHTIQW
              650 660 670 680
ARDKFESSFS HKPSLFNKFW QTYSSAEEVL QKIQSGHSLE
              690 700 710 720
GCFQVIKLLS RRPRNWSQCV ELARLKFEKY FNHKALQLLH
              730 740 750 760
CFPLDIRLKD GSLFWQSPKR PPSPIKFDLN EPLHLSFLQN
              770 780 790 800
AAKLYATVYC IPFAEEDLSA DALLNILSEV KIQEFKPSNK
              810 820 830 840
VVQTDETARK PDHVPISSED ERNAIFQLEK AILSNEATKS
              850 860 870 880
DLQMAVLSFE KDDDHNGHID FITAASNLRA KMYSIEPADR
              890 900 910 920
FKTKRIAGKI IPAIATTTAT VSGLVALEMI KVTGGYPFEA
              930 940 950 960
YKNCFLNLAI PIVVFTETTE VRKTKIRNGI SFTIWDRWTV
              970 980 990 1000
HGKEDFTLLD FINAVKEKYG IEPTMVVQGV KMLYVPVMPG
            1010 1020 1030 1040
HAKRLKLTMH KLVKPTTEKK YVDLTVSFAP DIDGDEDLPG
          1050
PPVRYYFSHD TD
```

This protein is encoded by a cDNA sequence with accession number AY359880 in the NCBI database.

An example of a human positive BTN3A1 regulator sequence for a MTG1 protein is shown below (Uniprot Q9BT17; SEQ ID NO:106).

```
                10 20 30 40
MRLTPRALCS AAQAAWRENF PLCGRDVARW FPGHMAKGLK
                50 60 70 80
KMQSSLKLVD CIIEVHDARI PLSGRNPLFQ ETLGLKPHLL
                90 100 110 120
VINKMDLADL TEQQKIMQHL EGEGLKNVIF INCVKDENVK
              130 140 150 160
QIIPMVTELI GRSHRYHRKE NLEYCIMVIG VPNVGKSSLI
              170 180 190 200
NSLRRQHLRK GKATRVGGEP GITRAVMSKI QVSERPLMFL
              210 220 230 240
LDTPGVLAPR IESVETGLKL ALCGTVLDHL VGEETMADYL
              250 260 270 280
LYTLNKHQRF GYVQHYGLGS ACDNVERVLK SVAVKLGKTQ
              290 300 310 320
KVKVLTGTGN VNIIQPNYPA AARDFLQTFR RGLLGSVMLD
              330
LDVLRGHPPA ETLP
```

This protein is encoded by a cDNA sequence with accession number AK074976 in the NCBI database.

An example of a human positive BTN3A1 regulator sequence for a KIAA0391 protein is shown below (Uniprot 015091; SEQ ID NO:107).

```
                10 20 30 40
MTFYLFGIRS FPKLWKSPYL GLGPGHSYVS LFLADRCGIR
                50 60 70 80
NQQRLFSLKT MSPQNTKATN LIAKARYLRK DEGSNKQVYS
                90 100 110 120
VPHFFLAGAA KERSQMNSQT EDHALAPVRN TIQLPTQPLN
              130 140 150 160
SEEWDKLKED LKENTGKTSF ESWIISQMAG CHSSIDVAKS
              170 180 190 200
ILAWVAAKNN GIVSYDLLVK YIYICVFHMQ TSEVIDVFEI
              210 220 230 240
MKARYKTLEP RGYSLLIRGL IHSDRWREAL LLLEDIKKVI
              250 260 270 280
TPSKKNYNDC IQGALLHQDV NTAWNLYQEL LGHDIVPMLE
              290 300 310 320
TIKAFFDFGK DIKDDNYSNK LLDILSYLRN NQLYPGESFA
              330 340 350 360
HSIKTWFESV PGKQWKGQFT TVRKSGQCSG CGKTIESIQL
              370 380 390 400
SPEEYECLKG KIMRDVIDGG DQYRKTTPQE LKRFENFIKS
              410 420 430 440
RPPFDVVIDG LNVAKMFPKV RESQLLLNVV SQLAKRNLRL
              450 460 470 480
LVLGRKHMLR RSSQWSRDEM EEVQKQASCF FADDISEDDP
              490 500 510 520
FLLYATLHSG NHCRFITRDL MRDHKACLPD AKTQRLFFKW
              530 540 550 560
QQGHQLAIVN RFPGSKLTFQ RILSYDTVVQ TTGDSWHIPY
              570 580
DEDLVERCSC EVPTKWICLH QKT
```

This protein is encoded by a cDNA sequence with accession number AB002389 in the NCBI database.

An example of a human positive BTN3A1 regulator sequence for a IRF9 protein is shown below (Uniprot Q00978; SEQ ID NO:108).

```
                10 20 30 40
MASGRARCTR KLRNWVVEQV ESGQFPGVCW DDTAKTMFRI
                50 60 70 80
PWKHAGKQDF REDQDAAFFK AWAIFKGKYK EGDTGGPAVW
                90 100 110 120
KTRLRCALNK SSEFKEVPER GRMDVAEPYK VYQLLPPGIV
              130 140 150 160
SGQPGTQKVP SKRQHSSVSS ERKEEEDAMQ NCTLSPSVLQ
              170 180 190 200
DSINNEEEGA SGGAVHSDIG SSSSSSSPEP QEVTDTTEAP
              210 220 230 240
FQGDQRSLEF LLPPEPDYSL LLTFIYNGRV VGEAQVQSLD
              250 260 270 280
CRLVAEPSGS ESSMEQVLFP KPGPLEPTQR LLSQLERGIL
              290 300 310 320
VASNPRGLFV QRLCPIPISW NAPQAPPGPG PHILPSNECV
              330 340 350 360
ELFRTAYFCR DLVRYFQGLG PPPKFQVTLN FWEESHGSSH
              370 380 390
TPQNLITVKM EQAFARYLLE QTPEQQAAIL SLV
```

This protein is encoded by a cDNA sequence with accession number BC035716.2 in the NCBI database.

The sequences provided herein are exemplary. Isoforms and variants of the sequences described herein and of any of regulators listed in Tables 1 and 2 can also be used in the methods and compositions described herein.

For example, isoforms and variants of the proteins and nucleic acids can be used in the methods and compositions described herein when they are substantially identical to the ‘reference’ sequences described herein and/or substantially identical to the any of the genes listed in Tables 1 or 2. The terms “substantially identity” indicates that a polypeptide or nucleic acid comprises a sequence with between 55-100% sequence identity to a reference sequence, for example with at least 55% sequence identity, preferably 60%, preferably 70%, preferably 80%, preferably at least 90%, preferably at least 95%, preferably at least 96%, preferably at least 97% sequence, preferably at least 98%, preferably at least 99% identity to a reference sequence over a specified comparison window. Optimal alignment may be ascertained or conducted using the homology alignment algorithm of Needleman and Wunsch, J. Mol. Biol. 48:443-53 (1970).

An indication that two polypeptide sequences are substantially identical is that both polypeptides have the same function—acting as a regulator of BTN3A1 expression or activity. The polypeptide that is substantially identical to a regulator of BTN3A1 sequence and may not have exactly the same level of activity as the regulator of BTN3A1. Instead, the substantially identical polypeptide may exhibit greater or lesser levels of regulator of BTN3A1 activity than the those listed in Table 1 or 2, or any of the sequences recited herein. For example, the substantially identical polypeptide or nucleic acid may have at least about 400%, or at least about 50%, or at least about 60%, or at least about 70%, or at least about 80%, or at least about 90%, or at least about 95%, or at least about 97%, or at least about 98%, or at least about 100%, or at least about 105%, or at least about 110%, or at least about 120%, or at least about 130%, or at least about 140%, or at least about 150%, or at least about 200% of the activity of a regulator of BTN3A1 described herein a when measured by similar assay procedures.

Alternatively, substantial identity is present when second polypeptide is immunologically reactive with antibodies raised against the first polypeptide (e.g., a polypeptide with encoded by any of the genes listed in Tables 1 and 2). Thus, a polypeptide is substantially identical to a first polypeptide, for example, where the two polypeptides differ only by a conservative substitution. In addition, a polypeptide can be substantially identical to a first polypeptide when they differ by a non-conservative change if the epitope that the antibody recognizes is substantially identical. Polypeptides that are “substantially similar” share sequences as noted above except that some residue positions, which are not identical, may differ by conservative amino acid changes.

### Expression Systems

Nucleic acid segments encoding one or more BTN3A1 proteins and/or one or more BTN3A1 regulator proteins, or nucleic acid segments that are BTN3A1 inhibitory nucleic acids, and/or nucleic acid segments that are BTN3A1 regulator inhibitory nucleic acids can be inserted into or employed with any suitable expression system. A useful quantity of one or more BTN3A1 proteins and/or BTN3A1 regulator proteins can be generated from such expression systems. A therapeutically effective amount of a BTN3A negative protein, a therapeutically effective amount of a BTN3A negative regulator nucleic, or a therapeutically effective amount of an inhibitory nucleic acid that binds BTN3A1 negative regulator nucleic acid can also be generated from such expression systems.

Recombinant expression of nucleic acids (or inhibitory nucleic acids) is usefully accomplished using a vector, such as a plasmid. The vector can include a promoter operably linked to nucleic acid segment encoding one or more BTN3A1 inhibitory nucleic acids or one or more BTN3A1 negative regulator proteins.

The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying, encoding and/or expressing BTN3A1 negative or positive regulator proteins. A variety of prokaryotic and eukaryotic expression vectors suitable for carrying, encoding and/or expressing BTN3A1 inhibitory nucleic acids or BTN3A1 regulator inhibitory nucleic acids can be employed. Such expression vectors include, for example, pET, pET3d, pCR2.1, pBAD, pUC, and yeast vectors. The vectors can be used, for example, in a variety of in vivo and in vitro situations.

The expression cassette, expression vector, and sequences in the cassette or vector can be heterologous. As used herein, the term “heterologous” when used in reference to an expression cassette, expression vector, regulatory sequence, promoter, or nucleic acid refers to an expression cassette, expression vector, regulatory sequence, or nucleic acid that has been manipulated in some way. For example, a heterologous promoter can be a promoter that is not naturally linked to a nucleic acid of interest, or that has been introduced into cells by cell transformation procedures. A heterologous nucleic acid or promoter also includes a nucleic acid or promoter that is native to an organism but that has been altered in some way (e.g., placed in a different chromosomal location, mutated, added in multiple copies, linked to a non-native promoter or enhancer sequence, etc.). Heterologous nucleic acids may comprise sequences that comprise cDNA forms; the cDNA sequences may be expressed in either a sense (to produce mRNA) or anti-sense orientation (to produce an anti-sense RNA transcript that is complementary to the mRNA transcript). Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that at linked to a coding region to which they are not linked in nature.

Viral vectors that can be employed include those relating to retroviruses, Moloney murine leukemia viruses (MMLV), lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in by Verma, I. M., Retroviral vectors for gene transfer. In Microbiology-1985, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

A variety of regulatory elements can be included in the expression cassettes and/or expression vectors, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleic acid segment encoding a BTN3A1 or BTN3A1 regulator protein. In another example, the promoter can be upstream of a BTN3A1 inhibitory nucleic acid segment or an inhibitory nucleic acid segment for one or more BTN3A1 regulators.

A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences for the termination of transcription, which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The expression of BTN3A1 proteins, one or more BTN3A1 regulator proteins, BTN3A1 inhibitory nucleic acid molecules, or any BTN3A1 regulator inhibitory nucleic acid molecules, from an expression cassette or expression vector can be controlled by any promoter capable of expression in prokaryotic cells or eukaryotic cells. Examples of prokaryotic promoters that can be used include, but are not limited to, SP6, T7, T5, tac, bla, trp, gal, lac, or maltose promoters. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. Vectors for bacterial expression include pGEX-5X-3, and for eukaryotic expression include pCIneo-CMV.

The expression cassette or vector can include nucleic acid sequence encoding a marker product. This marker product is used to determine if the gene has been delivered to the cell and once delivered is being expressed. Marker genes can include the E. coli lacZ gene which encodes β-galactosidase, and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990): and Wolff, J. A. Nature, 352, 815-818, (1991).

For example, the nucleic acid molecules, expression cassette and/or vectors encoding BTN3A1 proteins, encoding one or more BTN3A1 regulator proteins, or encoding BTN3A1 inhibitory nucleic acid molecules, or encoding BTN3A1 regulator inhibitory nucleic acid molecules, can be introduced to a cell by any method including, but not limited to, calcium-mediated transformation, electroporation, microinjection, lipofection, particle bombardment and the like. The cells can be expanded in culture and then administered to a subject, e.g. a mammal such as a human. The amount or number of cells administered can vary but amounts in the range of about 10⁶ to about 10⁹ cells can be used. The cells are generally delivered in a physiological solution such as saline or buffered saline. The cells can also be delivered in a vehicle such as a population of liposomes, exosomes or microvesicles.

In some cases, the transgenic cell can produce exosomes or microvesicles that contain nucleic acid molecules, expression cassettes and/or vectors encoding BTN3A1, one or more BTN3A1 regulator, or a combination thereof. In some cases, the transgenic cell can produce exosomes or microvesicles that contain inhibitory nucleic acid molecules that can target BTN3A1 nucleic acids, one or more nucleic acids for BTN3A1 regulator, or a combination thereof. Microvesicles can mediate the secretion of a wide variety of proteins, lipids, mRNAs, and micro RNAs, interact with neighboring cells, and can thereby transmit signals, proteins, lipids, and nucleic acids from cell to cell (see, e.g., Shen et al., J Biol Chem. 286(16): 14383-14395 (2011); Hu et al., Frontiers in Genetics 3 (April 2012); Pegtel et al., Proc. Nat'l Acad Sci 107(14): 6328-6333 (2010); WO/2013/084000; each of which is incorporated herein by reference in its entirety. Cells producing such microvesicles can be used to express the BTN3A1 protein, one or more BTN3A1 regulator protein, or a combination thereof and/or inhibitory nucleic acids for BTN3A1, one or more BTN3A1 regulator, or a combination thereof

Transgenic vectors or cells with a heterologous expression cassette or expression vector can expresses BTN3A1, one or more BTN3A1 regulator, or a combination thereof, can optionally also express BTN3A1 inhibitory nucleic acids, BTN3A1 regulator inhibitory nucleic acids, or a combination thereof. Any of these vectors or cells can be administered to a subject. Exosomes produced by transgenic cells can be used to administer BTN3A1 nucleic acids, BTN3A1 regulator nucleic acids, or a combination thereof to tumor and cancer cells in the subject. Exosomes produced by transgenic cells can be used to deliver BTN3A1 inhibitory nucleic acids, BTN3A1 regulator inhibitory nucleic acids, or a combination thereof to tumor and cancer cells in the subject.

Methods and compositions that include inhibitors of BTN3A1, a BTN3A1 regulator, or any combination thereof can involve use of CRISPR modification, or antibodies or inhibitory nucleic acids directed against BTN3A1, a BTN3A1 regulator, or any combination thereof. Antibodies, inhibitory nucleic acids, small molecules, and combinations thereof can be used to reduce tumor load, cancer symptoms, and/or progression of the cancer. In some cases, antibodies can be prepared to bind selectively to one or more BTN3A protein, or one or more BTN3A regulator (e.g., any of the positive regulators of BTN3A). Antibodies can also be prepared and used that target or enhance γδ T cell-cancer cell interactions.

### Treatment

Methods are described herein for treating cancer. Such methods can involve administering therapeutic agents that can treat cancer cells exhibiting increased levels of BTN3A or increased levels any of the positive regulators of BTN3A described herein, or a combination thereof. Examples of such therapeutic agents can include administration of T cells (e.g., γδ T cells, and/or Vγ9Vδ2 T cells). Additional examples of such therapeutic agents include inhibitors of BTN3A, inhibitors of any of the positive regulators of BTN3A described herein, the BTN3A negative regulators, agents that modulate (e.g., enhance) γδ T cell-cancer interactions, or combinations thereof.

In some cases, immune cells, including T cells, can be isolated from a subject whose sample(s) exhibit increased expression of BTN3A or any of the positive regulators of BTN3A described herein. The immune cells, including T cells, can be expanded in culture and then administered to a subject, e.g. a mammal such as a human. The amount or number of cells administered can vary but amounts in the range of about 10⁶ to about 10⁹ cells can be used. The cells are generally delivered in a physiological solution such as saline or buffered saline. The cells can also be delivered in a vehicle such as a population of liposomes, exosomes or microvesicles.

The T cells to be administered can be a mixture of T cells with some other immune cells. However, in some cases the T cells are substantially free of other cell types. For example, the population of T cells to be administered to a subject can be at least about 20%, or at least about 30%, or at least about 40%, or at least about 50%, or at least about 60%, or at least about 70%, or at least about 80%, or at least about 90%, or at least about 95%, or at least about 97%, or up to and including a 100% cells. In some cases the T cells are γδ T cells. However, in some cases the T cells that are administered are Vγ9Vδ2 T cells.

Treatment methods described herein can also include administering agents that reduce the expression or function of BTN3A or any of the positive regulators of BTN3A described herein. Suitable methods for reducing the expression or function of BTN3A or any of the positive regulators of BTN3A described herein can include: inhibiting transcription of mRNA; degrading mRNA by methods including, but not limited to, the use of interfering RNA (RNAi); blocking translation of mRNA by methods including, but not limited to, the use of antisense nucleic acids or ribozymes, or the like. In some embodiments, a suitable method for downregulating expression may include providing to the cancer a small interfering RNA (siRNA) targeted to of BTN3A or to any of the positive regulators of BTN3A described herein, or to a combination thereof. Suitable methods for reducing the function or activity of BTN3A, or any of the positive regulators of BTN3A described herein, or a combination thereof, may also include administering a small molecule inhibitor that inhibits the function or activity of BTN3A or any of the positive regulators of BTN3A described herein.

In some cases, one or more BTN3A inhibitors or one or more inhibitors of the positive regulators of BTN3A described herein can be administered to treat cancers identified as expressing increased levels of BTN3A or any of the positive regulators of BTN3A described herein.

Examples of suitable inhibitors include, but are not limited to antisense oligonucleotides, oligopeptides, interfering RNA e.g., small interfering RNA (siRNA), small hairpin RNA (shRNA), aptamers, ribozymes, small molecule inhibitors, or antibodies or fragments thereof, and combinations thereof.

In some cases, the cancer includes hematological cancers, solid tumors, and semi-solid tymors. For example, the cancer can be breast cancer, bile duct cancer (e.g., cholangiocarcinoma), brain cancer, cervical cancer, colon cancer, lung cancer, melanoma, ovarian cancer, pancreatic cancer, prostate cancer, and other cancers. In some embodiments, the cancer includes myeloid neoplasms, lymphoid neoplasms, mast cell disorders, histiocytic neoplasms, leukemias, myelomas, or lymphomas.

As used herein, “solid tumor” is intended to include, but not be limited to, the following sarcomas and carcinomas: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. Solid tumor is also intended to encompass epithelial cancers.

Any of the regulators of BTN3A1 (e.g., the negative BTN3A regulators), as well as the inhibitors thereof (e.g., inhibitors of the positive BTN3A regulators), can be used in the treatment methods and compositions described herein. The inhibitors of BTN3A1 or of BTN3A1 regulators can, for example, be small molecules, antibodies, nucleic acids, expression cassettes, expression vectors, inhibitory nucleic acids, guide RNAs, nucleases (e.g., one or more cas nucleases), or a combination thereof.

### Screening

BTN3A and/or any of the BTN3A regulators can be used to obtain new agents that are effective for treating cancer. Methods are described herein that can include contacting one or more BTN3A protein, one or more BTN3A nucleic acid, one or more BTN3A regulator protein, one or more BTN3A regulator nucleic acid, or a combination thereof with a test agent in an assay mixture. The assay mixture can be incubated for a time and under conditions sufficient for observing whether modulation of the expression or function of one or more of the BTN3A proteins, BTN3A nucleic acids, BTN3A regulator proteins, BTN3A regulator nucleic acids, or a combination thereof has occurred. The assay mixture can then be tested to determine whether the expression or function of one or more of the BTN3A proteins, BTN3A nucleic acids, BTN3A regulator proteins, BTN3A regulator nucleic acids, or a combination thereof is reduced or increased. In cases, T cells and/or cancer cells can be included in the assay mixture and the effects of the test agents on the T cells and/or cancer cells can be measured. Such assay procedures can also be used to identify new BTN3A1 regulators.

For example, test agents can include one or more of the BTN3A1 regulators described herein, one or more anti-BTN3A1 antibodies, one or more BTN3A1 inhibitory nucleic acids that can modulate the expression of the BTN3A1, one or more guide RNAs that can bind a BTN3A1 nucleic acid, one or more antibodies that can bind any of the BTN3A1 regulators described herein, one or more inhibitory nucleic acid that can modulate the expression of any of the BTN3A1 regulators described herein, one or more guide RNAs that can bind a nucleic acid encoding any of the BTN3A1 regulators described herein, one or more small molecules that can modulate BTN3A1, one or more small molecules that can modulate any of the BTN3A1 regulators, one or more guide RNAs, or a combination thereof. Examples of such antibodies are described hereinbelow.

The type, quantity, or extent of BTN3A1 activity or BTN3A1 regulator activity in the presence or absence of a test agent can be evaluated by various assay procedures, including those described herein. For example, in addition to the small molecules, antibodies, inhibitory nucleic acids, guide RNAs, peptides, and polypeptides described herein, new types of small molecules, antibodies, guide RNAs, cas nucleases (e.g., a cas9 nuclease), inhibitory nucleic acids, guide RNAs, peptides, and polypeptides can be used as test agents to identify and evaluate to determine the type (positive or negative) of activity, the quantity of activity, and/or extent of BTN3A1 regulatory activity using the assays described herein.

For example, a method for evaluating new and existing agents that can modulate to identify the type (positive or negative), quantity, and/or extent of BTN3A1 regulatory activity can involve contacting one or more cells (or a cell population) that expresses BTN3A1 with a test agent (e.g., cancer cells) to provide a test assay mixture, and evaluating at least one of:
Detecting BTN3A1 protein or BTN3A1 regulator protein on the surface of or within one or more cells in the test assay mixture;
Quantifying the amount of BTN3A1 protein or BTN3A1 regulator protein within one or more of the cells or on the surface of one or more of the cells within the test assay mixture;
Quantifying the number of cells that express BTN3A1 protein or BTN3A1 regulator protein in the population of cells;
Detecting and/or quantifying alpha-beta CD4 or CD8 T cell numbers in the test assay mixture;
Detecting and/or quantifying alpha-beta CD4 or CD8 T cell proliferation in the test assay mixture;
Detecting and/or quantifying Vgamma9Vdelta2 (Vγ9Vδ2) T cell numbers in the test assay mixture;
Detecting and/or quantifying Vgamma9Vdelta2 (Vγ9Vδ2) T cell responses in the test assay mixture;
Detecting and/or quantifying Vgamma9Vdelta2 (Vγ9Vδ2) T cell proliferation in the test assay mixture;
Quantifying cancer cell numbers in the test assay mixture;
Quantifying microbial cell or infectious agent numbers in the test assay mixture; or
A combination thereof.

BTN3A1 is ubiquitously expressed across tissues and cell types. A variety of cells and cell populations can be used in the assay mixtures. In some cases, the cells are modified to express or over-express BTN3A1. In some cases, the cells naturally express BTN3A1. In some cases, the cells have the potential to express BTN3A1 but when initially mixed with a test agent the cells do not express detectable amounts of BTN3A1.

The cells or cell populations that are contacted with the test agent can include a variety of BTN3A1-expressing cells such as healthy non-cancerous cells, disease cells, cancer cells, immune cells, or combinations thereof. Various types of healthy and/or diseased cells can be used in the methods. For example, the cells or tissues can be infected with bacteria, viruses, protozoa, or a combination thereof. Such infections can, for example, include infections by malaria (Plasmodium), Listeria (Listeria monocytogenes), tuberculosis (Mycobacterium tuberculosis), viruses, and combinations thereof can be employed. Immune cells that can be used include CD4 T cells, CD8 T cells, Vγ9Vδ2 T cells, other γδ T cells, monocytes, B cells, and/or alpha-beta T cells. The cancer cells employed can include leukemia cells, lymphoma cells, Hodgkin's disease cells, sarcomas of the soft tissue and bone, lung cancer cells, mesothelioma, esophagus cancer cells, stomach cancer cells, pancreatic cancer cells, hepatobiliary cancer cells, small intestinal cancer cells, colon cancer cells, colorectal cancer cells, rectum cancer cells, kidney cancer cells, urethral cancer cells, bladder cancer cells, prostate cancer cells, testis cancer cells, cervical cancer cells, ovarian cancer cells, breast cancer cells, endocrine system cancer cells, skin cancer cells, central nervous system cancer cells, melanoma cells of cutaneous and/or intraocular origin, cancer cells associated with AIDS, or a combination thereof. In addition, metastatic cancer cells at any stage of progression can be used in the assays, such as micrometastatic tumor cells, megametastatic tumor cells, and recurrent cancer cells.

The cells and the test agents can be incubated together for a time and under conditions effective to detect whether the test agent can modulate the expression or activity of BTN3A1, the expression or activity of a BTN3A1 regulator, or the expression or activity of at least one cell in the assay mixture. For example, the cells and test agents can be incubated for a time and under conditions effective for:
Detecting BTN3A1 protein expression on the surface of one or more cells in the test assay mixture;
Quantifying the amount of BTN3A1 protein within one or more of the cells or on the surface of one or more of the cells within the test assay mixture;
Quantifying the number of cells that express BTN3A1 protein in the population of cells;
Detecting and/or quantifying alpha-beta CD4 or CD8 T cell numbers in the test assay mixture;
Detecting and/or quantifying alpha-beta CD4 or CD8 T cell responses in the test assay mixture;
Detecting and/or quantifying Vgamma9Vdelta2 (Vγ9Vδ2) T cell numbers in the test assay mixture;
Detecting and/or quantifying Vgamma9Vdelta2 (Vγ9Vδ2) T cell responses in the test assay mixture;
Quantifying cancer cell numbers in the test assay mixture; or
A combination thereof.

Various procedures can be used to detect and quantify the assay mixtures after the cells are mixed with and incubated with the test agents. Examples of procedures include antibody staining of BTN3A1, antibody staining of one or more BTN3A1 regulator, cell flow cytometry, RNA detection, RNA quantification, RNA sequencing, protein detection, SDS-polyacrylamide gel electrophoresis, DNA sequencing, cytokine detection, interferon detection, and combinations thereof.

The test agents can be any of the BTN3A1 regulators described herein, one or more anti-BTN3A1 antibody, one or more BTN3A1 inhibitory nucleic acid that can modulate the expression of any of the BTN3A1, one or more antibody that can bind any of the BTN3A1 regulators described herein, one or more inhibitory nucleic acid that can modulate the expression of any of the BTN3A1 regulators described herein, one or more small molecules that can modulate BTN3A1, one or more small molecules that can modulate any of the BTN3A1 regulators described herein, or a combination thereof.

Test agents that exhibit in vitro activity for modulating the amount or activity of BTN3A1 or for modulating the amount or activity of any of the BTN3A1 regulators described herein can be evaluated in animal disease models. Such animal disease models can include cancer disease animal models, immune system disease animal models, infectious disease animal models, or combinations thereof.

Methods are also described herein for evaluating whether test agents can selectively modulate the proliferation or viability of cells exhibiting increased or decreased levels of BTN3A1 or exhibiting increased or decreased levels any of the regulators of BTN3A1.

If proliferation or viability of cells exhibiting increased or decreased levels BTN3A1 or exhibiting increased or decreased levels any of the positive regulators of BTN3A1 described herein is decreased in the presence of a test compound as compared to a normal control cell then that test compound has utility for reducing the growth and/or metastasis of cells exhibiting such increased chromosomal instability.

An assay can include determining whether a compound can specifically cause decreased or increased levels of BTN3A1 in various cell types. If the compound does cause decreased or increased levels of BTN3A1, then the compound can be selected/identified for further study, such as for its suitability as a therapeutic agent to treat a cancer. For example, the candidate compounds identified by the selection methods featured in the invention can be further examined for their ability to target a tumor or to treat cancer by, for example, administering the compound to an animal model.

The cells that are evaluated can include metastatic cells, benign cell samples, and cell lines including as cancer cell lines. The cells that are evaluated can also include cells from a patient with cancer (including a patient with metastatic cancer), or cells from a known cancer type or cancer cell line, or cells exhibiting an overproduction of BTN3A1 or any of the regulators of BTN3A1 described herein. A compound that can modulate the production or activity of BTN3A1 from any of these cell types can be administered to a patient.

For example, one method can include (a) obtaining a cell or tissue sample from a patient, (b) measuring the amount or concentration of BTN3A1 or BTN3A regulator produced from a known number or weight of cells or tissues from the sample to generate a reference BTN3A1 value or a BTN3A regulator reference value; (c) mixing the same known number or weight of cells or tissues from the sample with a test compound to generate a test assay, (d) measuring the BTN3A1 or BTN3A regulator amount or concentration in the test assay (e.g., on the cell surface) to generate a test assay BTN3A1 value or a test assay BTN3A regulator value; (e) optionally repeating steps (c) and (d); and selecting a test compound with a lower or higher test assay BTN3A1 value or selecting a test compound with a lower or higher test assay BTN3A regulator value than the reference BTN3A1 value or BTN3A regulator reference value. The method can further include administering a test compound to an animal model, for example, to further evaluate the toxicity and/or efficacy of the test compound. In some cases, the method can further include administering the test compound to the patent from whom the cell or tissue sample as obtained.

Compounds (e.g., top hits identified by any method described herein) can be used in a cell-based assay using cells that express BTN3A1 or any of the regulators of BTN3A1 as a readout of the efficacy of the compounds.

Assay methods are also described herein for identifying and assessing the potency of agents that may modulate BTN3A1 or that may modulate any of the regulators of BTN3A1 listed in Tables 1 and 2.

For example, BTN3A1 can regulate the release of cytokines and interferon γ by activated T-cells. Cells expressing BTN3A1 or modulators of BTN3A1 can be contacted with a test agent and the release of cytokines and/or interferon γ by activated T-cells can be measured. Such a test agent-related level of cytokines and/or interferon γ can be compared to the level observed for cells expressing BTN3A1 or modulators of BTN3A1 that were not contacted with a test agent.

In another example, inhibition of BTN3A1 or inhibition of positive regulators of BTN3A1 can increase T cell responses, gamma-delta T cell responses, Vgamma9Vdelta2 (Vγ9Vδ2) T cell responses, alpha-beta I cell responses, or CD8 T cell responses Test agents can be identified by screening assays that involve quantifying T cell responses to a population of cells that express BTN3A1 or a positive regulator of BTN3A1. The level of T cell responses can be the effect(s) that the T cells have on other cells, for example, cancer cells. For example, the level of T cell responses can be measured by measuring the percent or quantity of cancer cells killed in the assay mixture. The level of T cell responses observed when the test agent is present can be compared to control levels of T cell responses. Such a control can be the level of T cell responses observed when the test agent is not present but all other components in the assay are the same.

In another example, increases in BTN3A1 expression or activity, or increases in the expression or activity of any of the positive regulators of BTN3A1, can increase activation of a subset of human gamma-delta T cells called Vgamma9Vdelta2 (Vγ9Vδ2) T cells. The level of Vγ9Vδ2 T cell responses or proliferation observed when the test agent is present can be compared to control levels of Vγ9Vδ2 T cell responses. Such a control can be the level of Vγ9Vδ2 T cell responses observed when the test agent is not present but all other components in the assay are the same.

### CRISPR Modifications

In some cases, clustered regularly interspaced short palindromic repeats (CRISPR)/CRISPR-associated (Cas) systems can be used to create one or more modifications in genomic BTN3A1 alleles, in any of the BTN3A1 regulator genes, or in any combination thereof. Such CRISPR modifications can reduce the expression or functioning of the BTN3A1 and/or regulator gene products. CRISPR/Cas systems are useful, for example, for RNA-programmable genome editing (see e.g., Marraffini and Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties).

A CRISPR guide RNA can be used that can target a Cas enzyme to the desired location in the genome, where it can cleave the genomic DNA for generation of a genomic modification. This technique is described, for example, by Mali et al. Science 2013 339:823-6; which is incorporated by reference herein in its entirety. Kits for the design and use of CRISPR-mediated genome editing are commercially available, e.g. the PRECISION X CAS9 SMART NUCLEASE™ System (Cat No. CAS900A-1) from System Biosciences, Mountain View, CA.

In other cases, a cre-lox recombination system of bacteriophage P1, described by Abremski et al. 1983. Cell 32:1301 (1983), Sternberg et al., Cold Spring Harbor Symposia on Quantitative Biology, Vol. XLV 297 (1981) and others, can be used to promote recombination and alteration of the BTN3A1 and/or regulator genomic site(s). The cre-lox system utilizes the cre recombinase isolated from bacteriophage P1 in conjunction with the DNA sequences that the recombinase recognizes (termed lox sites). This recombination system has been effective for achieving recombination in plant cells (see, e.g., U.S. Pat. No. 5,658,772), animal cells (U.S. Pat. Nos. 4,959,317 and 5,801,030), and in viral vectors (Hardy et al., J. Virology 71:1842 (1997).

The genomic mutations so incorporated can alter one or more amino acids in the encoded BTN3A1 and/or regulator gene products. For example, genomic sites modified so that in the encoded BTN3A1 and/or regulator protein is more prone to degradation, is less stable so that the half-life of such protein(s) is reduced, or so that the BTN3A1 and/or regulator has improved expression or functioning. In another example, genomic sites can be modified so that at least one amino acid of a BTN3A1 and/or regulator polypeptide is deleted or mutated to alter its activity. For example, a conserved amino acid or a conserved domain can be modified to improve or reduce of the activity of the BTN3A1 and/or regulator polypeptide. For example, a conserved amino acid or several amino acids in a conserved domain of the BTN3A1 and/or regulator polypeptide can be replaced with one or more amino acids having physical and/or chemical properties that are different from the conserved amino acid(s). For example, to change the physical and/or chemical properties of the conserved amino acid(s), the conserved amino acid(s) can be deleted or replaced by amino acid(s) of another class, where the classes are identified in the following table.

| Classification | Genetically Encoded |
|---|---|
| Hydrophobic | A, G, F, I, L, M, P, V, W |
| Aromatic | F, Y, W |
| Apolar | M, G, P |
| Aliphatic | A, V, L, I |
| Hydrophilic | C, D, E, H, K, N, Q, R, S, T, Y |
| Acidic | D, E |
| Basic | H, K, R |
| Polar | Q, N, S, T, Y |
| Cysteine-Like | C |

The guide RNAs and nuclease can be introduced via one or more vehicles such as by one or more expression vectors (e.g., viral vectors), virus like particles, ribonucleoproteins (RNPs), via nanoparticles, liposomes, or a combination thereof. The vehicles can include components or agents that can target particular cell types (e.g., antibodies that recognize cell-surface markers), facilitate cell penetration, reduce degradation, or a combination thereof.

### Inhibitory Nucleic Acids

The expression of BTN3A1, a BTN3A1 regulator, or any combination thereof can be inhibited, for example by use of an inhibitory nucleic acid that specifically recognizes a nucleic acid that encodes the BTN3A1 or the BTN3A1 regulator.

An inhibitory nucleic acid can have at least one segment that will hybridize to a BTN3A1 nucleic acid and/or a BTN3A1 regulator nucleic acid under intracellular or stringent conditions. The inhibitory nucleic acid can reduce expression of a nucleic acid encoding BTN3A1 or a BTN3A1 regulator. A nucleic acid may hybridize to a genomic DNA, a messenger RNA, or a combination thereof. An inhibitory nucleic acid may be incorporated into a plasmid vector or viral DNA. It may be single stranded or double stranded, circular or linear.

An inhibitory nucleic acid is a polymer of ribose nucleotides or deoxyribose nucleotides having more than 13 nucleotides in length. An inhibitory nucleic acid may include naturally occurring nucleotides; synthetic, modified, or pseudo-nucleotides such as phosphorothiolates; as well as nucleotides having a detectable label such as P³², biotin or digoxigenin. An inhibitory nucleic acid can reduce the expression and/or activity of a BTN3A1 nucleic acid and/or a BTN3A1 regulator nucleic acid. Such an inhibitory nucleic acid may be completely complementary to a segment of an endogenous BTN3A1 nucleic acid (e.g., an RNA) or an endogenous BTN3A1 regulator nucleic acid (e.g., an RNA). Alternatively, some variability is permitted in the inhibitory nucleic acid sequences relative to BTN3A1 or a BTN3A1 regulator sequences. An inhibitory nucleic acid can hybridize to a BTN3A1 nucleic acid or a BTN3A1 regulator nucleic acid under intracellular conditions or under stringent hybridization conditions and is sufficiently complementary to inhibit expression of the endogenous BTN3A1 nucleic acid or the endogenous BTN3A1 regulator nucleic acid. Intracellular conditions refer to conditions such as temperature, pH and salt concentrations typically found inside a cell, e.g. an animal or mammalian cell. One example of such an animal or mammalian cell is a myeloid progenitor cell. Another example of such an animal or mammalian cell is a more differentiated cell derived from a myeloid progenitor cell. Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to a BTN3A1 coding sequence or a BTN3A1 regulator coding sequence, each separated by a stretch of contiguous nucleotides that are not complementary to adjacent coding sequences, can inhibit the function of a BTN3A1 nucleic acid and/or one or more nucleic acids for any of the regulators of BTN3A1. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme or an antisense nucleic acid molecule.

The inhibitory nucleic acid molecule may be single or double stranded (e.g. a small interfering RNA (siRNA)) and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce translation of BTN3A1 and/or any of the regulators of BTN3A1 such that translation of the encoded BTN3A1 and/or regulator polypeptide is reduced. SiRNAs mediate post-transcriptional gene silencing in a sequence-specific manner. See, for example, website at invitrogen com/site/us/en/home/Products-and-Services/Applications/rnai.html. Once incorporated into an RNA-induced silencing complex, siRNA mediate cleavage of the homologous endogenous mRNA transcript by guiding the complex to the homologous mRNA transcript, which is then cleaved by the complex. The siRNA may be homologous and/or complementary to any region of the BTN3A1 transcript and/or any of the transcripts of the regulators of BTN3A1. The region of homology may be 30 nucleotides or less in length, preferable less than 25 nucleotides, and more preferably about 21 to 23 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411: 494-498 (2001); Harborth et al. Antisense Nucleic Acid Drug Dev. 13: 83-106 (2003).

The pSuppressorNeo vector for expressing hairpin siRNA, commercially available from IMGENEX (San Diego, California), can be used to generate siRNA for inhibiting expression of BTN3A1 and/or any of the regulators of BTN3A1. The construction of the siRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26(2): p. 199-213. Accordingly, for synthesis of synthetic siRNA, a target region may be selected preferably 50 to 100 nucleotides downstream of the start codon. The 5′ and 3′ untranslated regions and regions close to the start codon should be avoided as these may be richer in regulatory protein binding sites. As siRNA can begin with AA, have 3′ UU overhangs for both the sense and antisense siRNA strands, and have an approximate 50% G/C content. An example of a sequence for a synthetic siRNA is 5′-AA(N19)UU, where N is any nucleotide in the mRNA sequence and should be approximately 50% G-C content. The selected sequence(s) can be compared to others in the human genome database to minimize homology to other known coding sequences (e.g., by Blast search, for example, through the NCBI website).

SiRNAs may be chemically synthesized, created by in vitro transcription, or expressed from an siRNA expression vector or a PCR expression cassette. See, e.g., website at invitrogen.com/site/us/en/home/Products-and-Services/Applications/rnai.html. When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, preferably, 3 to 23 nucleotides in length, and may be of various nucleotide sequences including, AUG, CCC, UUCG, CCACC, CTCGAG, AAGCUU, CCACACC and UUCAAGAGA (SEQ ID NO:109). SiRNAs also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target BTN3A1 nucleic acid or the target nucleic acid for any of the regulators of BTN3A1.

An inhibitory nucleic acid may be prepared using available methods, for example, by expression from an expression vector encoding a complementarity sequence of the BTN3A1 nucleic acid or the nucleic acids for any of the regulators of BTN3A1. Alternatively, it may be prepared by chemical synthesis using naturally occurring nucleotides, modified nucleotides or any mixture of combination thereof. In some embodiments, the BTN3A1 nucleic acids and in the nucleic acids of the regulators of BTN3A1 are made from modified nucleotides or non-phosphodiester bonds, for example, that are designed to increase biological stability of the nucleic acids or to increase intracellular stability of the duplex formed between the inhibitory nucleic acids and other (e.g., endogenous) nucleic acids.

For example, the BTN3A1 nucleic acids and the nucleic acids of the regulators of BTN3A1 can be peptide nucleic acids that have peptide bonds rather than phosphodiester bonds.

Naturally occurring nucleotides that can be employed in the BTN3A1 nucleic acids and in the nucleic acids of the regulators of BTN3A1 include the ribose or deoxyribose nucleotides adenosine, guanine, cytosine, thymine and uracil. Examples of modified nucleotides that can be employed in the BTN3A1 nucleic acids and in the nucleic acids of the regulators of BTN3A1 include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine.

Thus, inhibitory nucleic acids of the BTN3A1 and of the regulators of BTN3A1 described herein may include modified nucleotides, as well as natural nucleotides such as combinations of ribose and deoxyribose nucleotides. The inhibitory nucleic acids and may be of same length as wild type BTN3A1 or as any of the regulators of BTN3A1 described herein. The inhibitory nucleic acids of the BTN3A1 and of the regulators of BTN3A1 described herein can also be longer and include other useful sequences. In some embodiments, the inhibitory nucleic acids of the BTN3A1 and of the regulators of BTN3A1 described herein are somewhat shorter. For example, inhibitory nucleic acids of the BTN3A1 and of the regulators of BTN3A1 described herein can include a segment that has a nucleic acid sequence that can be missing up to 5 nucleotides, or missing up to 10 nucleotides, or missing up to nucleotides, or missing up to 30 nucleotides, or missing up to 50 nucleotides, or missing up to 100 nucleotides from the 5′ or 3′ end.

The inhibitory nucleic acids can be introduced via one or more vehicles such as via expression vectors (e.g., viral vectors), via virus like particles, via ribonucleoproteins (RNPs), via nanoparticles, via liposomes, or a combination thereof. The vehicles can include components or agents that can target particular cell types, facilitate cell penetration, reduce degradation, or a combination thereof

### Antibodies

Antibodies can be used as inhibitors and activators of BTN3A1 and any of the regulators of BTN3A1 described herein. Antibodies can be raised against various epitopes of the BTN3A1 or any of the regulators of BTN3A1 described herein. Some antibodies for BTN3A1 or any of the regulators of BTN3A1 described herein may also be available commercially. However, the antibodies contemplated for treatment pursuant to the methods and compositions described herein are preferably human or humanized antibodies and are highly specific for their targets.

In one aspect, the present disclosure relates to use of isolated antibodies that bind specifically to BTN3A1 or any of the regulators of BTN3A1 described herein. Such antibodies may be monoclonal antibodies. Such antibodies may also be humanized or fully human monoclonal antibodies. The antibodies can exhibit one or more desirable functional properties, such as high affinity binding to BTN3A1 or any of the regulators of BTN3A1 described herein, or the ability to inhibit binding of BTN3A1 or any of the regulators of BTN3A1 described herein.

Methods and compositions described herein can include antibodies that bind BTN3A1 or any of the regulators of BTN3A1 described herein, or a combination of antibodies where each antibody type can separately bind BTN3A1 or one of the regulators of BTN3A1 described herein.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g. a peptide or domain of BTN3A1 or any of the regulators of BTN3A1 described herein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains: (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds BTN3A1 or any of the regulators of BTN3A1 described herein is substantially free of antibodies that specifically bind antigens other than BTN3A1 or any of the regulators of BTN3A1 described herein. An isolated antibody that specifically binds BTN3A1 or any of the regulators of BTN3A1 described herein may, however, have cross-reactivity to other antigens, such as isoforms or related BTN3A1 and regulators of BTN3A1 proteins from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{L} and V_{H} regions of the recombinant antibodies are sequences that, while derived from and related to human germline V_{L} and V_{H} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to human BTN3A1 or any of the regulators of BTN3A1 described herein” is intended to refer to an antibody that binds to human BTN3A1 or any of the regulators of BTN3A1 described herein with a K_{D} of 1×10⁻⁷ M or less, more preferably 5×10⁻⁸ M or less, more preferably 1×10⁻⁸ M or less, more preferably 5×10⁻⁹ M or less, even more preferably between 1×10⁻⁸ M and 1×10⁻¹⁰ M or less.

The term “K_{assoc}” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D},” as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The antibodies of the invention are characterized by particular functional features or properties of the antibodies. For example, the antibodies bind specifically to human BTN3A1 or any of the regulators of BTN3A1 described herein. Preferably, an antibody of the invention binds to BTN3A1 or any of the regulators of BTN3A1 described herein with high affinity, for example with a K_{D} of 1×10⁻⁷ M or less. The antibodies can exhibit one or more of the following characteristics:
(a) binds to human BTN3A1 or any of the regulators of BTN3A1 described herein with a K_{D} of 1×10⁻⁷ M or less;
(b) inhibits the function or activity of BTN3A1 or any of the regulators of BTN3A1 described herein;
(c) inhibits cancer (e.g., cancer cells expressing BTN3A1 or any of the positive regulators of BTN3A1 described herein); or
(d) a combination thereof.

Assays to evaluate the binding ability of the antibodies toward BTN3A1 or any of the regulators of BTN3A1 described herein can be used, including for example, ELISAs, Western blots and RIAs. The binding kinetics (e.g., binding affinity) of the antibodies also can be assessed by standard assays known in the art, such as by Biacore™. analysis.

Given that each of the subject antibodies can bind to BTN3A1 or any of the regulators of BTN3A1 described herein, the V_{L} and V_{H} sequences can be “mixed and matched” to create other binding molecules that bind to BTN3A1 or any of the regulators of BTN3A1 described herein. The binding properties of such “mixed and matched” antibodies can be tested using the binding assays described above and assessed in assays described in the examples. When V_{L} and Vii chains are mixed and matched, a V_{H} sequence from a particular V_{H}/V_{L} pairing can be replaced with a structurally similar V_{H} sequence. Likewise, preferably a V_{L} sequence from a particular V_{H}/V_{L} pairing is replaced with a structurally similar V_{L} sequence.

Accordingly, in one aspect, the invention provides an isolated monoclonal antibody, or antigen binding portion thereof comprising:
(a) a heavy chain variable region comprising an amino acid sequence; and
(b) a light chain variable region comprising an amino acid sequence;
wherein the antibody specifically binds BTN3A1 or any of the regulators of BTN3A1 described herein.

In some cases, the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J. of Cancer 83(2):252-260 (2000) (describing the production of a humanized anti-CD30 antibody using only the heavy chain variable domain CDR3 of murine anti-CD30 antibody Ki-4): Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin alphaᵥbeta₃ antibodies using a heavy and light chain variable CDR3 domain. Hence, in some cases a mixed and matched antibody or a humanized antibody contains a CDR3 antigen binding domain that is specific for BTN3A1 or any of the regulators of BTN3A1 described herein.

### Small Molecule Modulators

Examples of small molecules that can directly or indirectly modulate BTN3A1 or any of the regulators of BTN3A1 described herein are shown in the table below.

| Compound | Class | Target |
|---|---|---|
| Rotenone | Inhibitor | Complex I (NADH:ubiquinone |
| | | oxidoreductase) |
| Piericidin A | Inhibitor | Complex I (NADH:ubiquinone |
| | | oxidoreductase) |
| Metformin | Inhibitor | Complex I (NADH:ubiquinone |
| | | oxidoreductase) |
| α-Keto-γ-(methylthio)bu- | Inhibitor | CTBP1 |
| tyric acid | | |
| 6-Mercaptopurine | Inhibitor | Purine metabolism |
| monohydrate | | |
| Mycophenolic Acid | Inhibitor | Purine metabolism |
| Zoledronate | Inhibitor | FDPS |
| Risedronate | Inhibitor | FDPS |
| Alendronate | Inhibitor | FDPS |
| AICAR | Activator | AMP-activated protein kinase |
| | | (AMPK) |
| Compound 991 | Activator | AMP-activated protein kinase |
| | | (AMPK) |
| A-769662 | Activator | AMP-activated protein kinase |
| | | (AMPK) |
| 2,4-Dinitrophenol | Activator | AMP-activated protein kinase |
| | | (AMPK) |
| Berberine | Activator | AMP-activated protein kinase |
| | | (AMPK) |
| Canagliflozin | Activator | AMP-activated protein kinase |
| | | (AMPK) |
| Metformin | Activator | AMP-activated protein kinase |
| | | (AMPK) |
| Methotrexate | Activator | AMP-activated protein kinase |
| | | (AMPK) |
| Phenformin | Activator | AMP-activated protein kinase |
| | | (AMPK) |
| PT-1 | Activator | AMP-activated protein kinase |
| | | (AMPK) |
| Quercetin | Activator | AMP-activated protein kinase |
| | | (AMPK) |
| R419 | Activator | AMP-activated protein kinase |
| | | (AMPK) |
| Resveratrol | Activator | AMP-activated protein kinase |
| | | (AMPK) |
| 3 (2-(2-(4-(trifluoromethyl) | Activator | AMP-activated protein kinase |
| phenylamino)thiazol-4- | | (AMPK) |
| yl)acetic acid | | |
| C2 | Activator | AMP-activated protein kinase |
| | | (AMPK) |
| BPA-CoA | Activator | AMP-activated protein kinase |
| | | (AMPK) |
| MK-8722 | Activator | AMP-activated protein kinase |
| | | (AMPK) |
| MT 63-78 | Activator | AMP-activated protein kinase |
| | | (AMPK) |
| O304 | Activator | AMP-activated protein kinase |
| | | (AMPK) |
| PF249 | Activator | AMP-activated protein kinase |
| | | (AMPK) |
| Salicylate | Activator | AMP-activated protein kinase |
| | | (AMPK) |
| SC4 | Activator | AMP-activated protein kinase |
| | | (AMPK) |
| ZMP | Activator | AMP-activated protein kinase |
| | | (AMPK) |

The structures and/or chemical formulae for many the compounds listed in this table are provided by Steinberg & Carling, AMP-activated protein kinase: the current landscape for drug development, Nature Reviews 18:527 (2019).

“Treatment” or “treating” refers to both therapeutic treatment and to prophylactic or preventative measures. Those in need of treatment include those already with the disorder as well as those prone to have the disorder, or those in whom the disorder is to be prevented.

“Subject” for purposes of administration of a test agent or composition described herein refers to any animal classified as a mammal or bird, including humans, domestic animals, farm animals, zoo animals, experimental animals, pet animals, such as dogs, horses, cats, cows, etc. The experimental animals can include mice, rats, guinea pigs, goats, dogs, monkeys, or a combination thereof. In some cases, the subject is human.

As used herein, the term “cancer” includes solid animal tumors as well as hematological malignancies. The terms “tumor cell(s)” and “cancer cell(s)” are used interchangeably herein.

“Solid animal tumors” include cancers of the head and neck, lung, mesothelioma, mediastinum, lung, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone: and melanoma of cutaneous and intraocular origin. In addition, a metastatic cancer at any stage of progression can be treated, such as micrometastatic tumors, megametastatic tumors, and recurrent cancers.

The term “hematological malignancies” includes adult or childhood leukemia and lymphomas, Hodgkin's disease, lymphomas of lymphocytic and cutaneous origin, acute and chronic leukemia, plasma cell neoplasm and cancers associated with AIDS.

The inventive methods and compositions can also be used to treat leukemias, lymph nodes, thymus tissues, tonsils, spleen, cancer of the breast, cancer of the lung, cancer of the adrenal cortex, cancer of the cervix, cancer of the endometrium, cancer of the esophagus, cancer of the head and neck, cancer of the liver, cancer of the pancreas, cancer of the prostate, cancer of the thymus, carcinoid tumors, chronic lymphocytic leukemia, Ewing's sarcoma, gestational trophoblastic tumors, hepatoblastoma, multiple myeloma, non-small cell lung cancer, retinoblastoma, or tumors in the ovaries. A cancer at any stage of progression can be treated or detected, such as primary, metastatic, and recurrent cancers. In some cases, metastatic cancers are treated but primary cancers are not treated. Information regarding numerous types of cancer can be found, e.g., from the American Cancer Society (cancer.org), or from, e.g., Wilson et al. (1991) Harrison's Principles of Internal Medicine, 12th Edition, McGraw-Hill, Inc.

In some embodiments, the cancer and/or tumors to be treated are hematological malignancies, or those of lymphoid origin such as cancers or tumors of lymph nodes, thymus tissues, tonsils, spleen, and cells related thereto. In some embodiments, the cancer and/or tumors to be treated are those that have been resistant to T cell therapies.

Treatment of, or treating, metastatic cancer can include the reduction in cancer cell migration or the reduction in establishment of at least one metastatic tumor. The treatment also includes alleviation or diminishment of more than one symptom of metastatic cancer such as coughing, shortness of breath, hemoptysis, lymphadenopathy, enlarged liver, nausea, jaundice, bone pain, bone fractures, headaches, seizures, systemic pain and combinations thereof. The treatment may cure the cancer, e.g., it may prevent metastatic cancer, it may substantially eliminate metastatic tumor formation and growth, and/or it may arrest or inhibit the migration of metastatic cancer cells.

Anti-cancer activity can reduce the progression of a variety of cancers (e.g., breast, lung, pancreatic, or prostate cancer) using methods available to one of skill in the art. Anti-cancer activity, for example, can determined by identifying the lethal dose (LD₁₀₀) or the 50% effective dose (ED50) or the dose that inhibits growth at 50% (GI₅₀) of an agent of the present invention that prevents the migration of cancer cells. In one aspect, anti-cancer activity is the amount of the agent that reduces 50%, 60%, 70%, 80%, 90%, 95%, 97%, 98%, 99% or 100% of cancer cell migration, for example, when measured by detecting expression of a cancer cell marker at sites proximal or distal from a primary tumor site, or when assessed using available methods for detecting metastases.

In another example, agents that increase or decrease BTN3A1 expression or function can be administered to sensitize tumor cells to immune therapies. Hence, by administering an agent that increase or decrease BTN3A1 expression or function, tumor cells can become more sensitive to the immune system and to various immune therapies.

### Compositions

The invention also relates to compositions containing T cells and/or other chemotherapeutic agents. Such agents can be polypeptides, nucleic acids encoding one or more polypeptides (e.g., within an expression cassette or expression vector), small molecules, compounds or agents identified by a method described herein, or a combination thereof. The compositions can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

The composition can be formulated in any convenient form. In some embodiments, the compositions can include a protein or polypeptide encoded by any of the genes listed in Table 1 or 2. In other embodiments, the compositions can include at least one nucleic acid or expression cassette encoding a polypeptide listed in Table 1 or 2. In other embodiments, the compositions can include at least one nucleic acid, guide RNA, or expression cassette that includes a nucleic acid segment encoding a guide RNA or an inhibitory nucleic acid complementarity to gene listed in Table 1 or 2. In other embodiments, the compositions can include at least one antibody that binds at least one protein encoded by at least one gene listed in Table 1 or 2. In other embodiments, the compositions can include at least one small molecule that binds, that activates, or that inhibits at least one gene listed in Table 1 or 2, or at least one small molecule that binds, that activates, or that inhibits at least one protein encoded by at least one gene listed in Table 1 or 2

In some embodiments, the chemotherapeutic agents of the invention (e.g., polypeptide, a nucleic acid encoding a polypeptide (e.g., within an expression cassette or expression vector), a guide RNA, an inhibitory nucleic acid, a small molecule, a compound identified by a method described herein, or a combination thereof), are administered in a “therapeutically effective amount.” Such a therapeutically effective amount is an amount sufficient to obtain the desired physiological effect, such a reduction of at least one symptom of cancer. For example, chemotherapeutic agents can reduce cell metastasis by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%.

Symptoms of cancer can also include tumor cachexia, tumor-induced pain conditions, tumor-induced fatigue, cancer cell growth, tumor growth, and metastatic spread. Hence, the chemotherapeutic agents may also reduce tumor cachexia, tumor-induced pain conditions, tumor-induced fatigue, cancer cell growth, tumor growth, metastatic spread, or a combination thereof by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%.

To achieve the desired effect(s), the chemotherapeutic agents may be administered as single or divided dosages. For example, chemotherapeutic agents can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the type of small molecules, compounds, peptides, expression system, or nucleic acid chosen for administration, the disease, the weight, the physical condition, the health, and the age of the mammal. Such factors can be readily determined by the clinician employing animal models or other test systems that are available in the art.

Administration of the chemotherapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the chemotherapeutic agents and compositions of the invention may be essentially continuous over a preselected period of time or may be in a series of spaced doses. Both local and systemic administration is contemplated.

To prepare the T cells, compositions, small molecules, compounds, polypeptides, nucleic acids, expression cassettes, and other agents are synthesized or otherwise obtained, purified as necessary or desired. These T cells, compositions, small molecules, compounds, polypeptides, nucleic acids, expression cassettes, and other agents can be suspended in a pharmaceutically acceptable carrier. In some cases, the compositions, small molecules, compounds, polypeptides, nucleic acids, expression cassette, and/or other agents can be lyophilized or otherwise stabilized. The T cells, compositions, small molecules, compounds, polypeptides, nucleic acids, expression cassettes, other agents, and combinations thereof can be adjusted to an appropriate concentration, and optionally combined with other agents. The absolute weight of a given T cell preparation, composition, small molecule, compound, polypeptide, nucleic acid, and/or other agents included in a unit dose can vary widely. For example, about 0.01 to about 2 g, or about 0.1 to about 500 mg, of at least one molecule, compound, polypeptide, nucleic acid, and/or other agent, or a plurality of molecules, compounds, polypeptides, nucleic acids, and/or other agents can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the chemotherapeutic agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amount of chemotherapeutic agent for use in treatment will vary not only with the particular carrier selected but also with the route of administration, the nature of the cancer condition being treated and the age and condition of the patient. Ultimately the attendant health care provider can determine proper dosage. In addition, a pharmaceutical composition can be formulated as a single unit dosage form.

Thus, one or more suitable unit dosage forms comprising the chemotherapeutic agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The chemotherapeutic agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the chemotherapeutic agent with liquid carriers, solid matrices, semi-solid carriers, finely divided solid carriers or combinations thereof, and then, if necessary, introducing or shaping the product into the desired delivery system. For example, the chemotherapeutic agent(s) can be linked to a convenient carrier such as a nanoparticle, albumin, polyalkylene glycol, or be supplied in prodrug form. The chemotherapeutic agent(s), and combinations thereof can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of inhibitors can also involve parenteral or local administration of the in an aqueous solution or sustained release vehicle.

Thus, while the chemotherapeutic agent(s) and/or other agents can sometimes be administered in an oral dosage form, that oral dosage form can be formulated so as to protect the small molecules, compounds, polypeptides, nucleic acids, expression cassettes, and combinations thereof from degradation or breakdown before the small molecules, compounds, polypeptides, nucleic acids encoding such polypeptides, and combinations thereof provide therapeutic utility. For example, in some cases the small molecules, compounds, polypeptides, nucleic acids encoding such polypeptide, and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The chemotherapeutic agent(s) and/or other agents can be formulated in dry form (e.g., in freeze-dried form), in the presence or absence of a carrier. If a carrier is desired, the carrier can be included in the pharmaceutical formulation, or can be separately packaged in a separate container, for addition to the inhibitor that is packaged in dry form, in suspension or in soluble concentrated form in a convenient liquid.

T cells, chemotherapeutic agent(s), other agents, or a combination thereof can be formulated for parenteral administration (e.g., by injection, for example, bolus injection or continuous infusion) and may be presented in unit dosage form in ampoules, prefilled syringes, small volume infusion containers or multi-dose containers with an added preservative.

The compositions can also contain other ingredients such as chemotherapeutic agents, anti-viral agents, antibacterial agents, antimicrobial agents and/or preservatives. Examples of additional therapeutic agents that may be used include, but are not limited to: alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin: enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel (Taxol®), docetaxel (Taxotere®), and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. The compositions can also be used in conjunction with radiation therapy.

The present description is further illustrated by the following examples, which should not be construed as limiting in any way. The contents of all cited references (including literature references, issued patents, published patent applications as cited throughout this application) are hereby expressly incorporated by reference.

### Example 1: CRISPR Knockout Screen for BTN3A1 Regulators

This Example describes a genome wide CRISPR knockout screen of a human cancer cell line (Daudi) for identifying genes in the human genome that positively regulate or that negatively regulate the levels of BTN3A1 on the cell surface.

Aliquots of Daudi cells that stably express Cas9 were lentivirally transduced with the Human Improved Genome-wide Knockout CRISPR Library multi-guide sgRNA library (Addgene, Pooled Library #67989). The cells were stained with labeled anti-BTN3A1 antibodies (clone BT3.1, Novus Biologicals) and cells exhibiting statistically significant increased or decreased BTN3A1 expression were identified and isolated by fluorescence-activated cell sorting. Their genomic DNA was isolated, and regions corresponding to the integrated sgRNA were amplified and sequenced to identify regulators of BTN3A1. Three replicates of the screen were performed, and the identified statistically significant hits were consistent across all the replicates.

### Example 2: Negative Regulators of BTN3A1

This Example provides a list of the gene products that reduce BTN3A1 expression

TABLE 1
Negative Regulators of BTN3A1

| | | False-discovery | | Log2 Fold |
|---|---|---|---|---|
| Gene ID | p-value | Rate | Rank | Change |
| CTBP1 | 2.75E−07 | 3.30E−05 | 1 | −4.973 |
| UBE2E1 | 2.75E−07 | 3.30E−05 | 2 | −1.4857 |
| RING1 | 2.75E−07 | 3.30E−05 | 3 | −1.825 |
| ZNF217 | 2.75E−07 | 3.30E−05 | 4 | −3.2144 |
| HDAC8 | 2.75E−07 | 3.30E−05 | 5 | −1.4131 |
| RUNX1 | 2.75E−07 | 3.30E−05 | 6 | −4.2266 |
| RBM38 | 2.75E−07 | 3.30E−05 | 7 | −1.63 |
| CBFB | 2.75E−07 | 3.30E−05 | 8 | −3.9976 |
| RER1 | 2.75E−07 | 3.30E−05 | 9 | −5.246 |
| IKZF1 | 2.75E−07 | 3.30E−05 | 10 | −1.8146 |
| KCTD5 | 2.75E−07 | 3.30E−05 | 11 | −3.3621 |
| ST6GAL1 | 2.75E−07 | 3.30E−05 | 12 | −1.3783 |
| ZNF296 | 2.75E−07 | 3.30E−05 | 13 | −2.2127 |
| NFKBIA | 2.75E−07 | 3.30E−05 | 14 | −1.5336 |
| ATIC | 2.75E−07 | 3.30E−05 | 15 | −3.1529 |
| TIAL1 | 2.75E−07 | 3.30E−05 | 16 | −3.1013 |
| CMAS | 2.75E−07 | 3.30E−05 | 17 | −3.0377 |
| CSRNP1 | 2.75E−07 | 3.30E−05 | 18 | −1.5267 |
| GADD45A | 2.75E−07 | 3.30E−05 | 19 | −0.89067 |
| EDEM3 | 2.75E−07 | 3.30E−05 | 20 | −0.95307 |
| AGO2 | 2.75E−07 | 3.30E−05 | 21 | −1.8141 |
| RNASEH2A | 2.75E−07 | 3.30E−05 | 22 | −2.7616 |
| SRD5A3 | 2.75E−07 | 3.30E−05 | 23 | −2.5498 |
| ZNF281 | 2.75E−07 | 3.30E−05 | 24 | −1.7587 |
| MAP2K3 | 2.75E−07 | 3.30E−05 | 25 | −2.0597 |
| SUPT7L | 2.75E−07 | 3.30E−05 | 26 | −3.2156 |
| SLC19A1 | 2.75E−07 | 3.30E−05 | 27 | −1.9897 |
| CCNL1 | 2.75E−07 | 3.30E−05 | 28 | −2.1885 |
| AUP1 | 2.75E−07 | 3.30E−05 | 29 | −2.4069 |
| ZRSR2 | 2.75E−07 | 3.30E−05 | 30 | −2.0246 |
| CDK13 | 2.75E−07 | 3.30E−05 | 31 | −1.6493 |
| RASA2 | 2.75E−07 | 3.30E−05 | 32 | −1.5589 |
| ERF | 2.75E−07 | 3.30E−05 | 33 | −2.0416 |
| EIF4ENIF1 | 2.75E−07 | 3.30E−05 | 34 | −1.6788 |
| PRMT7 | 2.75E−07 | 3.30E−05 | 35 | −1.0238 |
| MOCS3 | 2.75E−07 | 3.30E−05 | 36 | −1.609 |
| HSCB | 2.75E−07 | 3.30E−05 | 37 | −3.6334 |
| EDC4 | 2.75E−07 | 3.30E−05 | 38 | −1.7812 |
| CD79A | 2.75E−07 | 3.30E−05 | 39 | −1.3903 |
| SLC16A1 | 2.75E−07 | 3.30E−05 | 40 | −2.8619 |
| RBM10 | 2.75E−07 | 3.30E−05 | 41 | −1.6212 |
| GALE | 2.75E−07 | 3.30E−05 | 42 | −3.4433 |
| MEF2B | 2.75E−07 | 3.30E−05 | 43 | −2.0198 |
| FAM96B | 2.75E−07 | 3.30E−05 | 44 | −4.0264 |
| ATXN7 | 2.75E−07 | 3.30E−05 | 45 | −1.6552 |
| COG8 | 2.75E−07 | 3.30E−05 | 46 | −1.0713 |
| DERL1 | 2.75E−07 | 3.30E−05 | 47 | −2.0143 |
| TGFBR2 | 2.75E−07 | 3.30E−05 | 48 | −1.765 |
| CHTF8 | 2.75E−07 | 3.30E−05 | 49 | −1.4137 |
| AHCYL1 | 2.75E−07 | 3.30E−05 | 50 | −1.1134 |
| PGM3 | 2.75E−07 | 3.30E−05 | 51 | −1.688 |
| NUDT2 | 2.75E−07 | 3.30E−05 | 52 | −1.3824 |
| COG1 | 2.75E−07 | 3.30E−05 | 53 | −1.1923 |
| TK1 | 2.75E−07 | 3.30E−05 | 54 | −2.5332 |
| HMHA1 | 2.75E−07 | 3.30E−05 | 55 | −1.2717 |
| GPI | 2.75E−07 | 3.30E−05 | 56 | −2.1259 |
| KDM1A | 2.75E−07 | 3.30E−05 | 57 | −3.6146 |
| NANS | 2.75E−07 | 3.30E−05 | 58 | −2.5782 |
| CCDC71L | 2.75E−07 | 3.30E−05 | 59 | −1.1835 |
| MAPK14 | 2.75E−07 | 3.30E−05 | 60 | −2.5037 |
| SLC35A2 | 2.75E−07 | 3.30E−05 | 61 | −2.7731 |
| EHMT1 | 2.75E−07 | 3.30E−05 | 62 | −1.7462 |
| RPL28 | 2.75E−07 | 3.30E−05 | 63 | −1.1157 |
| TRIM33 | 2.75E−07 | 3.30E−05 | 64 | −2.8967 |
| CTU1 | 2.75E−07 | 3.30E−05 | 65 | −1.7287 |
| SLC35A1 | 2.75E−07 | 3.30E−05 | 66 | −2.3244 |
| TFDP2 | 2.75E−07 | 3.30E−05 | 67 | −1.6469 |
| GANAB | 2.75E−07 | 3.30E−05 | 68 | −1.8586 |
| IPO9 | 2.75E−07 | 3.30E−05 | 69 | −1.5781 |
| ZNF644 | 2.75E−07 | 3.30E−05 | 70 | −1.3426 |
| IKBKAP | 2.75E−07 | 3.30E−05 | 71 | −1.1569 |
| ADAT3 | 2.75E−07 | 3.30E−05 | 72 | −1.5648 |
| PTPRCAP | 2.75E−07 | 3.30E−05 | 73 | −1.2157 |
| PPAT | 2.75E−07 | 3.30E−05 | 74 | −5.6022 |
| RBM26 | 2.75E−07 | 3.30E−05 | 75 | −1.5903 |
| MAP3K4 | 2.75E−07 | 3.30E−05 | 76 | −1.2765 |
| EHMT2 | 2.75E−07 | 3.30E−05 | 77 | −1.1513 |
| MSI2 | 2.75E−07 | 3.30E−05 | 78 | −1.9962 |
| BSG | 2.75E−07 | 3.30E−05 | 79 | −1.2131 |
| SND1 | 2.75E−07 | 3.30E−05 | 80 | −0.87423 |
| MLLT1 | 2.75E−07 | 3.30E−05 | 81 | −0.91722 |
| NUBP2 | 2.75E−07 | 3.30E−05 | 82 | −4.0803 |
| ZNF532 | 2.75E−07 | 3.30E−05 | 83 | −1.3013 |
| DPH1 | 2.75E−07 | 3.30E−05 | 84 | −1.078 |
| UBE4B | 2.75E−07 | 3.30E−05 | 85 | −1.5406 |
| SSR2 | 2.75E−07 | 3.30E−05 | 86 | −1.634 |
| ZFR | 2.75E−07 | 3.30E−05 | 87 | −1.1508 |
| FDPS | 2.75E−07 | 3.30E−05 | 88 | −3.9018 |
| DCPS | 2.75E−07 | 3.30E−05 | 89 | −3.0815 |
| PPP2R4 | 2.75E−07 | 3.30E−05 | 90 | −1.8295 |
| TRMT61A | 2.75E−07 | 3.30E−05 | 91 | −2.3517 |
| ALG9 | 2.75E−07 | 3.30E−05 | 92 | −2.0991 |
| RBM4 | 2.75E−07 | 3.30E−05 | 93 | −1.0666 |
| ATXN7L3 | 2.75E−07 | 3.30E−05 | 94 | −2.987 |
| CIAO1 | 2.75E−07 | 3.30E−05 | 95 | −3.1344 |
| SLC4A7 | 2.75E−07 | 3.30E−05 | 96 | −2.7714 |
| UBA5 | 2.75E−07 | 3.30E−05 | 97 | −2.7186 |
| ALG12 | 2.75E−07 | 3.30E−05 | 98 | −2.4878 |
| MTHFD1 | 2.75E−07 | 3.30E−05 | 99 | −2.4228 |
| TCF3 | 2.75E−07 | 3.30E−05 | 100 | −1.8062 |
| MPI | 2.75E−07 | 3.30E−05 | 101 | −1.274 |
| CDK10 | 2.75E−07 | 3.30E−05 | 102 | −1.0362 |
| CAPRIN1 | 2.75E−07 | 3.30E−05 | 103 | −1.7487 |
| DAZAP1 | 2.75E−07 | 3.30E−05 | 104 | −1.2418 |
| COG3 | 2.75E−07 | 3.30E−05 | 105 | −1.3055 |
| PTBP1 | 2.75E−07 | 3.30E−05 | 106 | −1.8911 |
| ACIN1 | 2.75E−07 | 3.30E−05 | 107 | −1.7743 |
| MEN1 | 2.75E−07 | 3.30E−05 | 108 | −1.7556 |
| TAF6L | 2.75E−07 | 3.30E−05 | 109 | −2.1254 |
| DNTTIP1 | 2.75E−07 | 3.30E−05 | 110 | −1.4768 |
| COG4 | 2.75E−07 | 3.30E−05 | 111 | −1.5487 |
| PRR12 | 2.75E−07 | 3.30E−05 | 112 | −0.80453 |
| ZNF394 | 2.75E−07 | 3.30E−05 | 113 | −1.3311 |
| SERTAD2 | 2.75E−07 | 3.30E−05 | 114 | −1.1473 |
| POU2F2 | 2.75E−07 | 3.30E−05 | 115 | −0.96121 |
| MAD2L2 | 2.75E−07 | 3.30E−05 | 116 | −1.7216 |
| SFXN1 | 2.75E−07 | 3.30E−05 | 117 | −1.5188 |
| GATAD1 | 2.75E−07 | 3.30E−05 | 118 | −1.0485 |
| SLC25A32 | 2.75E−07 | 3.30E−05 | 119 | −2.2581 |
| CAPZB | 2.75E−07 | 3.30E−05 | 120 | −1.7273 |
| IMPDH2 | 2.75E−07 | 3.30E−05 | 121 | −2.4095 |
| TSR3 | 2.75E−07 | 3.30E−05 | 122 | −0.87243 |
| ARID1A | 2.75E−07 | 3.30E−05 | 123 | −1.0375 |
| C17orf70 | 2.75E−07 | 3.30E−05 | 124 | −0.96319 |
| SPAG7 | 2.75E−07 | 3.30E−05 | 125 | −1.0431 |
| ELP3 | 2.75E−07 | 3.30E−05 | 126 | −1.8762 |
| JADE1 | 2.75E−07 | 3.30E−05 | 127 | −1.032 |
| PHF12 | 2.75E−07 | 3.30E−05 | 128 | −1.2297 |
| TFAP4 | 2.75E−07 | 3.30E−05 | 129 | −0.99044 |
| CTNNBL1 | 2.75E−07 | 3.30E−05 | 130 | −2.7479 |
| GNE | 2.75E−07 | 3.30E−05 | 131 | −2.5231 |
| CCZ1B | 2.75E−07 | 3.30E−05 | 132 | −0.8782 |
| URM1 | 8.25E−07 | 8.30E−05 | 133 | −1.4014 |
| PRUNE | 2.75E−07 | 3.30E−05 | 134 | −2.1679 |
| DAXX | 2.75E−07 | 3.30E−05 | 135 | −2.3282 |
| MED16 | 2.75E−07 | 3.30E−05 | 136 | −1.0961 |
| FANCB | 2.75E−07 | 3.30E−05 | 137 | −1.395 |
| THRAP3 | 2.75E−07 | 3.30E−05 | 138 | −1.3108 |
| MTR | 2.75E−07 | 3.30E−05 | 139 | −1.7534 |
| HIST1H1B | 2.75E−07 | 3.30E−05 | 140 | −1.0088 |
| SLC39A1 | 2.75E−07 | 3.30E−05 | 141 | −0.93229 |
| UBE2G2 | 2.75E−07 | 3.30E−05 | 142 | −5.2261 |
| HSPA14 | 1.93E−06 | 0.000169 | 143 | −1.4927 |
| SURF4 | 2.75E−07 | 3.30E−05 | 144 | −0.86611 |
| MATR3 | 2.75E−07 | 3.30E−05 | 145 | −1.3659 |
| SLC29A1 | 2.75E−07 | 3.30E−05 | 146 | −0.82665 |
| MBNL1 | 2.75E−07 | 3.30E−05 | 147 | −1.9273 |
| NOB1 | 2.75E−07 | 3.30E−05 | 148 | −2.2714 |
| FANCA | 2.75E−07 | 3.30E−05 | 149 | −0.94526 |
| FDXR | 2.75E−07 | 3.30E−05 | 150 | −2.3416 |
| UGGT1 | 8.25E−07 | 8.30E−05 | 151 | −1.053 |
| G6PD | 8.25E−07 | 8.30E−05 | 152 | −1.1959 |
| LSM10 | 8.25E−07 | 8.30E−05 | 153 | −2.7856 |
| MMP23B | 8.25E−07 | 8.30E−05 | 154 | −0.5305 |
| PTPN2 | 8.25E−07 | 8.30E−05 | 155 | −1.7627 |
| ZC3H18 | 8.25E−07 | 8.30E−05 | 156 | −1.3137 |
| TELO2 | 8.25E−07 | 8.30E−05 | 157 | −2.0897 |
| ENO1 | 8.25E−07 | 8.30E−05 | 158 | −1.3875 |
| HIRA | 8.25E−07 | 8.30E−05 | 159 | −1.4647 |
| TADA2B | 8.25E−07 | 8.30E−05 | 160 | −1.9283 |
| MMACHC | 8.25E−07 | 8.30E−05 | 161 | −0.64598 |
| DSCC1 | 8.25E−07 | 8.30E−05 | 162 | −1.2685 |
| SEC63 | 8.25E−07 | 8.30E−05 | 163 | −1.4483 |
| SYK | 8.25E−07 | 8.30E−05 | 164 | −1.3841 |
| ALDOA | 8.25E−07 | 8.30E−05 | 165 | −4.1492 |
| UFL1 | 8.25E−07 | 8.30E−05 | 166 | −1.2024 |
| TCEB3 | 8.25E−07 | 8.30E−05 | 167 | −1.0778 |
| WNK1 | 8.25E−07 | 8.30E−05 | 168 | −1.0803 |
| FNTB | 8.25E−07 | 8.30E−05 | 169 | −1.2109 |
| UBE2T | 8.25E−07 | 8.30E−05 | 170 | −2.4549 |
| DDX47 | 8.25E−07 | 8.30E−05 | 171 | −4.1438 |
| TMED10 | 8.25E−07 | 8.30E−05 | 172 | −1.5354 |
| TNRC6A | 8.25E−07 | 8.30E−05 | 173 | −0.82822 |
| UFC1 | 8.25E−07 | 8.30E−05 | 174 | −2.1306 |
| ZC3H4 | 1.38E−06 | 0.000129 | 175 | −1.1836 |
| R3HCC1L | 2.75E−07 | 3.30E−05 | 176 | −0.48394 |
| PPIH | 8.25E−07 | 8.30E−05 | 177 | −1.5858 |
| RPIA | 8.25E−07 | 8.30E−05 | 178 | −1.4533 |
| PDCD2 | 2.48E−06 | 0.000212 | 179 | −1.4438 |
| WDR48 | 8.25E−07 | 8.30E−05 | 180 | −1.2387 |
| ZW10 | 8.25E−07 | 8.30E−05 | 181 | −0.74188 |
| CCM2 | 8.25E−07 | 8.30E−05 | 182 | −1.1396 |
| SRM | 1.38E−06 | 0.000129 | 183 | −1.1766 |
| POT1 | 1.38E−06 | 0.000129 | 184 | −1.8236 |
| DNAJC11 | 1.38E−06 | 0.000129 | 185 | −1.2337 |
| PUM1 | 1.38E−06 | 0.000129 | 186 | −1.0753 |
| ZFC3H1 | 1.38E−06 | 0.000129 | 187 | −1.0359 |
| NDOR1 | 1.38E−06 | 0.000129 | 188 | −2.4355 |
| MMS19 | 1.38E−06 | 0.000129 | 189 | −2.5541 |
| TRNAU1AP | 1.38E−06 | 0.000129 | 190 | −1.7469 |
| METTL16 | 1.38E−06 | 0.000129 | 191 | −3.8202 |
| WDR1 | 1.38E−06 | 0.000129 | 192 | −1.7337 |
| CHD1 | 1.38E−06 | 0.000129 | 193 | −1.679 |
| OSBPL3 | 1.38E−06 | 0.000129 | 194 | −1.0057 |
| MARK2 | 1.93E−06 | 0.000169 | 195 | −0.71423 |
| USP34 | 1.93E−06 | 0.000169 | 196 | −1.4096 |
| UBE2J1 | 1.93E−06 | 0.000169 | 197 | −2.8219 |
| PGP | 1.93E−06 | 0.000169 | 198 | −1.1174 |
| MED13 | 1.93E−06 | 0.000169 | 199 | −1.7154 |
| ZXDC | 1.93E−06 | 0.000169 | 200 | −0.63222 |
| ZNF142 | 1.93E−06 | 0.000169 | 201 | −0.83779 |
| SAP18 | 1.93E−06 | 0.000169 | 202 | −2.4013 |
| ALG5 | 1.93E−06 | 0.000169 | 203 | −3.1391 |
| CBX3 | 1.93E−06 | 0.000169 | 204 | −1.5797 |
| PUS1 | 2.20E−06 | 0.000192 | 205 | −0.77848 |
| MAEA | 2.48E−06 | 0.000212 | 206 | −0.7623 |
| AHCY | 1.93E−06 | 0.000169 | 207 | −3.0859 |
| TPI1 | 6.88E−06 | 0.000491 | 208 | −1.1744 |
| YTHDF2 | 2.48E−06 | 0.000212 | 209 | −2.3588 |
| TGFBR1 | 2.48E−06 | 0.000212 | 210 | −1.956 |
| CTU2 | 3.03E−06 | 0.000258 | 211 | −1.0233 |
| GNB1L | 3.58E−06 | 0.000293 | 212 | −2.0193 |
| RTEL1 | 3.58E−06 | 0.000293 | 213 | −1.9433 |
| NFKBIB | 3.58E−06 | 0.000293 | 214 | −0.72321 |
| USP22 | 3.58E−06 | 0.000293 | 215 | −3.6949 |
| PCGF1 | 1.93E−06 | 0.000169 | 216 | −0.98357 |
| ILF3 | 3.58E−06 | 0.000293 | 217 | −1.1324 |
| PGD | 3.58E−06 | 0.000293 | 218 | −2.7281 |
| RBM33 | 3.58E−06 | 0.000293 | 219 | −0.91397 |
| CYLD | 3.58E−06 | 0.000293 | 220 | −0.78023 |
| FANCL | 4.13E−06 | 0.00032 | 221 | −1.6086 |
| CD79B | 1.02E−05 | 0.000707 | 222 | −1.0305 |
| HIPK1 | 4.13E−06 | 0.00032 | 223 | −1.3159 |
| PPCDC | 4.13E−06 | 0.00032 | 224 | −1.5928 |
| C19orf52 | 4.13E−06 | 0.00032 | 225 | −1.1541 |
| KDM5C | 4.13E−06 | 0.00032 | 226 | −1.582 |
| NSMCE1 | 4.13E−06 | 0.00032 | 227 | −0.90929 |
| TSC22D2 | 4.13E−06 | 0.00032 | 228 | −0.90812 |
| PMVK | 4.13E−06 | 0.00032 | 229 | −0.76664 |
| RHOH | 4.13E−06 | 0.00032 | 230 | −0.72967 |
| NDRG3 | 3.58E−06 | 0.000293 | 231 | −2.6004 |
| CORO1A | 4.13E−06 | 0.00032 | 232 | −1.142 |
| CCDC101 | 4.68E−06 | 0.000352 | 233 | −1.1866 |
| EIF4H | 4.68E−06 | 0.000352 | 234 | −1.9236 |
| DEAF1 | 4.68E−06 | 0.000352 | 235 | −1.0271 |
| DIS3 | 4.68E−06 | 0.000352 | 236 | −1.9908 |
| TFDP1 | 4.68E−06 | 0.000352 | 237 | −0.85198 |
| GADD45B | 4.68E−06 | 0.000352 | 238 | −0.74163 |
| KAT2B | 4.68E−06 | 0.000352 | 239 | −0.55243 |
| ENY2 | 4.13E−06 | 0.00032 | 240 | −4.3664 |
| POP7 | 4.13E−06 | 0.00032 | 241 | −1.6283 |
| GCN1L1 | 5.78E−06 | 0.000433 | 242 | −1.0864 |
| RPP30 | 6.33E−06 | 0.000467 | 243 | −2.0147 |
| BOD1L1 | 6.33E−06 | 0.000467 | 244 | −0.77896 |
| TIMM10 | 6.33E−06 | 0.000467 | 245 | −1.9234 |
| CWC27 | 6.60E−06 | 0.000485 | 246 | −1.0861 |
| CSNK1D | 6.88E−06 | 0.000491 | 247 | −0.43505 |
| DCP2 | 6.88E−06 | 0.000491 | 248 | −1.2729 |
| ETV3 | 1.84E−05 | 0.001185 | 249 | −0.47516 |
| DDX6 | 6.88E−06 | 0.000491 | 250 | −3.0595 |
| RAB7A | 6.88E−06 | 0.000491 | 251 | −1.6591 |
| MGAT2 | 6.88E−06 | 0.000491 | 252 | −0.61632 |
| ADSL | 6.88E−06 | 0.000491 | 253 | −4.0532 |
| DDRGK1 | 6.33E−06 | 0.000467 | 254 | −0.84322 |
| FANCD2 | 7.43E−06 | 0.000522 | 255 | −1.3503 |
| INTS10 | 7.43E−06 | 0.000522 | 256 | −0.76646 |
| SRSF11 | 7.43E−06 | 0.000522 | 257 | −1.4732 |
| DYNLRB1 | 7.43E−06 | 0.000522 | 258 | −1.4566 |
| SOD2 | 8.25E−06 | 0.000578 | 259 | −1.8836 |
| COG2 | 9.08E−06 | 0.000633 | 260 | −1.373 |
| TUBD1 | 1.95E−05 | 0.001242 | 261 | −1.2159 |
| MED23 | 1.13E−05 | 0.000781 | 262 | −3.0312 |
| RINT1 | 1.18E−05 | 0.000816 | 263 | −1.2159 |
| NRBP1 | 1.24E−05 | 0.000844 | 264 | −2.0701 |
| TRIP12 | 1.24E−05 | 0.000844 | 265 | −0.62476 |
| TIMM22 | 1.24E−05 | 0.000844 | 266 | −1.0791 |
| MED15 | 1.29E−05 | 0.000875 | 267 | −0.939 |
| UNC50 | 1.29E−05 | 0.000875 | 268 | −1.0737 |
| APEX2 | 1.40E−05 | 0.000932 | 269 | −0.53235 |
| LCMT1 | 1.40E−05 | 0.000932 | 270 | −1.3138 |
| TADA1 | 1.40E−05 | 0.000932 | 271 | −0.89377 |
| HIST1H1E | 1.40E−05 | 0.000932 | 272 | −0.57782 |
| ZC3H10 | 1.40E−05 | 0.000932 | 273 | −1.0663 |
| FIZ1 | 1.46E−05 | 0.000965 | 274 | −0.4719 |
| DOLPP1 | 1.51E−05 | 0.000997 | 275 | −1.8881 |
| ERCC4 | 1.62E−05 | 0.001066 | 276 | −1.4032 |
| EIF4E2 | 1.73E−05 | 0.001126 | 277 | −2.936 |
| CARM1 | 1.73E−05 | 0.001126 | 278 | −1.0542 |
| ARFRP1 | 4.15E−05 | 0.002286 | 279 | −1.0721 |
| AKT2 | 1.84E−05 | 0.001185 | 280 | −0.58778 |
| DPM1 | 1.68E−05 | 0.001098 | 281 | −1.1977 |
| SOCS1 | 1.90E−05 | 0.001211 | 282 | −1.9262 |
| UGP2 | 1.84E−05 | 0.001185 | 283 | −2.6488 |
| MRGBP | 1.90E−05 | 0.001211 | 284 | −1.2352 |
| PRKCSH | 2.01E−05 | 0.001272 | 285 | −0.87391 |
| DICER1 | 2.12E−05 | 0.001333 | 286 | −0.90221 |
| ELP6 | 2.12E−05 | 0.001333 | 287 | −1.083 |
| MED18 | 2.23E−05 | 0.001397 | 288 | −2.3408 |
| FBXW11 | 2.28E−05 | 0.001417 | 289 | −1.1753 |
| BTG2 | 2.39E−05 | 0.00148 | 290 | −0.5946 |
| RPN2 | 2.45E−05 | 0.001488 | 291 | −1.0166 |
| LSM14A | 2.45E−05 | 0.001488 | 292 | −1.5495 |
| SETD1A | 2.45E−05 | 0.001488 | 293 | −1.3544 |
| ERCC1 | 2.45E−05 | 0.001488 | 294 | −1.0283 |
| FAM60A | 2.45E−05 | 0.001488 | 295 | −1.0911 |
| TRAF2 | 2.56E−05 | 0.00155 | 296 | −0.77015 |
| ZEB1 | 2.61E−05 | 0.001573 | 297 | −0.88487 |
| HNRNPK | 2.28E−05 | 0.001417 | 298 | −2.9217 |
| MTRR | 2.61E−05 | 0.001573 | 299 | −1.4078 |
| HNRNPD | 2.72E−05 | 0.001634 | 300 | −1.0175 |
| DHRSX | 2.28E−05 | 0.001417 | 301 | −1.6622 |
| ABCC1 | 2.94E−05 | 0.001748 | 302 | −0.6192 |
| KAT7 | 3.11E−05 | 0.001834 | 303 | −1.7226 |
| SMARCC1 | 3.11E−05 | 0.001834 | 304 | −0.6963 |
| GART | 3.16E−05 | 0.00186 | 305 | −2.7771 |
| PNRC2 | 3.22E−05 | 0.001881 | 306 | −0.99935 |
| UBE2M | 3.22E−05 | 0.001881 | 307 | −2.7775 |
| PPP2R1A | 3.33E−05 | 0.001932 | 308 | −0.75588 |
| POP5 | 3.38E−05 | 0.001958 | 309 | −2.9343 |
| GTF2E2 | 2.89E−05 | 0.001721 | 310 | −3.186 |
| SAE1 | 4.32E−05 | 0.002334 | 311 | −1.9348 |
| TXNDC5 | 3.66E−05 | 0.002104 | 312 | −0.49974 |
| NPM1 | 2.89E−05 | 0.001721 | 313 | −2.1032 |
| MPDU1 | 3.77E−05 | 0.002153 | 314 | −1.1717 |
| DHX33 | 3.27E−05 | 0.001907 | 315 | −2.8277 |
| SSR3 | 3.77E−05 | 0.002153 | 316 | −0.70963 |
| HERPUD1 | 3.82E−05 | 0.002171 | 317 | −0.63459 |
| TBC1D20 | 3.82E−05 | 0.002171 | 318 | −0.93728 |
| PARP16 | 3.88E−05 | 0.002188 | 319 | −0.76575 |
| IPO5 | 3.88E−05 | 0.002188 | 320 | −0.34486 |
| PPCS | 6.68E−05 | 0.003243 | 321 | −2.22 |
| CNOT3 | 3.49E−05 | 0.002015 | 322 | −2.9451 |
| FANCI | 3.99E−05 | 0.002243 | 323 | −1.3331 |
| OTUD5 | 4.10E−05 | 0.002284 | 324 | −0.58683 |
| HK2 | 4.10E−05 | 0.002284 | 325 | −1.2069 |
| TCEB2 | 4.10E−05 | 0.002284 | 326 | −2.3383 |
| DRAP1 | 4.15E−05 | 0.002286 | 327 | −0.67686 |
| CRAMP1L | 4.15E−05 | 0.002286 | 328 | −0.85483 |
| SERBP1 | 4.29E−05 | 0.002334 | 329 | −0.83219 |
| WHSC1 | 4.32E−05 | 0.002334 | 330 | −0.91061 |
| P2RX5 | 4.32E−05 | 0.002334 | 331 | −0.57514 |
| NBAS | 4.32E−05 | 0.002334 | 332 | −0.77217 |
| SUZ12 | 4.32E−05 | 0.002334 | 333 | −1.434 |
| TCF4 | 4.43E−05 | 0.002386 | 334 | −0.69747 |
| AGPAT6 | 4.48E−05 | 0.002402 | 335 | −1.0721 |
| ATMIN | 4.48E−05 | 0.002402 | 336 | −0.62337 |
| MORF4L1 | 4.13E−05 | 0.002286 | 337 | −1.2004 |
| DERL2 | 4.81E−05 | 0.002563 | 338 | −3.0728 |
| UXS1 | 4.81E−05 | 0.002563 | 339 | −1.2275 |
| DPH3 | 6.46E−05 | 0.003205 | 340 | −1.9761 |
| CAND1 | 4.92E−05 | 0.002591 | 341 | −1.0094 |
| SARNP | 4.92E−05 | 0.002591 | 342 | −1.3906 |
| CCDC6 | 4.92E−05 | 0.002591 | 343 | −0.45919 |
| SETDB1 | 4.92E−05 | 0.002591 | 344 | −0.75854 |
| MED25 | 4.98E−05 | 0.002612 | 345 | −0.71998 |
| USP48 | 5.09E−05 | 0.002662 | 346 | −0.75815 |
| SLC7A3 | 5.14E−05 | 0.002676 | 347 | −0.5237 |
| KLHL8 | 5.14E−05 | 0.002676 | 348 | −0.77897 |
| VHL | 5.20E−05 | 0.002689 | 349 | −1.2454 |
| KHSRP | 5.20E−05 | 0.002689 | 350 | −0.76539 |
| SNRNP40 | 5.25E−05 | 0.002709 | 351 | −1.7692 |
| CDK11A | 5.36E−05 | 0.002758 | 352 | −0.98443 |
| JOSD2 | 7.78E−05 | 0.003716 | 353 | −0.46882 |
| MBD6 | 5.58E−05 | 0.002847 | 354 | −0.41141 |
| RNASEH2C | 5.69E−05 | 0.002887 | 355 | −1.2672 |
| PLCG2 | 5.69E−05 | 0.002887 | 356 | −0.36796 |
| ELMSAN1 | 5.53E−05 | 0.002827 | 357 | −0.99941 |
| SKP2 | 7.84E−05 | 0.003733 | 358 | −0.83528 |
| CPSF6 | 5.53E−05 | 0.002827 | 359 | −1.153 |
| ZNF384 | 5.80E−05 | 0.002926 | 360 | −0.96619 |
| ACTR5 | 5.97E−05 | 0.003001 | 361 | −0.87108 |
| BCL11A | 6.02E−05 | 0.00302 | 362 | −0.63571 |
| EED | 5.75E−05 | 0.002906 | 363 | −1.6589 |
| RC3H1 | 6.19E−05 | 0.003094 | 364 | −0.92952 |
| CSRP2BP | 6.30E−05 | 0.00314 | 365 | −1.2432 |
| VRK1 | 6.35E−05 | 0.003159 | 366 | −1.0144 |
| WDR81 | 6.52E−05 | 0.003214 | 367 | −0.52531 |
| TOX4 | 6.52E−05 | 0.003214 | 368 | −0.78022 |
| WDR77 | 6.57E−05 | 0.003224 | 369 | −1.0444 |
| POP1 | 6.57E−05 | 0.003224 | 370 | −1.9041 |
| RIF1 | 6.63E−05 | 0.003225 | 371 | −0.8925 |
| GNPNAT1 | 6.63E−05 | 0.003225 | 372 | −1.7119 |
| ARHGAP17 | 6.63E−05 | 0.003225 | 373 | −0.41095 |
| FEN1 | 6.85E−05 | 0.003305 | 374 | −0.96274 |
| MOGS | 6.85E−05 | 0.003305 | 375 | −0.85852 |
| STAG1 | 7.34E−05 | 0.003534 | 376 | −0.78582 |
| YKT6 | 7.51E−05 | 0.003594 | 377 | −2.1675 |
| FANCC | 7.51E−05 | 0.003594 | 378 | −1.0424 |
| ASXL1 | 7.89E−05 | 0.003749 | 379 | −0.8933 |
| BRIP1 | 8.00E−05 | 0.003791 | 380 | −1.4437 |
| CHKA | 8.28E−05 | 0.003901 | 381 | −1.1545 |
| ALG6 | 8.28E−05 | 0.003901 | 382 | −1.7692 |
| CXorf56 | 0.00012019 | 0.005422 | 383 | −0.73568 |
| PPP1R8 | 0.00018289 | 0.00771 | 384 | −0.59577 |
| PELO | 8.39E−05 | 0.003942 | 385 | −1.838 |
| TMEM222 | 8.61E−05 | 0.004019 | 386 | −0.49223 |
| TRMT6 | 8.64E−05 | 0.004019 | 387 | −1.4807 |
| LARP4 | 8.66E−05 | 0.004019 | 388 | −0.70372 |
| FXN | 8.66E−05 | 0.004019 | 389 | −1.2868 |
| C11orf57 | 8.72E−05 | 0.004034 | 390 | −0.74768 |
| RAD51B | 8.44E−05 | 0.003958 | 391 | −0.86854 |
| LIG1 | 8.99E−05 | 0.004151 | 392 | −0.65608 |
| MORC3 | 9.32E−05 | 0.004292 | 393 | −1.4851 |
| CCND3 | 0.00017712 | 0.007531 | 394 | −1.1766 |
| CHD8 | 9.60E−05 | 0.004407 | 395 | −0.83168 |
| PCIF1 | 0.00010754 | 0.004913 | 396 | −0.74087 |
| FAF2 | 9.76E−05 | 0.004472 | 397 | −2.2193 |
| ACACA | 0.00011579 | 0.005263 | 398 | −1.2432 |
| DOHH | 0.00011964 | 0.005411 | 399 | −1.3502 |
| METTL1 | 0.00012074 | 0.005433 | 400 | −0.74513 |
| DHX36 | 0.00012404 | 0.005568 | 401 | −1.4652 |
| HLA-DRA | 0.00012459 | 0.005579 | 402 | −0.59667 |
| UBE2N | 0.00010919 | 0.004976 | 403 | −1.9083 |
| GLS | 0.00012734 | 0.005688 | 404 | −0.83085 |
| SYVN1 | 0.00012899 | 0.005733 | 405 | −2.3372 |
| OS9 | 0.00012899 | 0.005733 | 406 | −0.93882 |
| BTAF1 | 0.00013009 | 0.005767 | 407 | −1.2216 |
| FANCF | 0.00013119 | 0.005802 | 408 | −0.54162 |
| ADAT2 | 0.00013449 | 0.005933 | 409 | −2.0191 |
| KCTD10 | 0.00013889 | 0.006098 | 410 | −0.80267 |
| CD74 | 0.00013889 | 0.006098 | 411 | −0.37099 |
| TASP1 | 0.00014769 | 0.006468 | 412 | −0.64097 |
| POLR2M | 0.00015209 | 0.006645 | 413 | −0.54699 |
| ALG8 | 0.00015319 | 0.006677 | 414 | −1.7448 |
| UBTF | 0.00015484 | 0.006732 | 415 | −2.6903 |
| BLNK | 0.00015979 | 0.006931 | 416 | −0.48042 |
| PPIL1 | 0.00016364 | 0.007081 | 417 | −1.426 |
| E2F5 | 0.00018564 | 0.007789 | 418 | −0.77806 |
| CLPTM1 | 0.00016474 | 0.007111 | 419 | −0.39767 |
| SEC62 | 0.00016804 | 0.007236 | 420 | −1.305 |
| TRAF3 | 0.00017354 | 0.007455 | 421 | −0.78055 |
| EZH2 | 0.00017409 | 0.007461 | 422 | −0.99815 |
| PGAM1 | 0.00011964 | 0.005411 | 423 | −2.864 |
| CCNL2 | 0.00017464 | 0.007467 | 424 | −0.58207 |
| DR1 | 0.00018289 | 0.00771 | 425 | −1.8877 |
| ILF2 | 0.00018289 | 0.00771 | 426 | −2.1921 |
| SENP8 | 0.00018839 | 0.007886 | 427 | −0.65142 |
| TMEM41B | 0.000206 | 0.008485 | 428 | −1.8748 |
| DHX29 | 0.00019169 | 0.007987 | 429 | −1.0628 |
| WDR4 | 0.00019719 | 0.008197 | 430 | −0.7053 |
| DPM3 | 0.00030666 | 0.011794 | 431 | −0.68484 |
| EDF1 | 0.00019994 | 0.008274 | 432 | −1.5976 |
| ATRX | 0.00019994 | 0.008274 | 433 | −0.73698 |
| ABCD4 | 0.0002005 | 0.008277 | 434 | −0.55888 |
| PNKP | 0.00021095 | 0.008669 | 435 | −0.94698 |
| METTL3 | 0.0002115 | 0.008672 | 436 | −1.3147 |
| ZEB2 | 0.0002181 | 0.008922 | 437 | −0.56151 |
| ZNRD1 | 0.0002192 | 0.008947 | 438 | −0.64068 |
| DTNBP1 | 0.00017739 | 0.007531 | 439 | −0.61908 |
| RAD51D | 0.00022195 | 0.009039 | 440 | −1.8715 |
| IFNL3 | 0.00018454 | 0.007761 | 441 | −0.48373 |
| INIP | 0.00022635 | 0.009197 | 442 | −0.68589 |
| KIAA1432 | 0.00022855 | 0.009265 | 443 | −0.7149 |
| SPATA2 | 0.0002313 | 0.009356 | 444 | −0.48567 |
| RNASEH2B | 0.00024065 | 0.009712 | 445 | −1.2977 |
| PATZ1 | 0.00024285 | 0.009779 | 446 | −0.55913 |
| SSR1 | 0.00024725 | 0.009912 | 447 | −0.59852 |
| RBM14 | 0.0002478 | 0.009912 | 448 | −1.4979 |
| TRA2B | 0.0002819 | 0.011007 | 449 | −0.34691 |
| ZNF131 | 0.00025055 | 0.01 | 450 | −0.89448 |
| CNOT2 | 0.0002511 | 0.01 | 451 | −1.1232 |
| SHMT2 | 0.00025275 | 0.010043 | 452 | −1.6048 |
| DNAJB6 | 0.00017684 | 0.007531 | 453 | −1.6977 |
| CCAR1 | 0.0002643 | 0.010456 | 454 | −0.7193 |
| KIAA1429 | 0.0002654 | 0.010476 | 455 | −2.5294 |
| CMIP | 0.00027695 | 0.010908 | 456 | −0.5693 |
| TIMM9 | 0.00019114 | 0.007983 | 457 | −2.4545 |
| ATP1A1 | 0.0002786 | 0.010949 | 458 | −1.088 |
| UBQLN1 | 0.0002797 | 0.010969 | 459 | −0.48244 |
| BRPF1 | 0.0002819 | 0.011007 | 460 | −0.72453 |
| XRCC3 | 0.0002841 | 0.011069 | 461 | −2.1848 |
| DYNLL1 | 0.0002456 | 0.009868 | 462 | −1.0687 |
| ASF1B | 0.00028795 | 0.011189 | 463 | −0.38041 |
| MCTS1 | 0.0002885 | 0.011189 | 464 | −1.5776 |
| ELP5 | 0.00028905 | 0.011189 | 465 | −1.074 |
| DOLK | 0.00029345 | 0.011335 | 466 | −0.92542 |
| CUL3 | 0.00026265 | 0.010413 | 467 | −2.244 |
| TAFSL | 0.00031216 | 0.01198 | 468 | −1.1914 |
| NUBP1 | 0.00032701 | 0.012524 | 469 | −1.9279 |
| GTF3C5 | 0.00033471 | 0.012791 | 470 | −1.5988 |
| HGS | 0.00033581 | 0.012794 | 471 | −0.7379 |
| MBTD1 | 0.00033691 | 0.012794 | 472 | −0.51835 |
| BNIP1 | 0.00033828 | 0.012819 | 473 | −1.5931 |
| EXOSC10 | 0.00033966 | 0.012844 | 474 | −0.86987 |
| TMEM203 | 0.00034461 | 0.013004 | 475 | −0.79811 |
| STX5 | 0.00029785 | 0.01148 | 476 | −1.1301 |
| CYB561A3 | 0.00035396 | 0.013329 | 477 | −1.4264 |
| DDX59 | 0.00036111 | 0.01357 | 478 | −1.6059 |
| CHAF1B | 0.00036331 | 0.013596 | 479 | −3.6354 |
| UBA3 | 0.00038916 | 0.014503 | 480 | −0.89871 |
| PAN2 | 0.00039301 | 0.014616 | 481 | −0.44235 |
| LARP7 | 0.00039631 | 0.014709 | 482 | −0.8863 |
| YLPM1 | 0.00040127 | 0.014862 | 483 | −0.7158 |
| WIZ | 0.00033691 | 0.012794 | 484 | −0.7112 |
| RANBP1 | 0.00040347 | 0.014912 | 485 | −1.063 |
| C11orf73 | 0.00041337 | 0.015216 | 486 | −0.98562 |
| ZNF592 | 0.00041832 | 0.015367 | 487 | −0.42683 |
| SIN3B | 0.00042052 | 0.015416 | 488 | −0.79219 |
| SMG6 | 0.00042382 | 0.015506 | 489 | −1.7488 |
| ICMT | 0.00043042 | 0.015715 | 490 | −0.6528 |
| PUM2 | 0.00043207 | 0.015743 | 491 | −0.59867 |
| ATF4 | 0.00036276 | 0.013596 | 492 | −0.74392 |
| CHP1 | 0.00043482 | 0.015808 | 493 | −0.69057 |
| POLE4 | 0.00043647 | 0.015808 | 494 | −0.35748 |
| RPP38 | 0.00043647 | 0.015808 | 495 | −0.71939 |
| BTK | 0.00044142 | 0.015955 | 496 | −0.36394 |
| DPH2 | 0.00044252 | 0.015963 | 497 | −0.43537 |
| CCNC | 0.00044362 | 0.01597 | 498 | −3.7364 |
| BCL6 | 0.00044582 | 0.016017 | 499 | −0.89838 |
| PTP4A2 | 0.00058773 | 0.019886 | 500 | −0.7186 |
| SEC61B | 0.00044692 | 0.016025 | 501 | −0.71694 |
| IDH3A | 0.00038091 | 0.014225 | 502 | −1.2479 |
| ZFAND6 | 0.00057398 | 0.019568 | 503 | −0.56026 |
| POLR1E | 0.00047937 | 0.017087 | 504 | −0.87331 |
| NIPBL | 0.00048212 | 0.017151 | 505 | −1.4923 |
| EDEM2 | 0.00048377 | 0.017175 | 506 | −0.49869 |
| GNB2L1 | 0.00049037 | 0.017375 | 507 | −1.3745 |
| PDPK1 | 0.00041062 | 0.015146 | 508 | −2.0951 |
| LSM11 | 0.00049147 | 0.01738 | 509 | −0.9537 |
| CDK6 | 0.00050083 | 0.017661 | 510 | −1.2721 |
| SETD2 | 0.00050138 | 0.017661 | 511 | −0.78448 |
| FAM208B | 0.00050303 | 0.017684 | 512 | −0.62416 |
| STK11 | 0.00050798 | 0.017789 | 513 | −0.45095 |
| UBR5 | 0.00051073 | 0.017851 | 514 | −0.7923 |
| ZMYND8 | 0.00051513 | 0.017935 | 515 | −2.5594 |
| C1orf74 | 0.00051513 | 0.017935 | 516 | −0.43419 |
| RAB18 | 0.00063284 | 0.021095 | 517 | −1.1507 |
| STAM | 0.00052173 | 0.01813 | 518 | −0.76301 |
| GOLT1B | 0.00053438 | 0.018465 | 519 | −1.9171 |
| E2F1 | 0.00053548 | 0.018465 | 520 | −0.42129 |
| CCAR2 | 0.00053548 | 0.018465 | 521 | −0.36749 |
| MKLN1 | 0.00054153 | 0.018638 | 522 | −0.73197 |
| SERP1 | 0.00046837 | 0.016761 | 523 | −0.57836 |
| CHMP4B | 0.00047332 | 0.016904 | 524 | −1.647 |
| EFTUD1 | 0.00055968 | 0.019189 | 525 | −0.82679 |
| METTL14 | 0.00056078 | 0.01919 | 526 | −3.0256 |
| AEBP2 | 0.00058058 | 0.019755 | 527 | −0.54415 |
| SHISA5 | 0.00058196 | 0.019765 | 528 | −0.55852 |
| BCOR | 0.00058333 | 0.019774 | 529 | −0.6257 |
| RPRD1B | 0.00058883 | 0.019886 | 530 | −0.56666 |
| KAT6A | 0.00059378 | 0.020015 | 531 | −0.53701 |
| MANF | 0.00060259 | 0.020274 | 532 | −1.7996 |
| MED31 | 0.00050633 | 0.017766 | 533 | −3.5067 |
| TMEM57 | 0.00060919 | 0.020458 | 534 | −0.68348 |
| LARP4B | 0.00061964 | 0.02077 | 535 | −0.35135 |
| RCOR1 | 0.00052833 | 0.018324 | 536 | −2.2571 |
| PFAS | 0.00063009 | 0.02106 | 537 | −0.75112 |
| C1orf27 | 0.00063064 | 0.02106 | 538 | −0.89463 |
| TADA3 | 0.00063889 | 0.021257 | 539 | −0.8885 |
| TGDS | 0.00064604 | 0.021455 | 540 | −1.1051 |
| UFM1 | 0.0007918 | 0.025496 | 541 | −2.0203 |
| MAN2A1 | 0.00066859 | 0.022163 | 542 | −0.8632 |
| LGALS7 | 0.00057013 | 0.019473 | 543 | −0.56998 |
| RMI1 | 0.00069279 | 0.022923 | 544 | −1.1459 |
| IKZF5 | 0.0007005 | 0.023093 | 545 | −0.72461 |
| POLE3 | 0.0007038 | 0.02316 | 546 | −0.58163 |
| MPHOSPH6 | 0.00071865 | 0.023605 | 547 | −1.463 |
| KDM8 | 0.0007236 | 0.023725 | 548 | −0.55903 |
| ZC3H15 | 0.00072525 | 0.023735 | 549 | −1.0095 |
| PRR14 | 0.00074065 | 0.024108 | 550 | −0.43541 |
| ORC3 | 0.00074065 | 0.024108 | 551 | −1.2725 |
| UNC45A | 0.00074835 | 0.024315 | 552 | −0.61625 |
| RIOK2 | 0.00075935 | 0.024628 | 553 | −1.6965 |
| MED1 | 0.0007687 | 0.024886 | 554 | −0.59862 |
| SMCHD1 | 0.0007918 | 0.025496 | 555 | −0.46963 |
| UBN2 | 0.00080061 | 0.025734 | 556 | −0.46977 |
| FANCG | 0.00080391 | 0.025794 | 557 | −0.7092 |
| BCAR1 | 0.00081381 | 0.026065 | 558 | −0.43503 |
| KNTC1 | 0.00081573 | 0.02608 | 559 | −0.65164 |
| SNW1 | 0.00081931 | 0.026148 | 560 | −1.7169 |
| EIF4A1 | 0.00055473 | 0.019056 | 561 | −4.0269 |
| CDK5RAP3 | 0.00084186 | 0.02682 | 562 | −0.48263 |
| BLOC1S1 | 0.00086001 | 0.027337 | 563 | −0.41902 |
| USE1 | 0.00086111 | 0.027337 | 564 | −0.46918 |
| C19orf40 | 0.00087156 | 0.02762 | 565 | −0.50193 |
| TRMT12 | 0.00069609 | 0.02299 | 566 | −0.51409 |
| GABPB1 | 0.00087816 | 0.027731 | 567 | −1.8477 |
| CD19 | 0.00087816 | 0.027731 | 568 | −0.81505 |
| VPS52 | 0.00089521 | 0.028188 | 569 | −1.352 |
| C18orf8 | 0.00089576 | 0.028188 | 570 | −0.63578 |
| CDC37 | 0.00090567 | 0.028401 | 571 | −1.2308 |
| UBE2L3 | 0.001107 | 0.033602 | 572 | −0.88291 |
| UAP1 | 0.00090952 | 0.028472 | 573 | −0.89737 |
| FANCM | 0.0007313 | 0.02389 | 574 | −0.62576 |
| SUV420H2 | 0.00095297 | 0.029677 | 575 | −0.47283 |
| PKM | 0.00077585 | 0.025072 | 576 | −1.431 |
| PPP2R2A | 0.00096287 | 0.029882 | 577 | −1.9237 |
| MTA1 | 0.00098157 | 0.03041 | 578 | −0.70507 |
| SASH3 | 0.0010047 | 0.031072 | 579 | −0.49444 |
| GSK3A | 0.0010135 | 0.031291 | 580 | −0.3179 |
| RAD9A | 0.0010393 | 0.031979 | 581 | −0.88926 |
| SMARCB1 | 0.0010465 | 0.032144 | 582 | −1.3679 |
| CHMP5 | 0.001052 | 0.032258 | 583 | −1.2791 |
| C11orf30 | 0.0010553 | 0.032305 | 584 | −0.7227 |
| SLC2A1 | 0.0010597 | 0.032384 | 585 | −1.2545 |
| POLE | 0.0010619 | 0.032396 | 586 | −1.2409 |
| ATAD5 | 0.0010866 | 0.033095 | 587 | −0.57748 |
| LIN54 | 0.0010916 | 0.03319 | 588 | −0.47577 |
| NCBP1 | 0.0011092 | 0.033612 | 589 | −2.1559 |
| GID8 | 0.0011246 | 0.034021 | 590 | −0.76826 |
| HEATR1 | 0.00090567 | 0.028401 | 591 | −0.65755 |
| RABL6 | 0.0011499 | 0.034728 | 592 | −0.3821 |
| AHCYL2 | 0.0011537 | 0.034745 | 593 | −0.99063 |
| NOP9 | 0.0011543 | 0.034745 | 594 | −0.85164 |
| C16orf59 | 0.0011634 | 0.034901 | 595 | −0.63623 |
| KMT2B | 0.0011763 | 0.03523 | 596 | −0.74079 |
| DHX9 | 0.001184 | 0.035402 | 597 | −2.088 |
| DDX49 | 0.00094967 | 0.029643 | 598 | −1.5074 |
| SPIN1 | 0.00095022 | 0.029643 | 599 | −0.68574 |
| ASB7 | 0.0012005 | 0.035836 | 600 | −0.59411 |
| NCOA3 | 0.0012087 | 0.036022 | 601 | −0.54811 |
| GIGYF2 | 0.0012153 | 0.036159 | 602 | −0.79034 |
| TPT1 | 0.00096177 | 0.029882 | 603 | −1.7191 |
| CTRB2 | 0.0012335 | 0.036639 | 604 | −0.38092 |
| ZNHIT3 | 0.0014772 | 0.042955 | 605 | −0.90547 |
| MTOR | 0.0012467 | 0.03697 | 606 | −1.201 |
| SCAF4 | 0.0013309 | 0.039336 | 607 | −1.5738 |
| GTF3A | 0.0013375 | 0.039466 | 608 | −0.47147 |
| MED13L | 0.0013529 | 0.039855 | 609 | −0.82635 |
| TFG | 0.0013738 | 0.040405 | 610 | −1.2412 |
| GINS4 | 0.0014029 | 0.041195 | 611 | −0.99145 |
| RCSD1 | 0.0014068 | 0.041241 | 612 | −0.59781 |
| PGLS | 0.0014453 | 0.042232 | 613 | −0.49264 |
| THAP4 | 0.0020729 | 0.056448 | 614 | −0.31278 |
| XPO5 | 0.0014596 | 0.042576 | 615 | −0.95085 |
| KIF17 | 0.0014618 | 0.042576 | 616 | −0.2644 |
| SP2 | 0.0014997 | 0.04354 | 617 | −0.3686 |
| MTHFD2 | 0.0015058 | 0.043646 | 618 | −1.5146 |
| MRFAP1 | 0.0015113 | 0.043735 | 619 | −0.44287 |
| CMTR1 | 0.0015168 | 0.043823 | 620 | −1.6681 |
| NAT10 | 0.0015349 | 0.044277 | 621 | −1.0751 |
| WBSCR22 | 0.001558 | 0.044871 | 622 | −1.0016 |
| TTI2 | 0.0015657 | 0.044925 | 623 | −0.72625 |
| RNPS1 | 0.0015668 | 0.044925 | 624 | −1.0253 |
| MED24 | 0.0015674 | 0.044925 | 625 | −1.3534 |
| AFF2 | 0.0015762 | 0.045105 | 626 | −0.68116 |
| SF1 | 0.0012555 | 0.037169 | 627 | −2.9684 |
| OSTC | 0.0016691 | 0.04763 | 630 | −0.42648 |
| DKC1 | 0.0016697 | 0.04763 | 631 | −1.9844 |
| EIF4G1 | 0.0016763 | 0.047743 | 632 | −0.8781 |
| CLCC1 | 0.0016884 | 0.048011 | 633 | −0.74632 |
| WDR18 | 0.0017038 | 0.048373 | 634 | −0.94539 |
| XPR1 | 0.0017219 | 0.048811 | 635 | −0.48846 |
| KIAA0922 | 0.0017478 | 0.049466 | 636 | −0.49966 |
| PCNA | 0.0011587 | 0.034819 | 637 | −3.3843 |
| KIN | 0.0014255 | 0.041721 | 640 | −1.4474 |

### Example 3: Positive Regulators of BTN3A1

This Example provides a list of the gene products that increase BTN3A1 expression.

TABLE 2
Positive Regulators of BTN3A1

| | | False-discovery | | Log2 Fold |
|---|---|---|---|---|
| Gene ID | p-value | Rate | Rank | Change |
| BTN3A1 | 2.75E−07 | 4.00E−05 | 1 | 3.2503 |
| ECSIT | 2.75E−07 | 4.00E−05 | 2 | 1.9636 |
| FBXW7 | 2.75E−07 | 4.00E−05 | 3 | 1.2999 |
| SPIB | 2.75E−07 | 4.00E−05 | 4 | 1.4043 |
| IRF1 | 2.75E−07 | 4.00E−05 | 5 | 3.3807 |
| NLRC5 | 2.75E−07 | 4.00E−05 | 6 | 2.9447 |
| IRF8 | 2.75E−07 | 4.00E−05 | 7 | 2.2276 |
| NDUFA2 | 2.75E−07 | 4.00E−05 | 8 | 2.2492 |
| NDUFV1 | 2.75E−07 | 4.00E−05 | 9 | 2.2077 |
| NDUFA13 | 2.75E−07 | 4.00E−05 | 10 | 2.2471 |
| USP7 | 2.75E−07 | 4.00E−05 | 11 | 2.6988 |
| C17orf89 | 2.75E−07 | 4.00E−05 | 12 | 2.7763 |
| RFXAP | 2.75E−07 | 4.00E−05 | 13 | 2.3058 |
| UBE2A | 2.75E−07 | 4.00E−05 | 14 | 2.0448 |
| SRPK1 | 2.75E−07 | 4.00E−05 | 15 | 1.8136 |
| NDUFS7 | 2.75E−07 | 4.00E−05 | 16 | 1.8325 |
| PDS5B | 2.75E−07 | 4.00E−05 | 17 | 1.4582 |
| CNOT11 | 2.75E−07 | 4.00E−05 | 18 | 1.6799 |
| NDUFB7 | 2.75E−07 | 4.00E−05 | 19 | 1.8706 |
| BTN3A2 | 2.75E−07 | 4.00E−05 | 20 | 3.6559 |
| FOXRED1 | 2.75E−07 | 4.00E−05 | 21 | 1.2212 |
| NDUFS8 | 2.75E−07 | 4.00E−05 | 22 | 2.2644 |
| JMJD6 | 2.75E−07 | 4.00E−05 | 23 | 1.599 |
| NDUFS2 | 2.75E−07 | 4.00E−05 | 24 | 2.0221 |
| NDUFC2 | 2.75E−07 | 4.00E−05 | 25 | 2.1978 |
| HSF1 | 2.75E−07 | 4.00E−05 | 26 | 1.172 |
| ACAD9 | 2.75E−07 | 4.00E−05 | 27 | 1.844 |
| NDUFAF5 | 2.75E−07 | 4.00E−05 | 28 | 1.6674 |
| TIMMDC1 | 2.75E−07 | 4.00E−05 | 29 | 2.7627 |
| HSD17B10 | 2.75E−07 | 4.00E−05 | 30 | 1.6516 |
| BRD2 | 2.75E−07 | 4.00E−05 | 31 | 2.1807 |
| NDUFA6 | 2.75E−07 | 4.00E−05 | 32 | 1.4508 |
| CNOT4 | 2.75E−07 | 4.00E−05 | 33 | 1.7671 |
| SPI1 | 2.75E−07 | 4.00E−05 | 34 | 1.1901 |
| MDH2 | 2.75E−07 | 4.00E−05 | 35 | 1.1456 |
| DARS2 | 2.75E−07 | 4.00E−05 | 36 | 1.3212 |
| TMEM261 | 2.75E−07 | 4.00E−05 | 37 | 1.1035 |
| STIP1 | 2.75E−07 | 4.00E−05 | 38 | 1.4601 |
| FIBP | 2.75E−07 | 4.00E−05 | 39 | 1.2667 |
| FXR1 | 2.75E−07 | 4.00E−05 | 40 | 1.0088 |
| NFU1 | 2.75E−07 | 4.00E−05 | 41 | 2.1101 |
| GGNBP2 | 2.75E−07 | 4.00E−05 | 42 | 1.8752 |
| STAT2 | 2.75E−07 | 4.00E−05 | 43 | 1.3171 |
| TRUB2 | 2.75E−07 | 4.00E−05 | 44 | 1.2665 |
| BIRC6 | 2.75E−07 | 4.00E−05 | 45 | 2.1373 |
| MARS2 | 2.75E−07 | 4.00E−05 | 46 | 1.4526 |
| NDUFA9 | 2.75E−07 | 4.00E−05 | 47 | 1.7243 |
| USP19 | 2.75E−07 | 4.00E−05 | 48 | 0.9147 |
| UBA6 | 2.75E−07 | 4.00E−05 | 49 | 1.8512 |
| MTG1 | 2.75E−07 | 4.00E−05 | 50 | 1.14 |
| KIAA0391 | 2.75E−07 | 4.00E−05 | 51 | 1.2522 |
| RIC8A | 2.75E−07 | 4.00E−05 | 52 | 1.5867 |
| FCGR2B | 2.75E−07 | 4.00E−05 | 53 | 1.5571 |
| PARS2 | 2.75E−07 | 4.00E−05 | 54 | 1.5132 |
| PPP2R5C | 2.75E−07 | 4.00E−05 | 55 | 1.4335 |
| NDUFB9 | 2.75E−07 | 4.00E−05 | 56 | 2.2844 |
| NDUFA3 | 2.75E−07 | 4.00E−05 | 57 | 2.0935 |
| NDUFAF3 | 2.75E−07 | 4.00E−05 | 58 | 1.6226 |
| NDUFAF1 | 2.75E−07 | 4.00E−05 | 59 | 1.833 |
| NOSIP | 2.75E−07 | 4.00E−05 | 60 | 1.4324 |
| BCS1L | 2.75E−07 | 4.00E−05 | 61 | 1.4855 |
| GTPBP8 | 2.75E−07 | 4.00E−05 | 62 | 0.98385 |
| NDUFA8 | 2.75E−07 | 4.00E−05 | 63 | 2.0184 |
| BTN2A2 | 2.75E−07 | 4.00E−05 | 64 | 0.50146 |
| NDUFA11 | 2.75E−07 | 4.00E−05 | 65 | 1.387 |
| GATAD2B | 2.75E−07 | 4.00E−05 | 66 | 0.9237 |
| PET112 | 2.75E−07 | 4.00E−05 | 67 | 1.1207 |
| NDUFB2 | 2.75E−07 | 4.00E−05 | 68 | 0.85003 |
| ING2 | 2.75E−07 | 4.00E−05 | 69 | 1.1431 |
| GATAD2A | 2.75E−07 | 4.00E−05 | 70 | 1.1768 |
| MBD3 | 2.75E−07 | 4.00E−05 | 71 | 0.8546 |
| EPC1 | 2.75E−07 | 4.00E−05 | 72 | 1.3642 |
| NDUFB10 | 2.75E−07 | 4.00E−05 | 73 | 1.9309 |
| ZNF699 | 2.75E−07 | 4.00E−05 | 74 | 1.2701 |
| DMTF1 | 2.75E−07 | 4.00E−05 | 75 | 1.4086 |
| MRPL24 | 2.75E−07 | 4.00E−05 | 76 | 1.5047 |
| KHDRBS1 | 2.75E−07 | 4.00E−05 | 77 | 1.0224 |
| PDHA1 | 2.75E−07 | 4.00E−05 | 78 | 1.989 |
| FASN | 2.75E−07 | 4.00E−05 | 79 | 1.121 |
| IKBKG | 2.75E−07 | 4.00E−05 | 80 | 0.70032 |
| FTSJ2 | 2.75E−07 | 4.00E−05 | 81 | 1.3486 |
| VARS2 | 2.75E−07 | 4.00E−05 | 82 | 1.7517 |
| SCO2 | 2.75E−07 | 4.00E−05 | 83 | 1.4507 |
| NDUFB8 | 2.75E−07 | 4.00E−05 | 84 | 2.0957 |
| CREBBP | 2.75E−07 | 4.00E−05 | 85 | 0.65367 |
| JAK1 | 2.75E−07 | 4.00E−05 | 86 | 1.2715 |
| STK4 | 2.75E−07 | 4.00E−05 | 87 | 1.1563 |
| PPM1A | 2.75E−07 | 4.00E−05 | 88 | 1.1982 |
| CDKN2AIP | 2.75E−07 | 4.00E−05 | 89 | 0.69263 |
| RFX5 | 2.75E−07 | 4.00E−05 | 90 | 1.8284 |
| KDM3B | 2.75E−07 | 4.00E−05 | 91 | 0.93413 |
| NDUFB11 | 2.75E−07 | 4.00E−05 | 92 | 1.5467 |
| NDUFS1 | 2.75E−07 | 4.00E−05 | 93 | 1.6891 |
| HSPA13 | 2.75E−07 | 4.00E−05 | 94 | 1.4681 |
| GLTSCR1 | 2.75E−07 | 4.00E−05 | 95 | 0.63882 |
| MGA | 2.75E−07 | 4.00E−05 | 96 | 0.63655 |
| MIPEP | 2.75E−07 | 4.00E−05 | 97 | 0.98897 |
| NUBPL | 2.75E−07 | 4.00E−05 | 98 | 1.2291 |
| MRPL21 | 2.75E−07 | 4.00E−05 | 99 | 1.0894 |
| GLRX5 | 2.75E−07 | 4.00E−05 | 100 | 1.4278 |
| EXOC5 | 2.75E−07 | 4.00E−05 | 101 | 0.94047 |
| ALAD | 2.75E−07 | 4.00E−05 | 102 | 1.062 |
| RSBN1L | 2.75E−07 | 4.00E−05 | 103 | 0.78976 |
| SIRT1 | 2.75E−07 | 4.00E−05 | 104 | 1.1637 |
| UBR4 | 2.75E−07 | 4.00E−05 | 105 | 1.3548 |
| C10orf2 | 2.75E−07 | 4.00E−05 | 106 | 1.4335 |
| RCE1 | 2.75E−07 | 4.00E−05 | 107 | 1.0632 |
| MRPS18B | 2.75E−07 | 4.00E−05 | 108 | 1.4971 |
| NDUFB4 | 2.75E−07 | 4.00E−05 | 109 | 1.1581 |
| METTL17 | 2.75E−07 | 4.00E−05 | 110 | 1.5537 |
| SSBP1 | 2.75E−07 | 4.00E−05 | 111 | 1.3962 |
| CNOT1 | 2.75E−07 | 4.00E−05 | 112 | 1.7343 |
| C2CD5 | 2.75E−07 | 4.00E−05 | 113 | 1.0848 |
| SPCS3 | 2.75E−07 | 4.00E−05 | 114 | 1.7741 |
| TEFM | 2.75E−07 | 4.00E−05 | 115 | 1.3711 |
| PRRC2A | 2.75E−07 | 4.00E−05 | 116 | 1.0004 |
| HSP90AB1 | 2.75E−07 | 4.00E−05 | 117 | 1.0945 |
| MTIF2 | 2.75E−07 | 4.00E−05 | 118 | 1.3871 |
| GLTSCR1L | 2.75E−07 | 4.00E−05 | 119 | 0.91588 |
| FADD | 2.75E−07 | 4.00E−05 | 120 | 0.6723 |
| NDUFB3 | 8.25E−07 | 0.0001 | 121 | 2.6153 |
| POLG2 | 2.75E−07 | 4.00E−05 | 122 | 1.1903 |
| RAD54L2 | 2.75E−07 | 4.00E−05 | 123 | 0.64305 |
| COQ7 | 2.75E−07 | 4.00E−05 | 124 | 0.98461 |
| ERAL1 | 8.25E−07 | 0.0001 | 125 | 1.5519 |
| GATC | 8.25E−07 | 0.0001 | 126 | 0.94912 |
| NDUFS3 | 8.25E−07 | 0.0001 | 127 | 1.9439 |
| CPSF7 | 8.25E−07 | 0.0001 | 128 | 0.62461 |
| MTF1 | 8.25E−07 | 0.0001 | 129 | 1.5337 |
| HMBS | 8.25E−07 | 0.0001 | 130 | 0.83226 |
| PTCD3 | 8.25E−07 | 0.0001 | 131 | 1.2929 |
| ZBTB12 | 8.25E−07 | 0.0001 | 132 | 1.2737 |
| POLG | 8.25E−07 | 0.0001 | 133 | 1.4916 |
| GNA13 | 8.25E−07 | 0.0001 | 134 | 1.1661 |
| PDHB | 8.25E−07 | 0.0001 | 135 | 1.3849 |
| COQ5 | 8.25E−07 | 0.0001 | 136 | 1.3227 |
| ARHGEF1 | 8.25E−07 | 0.0001 | 137 | 0.9632 |
| CIR1 | 8.25E−07 | 0.0001 | 138 | 1.0649 |
| HDAC3 | 8.25E−07 | 0.0001 | 139 | 1.9537 |
| ECHS1 | 8.25E−07 | 0.0001 | 140 | 0.89342 |
| COX11 | 8.25E−07 | 0.0001 | 141 | 1.7289 |
| TFB1M | 8.25E−07 | 0.0001 | 142 | 1.4143 |
| ARMC5 | 8.25E−07 | 0.0001 | 143 | 0.79994 |
| PITPNC1 | 8.25E−07 | 0.0001 | 144 | 0.8658 |
| PDSS2 | 8.25E−07 | 0.0001 | 145 | 1.0256 |
| SLC25A1 | 8.25E−07 | 0.0001 | 146 | 1.6003 |
| RFXANK | 1.38E−06 | 0.000155 | 147 | 1.5318 |
| MTA2 | 8.25E−07 | 0.0001 | 148 | 0.87504 |
| COQ3 | 8.25E−07 | 0.0001 | 149 | 1.5379 |
| MRPL53 | 8.25E−07 | 0.0001 | 150 | 1.009 |
| TXLNG | 1.38E−06 | 0.000155 | 151 | 0.66772 |
| LRPPRC | 1.38E−06 | 0.000155 | 152 | 0.83873 |
| SRF | 1.38E−06 | 0.000155 | 153 | 0.85793 |
| AARS2 | 1.38E−06 | 0.000155 | 154 | 1.1102 |
| ATP11C | 1.38E−06 | 0.000155 | 155 | 1.0945 |
| MRPL23 | 1.38E−06 | 0.000155 | 156 | 1.3031 |
| COA3 | 1.38E−06 | 0.000155 | 157 | 0.8802 |
| COQ2 | 1.38E−06 | 0.000155 | 158 | 1.1343 |
| FARS2 | 1.38E−06 | 0.000155 | 159 | 1.0447 |
| NKTR | 1.38E−06 | 0.000155 | 160 | 0.73127 |
| PHF20L1 | 1.93E−06 | 0.000213 | 161 | 0.74243 |
| VCPIP1 | 1.93E−06 | 0.000213 | 162 | 0.76659 |
| SELRC1 | 1.93E−06 | 0.000213 | 163 | 1.0403 |
| MRPS26 | 2.48E−06 | 0.000265 | 164 | 0.63837 |
| AFF3 | 2.48E−06 | 0.000265 | 165 | 0.73481 |
| GFM2 | 2.48E−06 | 0.000265 | 166 | 1.1922 |
| STAT1 | 2.48E−06 | 0.000265 | 167 | 1.0741 |
| SEC11A | 3.03E−06 | 0.000322 | 168 | 0.94352 |
| COX8A | 3.30E−06 | 0.000349 | 169 | 1.3926 |
| NDUFA10 | 3.58E−06 | 0.000366 | 170 | 1.735 |
| MRPL43 | 3.58E−06 | 0.000366 | 171 | 0.92592 |
| NUFIP2 | 3.58E−06 | 0.000366 | 172 | 1.6225 |
| PDAP1 | 2.48E−06 | 0.000265 | 173 | 2.6636 |
| FRYL | 3.58E−06 | 0.000366 | 174 | 0.60806 |
| NGRN | 4.13E−06 | 0.00041 | 175 | 1.1824 |
| IRF9 | 4.13E−06 | 0.00041 | 176 | 0.74616 |
| MYL6 | 3.58E−06 | 0.000366 | 177 | 0.87747 |
| TMEM189 | 4.13E−06 | 0.00041 | 178 | 0.85096 |
| SLIRP | 4.13E−06 | 0.00041 | 179 | 0.91254 |
| MIER3 | 4.13E−06 | 0.00041 | 180 | 0.75921 |
| FASTKDS | 4.68E−06 | 0.000457 | 181 | 1.5298 |
| INTS12 | 4.68E−06 | 0.000457 | 182 | 0.98036 |
| MRPS34 | 3.58E−06 | 0.000366 | 183 | 0.95445 |
| USP42 | 4.68E−06 | 0.000457 | 184 | 1.2101 |
| PDSS1 | 5.78E−06 | 0.000556 | 185 | 1.158 |
| DLAT | 5.78E−06 | 0.000556 | 186 | 0.58476 |
| FLII | 5.78E−06 | 0.000556 | 187 | 0.82006 |
| MRPS11 | 6.33E−06 | 0.000602 | 188 | 0.74147 |
| PCBP1 | 6.33E−06 | 0.000602 | 189 | 1.3348 |
| COX10 | 6.88E−06 | 0.000638 | 190 | 1.2681 |
| LARS2 | 6.88E−06 | 0.000638 | 191 | 1.3263 |
| METAP1 | 6.88E−06 | 0.000638 | 192 | 0.87399 |
| RTN4IP1 | 6.88E−06 | 0.000638 | 193 | 1.746 |
| ASB3 | 7.43E−06 | 0.000685 | 194 | 0.55158 |
| NDUFA1 | 6.88E−06 | 0.000638 | 195 | 1.9145 |
| PDE12 | 1.02E−05 | 0.00093 | 196 | 0.9456 |
| RPUSD4 | 1.02E−05 | 0.00093 | 197 | 1.1846 |
| UBE3D | 1.07E−05 | 0.000975 | 198 | 0.70074 |
| TRIM39 | 1.24E−05 | 0.001119 | 199 | 0.50025 |
| MTO1 | 1.35E−05 | 0.001207 | 200 | 1.0509 |
| SLC30A1 | 1.35E−05 | 0.001207 | 201 | 0.45274 |
| NDUFAF7 | 1.40E−05 | 0.001226 | 202 | 1.5655 |
| KMT2E | 1.40E−05 | 0.001226 | 203 | 0.74201 |
| MRPL49 | 1.40E−05 | 0.001226 | 204 | 0.87591 |
| EIF1 | 1.40E−05 | 0.001226 | 205 | 1.4483 |
| MRPL52 | 2.67E−05 | 0.002088 | 206 | 0.80018 |
| PRMT10 | 1.40E−05 | 0.001226 | 207 | 0.61256 |
| NUP188 | 1.46E−05 | 0.001261 | 208 | 0.49971 |
| ZBTB14 | 1.46E−05 | 0.001261 | 209 | 0.89044 |
| FBXO11 | 1.51E−05 | 0.001303 | 210 | 1.4641 |
| COA6 | 2.28E−05 | 0.001859 | 211 | 1.46 |
| COX15 | 1.68E−05 | 0.001438 | 212 | 1.4979 |
| IFNAR2 | 1.73E−05 | 0.001471 | 213 | 1.5125 |
| MRPS15 | 1.73E−05 | 0.001471 | 214 | 0.63107 |
| MRPS16 | 1.79E−05 | 0.001511 | 215 | 0.8386 |
| MRPL17 | 1.90E−05 | 0.001574 | 216 | 1.0584 |
| DDX26B | 1.90E−05 | 0.001574 | 217 | 0.97127 |
| OTUD6B | 1.90E−05 | 0.001574 | 218 | 1.081 |
| HERC2 | 1.90E−05 | 0.001574 | 219 | 0.4355 |
| TGFBRAP1 | 1.95E−05 | 0.001598 | 220 | 0.71503 |
| COX18 | 1.95E−05 | 0.001598 | 221 | 0.70405 |
| NDUFB6 | 1.95E−05 | 0.001598 | 222 | 1.0527 |
| NXT1 | 2.39E−05 | 0.001889 | 223 | 0.52237 |
| SMS | 2.39E−05 | 0.001889 | 224 | 0.71349 |
| SS18 | 2.39E−05 | 0.001889 | 225 | 0.66809 |
| BRD9 | 2.39E−05 | 0.001889 | 226 | 0.57432 |
| CARS2 | 2.39E−05 | 0.001889 | 227 | 1.5349 |
| DUSP10 | 4.92E−05 | 0.003503 | 228 | 0.39422 |
| NDUFB5 | 2.45E−05 | 0.001924 | 229 | 1.6449 |
| RBFA | 2.34E−05 | 0.001889 | 230 | 1.1732 |
| PET117 | 2.39E−05 | 0.001889 | 231 | 1.2156 |
| PPP1R12A | 2.94E−05 | 0.002293 | 232 | 0.74294 |
| ACLY | 3.00E−05 | 0.002326 | 233 | 0.68005 |
| PPM1B | 5.97E−05 | 0.004132 | 234 | 0.59881 |
| PDCL | 3.05E−05 | 0.002358 | 235 | 0.6778 |
| SMYD5 | 3.11E−05 | 0.002391 | 236 | 0.64613 |
| XPO4 | 3.27E−05 | 0.002507 | 237 | 0.80512 |
| SPCS1 | 3.33E−05 | 0.002538 | 238 | 1.9368 |
| HSPA4 | 3.38E−05 | 0.002569 | 239 | 0.92399 |
| LRRC8B | 3.66E−05 | 0.002755 | 240 | 0.40742 |
| EPC2 | 3.71E−05 | 0.002773 | 241 | 1.0618 |
| MTG2 | 3.71E−05 | 0.002773 | 242 | 0.79797 |
| COQ6 | 3.88E−05 | 0.002884 | 243 | 0.78365 |
| NSUN4 | 3.93E−05 | 0.002913 | 244 | 0.93282 |
| SUGT1 | 4.04E−05 | 0.002982 | 245 | 2.3048 |
| TMEM126B | 3.66E−05 | 0.002755 | 246 | 2.2207 |
| RARS2 | 4.32E−05 | 0.003159 | 247 | 1.4435 |
| E2F8 | 4.32E−05 | 0.003159 | 248 | 0.53213 |
| TRIM15 | 4.54E−05 | 0.003294 | 249 | 0.44036 |
| RAB5C | 4.81E−05 | 0.003479 | 250 | 0.52031 |
| ZNF687 | 4.92E−05 | 0.003503 | 251 | 0.47252 |
| SLC35F2 | 4.92E−05 | 0.003503 | 252 | 0.62627 |
| TMOD3 | 4.92E−05 | 0.003503 | 253 | 0.5931 |
| SCO1 | 4.54E−05 | 0.003294 | 254 | 0.98909 |
| MRPS23 | 5.14E−05 | 0.003645 | 255 | 0.80188 |
| SURF1 | 5.25E−05 | 0.003708 | 256 | 0.62056 |
| ALAS1 | 5.58E−05 | 0.003926 | 257 | 0.95591 |
| PEX2 | 5.64E−05 | 0.003949 | 258 | 0.78942 |
| YTHDC1 | 5.69E−05 | 0.003957 | 259 | 0.72988 |
| COX16 | 5.69E−05 | 0.003957 | 260 | 1.9692 |
| NDUFV2 | 6.08E−05 | 0.004192 | 261 | 1.232 |
| MRPL12 | 6.19E−05 | 0.004235 | 262 | 0.90792 |
| SETD5 | 6.19E−05 | 0.004235 | 263 | 0.60779 |
| ERN1 | 6.24E−05 | 0.004257 | 264 | 0.39391 |
| CDK5 | 6.30E−05 | 0.004278 | 265 | 0.96174 |
| KCMF1 | 6.52E−05 | 0.004411 | 266 | 1.0674 |
| SON | 6.68E−05 | 0.004506 | 267 | 1.103 |
| MRPL38 | 6.85E−05 | 0.0046 | 268 | 1.2067 |
| MCAT | 6.90E−05 | 0.004619 | 269 | 0.53295 |
| STK40 | 7.01E−05 | 0.004675 | 270 | 0.42554 |
| C16orf72 | 7.18E−05 | 0.004768 | 271 | 0.92507 |
| U2AF2 | 7.62E−05 | 0.005023 | 272 | 1.0856 |
| HM13 | 7.62E−05 | 0.005023 | 273 | 0.90419 |
| XPNPEP1 | 8.28E−05 | 0.005399 | 274 | 0.68478 |
| ATP11A | 8.28E−05 | 0.005399 | 275 | 0.39624 |
| DNAJC8 | 7.78E−05 | 0.005113 | 276 | 1.2588 |
| EHD1 | 8.55E−05 | 0.005558 | 277 | 0.62509 |
| HELZ | 8.66E−05 | 0.005609 | 278 | 0.52657 |
| WARS2 | 8.77E−05 | 0.00566 | 279 | 1.8499 |
| COX4I1 | 8.83E−05 | 0.005675 | 280 | 1.5658 |
| AURKAIP1 | 8.88E−05 | 0.00569 | 281 | 0.60515 |
| FZR1 | 9.27E−05 | 0.005916 | 282 | 0.52991 |
| MRP63 | 9.38E−05 | 0.005965 | 283 | 0.92202 |
| DDX39B | 9.60E−05 | 0.006084 | 284 | 0.63156 |
| AP2B1 | 0.00010259 | 0.006479 | 285 | 0.80132 |
| LPAR5 | 0.00010369 | 0.006526 | 286 | 0.56598 |
| ARL15 | 0.00010534 | 0.006606 | 287 | 0.57267 |
| CS | 0.00010919 | 0.006801 | 288 | 1.5006 |
| PEX6 | 0.00011139 | 0.006914 | 289 | 0.51476 |
| SARS2 | 0.00011469 | 0.007046 | 290 | 1.1351 |
| RRM2B | 0.00011469 | 0.007046 | 291 | 0.53513 |
| NFE2L1 | 0.00011964 | 0.0073 | 292 | 0.38897 |
| SNRPB2 | 0.00011964 | 0.0073 | 293 | 0.76809 |
| DDX5 | 0.00012019 | 0.007309 | 294 | 0.82243 |
| TUFM | 0.00012239 | 0.007417 | 295 | 1.041 |
| QTRTD1 | 0.00012569 | 0.007566 | 296 | 0.82307 |
| ATP5F1 | 0.00012899 | 0.007739 | 297 | 1.4054 |
| EIF3H | 0.00013229 | 0.007911 | 298 | 0.53908 |
| PEX10 | 0.00013339 | 0.00795 | 299 | 0.47176 |
| SLC25A51 | 0.00011469 | 0.007046 | 300 | 0.60285 |
| BTN3A3 | 0.00014219 | 0.008424 | 301 | 0.57439 |
| MRPS25 | 0.00014274 | 0.008424 | 302 | 0.98062 |
| BAP1 | 0.00014274 | 0.008424 | 303 | 0.83223 |
| MBD2 | 0.00014714 | 0.008655 | 304 | 0.44026 |
| API5 | 0.00014989 | 0.008788 | 305 | 0.55055 |
| MRPS35 | 0.00012459 | 0.007525 | 306 | 1.3146 |
| FBXO48 | 0.00015649 | 0.009146 | 307 | 0.70986 |
| DAP3 | 0.00016199 | 0.009406 | 308 | 0.84336 |
| CIITA | 0.00016529 | 0.009567 | 309 | 0.68005 |
| CCNI | 0.00016914 | 0.009758 | 310 | 0.54436 |
| MRPS6 | 0.00017409 | 0.010012 | 311 | 1.1872 |
| ATP5C1 | 0.00017904 | 0.010262 | 312 | 0.82461 |
| BRWD1 | 0.00017959 | 0.010262 | 313 | 0.67751 |
| FBXO21 | 0.00018344 | 0.010416 | 314 | 0.44163 |
| PEX3 | 0.00018729 | 0.010568 | 315 | 0.69772 |
| NUDCD1 | 0.00019389 | 0.010907 | 316 | 1.1373 |
| EARS2 | 0.00019554 | 0.010965 | 317 | 0.89512 |
| COX5A | 0.00019884 | 0.011116 | 318 | 1.0876 |
| ANKRD11 | 0.00019994 | 0.011142 | 319 | 0.83141 |
| RPUSD3 | 0.0002071 | 0.01147 | 320 | 0.35058 |
| LCP1 | 0.0002093 | 0.011516 | 321 | 0.5388 |
| LEMD3 | 0.00020985 | 0.011516 | 322 | 0.37425 |
| MRPS24 | 0.00020985 | 0.011516 | 323 | 0.81886 |
| MRPL19 | 0.00021095 | 0.011541 | 324 | 0.48228 |
| IFNAR1 | 0.0002214 | 0.012076 | 325 | 1.0615 |
| NDUFAF4 | 0.00018344 | 0.010416 | 326 | 0.76702 |
| LMNB1 | 0.0002258 | 0.012279 | 327 | 0.48111 |
| NCOR1 | 0.00018509 | 0.010477 | 328 | 0.83242 |
| HNRNPU | 0.00022745 | 0.012294 | 329 | 1.3184 |
| JAZF1 | 0.00022855 | 0.012317 | 330 | 0.71384 |
| EPT1 | 0.0002313 | 0.012428 | 331 | 0.80741 |
| ATP5SL | 0.00023735 | 0.01264 | 332 | 1.1365 |
| LIG3 | 0.00023735 | 0.01264 | 333 | 0.4722 |
| C12orf65 | 0.00023735 | 0.01264 | 334 | 0.39954 |
| UQCRB | 0.0002665 | 0.014026 | 335 | 1.5416 |
| ACTB | 0.0002016 | 0.0112 | 336 | 1.2972 |
| SRSF5 | 0.00024395 | 0.012953 | 337 | 0.81548 |
| PLAA | 0.00026375 | 0.013951 | 338 | 0.64344 |
| RBM6 | 0.0002676 | 0.014043 | 339 | 0.49739 |
| RABEPK | 0.0002698 | 0.014118 | 340 | 0.57453 |
| MTPAP | 0.0002709 | 0.014134 | 341 | 0.50098 |
| ING1 | 0.00043757 | 0.02123 | 342 | 0.34245 |
| NDUFC1 | 0.00010809 | 0.006755 | 343 | 1.7497 |
| MTFMT | 0.0002797 | 0.014538 | 344 | 0.70013 |
| DDHD1 | 0.00028025 | 0.014538 | 345 | 0.3256 |
| MRPL46 | 0.00028685 | 0.014837 | 346 | 0.8653 |
| AGPS | 0.0002907 | 0.014993 | 347 | 0.40133 |
| ANKRD31 | 0.00030336 | 0.015601 | 348 | 0.59314 |
| ARRDC3 | 0.00030446 | 0.015613 | 349 | 0.62556 |
| QRSL1 | 0.00030666 | 0.015681 | 350 | 1.0474 |
| COX20 | 0.0002643 | 0.013951 | 351 | 0.99402 |
| LIPT2 | 0.00032041 | 0.016338 | 352 | 0.91941 |
| USP15 | 0.00033251 | 0.016907 | 353 | 0.62367 |
| ZSWIM8 | 0.00033966 | 0.017222 | 354 | 0.42915 |
| H2AFZ | 0.00035286 | 0.017841 | 355 | 0.91883 |
| ATP5O | 0.00036001 | 0.018152 | 356 | 0.83548 |
| PHF23 | 0.00036716 | 0.018358 | 357 | 0.62721 |
| COX14 | 0.00015869 | 0.009244 | 358 | 1.1937 |
| ZBED1 | 0.00038421 | 0.019157 | 359 | 0.40342 |
| S1PR2 | 0.00038916 | 0.019325 | 360 | 0.32484 |
| TMEM30A | 0.00038971 | 0.019325 | 361 | 0.90146 |
| MPC2 | 0.00039576 | 0.019571 | 362 | 0.60143 |
| MRPL18 | 0.00040127 | 0.019788 | 363 | 1.0074 |
| NDUFS5 | 0.00041227 | 0.020275 | 364 | 1.5344 |
| PPME1 | 0.00041942 | 0.020569 | 365 | 0.52214 |
| FCHSD2 | 0.00042052 | 0.020569 | 366 | 0.5616 |
| DHX15 | 0.00042877 | 0.020916 | 367 | 1.2515 |
| DOCK8 | 0.00043262 | 0.021046 | 368 | 0.42031 |
| PEX13 | 0.00036386 | 0.018295 | 369 | 0.71585 |
| FCGR2A | 0.00036716 | 0.018358 | 370 | 0.82869 |
| MRPL11 | 0.00045297 | 0.021918 | 371 | 0.94543 |
| DHX30 | 0.00045847 | 0.022125 | 372 | 1.0212 |
| RBBP7 | 0.00046672 | 0.022463 | 373 | 0.77062 |
| SUV39H1 | 0.00047882 | 0.022983 | 374 | 0.38956 |
| SLC25A11 | 0.00048762 | 0.023282 | 375 | 0.36393 |
| SHROOM1 | 0.00049367 | 0.023508 | 376 | 0.36261 |
| COX7C | 0.00022745 | 0.012294 | 377 | 2.7817 |
| MRPS33 | 0.00054043 | 0.025667 | 378 | 0.94842 |
| CLCN5 | 0.00082866 | 0.037666 | 379 | 0.34209 |
| GPR182 | 0.00054923 | 0.026016 | 380 | 0.29674 |
| FOXP4 | 0.00058003 | 0.027403 | 381 | 0.28404 |
| MRPS21 | 0.00058278 | 0.027461 | 382 | 0.93872 |
| PEX7 | 0.00059598 | 0.02801 | 383 | 0.64332 |
| NPC1 | 0.00060644 | 0.028427 | 384 | 0.50124 |
| PRDX1 | 0.00063064 | 0.029484 | 385 | 0.69438 |
| MRPL2 | 0.00063779 | 0.029741 | 386 | 0.68449 |
| CYC1 | 0.00064659 | 0.030074 | 387 | 0.67914 |
| EIF1AX | 0.0007071 | 0.032719 | 388 | 0.58446 |
| HIST1H4K | 0.00048157 | 0.023054 | 389 | 1.0735 |
| ELOF1 | 0.00072085 | 0.03327 | 390 | 0.95725 |
| ATP5J | 0.00088146 | 0.039468 | 391 | 1.0133 |
| CTDNEP1 | 0.0007247 | 0.033362 | 392 | 0.60851 |
| KIAA0195 | 0.0007434 | 0.034136 | 393 | 0.48824 |
| TARS2 | 0.00074945 | 0.034326 | 394 | 0.76566 |
| PPP5C | 0.0007566 | 0.034566 | 395 | 0.42889 |
| NAT6 | 0.00080501 | 0.036684 | 396 | 0.46719 |
| GTPBP10 | 0.00066584 | 0.03089 | 397 | 1.618 |
| MRPL9 | 0.00083966 | 0.03807 | 398 | 0.54521 |
| C5orf30 | 0.00084626 | 0.038273 | 399 | 0.28907 |
| NUP153 | 0.00086001 | 0.038797 | 400 | 0.53941 |
| ZNF292 | 0.00086661 | 0.038998 | 401 | 0.45978 |
| SMARCD1 | 0.00087871 | 0.039443 | 402 | 0.66438 |
| NDUFAF6 | 0.00090127 | 0.040155 | 403 | 0.89338 |
| MAZ | 0.0013193 | 0.054994 | 404 | 0.30628 |
| UQCRC2 | 0.00092987 | 0.041327 | 405 | 0.72243 |
| SLAMF6 | 0.00093702 | 0.041441 | 406 | 0.52606 |
| IPPK | 0.00094857 | 0.041849 | 407 | 0.56333 |
| ZC3H12A | 0.00096067 | 0.0422 | 408 | 0.46648 |
| MRPL51 | 0.00096122 | 0.0422 | 409 | 0.75373 |
| C6orf47 | 0.00097552 | 0.042724 | 410 | 0.3603 |
| AMMECR1 | 0.00099367 | 0.043413 | 411 | 0.36312 |
| CNOT10 | 0.0010223 | 0.044447 | 412 | 0.83226 |
| TBL1XR1 | 0.0010575 | 0.045867 | 414 | 1.1116 |
| PACSIN2 | 0.001091 | 0.047208 | 415 | 0.37208 |
| WAC | 0.0010943 | 0.047237 | 416 | 0.98453 |
| FAM13B | 0.0010987 | 0.047314 | 417 | 0.49867 |
| ANKHD1- | 0.0011163 | 0.047957 | 418 | 0.47337 |
| EIF4EBP3 | | | | |
| THUMPD1 | 0.0011339 | 0.048597 | 420 | 0.47765 |
| ATP5L | 0.00093372 | 0.041396 | 421 | 0.56458 |

### Example 4: T Cell Killing Enhanced or Reduced by Cancer Cell Knockouts

To identify comprehensively genetic knockouts (KOs) in cancer cells that enhance or reduce killing by human Vγ9Vδ2 T cells, CRISPR was used to create a genome-wide pool of KG cancer target cells.

Vγ9Vδ2 T cells were selected as non-conventional T cells, half-way between adaptive and innate immunity, with a natural inclination to react against malignant B cells, including malignant myeloma cells. The Vγ9Vδ2 T cells were expanded from healthy donors' peripheral blood mononuclear cells (PBMCs) supplemented with interleukin-2 (IL-2) and with a single dose of zoledronate (ZOL).

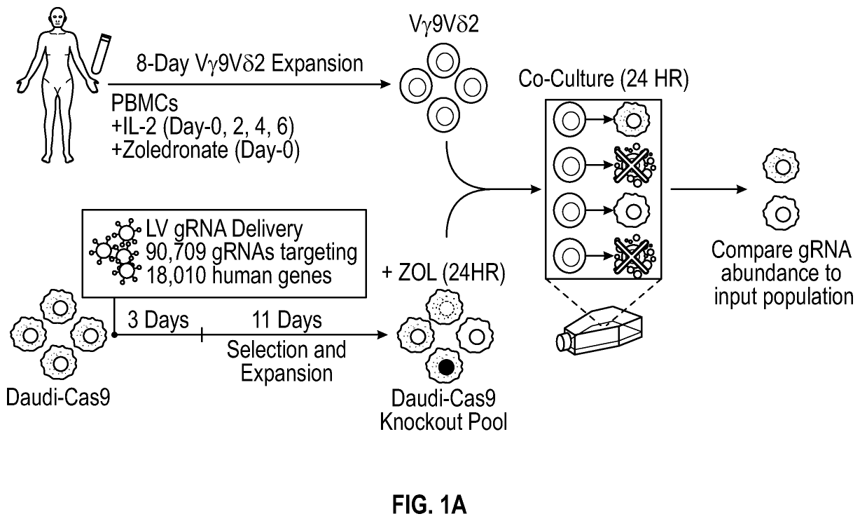

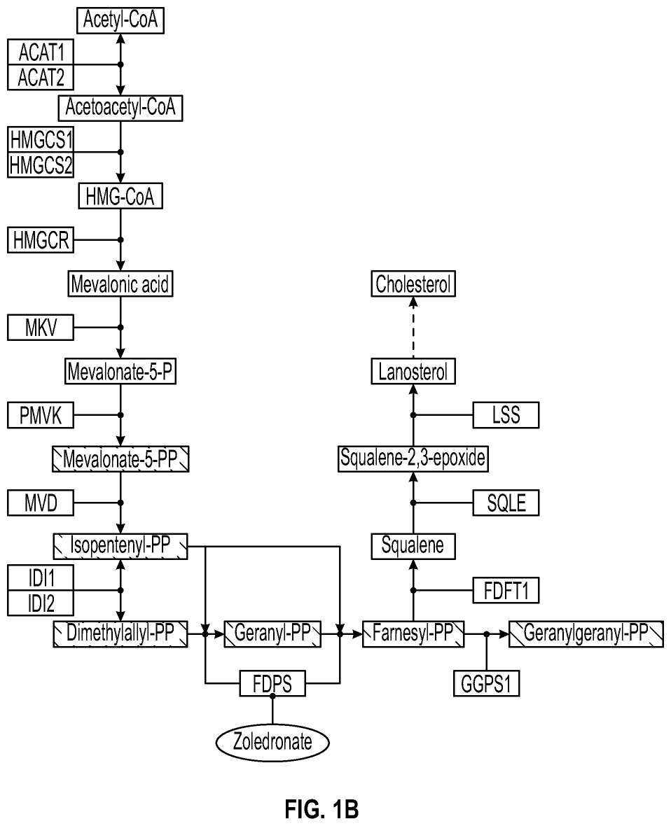

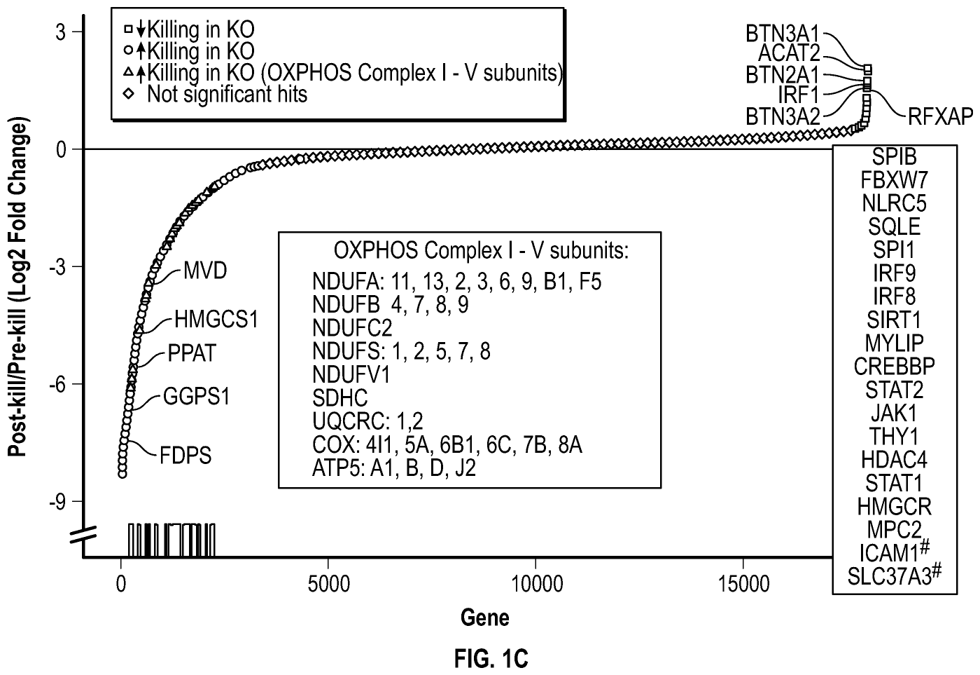

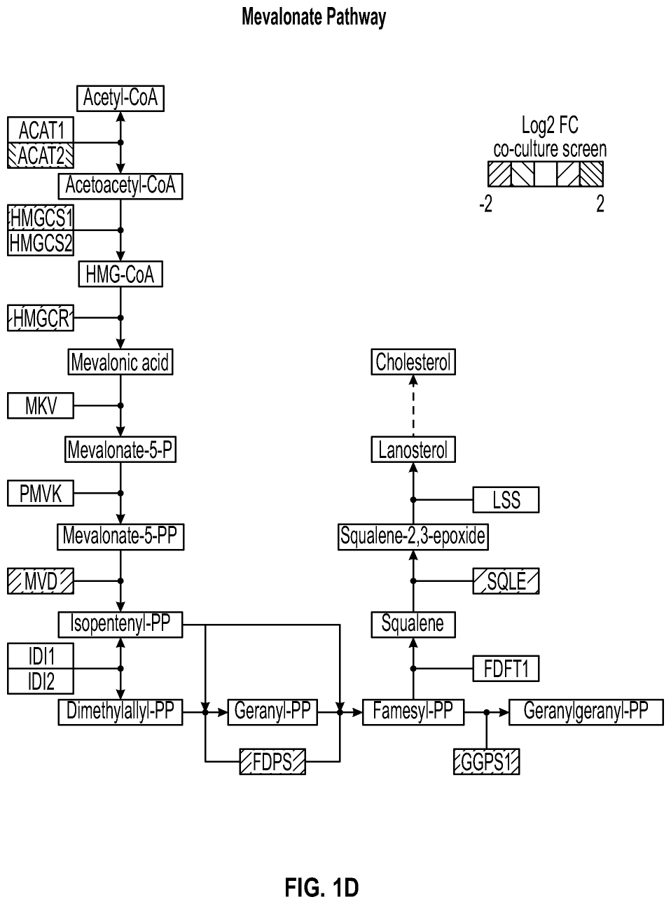

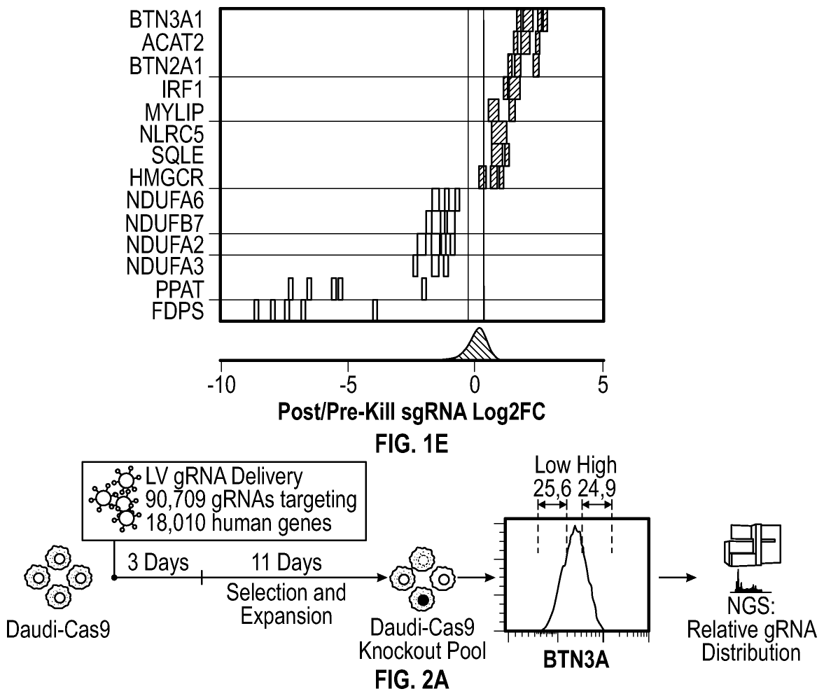

Daudi (Burkitt's lymphoma) cells that constitutively express Cas9 (Daudi-Cas9) were transduced with a lentiviral genome-wide knockout (KO) CRISPR library (90,709 guide RNAs against 18,010 human genes). The transduced cells were expanded and treated with zoledronate for 24 hours prior to the γδ T cell co-culture. Zoledronate (ZOL), artificially elevates phosphoantigen levels by inhibiting a downstream step of the mevalonate pathway (FIG. 1B).

The KO cancer target cells were co-cultured with Vγ9Vδ2 T cells, allowing the Vγ9Vδ2 T cells to recognize phosphoantigen accumulation in target cells. Accounting for donor-to-donor variability in Vγ9Vδ2 T cell cytotoxicity, each donor's Vγ9Vδ2 T cells were co-cultured with the genome-wide KO Daudi-Cas9 cells at two different effector-to-target (E:T) ratios (1:2, 1:4) for 24 hours in the presence of zoledronate.

After isolating surviving cells from the co-culture, loss and enrichment of different single-gene KO cells were determined by detecting gRNA sequences among the surviving population relative to baseline KO cell distribution among the genome-wide KO Daudi-Cas9 cells (FIG. 1A). For each of the three T cell donors, the effector-to-target (E:T) ratio was chosen that yielded Daudi cell survival matching the other two donors (approximately 50%). The screen hits (false discovery rate [FDR]<0.05) were consistent among the three donors, with the expected variability that occurs in cell-cell interaction screens (Patel et al., Nature 548, 537-542 (2017)). Exemplary results are shown in Table 3.

TABLE 3
Exemplary Co-culture Screen Results (sgRNA)

| | | treat | | | | high_in_ |
|---|---|---|---|---|---|---|
| sgRNA | Gene | mean | LFC | score | FDR | treatment |
| BTN3A1_GGGAGCCGGTTACTTCCTG | BTN3A1 | 7249.9 | 2.5697 | 21.24 | 3.68E−95 | TRUE |
| SEQ ID NO: 110 | | | | | | |
| BTN3A1_CTTCTTCAGGAGCGCCCAG | BTN3A1 | 9150.3 | 2.2076 | 19.78 | 2.02E−82 | TRUE |
| SEQ ID NO: 111 | | | | | | |
| BTN2A1_TCTTGGAAGTAACAGCGGT | BTN2A1 | 6366.6 | 2.4492 | 18.758 | 5.04E−74 | TRUE |
| SEQ ID NO: 112 | | | | | | |
| BTN3A1_AGAGTTGAGAGAAATGGCA | BTN3A1 | 4251.1 | 2.7951 | 18.173 | 1.92E−69 | TRUE |
| SEQ ID NO: 113 | | | | | | |
| BTN3A2_ACGTCACAGCCTCTGACAG | BTN3A2 | 7396.6 | 2.2137 | 17.862 | 4.20E−67 | TRUE |
| SEQ ID NO: 114 | | | | | | |
| BTN3A1_TGCTGCTTCTTGGGGGAGC | BTN3A1 | 8413.1 | 2.0651 | 17.532 | 1.23E−64 | TRUE |
| SEQ ID NO: 115 | | | | | | |
| BTN3A2_GCGGGATGGCATCACTGCA | BTN3A2 | 5012.6 | 2.3489 | 15.831 | 2.46E−52 | TRUE |
| SEQ ID NO: 116 | | | | | | |
| BTN2A2_TGTGCACTGGTCTCAGGTA | BTN2A2 | 4689.1 | 2.0744 | 13.201 | 9.87E−36 | TRUE |
| SEQ ID NO: 117 | | | | | | |
| ACAT2_CAGTCCAGTCAATAGGGAT | ACAT2 | 4335.4 | 2.0827 | 12.759 | 2.81E−33 | TRUE |
| SEQ ID NO: 118 | | | | | | |
| SPIB_CTGGGGCTACTGACGCGCG | SPIB | 7610.7 | 1.6241 | 12.692 | 5.96E−33 | TRUE |
| SEQ ID NO: 119 | | | | | | |
| IRF1_TGCCTGTTTGTTCCGGAGC | IRF1 | 8072.2 | 1.4325 | 11.386 | 4.07E−26 | TRUE |
| SEQ ID NO: 120 | | | | | | |
| BTN3A1_CAGGGCGGCGATCCACCTC | BTN3A1 | 3523.7 | 2.049 | 11.298 | 1.02E~25 | TRUE |
| SEQ ID NO: 121 | | | | | | |
| BTN2A1_TCTCCATGCCTGATGCAGA | BTN2A1 | 5566.5 | 1.6551 | 11.104 | 8.40E−25 | TRUE |
| SEQ ID NO: 122 | | | | | | |
| RFXAP_AGACACTTCGGACCCTCCG | RFXAP | 6378.3 | 1.5343 | 10.922 | 5.87E−24 | TRUE |
| SEQ ID NO: 123 | | | | | | |
| SPIB_GGGTACGGGGCATATGCCG | SPIB | 4360 | 1.7824 | 10.693 | 6.63E−23 | TRUE |
| SEQ ID NO: 124 | | | | | | |
| SCO1_CACCCCCGTGGTCGCAGAA | SCO1 | 714.32 | −3.6001 | 10.413 | 1.23E−21 | FALSE |
| SEQ ID NO: 125 | | | | | | |
| RFXAP_ACAGGGTTGCATCACTAGC | RFXAP | 4884.6 | 1.6018 | 10.037 | 5.58E−20 | TRUE |
| SEQ ID NO: 126 | | | | | | |
| BTN3A1_GTTGATGTGAAGGGTTACA | BTN3A1 | 2842.7 | 1.9969 | 9.8596 | 3.14E−19 | TRUE |
| SEQ ID NO: 127 | | | | | | |
| IRF1_CTAGGCCGATACAAAGCAG | IRF1 | 4103.9 | 1.6906 | 9.7786 | 6.64E−19 | TRUE |
| SEQ ID NO: 128 | | | | | | |
| SPI1_CACGTCCTCGATACCCCCA | SPI1 | 5441.8 | 1.4776 | 9.6891 | 1.52E~18 | TRUE |
| SEQ ID NO: 129 | | | | | | |
| IRF1_CACCTCCTCGATATCTGGC | IRF1 | 7029.4 | 1.2122 | 8.8869 | 2.71E−15 | TRUE |
| SEQ ID NO: 130 | | | | | | |
| SPIB_GCTAGCGAAGTTCTCCGTG | SPIB | 4447.4 | 1.4916 | 8.8597 | 3.31E−15 | TRUE |
| SEQ ID NO: 131 | | | | | | |
| BTN3A1_AGGGAACTTCTGATGGTAC | BTN3A1 | 3095 | 1.7308 | 8.7326 | 9.82E−15 | TRUE |
| SEQ ID NO: 132 | | | | | | |
| LUM_TAGAAAACTCCAAGATAAA | LUM | 171.75 | 4.64 | 8.61 | 4.27E−14 | TRUE |
| SEQ ID NO: 133 | | | | | | |
| IRF1_GGAAGCATGCTGCCAAGCA | IRF1 | 3499.5 | 1.6107 | 8.5638 | 4.13E−14 | TRUE |
| SEQ ID NO: 134 | | | | | | |
| UGGT2_TTCGCAATCTTGGGATCAA | UGGT2 | 3035.8 | 1.6772 | 8.3503 | 2.38E−13 | TRUE |
| SEQ ID NO: 135 | | | | | | |
| IRF1_AGCCGAGATGCTAAGAGCA | IRF1 | 3151.9 | 1.6173 | 8.1693 | 1.04E−12 | TRUE |
| SEQ ID NO: 136 | | | | | | |
| SPI1_ATACTCGTGCGTTTGGCGT | SPI1 | 6915.7 | 1.1261 | 8.1546 | 1.13E−12 | TRUE |
| SEQ ID NO: 137 | | | | | | |
| SPIB_CCTCGTGGCTGGCCCCGAG | SPIB | 5523.4 | 1.2165 | 7.9175 | 7.58E−12 | TRUE |
| SEQ ID NO: 138 | | | | | | |
| WDR59_TATCCGCACATCGCCGTCA | WDR59 | 327.58 | −3.8469 | 7.8204 | 1.58E−11 | FALSE |
| SEQ ID NO: 139 | | | | | | |
| RPP38_CGATTCTCTCACTGAGCCG | RPP38 | 558.49 | −3.1996 | 7.8058 | 1.73E−11 | FALSE |
| SEQ ID NO: 140 | | | | | | |
| SUGT1_TTTGACTGATGAGTCCACT | SUGT1 | 3294.2 | 1.5136 | 7.7611 | 2.39E−11 | TRUE |
| SEQ ID NO: 141 | | | | | | |
| FBXW7_AGGTTTCATACACAGTCCA | FBXW7 | 3314.5 | 1.4859 | 7.629 | 6.50B-11 | TRUE |
| SEQ ID NO: 142 | | | | | | |
| FBXW7_TTCTTCCAACTGTCCTTGC | FBXW7 | 6114.5 | 1.1042 | 7.5154 | 1.51E−10 | TRUE |
| SEQ ID NO: 143 | | | | | | |
| ACACA_GTTAGAGACGCTATTCCGC | ACACA | 104.65 | −5.2135 | 7.4534 | 1.87E−10 | FALSE |
| SEQ ID NO: 144 | | | | | | |
| MRPS26_CCCCCGGCCGCACACCTGA | MRPS26 | 431.96 | −3.3597 | 7.3571 | 4.60E−10 | FALSE |
| SEQ ID NO: 145 | | | | | | |
| CCDC82_AAGAGCTTGATAGTAACAA | CCDC82 | 83.034 | 4.8194 | 7.3303 | 9.36E−10 | TRUE |
| SEQ ID NO: 146 | | | | | | |
| BTN2A1_ATGAGGGGCCATGAAGACG | BTN2A1 | 1007.3 | 2.3876 | 7.3174 | 6.30E−10 | TRUE |
| SEQ ID NO: 147 | | | | | | |
| MRPL28_TTCCCCCCGAATCCCAGCG | MRPL28 | 469.77 | −3.2156 | 7.2159 | 1.21E−09 | FALSE |
| SEQ ID NO: 148 | | | | | | |
| ARL14EPL_TTAATAGCAACAAATAGAG | ARL14-EPL | 227.01 | 3.9422 | 7.215 | 1.75E−09 | TRUE |
| SEQ ID NO: 149 | | | | | | |
| SAE1_TGCTTCTTGTCGGCTTGAA | SAE1 | 19.337 | −7.4222 | 7.1879 | 4.23E−10 | FALSE |
| SEQ ID NO: 150 | | | | | | |
| SPIB_GAGGTCTCGGACAGCGAGT | SPIB | 3907.1 | 1.2994 | 7.1579 | 1.77E−09 | TRUE |
| SEQ ID NO: 151 | | | | | | |
| IFNAR1_TCCATCAGATGCTTGTACG | IFNARI | 4399.3 | 1.2274 | 7.1422 | 1.94E−09 | TRUE |
| SEQ ID NO: 152 | | | | | | |
| RFXAP_CGTTAGGTACCTGTGCGAA | RFXAP | 2592.9 | 1.5428 | 7.0414 | 3.84E−09 | TRUE |
| SEQ ID NO: 153 | | | | | | |
| BTN2A1_AGCCCCTCATTTCAATGAG | BTN2A1 | 1965.3 | 1.7442 | 7.0382 | 3.85E−09 | TRUE |
| SEQ ID NO: 154 | | | | | | |
| IRF9_TGTATCAGTTGCTGCCACC | IRF9 | 4293.9 | 1.2192 | 7.0068 | 4.71E−09 | TRUE |
| SEQ ID NO: 155 | | | | | | |
| PNLIPRP1_GCCCCTGAAAATTCTCCCC | PNL- | 1182.9 | −2.1878 | 7.0018 | 4.78E−09 | FALSE |
| SEQ ID NO: 156 | IPRP1 | | | | | |
| RFXAP_ACGAGGAGACTCACTCGGG | RFXAP | 1110.2 | 2.2103 | 6.9897 | 5.24E−09 | TRUE |
| SEQ ID NO: 157 | | | | | | |
| SPIB_CGGGTCGAAGGCTTCATAG | SPIB | 1900.6 | 1.7479 | 6.9394 | 7.15E−09 | TRUE |
| SEQ ID NO: 158 | | | | | | |
| ALCAM_GTGTGCATGCTAGTAACTG | ALCAM | 2462.2 | 1.5404 | 6.8519 | 1.27E−08 | TRUE |
| SEQ ID NO: 159 | | | | | | |
| FBXW7_TGAAGTCTCGTTGAAACTG | FBXW7 | 2652.5 | 1.4811 | 6.8093 | 1.68E−08 | TRUE |
| SEQ ID NO: 160 | | | | | | |
| PRMT1_TGGTGCTGGACGTCGGCTC | PRMT1 | 6.7801 | −8.6196 | 6.7595 | 2.81E−09 | FALSE |
| SEQ ID NO: 161 | | | | | | |
| AARS2_ATCCGCCTACCCCGCTCCA | AARS2 | 99.765 | −4.9928 | 6.7194 | 2.34E−08 | FALSE |
| SEQ ID NO: 162 | | | | | | |
| XPNPEP1_GGACTTGTAGGGATGCACC | XPN-PEP1 | 2841.3 | 1.4173 | 6.7154 | 3.04E−08 | TRUE |
| SEQ ID NO: 163 | | | | | | |
| GTF2A2_AGCACTGGCTCAGAGGGTC | GTF2A2 | 29.574 | −6.616 | 6.6409 | 1.90E−08 | FALSE |
| SEQ ID NO: 164 | | | | | | |
| MRPL9_CTCCACGATGACCGTGCCC | MRPL9 | 152.42 | −4.3743 | 6.5755 | 6.93E−08 | FALSE |
| SEQ ID NO: 165 | | | | | | |
| EEFSEC_TCACGCTGGTCGACTGCCC | EEFSEC | 6.8247 | −8.5255 | 6.5622 | 9.36E−09 | FALSE |
| SEQ ID NO: 166 | | | | | | |
| MTG2_ATGAGTACATTGCCGCGCT | MTG2 | 163.79 | −4.2612 | 6.5262 | 9.47E−08 | FALSE |
| SEQ ID NO: 167 | | | | | | |
| NUDCD3_TCACCACGTGCTTGGGTAC | NUD- | 262.75 | −3.6651 | 6.5243 | 9.95E~08 | FALSE |
| SEQ ID NO: 168 | CD3 | | | | | |
| ZC3H12A_CCGTGACCTCCAAGGCGAG | ZC3H-12A | 3712.6 | 1.2168 | 6.507 | 1.12E−07 | TRUE |
| SEQ ID NO: 169 | | | | | | |
| GMPPB_GCCGTGAGCTACATGTCGC | GMPPB | 627.87 | −2.6559 | 6.4797 | 1.31E−07 | FALSE |
| SEQ ID NO: 170 | | | | | | |
| SNF8_ACCATTGGCGTGGATCCGC | SNF8 | 35.261 | −6.2459 | 6.4788 | 1.31E−07 | FALSE |
| SEQ ID NO: 171 | | | | | | |
| NLRC5_AGTCACGTGTCCTACCGTC | NLRC5 | 5262.4 | 1.0275 | 6.4705 | 1.36E−07 | TRUE |
| SEQ ID NO: 172 | | | | | | |
| GGNBP2_GTATGGGAACTAATGTCGC | GGN-BP2 | 2491.2 | 1.4472 | 6.4369 | 1.68E−07 | TRUE |
| SEQ ID NO: 173 | | | | | | |
| OIP5_TATTCTACCCATGCTGCCC | OIP5 | 45.034 | −5.8943 | 6.4136 | 1.92E−07 | FALSE |
| SEQ ID NO: 174 | | | | | | |
| NAPG_GCAAAAGATGCCTGCCTGA | NAPG | 33.086 | −6.2732 | 6.3569 | 2.75E−07 | FALSE |
| SEQ ID NO: 175 | | | | | | |
| TRMT61A_CACGTCACCTTGGAGCCGA | TRMT-61A | 242.28 | −3.6911 | 6.3388 | 3.04E−07 | FALSE |
| SEQ ID NO: 176 | | | | | | |
| BCCIP_AATCTCTTACTGAAGCTGC | BCCIP | 64.834 | −5.3797 | 6.3357 | 3.06E−07 | FALSE |
| SEQ ID NO: 177 | | | | | | |
| MRPL55_CGACTCTACCCCGTGCTGC | MRPL-55 | 104.65 | −4.7404 | 6.305 | 3.68E−07 | FALSE |
| SEQ ID NO: 178 | | | | | | |
| OIP5_CGACTCGGTGCACCTCGCC | OIP5 | 16.543 | −7.255 | 6.3045 | 9.80E−08 | FALSE |
| SEQ ID NO: 179 | | | | | | |
| SPIB_GGGGGGTTCGTAGCAGAGC | SPIB | 3280.6 | 1.2452 | 6.2733 | 4.45E−07 | TRUE |
| SEQ ID NO: 180 | | | | | | |
| DNLZ_CAGCTCGTCTACACCTGCA | DNLZ | 6.7801 | −8.2244 | 6.2683 | 4.54E−07 | FALSE |
| SEQ ID NO: 181 | | | | | | |
| RPP21_GCACTCACGTCTCTGGCGC | RPP21 | 9.6859 | −7.9412 | 6.2576 | 8.45E−08 | FALSE |
| SEQ ID NO: 182 | | | | | | |
| RAB7A_CGGTTCCAGTCTCTCGGTG | RAB7A | 175.17 | −4.046 | 6.2219 | 6.02E−07 | FALSE |
| SEQ ID NO: 183 | | | | | | |
| SARS2_GCACGGTGCTCACCACGTC | SARS2 | 200.19 | −3.8724 | 6.2052 | 6.61E−07 | FALSE |
| SEQ ID NO: 184 | | | | | | |
| WDR61_ATTCCATCTATGGCTCCAC | WDR61 | 2.9058 | −9.1046 | 6.193 | 7.05E−07 | FALSE |
| SEQ ID NO: 185 | | | | | | |
| EEFSEC_TCATCCGGACCATCATCGG | EEFSEC | 83.299 | −4.9819 | 6.18 | 7.55E−07 | FALSE |
| SEQ ID NO: 186 | | | | | | |
| GSS_ACCCCAGCTGTGCACCGGT | GSS | 169.48 | −4.0666 | 6.1722 | 7.83E−07 | FALSE |
| SEQ ID NO: 187 | | | | | | |
| FCRL2_ACTATTTCTGTAGTACCAA | FCRL2 | 417.46 | 2.9186 | 6.1577 | 1.01E−06 | TRUE |
| SEQ ID NO: 188 | | | | | | |
| SHMT2_TGCTCGACTTTTCCGGCCA | SHMT2 | 220.66 | −3.7292 | 6.1486 | 8.97E−07 | FALSE |
| SEQ ID NO: 189 | | | | | | |
| PSMG4_CACCTGCGCAACCTCGCCG | PSMG4 | 279.92 | −3.4412 | 6.1467 | 8.97E−07 | FALSE |
| SEQ ID NO: 190 | | | | | | |
| ACAT2_CAAGTGAGTAGAGAAGATC | ACAT2 | 1085.8 | 1.9893 | 6.1049 | 1.16E−06 | TRUE |
| SEQ ID NO: 191 | | | | | | |
| N6AMT1_AGCAGAAACGTGTCCTCCG | N6A-MT1 | 224.71 | −3.683 | 6.0901 | 1.23E−06 | FALSE |
| SEQ ID NO: 192 | | | | | | |
| DKK1_CGCTAGTCCCACCCGCGGA | DKK1 | 4197.4 | 1.0785 | 6.0866 | 1.25E−06 | TRUE |
| SEQ ID NO: 193 | | | | | | |
| ALG12_TGCGATCACCACTGGCCCG | ALG12 | 374.84 | −3.0699 | 6.0622 | 1.43E−06 | FALSE |
| SEQ ID NO: 194 | | | | | | |
| SH3GL1_ACTTCTGTCACCGCCTTGC | SH3GL1 | 11.374 | −7.484 | 6.045 | 1.58E−06 | FALSE |
| SEQ ID NO: 195 | | | | | | |
| HISTIH3J_CACGCAAGGCCACGGTGCC | HIST- | 4138.5 | 1.0769 | 6.0349 | 1.66E−06 | TRUE |
| SEQ ID NO: 196 | 1H3J | | | | | |
| TTC7A_CAGTACGTCATGCTCTCGG | TTC7A | 63.697 | −5.2629 | 6.0307 | 1.68E−06 | FALSE |
| SEQ ID NO: 197 | | | | | | |
| TSC2_AGCATCTCATACACACGCG | TSC2 | 463.96 | −2.8218 | 6.0284 | 1.69E−06 | FALSE |
| SEQ ID NO: 198 | | | | | | |
| MED26_CCTCGGAACTCACGGCATG | MED26 | 1185.6 | −1.9192 | 6.025 | 1.70E−06 | FALSE |
| SEQ ID NO: 199 | | | | | | |
| RPP25L_TGGCTCTGGGTCGGTTGGA | RPP25L | 9.1906 | −7.7316 | 6.0209 | 1.73E−06 | FALSE |
| SEQ ID NO: 200 | | | | | | |
| BLQC1S1_ACCAAAGCTTCTGTCAGGC | BLQC-1S1 | 1264.6 | −1.8638 | 6.0173 | 1.75E−06 | FALSE |
| SEQ ID NO: 201 | | | | | | |
| SLC22A3_GCCTTCCTCTTCGTCGGCG | SLC-22A3 | 4633 | 1.0179 | 6.0158 | 1.75E−06 | TRUE |
| SEQ ID NO: 202 | | | | | | |
| SLC2A4_CAGGTCTGAAGCGCCTGAC | SLC- | 47.773 | 4.6166 | 6.0044 | 2.90E−06 | TRUE |
| SEQ ID NO: 203 | 2A4 | | | | | |
| PHB_GACCGATTCCGTGGAGTGC | PHB | 202.44 | −3.7742 | 6.0021 | 1.88E−06 | FALSE |
| SEQ ID NO: 204 | | | | | | |
| SHMT2_CAACCTCACGACCGGATCA | SHMT2 | 63.415 | −5.253 | 5.9975 | 1.91E−06 | FALSE |
| SEQ ID NO: 205 | | | | | | |
| ABCF1_AGCATCTCCGCTCATGGCA | ABCF1 | 504.57 | −2.7126 | 5.9741 | 2.19E−06 | FALSE |
| SEQ ID NO: 206 | | | | | | |
| IFFO1_GGCCTGGGTCGTCGCGACC | IFFO1 | 68.011 | 4.5353 | 5.9717 | 3.19E−06 | TRUE |
| SEQ ID NO: 207 | | | | | | |
| NUP37_GCCAGCACACACTCATGCC | NUP37 | 1092.8 | −1.9711 | 5.9656 | 2.28E−06 | FALSE |
| SEQ ID NO: 208 | | | | | | |

Pursuant to Gene Set Enrichment Analysis (GSEA), knockouts conferring a survival disadvantage to cancer cells in the Vγ9Vδ2 T cell co-culture included genes involved in various metabolic pathways, especially genes involved in OXPHOS, the tricarboxylic acid (TCA) cycle, and purine metabolism KEGG pathways, all of which are essential for maintaining a proper ATP balance (FIG. 1C; Table 4).

TABLE 4
Negatively Enriched Pathways

| KEGG Gene Set | # Genes | FDR. q-val |
|---|---|---|
| Aminoacyl tRNA Biosynthesis | 22 | 0 |
| Spliceosome | 119 | 0 |
| Nucleotide Excision Repair | 44 | 0 |
| Ribosome | 81 | 0 |
| RNA Polymerase | 25 | 0.000071 |
| Mismatch Repair | 23 | 0.000065 |
| DNA Replication | 34 | 0.000121 |
| Basal Transcription Factors | 35 | 0.000168 |
| Proteasome | 43 | 0.000158 |
| Pyrimidine Metabolism | 93 | 0.000295 |
| Oxidative Phosphorylation | 100 | 0.000739 |
| RNA Degradation | 51 | 0.000700 |
| Homologous Recombination | 26 | 0.000915 |
| N-Glycan Biosynthesis | 46 | 0.001468 |
| One Carbon Pool By Folate | 17 | 0.002199 |
| Purine Metabolism | 149 | 0.004278 |
| Parkinsons Disease | 98 | 0.004517 |
| Cell Cycle | 123 | 0.005302 |
| TCA Cycle | 30 | 0.006223 |
| Protein Export | 22 | 0.008706 |

Loss of OXPHOS, TCA, and purine metabolism functions in cancer cells can make those cancer cells more vulnerable to Vγ9Vδ2 T cell killing. Analyses described herein reveal that loss of structural subunits of Complexes I-V of the electron transport chain (ETC) driving OXPHOS significantly enhanced killing of cancer cells by T cells (FIG. 1C). The vertical lines on the x-axis of the FIG. 1C graph identify the rank positions of OXPHOS Complex I-V subunits listed in the green box—note that knockout of these OXPHOS genes makes cancer cells more vulnerable to T cell killing. The OXPHOS system comprises five multi-subunit protein complexes, of which NADH-ubiquinone oxidoreductase (complex 1, CI) is a major electron entry point into the electron transport chain (ETC) that is central to mitochondrial ATP synthesis. Knockouts of certain mevalonate pathway enzymes (HMGCS1, MVD, GGPS1) also significantly enhanced killing (FIG. 1C-ID), two of which would be expected to upregulate phosphoantigen concentrations (MVD, GGPS1).

Confirming the screen's accuracy, enhanced survival was observed among knockouts of (1) the components of the butyrophilin complex (BTN2A1, BTN3A1, BTN3A2) that activates Vγ9Vδ2 T cell receptors (TCRs); (2) mevalonate pathway enzymes (ACAT2, HMGCR, SQLE), two of which are upstream of phosphoantigen synthesis; (3) SLC37A3 (FDR<0.1), a transporter of zoledronate into the cytosol; (4) NLRC5, a transactivator of BTN3A1-3 genes; and (5) ICAM1 (FDR<0.1), a surface protein important for Vγ9Vδ2 T cell recognition of target cells (FIG. 1C-1D). Knockouts of various type I interferon (IFN-I) signaling components (IRF1, IRF8, IRF9, JAK1, STAT1, STAT2) also enhanced Daudi cell survival in the co-culture (FIG. 1C). Across thousands of healthy samples in a public database, the gene ontology pathways characterized by the response to IFN-I and IFN-γ are highly correlated to BTN3AJ gene expression. Confidence in significant hits (FDR<0.05) was further bolstered by consistent enrichment or depletion of separate sgRNAs targeting the same genes (FIG. 1E). As illustrated in FIG. 1E, cells with knockout of some genes (e.g., FDPS, PPAT, NDUFA3, NDUFA2, NDUFB7, NDUFA6) were frequently killed by the T cells, so the sgRNAs for these genes were detected in only small numbers of cells. However, cells with knockout of other genes (BTN3A1, ACAT2, BTN2A1, IRF1) were not killed so frequently by the T cells, so the sgRNAs for these genes were detected in significantly greater numbers of cells (FIG. 1E).

### Example 5: Genetic Modifications that Modulate BTN3A1

This Example describes experiments designed to determine if any of the enrichments or depletions observed in the co-culture screen were due to effects on BTN3A1.

Using publicly available data from healthy tissue, the inventors identified several positively enriched screen hits with strong (NLRC5, IRF1, IRF9, SPI1) or moderate (MYLIP) correlations to BTN3A1, while enriched upstream mevalonate pathway enzyme ACAT2 whose KO presumably would only deplete phosphoantigens showed no such correlation. In the case of the entire KEGG Oxidative Phosphorylation gene set, the vast majority of OXPHOS genes are negatively correlated to BTN3A1 in immune tissue, while the distribution of genome-wide pairwise BTN3A1 correlations followed a normal distribution centered at zero. This skewing further indicated that BTN3AJ expression could be affected by the cellular energy state and OXPHOS in particular.

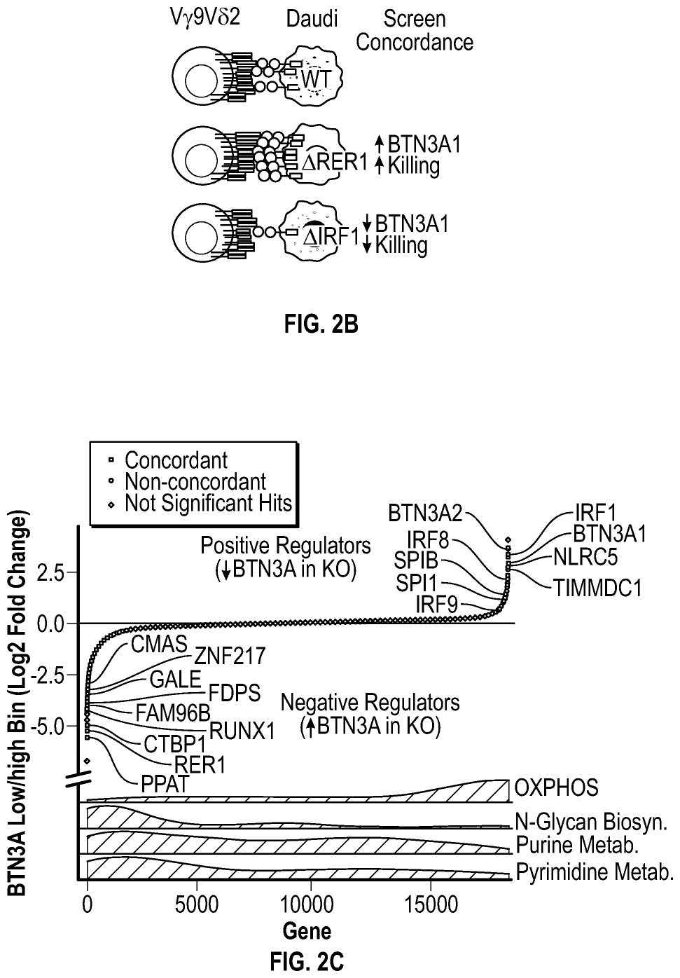

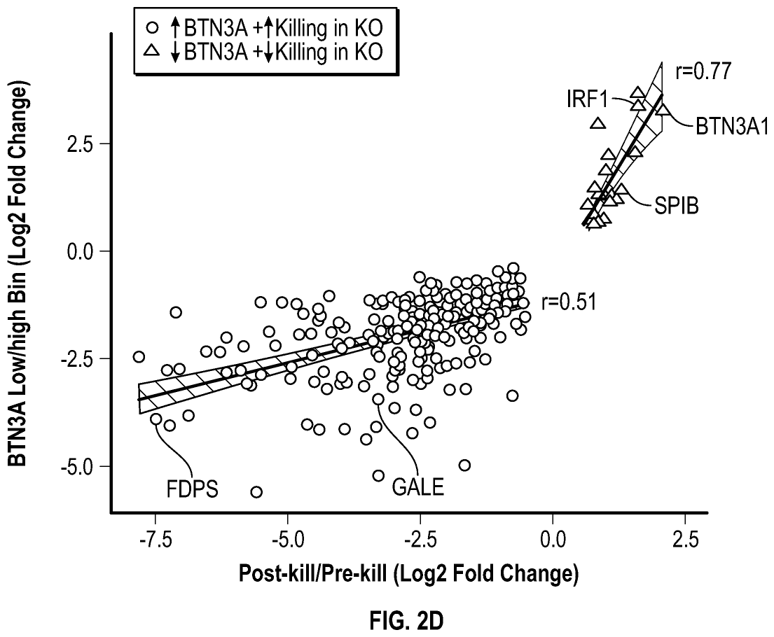

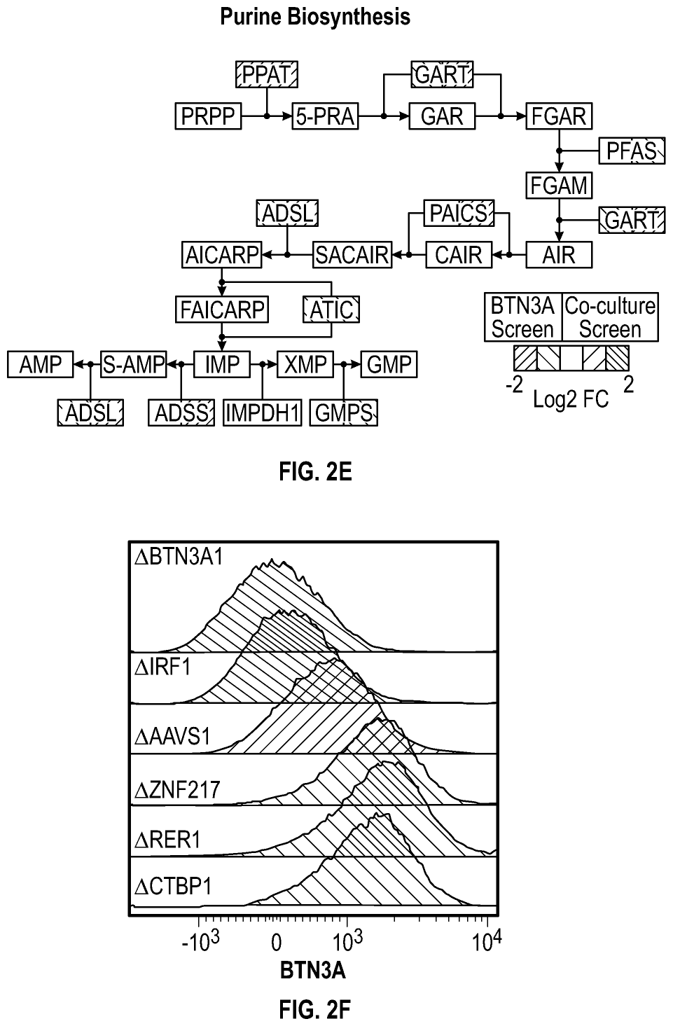

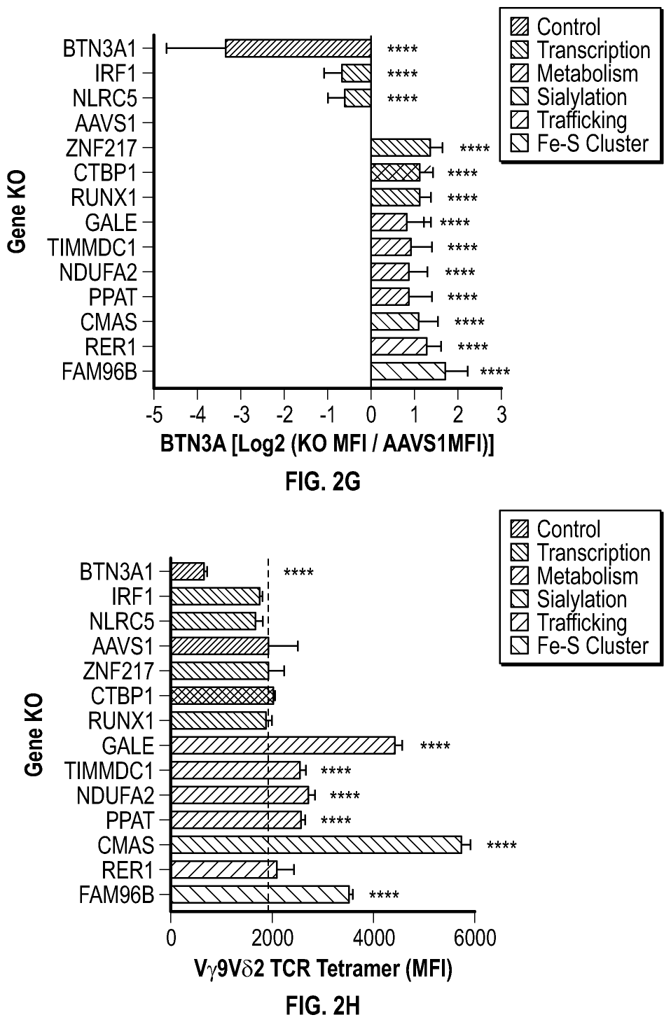

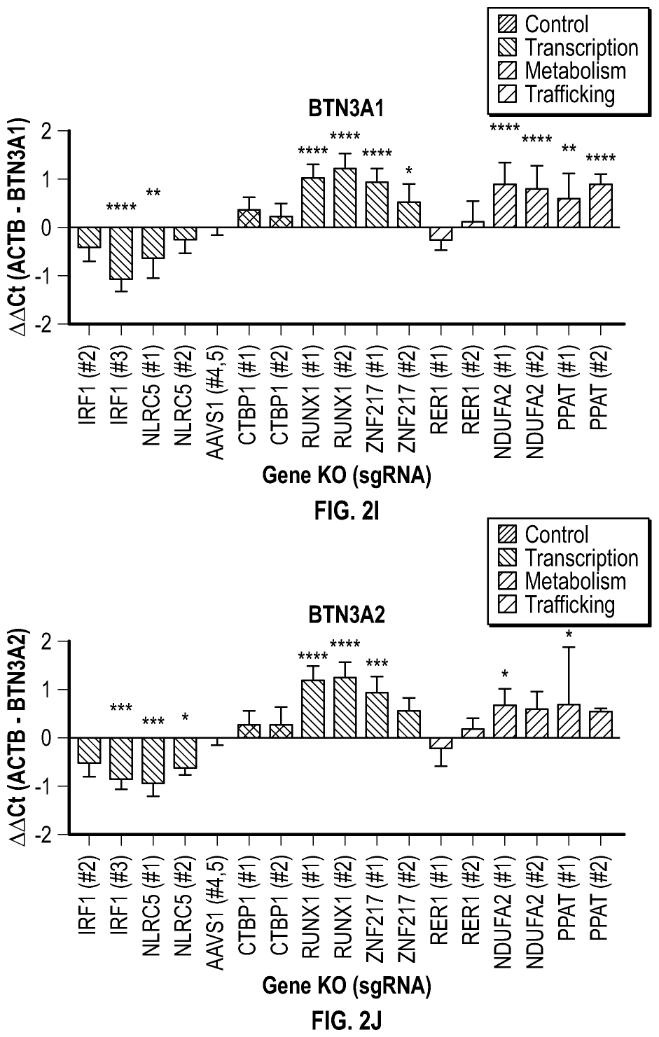

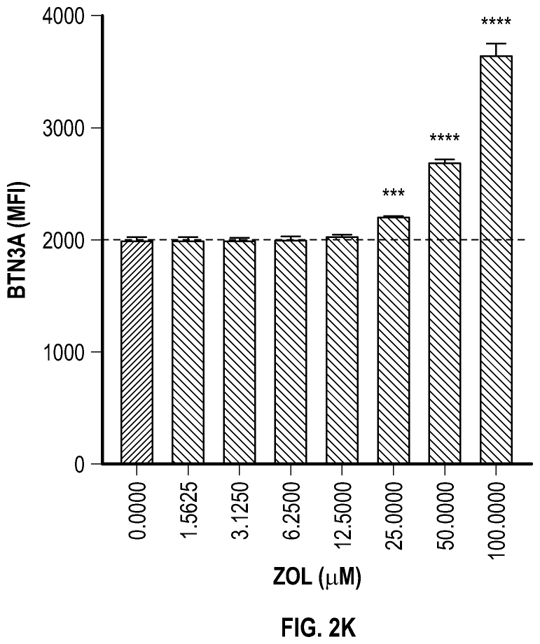

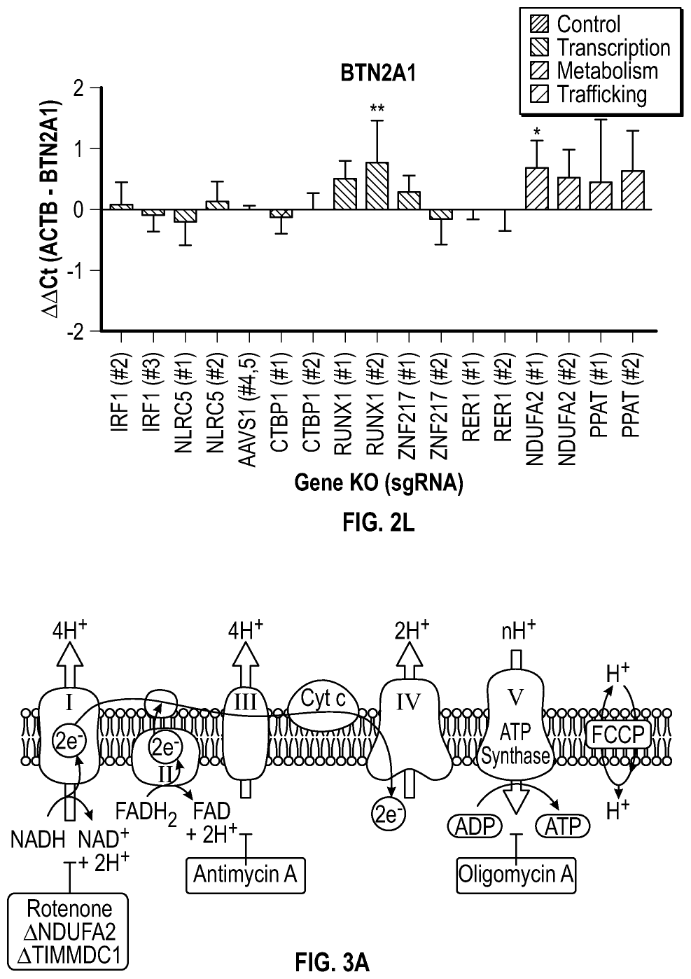

To comprehensively understand which of the co-culture screen hits act through regulation of BTN3A1 abundance, an unbiased genome-wide screen was performed to identify positive and negative regulators of BTN3A surface levels. The lentiviral genome-wide sgRNA library transduction was repeated in Daudi-Cas9 cells, while also using selection and outgrowth of transduced cells. The genome-wide pool of Daudi KO cells was stained for cell surface BTN3A (combined expression of BTN3A1, BTN3A2, and BTN3A3, which have identical ectodomains). Cells in the top and bottom BTN3A expression quartiles were FACS sorted to identify genetic KO enrichments in each bin (FIG. 2A). Starting from transduction through next generation sequencing (NGS) library preparation, the entire screen was performed in three separate replicates, whose hits strongly correlated with each other.

Significant hits from the BTN3A regulator screen were compared to those of the co-culture screen. A hit was considered concordant between the two screens if its knockout either (1) conferred a survival advantage against T cells and downregulated BTN3A, or (2) conferred a survival disadvantage against T cells and upregulated BTN3A (FIG. 2B). A large fraction of significant hits (FDR<0.01) in the BTN3A screen were concordant with the co-culture screen (FIG. 2C). A number of knockouts that conferred a survival advantage in the co-culture screen were confirmed to be positive regulators of BTN3A, such as transcriptional regulators NLRC5, IRF1, IRF8, IRF9, SPI1, SPIB, and so on. To determine an effect size correlation between the two screens, the log-fold changes (LFC) of the co-culture screen and the BTN3A screen were compared. Concordant hit knockouts that protected against Vγ9Vδ2 T cell killing and downregulated BTN3A showed a strong effect size correlation (Pearson's r=0.77), while the concordant hit knockouts that enhanced T cell killing and upregulated BTN3A showed a moderate correlation (r=0.51) (FIG. 2D).

GSEA showed that several highly enriched metabolic pathways were concordant between screens, specifically the N-glycan biosynthesis, the purine metabolism, the pyrimidine metabolism, and the one carbon pool by folate KEGG pathways (FIG. 2C, Table 5).

TABLE 5
GSEA of KEGG gene sets that positively or negatively
regulate surface BTN3A expression

| BTN3A Positive Regulation KEGG Gene Set | # Genes | q-val |
|---|---|---|
| Oxidative Phosphorylation | 100 | 0 |
| Alzheimer's Disease | 144 | 0 |
| Parkinsons Disease | 98 | 0 |
| Huntingtons Disease | 156 | 0 |
| Aminoacyl tRNA Biosynthesis | 22 | 0 |
| Cardiac Muscle Contraction | 72 | 0.0005 |
| Antigen Processing and Presentation | 78 | 0.0366 |
| N-Glycan Biosynthesis | 46 | 0 |
| Amino and Nucleotide Sugar Metabolism | 42 | 0 |
| Purine Metabolism | 149 | 0 |
| RNA Polymerase | 25 | 0 |
| Pyrimidine Metabolism | 93 | 0 |
| One Carbon Pool by Folate | 16 | 0.001 |
| Proteasome | 43 | 0.001 |
| DNA Replication | 34 | 0.001 |
| Ribosome | 81 | 0.002 |
| Base Excision Repair | 33 | 0.002 |
| Nucleotide Excision Repair | 44 | 0.002 |
| Amyotrophic Lateral Sclerosis (ALS) | 52 | 0.006 |
| Pentose Phosphate Pathway | 26 | 0.007 |
| RNA Degradation | 51 | 0.007 |
| Homologous Recombination | 26 | 0.007 |
| mTOR Signaling Pathway | 50 | 0.008 |
| Cell Cycle | 122 | 0.008 |
| Alanine, Aspartate, and Glutamate Metabolism | 30 | 0.015 |
| Galactose Metabolism | 26 | 0.030 |
| Ubiquitin Mediated Proteolysis | 129 | 0.033 |
| Cysteine and Methionine Metabolism | 34 | 0.039 |
| Pantothenate and CoA Biosynthesis | 16 | 0.038 |
| Glutathione Metabolism | 49 | 0.039 |
| Glycolysis and Gluconeogenesis | 60 | 0.039 |
| Chronic Myeloid Leukemia | 73 | 0.045 |

OXPHOS was the most enriched pathway among Daudi cells with downregulated surface BTN3A, which was unexpected. The opposite effect was expected because this pathway was enriched among Daudi KOs with a survival disadvantage in the co-culture screen. The strong divergent effects indicated that the relationship between OXPHOS and BTN3A was a complex biological phenomenon that was likely context dependent.

While the mevalonate pathway is not known to regulate BTN3A surface abundance, the screen revealed an upregulation of BTN3A among cells with an FDPS deletion (FIG. 2C). To validate this result, a ZOL (FDPS inhibitor) dose response was performed in Daudi-Cas9 cells, which resulted in a substantial and dose-dependent increase in BTN3A (FIG. 2K).

For a subset of the enriched pathways, the inventors performed analyses to determine how much of each pathway was captured in by the two CRISPR screens and the level of screen concordance for those pathway components. The inventors mapped the LFC and significance (FDR<0.05) from both screens for de novo purine biosynthesis (FIG. 2E), OXPHOS, iron-sulfur (Fe-S) cluster formation, N-glycan biosynthesis, and sialylation.

The purine biosynthesis pathway was captured almost in its entirety with all the hits showing concordance between the two screens as negative regulators of BTN3A and lowering survival in the Vγ9Vδ2 T cell co-culture. This pathway produces IMP, GMP, and AMP nucleotides, the latter of which is important in maintaining proper energy homeostasis both by regulating AMP-activated protein kinase (AMPK) activity and by being regenerated into ATP. Most of the subunits comprising the five electron transport chain (ETC) complexes driving ATP-producing OXPHOS were significant hits with opposing effects in the two screens, indicating that this pathway's effects on BTN3A levels could depend on exogenous culture conditions. The screens also reveal mostly concordant and significant hits in the Fe—S cluster formation machinery that produces this prosthetic group for both mitochondrial and cytosolic proteins. The enzyme catalyzing the first step in purine biosynthesis (PPAT) and OXPHOS Complexes I, II, and III contain Fe—S clusters. Finally, both the N-glycan biosynthesis pathway responsible for glycosylation of proteins in the endoplasmic reticulum and the Golgi apparatus, as well as the pathway that sialylates glycosylated proteins, came up as strongly enriched pathways with a number of concordant hits throughout the pathways.

Interestingly, the initial approach that led to the discovery of BTN2A1 as the cognate ligand of Vγ9Vδ2 TCRs identified two gene KOs that caused the highest disruption of Vγ9Vδ2 TCR tetramer-ligand interactions among all KOs—BTN2A1 itself and SPPL3. Downregulation of SPPL3 leads to global hyperglycosylation, and SPPL3 deletion has been shown to limit HLA-I accessibility to its interaction partners.

Together, these observations bolster the finding from the inventors' two screens that decreased N-linked glycosylation increases BTN3A surface staining and increases γδ T cell killing of target cells. In total, pathway visualization reveals that the screens described herein capture large portions of different pathways, further enhancing confidence that these pathways play important roles in BTN3A expression and susceptibility to Vγ9Vδ2 T cell targeting.

### Example 6: Gene Products that Regulate BTN3A

To validate a subset of BTN3A regulators, a lentiviral sgRNA approach was used to generate one BTN3AJ KO and two distinct KOs for every other gene target, including the AAVS1 safe-harbor cutting site with no relevance to BTN3A regulation that is used as a control for CRISPR cutting. The inventors confirmed that edited cells had disruptive indels in >90% of the cells. These Daudi-Cas9 KO cells were stained for BTN3A at 13 days post-transduction, matching the screen readout time-point.

For each target, the BTN3A median fluorescence intensity (MFI) was consistent between the two distinct KO cell lines. Deletion of IRF1 had as strong of an effect on surface BTN3A abundance as deletion of NLRC5, the only known transcriptional regulator of BTN3A1-3.

The inventors confirmed that the transcriptional repressors ZNF217, CtBP1, and RUNX1 negatively regulate BTN3A abundance (FIG. 2F-2G). Interestingly, CtBP1—a metabolic sensor whose transcriptional and trafficking regulation depend on the cellular NAD+/NADH ratio—was the top ranked KO among Daudi-Cas9 cells with upregulated BTN3A in the CRISPR screen (Supplementary Table 3).

Increased BTN3A surface abundance was also observed after disruption of the sialylation machinery (CMAS), after disruption of the retention in endoplasmic reticulum sorting receptor 1 (RER1), and after disruption of the Fe—S cluster formation (FAM96B) (FIG. 2F-2G). RER1 can control egress of multiprotein complexes out of the endoplasmic reticulum (ER) to the Golgi apparatus, indicating that it could control BTN3A intracellular trafficking and maintain proper complex assembly prior to endoplasmic reticulum egress of the BTN2A1-BTN3A1-BTN3A2 complex.

The inventors then confirmed that surface BTN3A abundance increases with deletions in galactose catabolism (GALE), de novo purine biosynthesis (PPA7), and OXPHOS complex I (NDUFA2, TIMMDC1) (FIG. 2G). Validation results for complex I knockouts contradicted the BTN3A screen results and were concordant with the co-culture screen findings. These data further indicated that a complex relationship exists between OXPHOS and BTN3A expression that could be dependent on culture conditions, given the different requirements of a high-coverage genome-wide screen and culturing individual KO cells. Using a tetramer of the G115 Vγ9Vδ2 TCR clone, the inventors determined that GALE, NDUFA2, PPAT, CMAS, and FAM96B KOs showed consistently higher TCR binding relative to the AAVS1 deletion controls (FIG. 2H).

### Example 7: Genes that Modulate BTN3A Expression

This Example describes experiments designed to help determine the mechanism by which some of the validated hits regulate BTN3A.

BTN2A1, BTN3A1, and BTN3A2 transcript levels were measured in a subset of the Daudi-Ca9 KO cell lines. RER1 KO cells served as a negative control. KO cell lines of transcriptional activators IRF1 and NLRC5 were confirmed to cause downregulation of BTN3A1/2 transcripts. BTN3A1/2 transcripts were upregulated in cells knocked out for transcriptional repressors ZNF217 and RUNX1. CTBP1 KO cells showed a weak upregulation of BTN3A1-2 transcripts that was not statistically significant, indicating that its effects on BTN3A surface abundance could be indirect or through its trafficking regulation.

The inventors also determined that knockout of NDUFA2 (OXPHOS) and PPAT (purine biosynthesis) caused upregulation of BTN3A1/2 transcripts, providing insights that allowed the inventors to dissect how metabolic perturbations in the cell are regulating BTN3A (FIG. 2I-2J). RUNX1 was the only transcriptional regulator that had a significant effect on BTN2A1 transcription, and while the two NDUFA2 and the two PPAT KOs increased BTN2A1 transcript levels, only one NDUFA2 KO reached statistical significance (FIG. 2L).

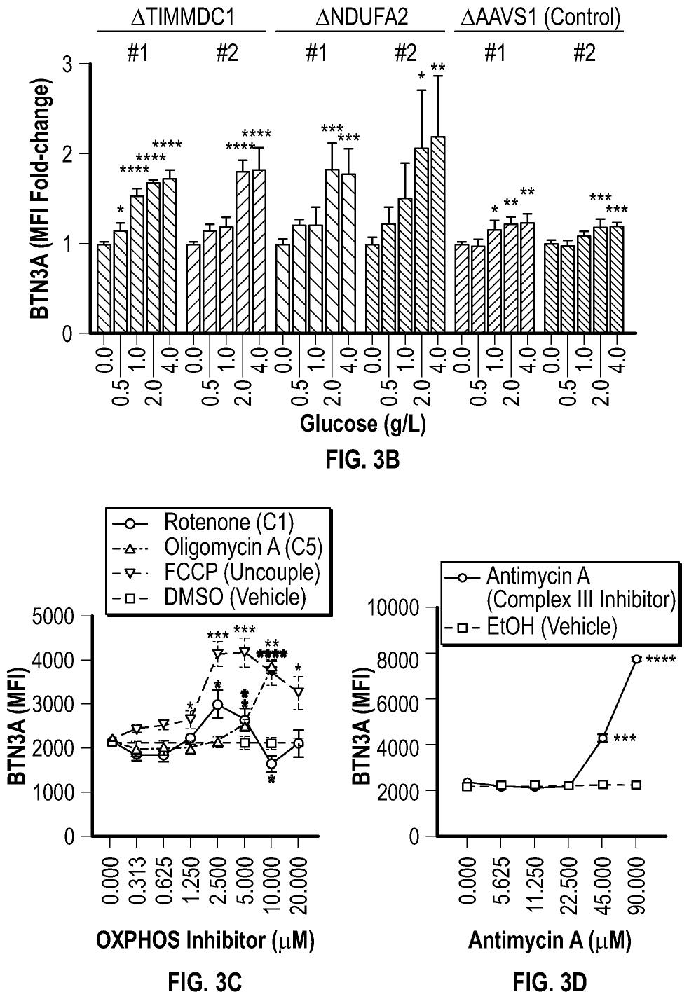

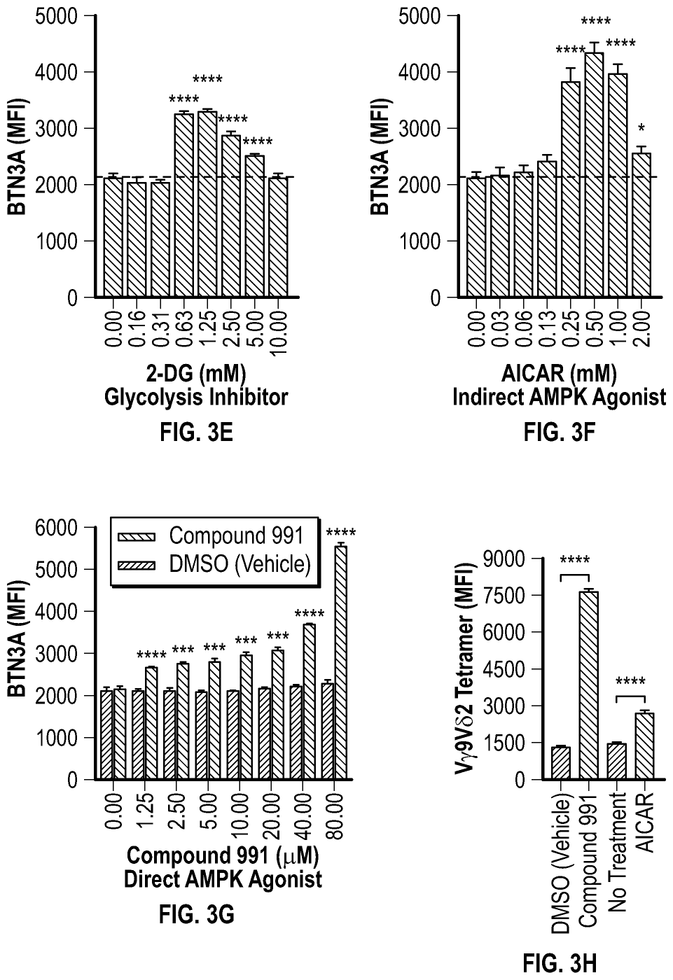

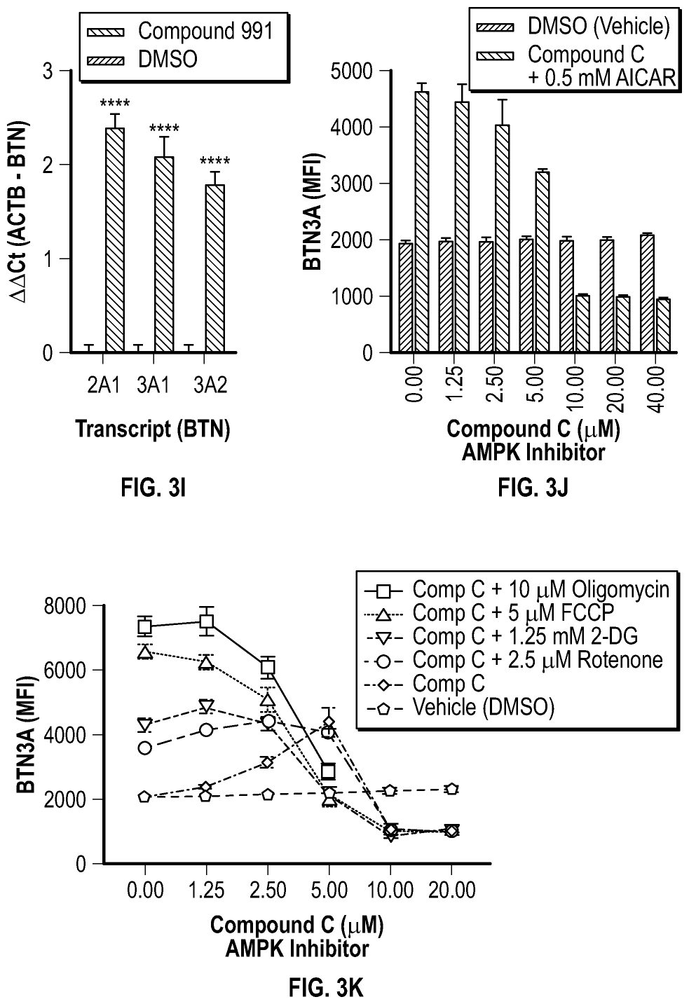

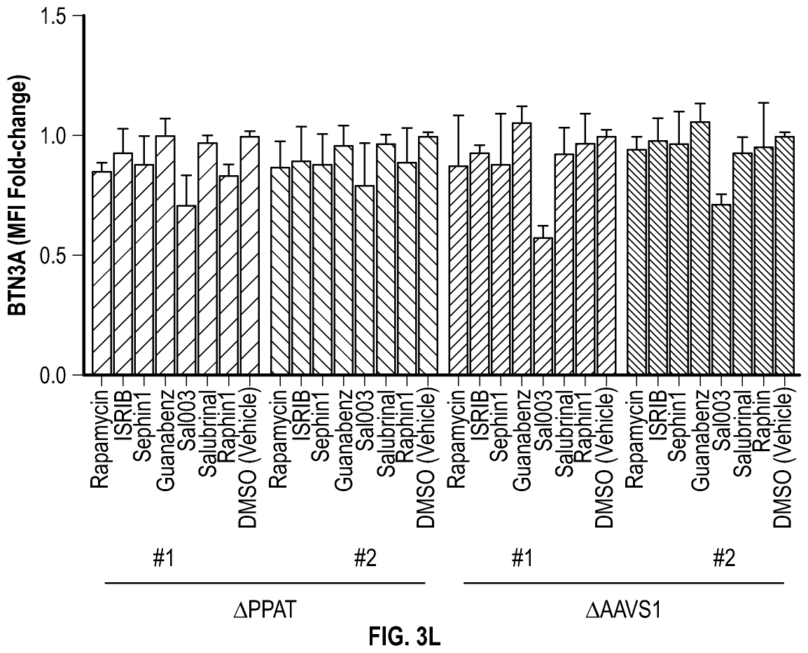

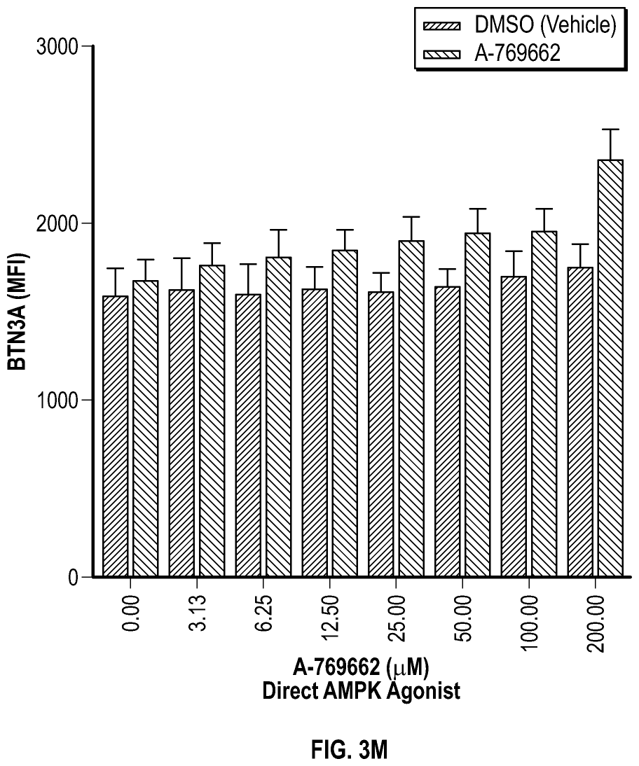

The relationship between OXPHOS and BTN3A surface abundance was evaluated by testing whether energy state imbalances or redox state imbalances in the OXPHOS KO cells were causing BTN3A expression changes. Impairments in Complex I (NDUFA2 KO, TIMMDC1 KO) can lead both to an energy state imbalance via deficient ATP production and to a redox state imbalance due to an elevated NADH/NAD+ ratio (FIG. 3A).

When cells were cultured in glutamine-containing media lacking glucose and pyruvate, increasing glucose levels caused upregulated BTN3A surface expression in OXPHOS KOs (TIMMDC1, NDUFA2), with a much lower effect in control AAVS1 KO cells (FIG. 3B). No such effect was observed in cells grown in increasing levels of pyruvate, which should have alleviated the redox imbalance by depleting excess NADH during the conversion of pyruvate to lactate.

These results indicated that a strong link exists between the ATP levels in the OXPHOS KO cells and the expression of BTN3A. When glucose levels increase in these OXPHOS KO cells, BTN3A expression levels increase.

This dependence on glucose levels in the media also helps explain the OXPHOS signature divergence between the two screens, which could have had appreciably distinct nutrient conditions due to markedly different cell concentrations in the two screens and the presence of highly proliferative T cells in the co-culture screen.

The effects of inhibitors targeting separate OXPHOS complexes on BTN3A expression were tested in wildtype (WT) Daudi-Cas9 cells. Complex I inhibition (rotenone) caused a BTN3A upregulation at two lower doses and a downregulation at one higher dose. Strikingly, directly inhibiting Complex III (antimycin A), Complex V/ATP synthase (oligomycin A), or uncoupling ATP synthesis from the electron transport chain (using FCCP) led to the highest BTN3A upregulation (FIG. 3C-3D). Furthermore, wildtype cells treated with glycolysis-blocking 2-deoxy-D-glucose (2-DG) showed upregulated BTN3A levels (FIG. 3E), confirming the GSEA identification of glycolysis as negatively regulating BTN3A in the genome-wide screen (Table 5).

These data indicate that cells undergoing energy crises change their expression of BTN3A. The dose-dependent variable effects of Complex 1 inhibition on BTN3A expression mirror the variable results observed with Complex I knockouts (NDUFA2, TIMMDC1) in the screen and the validations. These results indicate that inhibiting Complex I, which is most distal from ATP synthesis, has complicated effects on BTN3A regulation.

### Example 8: AMPK Activation Upregulates BTN3A

Nutrient and OXPHOS deprivation are detected by several stress sensors, including AMP-activated protein kinase (AMPK), mTOR, and those of the integrated stress response (ISR) pathway. This Example describes experiments designed to determine which of these is most relevant to regulation of BTN3A levels in transformed cells.

AICAR-mediated activation of AMPK, which senses elevated AMP:ATP ratios that occur during an energy crisis, led to a dramatic increase in surface BTN3A in WT Daudi-Cas9 cells (FIG. 3F). Inhibition of mTOR (rapamycin), inhibition of ISR (ISRIB), and activation of ISR (guanabenz, Sal003, salubrinal, raphin1) had negligible effects on BTN3A surface expression in control KO (AAVS1) and purine biosynthesis KO (PPAT) Daudi-Cas9 cells (FIG. 3L). The exception was a downregulation caused by the integrated stress response (ISR) agonist Sal003 (FIG. 3L).

Upregulation of surface BTN3A by AMPK activation was confirmed using two direct agonists of AMPK, the highly potent Compound 991 and the less potent A-769662 (FIG. 3G, 3M). Structures for Compound 991 and A-769662 are shown below.

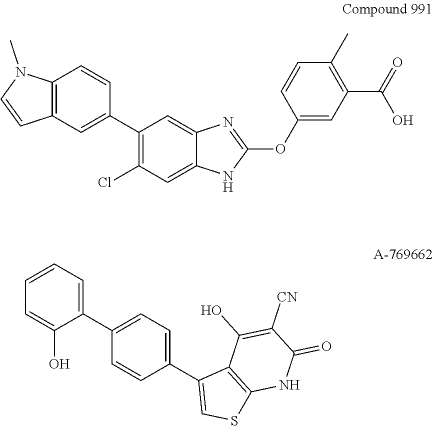 [SMILES: Cc1ccc(Oc2nc3cc(-c4ccc5c(ccn5C)c4)c(Cl)cc3[nH]2)cc1C(=O)O ; N#Cc1c(O)c2c(-c3ccc(-c4ccccc4O)cc3)csc2[nH]c1=O]

Cells treated with Compound 991 exhibited about five times higher staining with G115 Vγ9Vδ2 TCR tetramer compared to the vehicle control-treated cells, while AICAR treatment increased tetramer staining by 40-80% (FIG. 3H). Compound 991 treatment transcriptionally upregulated BTN2A1, as well as BTN3A1 and BTN3A2, as detected by qPCR (FIG. 3I). These results explained the high Vγ9Vδ2 TCR tetramer staining. A cell surface abundance of EphA2, a ligand of an unrelated Vγ9Vδ1 TCR MAU clone, has also recently shown to be upregulated by AMPK activation (Harly et al., Sci. Immunol. 6, eaba9010 (2021)), suggesting a common mechanism of engaging various human γδ T cell subsets.

AICAR is an indirect AMPK agonist. The inventors tested the effects of AICAR on BTN3A to ascertain whether those effects are AMPK-dependent by using Compound C, an AMPK inhibitor. Increasing amounts of Compound C decreased the AICAR-induced BTN3A upregulation, with BTN3A levels falling well below those observed in the vehicle control at 10 mM Compound C and greater (FIG. 3J). Similarly, BTN3A upregulation caused by OXPHOS inhibition (rotenone, oligomycin, FCCP) or glycolysis inhibition (2-DG) was neutralized by AMPK inhibition by Compound C (FIG. 3K).

These results show that cancer cells undergoing an energy crisis upregulate BTN3A through an AMPK-dependent process, which can be phenocopied by directly activating AMPK.

### Example 9: Genome-Wide Screen Hits Regulate γδ T Cell Activity

This Example describes tests to evaluate whether hits from the two genome-wide screens regulate γδ T cell activity in patient tumors and correlate with patient survival.

A co-culture screen signature was generated that involved obtaining weighted average expression values of each significant hit (FDR<0.01) with the magnitude of each weight proportional to the p-value of the particular hit and the positive or negative sign according to the direction of the hit's LFC value (Jiang et al., Nat. Med 24, 1550-1558 (2018)). The inventors estimated levels of the signature in tumors and correlated them with patient survival within each cancer type using data from The Cancer Genome Atlas (TCGA), altogether constituting over 11,000 patients and 33 cancer types.

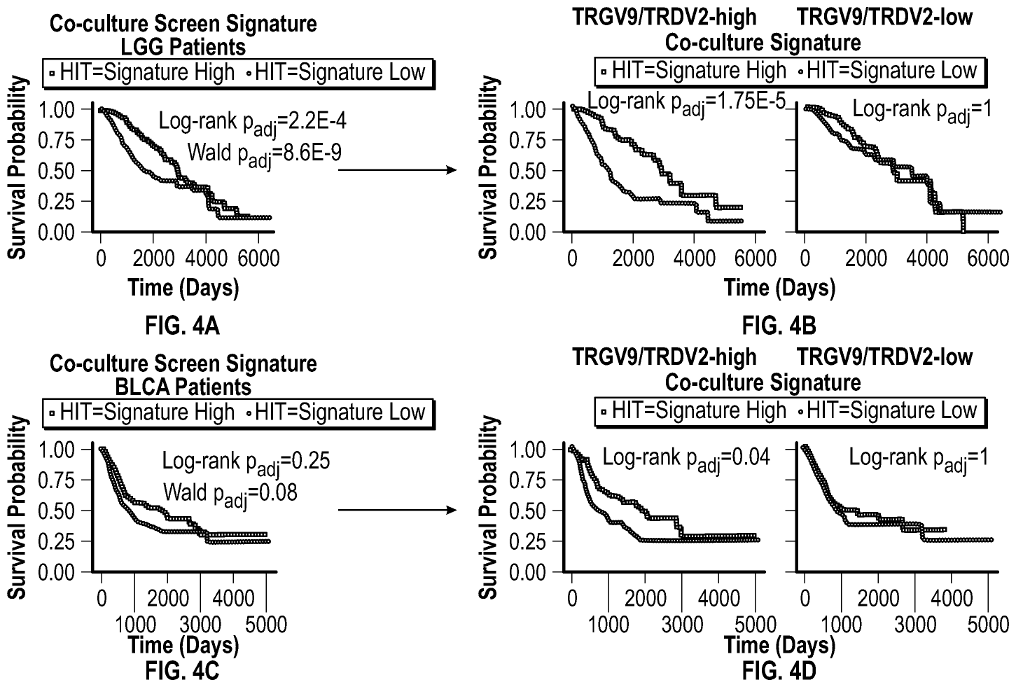

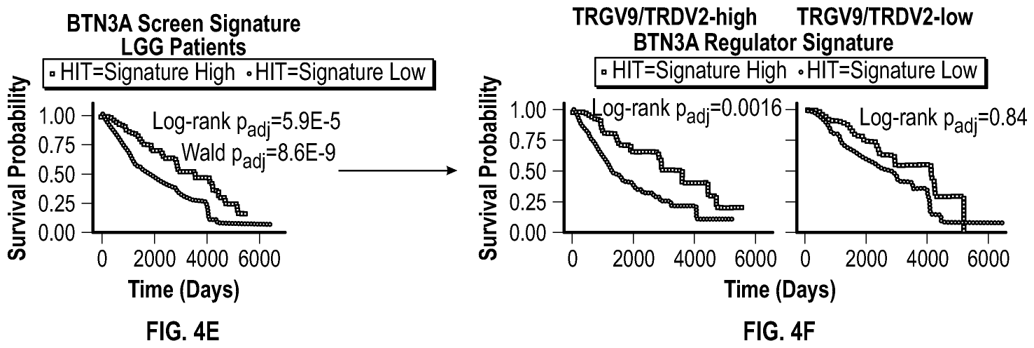

Across these cancer types, the strongest correlation was observed in low-grade glioma (LGG) tumors (FIG. 4A). LGG patients whose tumors exhibited high levels of the signature had significantly better overall survival compared to those with low signature levels. High levels of the signature had high expression of genes that upon KO diminished γδ T cell killing, and low levels of expression of genes whose KO increased γδ T cell killing. This association was also confirmed using Cox regression analysis.

The inventors then examined if the association of the co-culture signature with patient survival depends on the presence or absence of γδ T cells in patient tumors. The 529 LGG patients were split into two groups according to their TRGV9 (Vγ9) and TRDV2 (Vδ2) transcript abundance in the tumors. The survival association in each group was then separately evaluated.

As shown in FIG. 4B, the survival advantage conferred by high signature levels is seen only in the patient group with high Vγ9Vδ2 T cell infiltration. A similar pattern was found in the bladder urothelial carcinoma (BLCA) cohort with 433 patients, with the difference that the signature did not significantly correlate with better survival until the cohort was split by TRGV9/TRDV2 expression levels (FIG. 4C-4D).

The inventors generated another signature from the BTN3A screen and observed that LGG patients whose tumors had high BTN3A signature levels (high/low tumor expression of positive/negative regulators of BTN3A1, respectively) had a more prominent survival advantage when the tumors exhibited high Vγ9Vδ2 T cell infiltration (FIG. 4E-4F).

Recently, analysis of TCGA and Chinese Glioma Genome Atlas (CGGA) data revealed that CD4 and CD8 T cell infiltration correlates with poor outcomes in LGG, while γδ T cell infiltration correlates with better survival in LGG patients (Park et al. Nat. Immunol. 22, 336-346 (2021)). The results described herein indicate that LGG patient survival can be modulated in a Vγ9Vδ2 T cell-dependent manner by the activities of BTN3A regulators.

### Example 10: Materials and Methods

This Example describes some of the materials and methods used in the experiments described herein.

### Cancer-T Cell Co-Culture Screen

Human Improved Genome-wide Knockout CRISPR Library (Addgene Pooled Library #67989 from Kosuke Yusa; 90,709 gRNAs targeting 18,010 genes)(Tzelepis et al., Cell Rep. 17, 1193-1205 (2016)) was transformed into Endura ElectroCompetent E. coli cells (Lucigen) following the manufacturer's instructions. Briefly, nine transformations were performed for appropriate coverage (1 transformation per ˜10,000 sgRNA). For each transformation, 2 μL of library DNA was mixed with the cells. The mixture was loaded into a 1.0-mm cuvette and electroporated (1800 V, 10 μF, 600 Ohms) in a Gene Pulser Xcell (Biorad). Electroporated cells were rescued with 975 μL of Recovery Medium (Lucigen) and incubated at 37° C. with agitation for 1 hour. Transformed cells were grown overnight at 30° C. in 150 mL Luria broth (LB) with ampicillin. Appropriate transformation efficiency and library coverage (2250-fold) was confirmed by plating various dilutions of the transformed cells on LB agar plates with ampicillin. Library diversity was measured by PCR amplifying (3 min at 98° C.; 15 cycles of 10 sec at 98° C., 10 sec at 62° C., and 25 sec at 72° C.; 5 min at 72° C.) around the gRNA site with reactions made up of 10 ng DNA template, 25 μL NEBNext Ultra II Q5 Master Mix (NEB), 1 μL Read1-Stagger equimolar primer mix (10 μM) (NxTRd1.Stgr0-7 primers), 1 μL Read2-TRACR primer (10 μM), and water bringing the total volume to 50 μL. The PCR product was used in a second PCR reaction with the same PCR conditions and a reaction mix consisting of a 1 μL of PCR product (1:20 dilution), 25 μL NEBNext Ultra II Q5 Master Mix, 1 μL P7.i701 (10 μL) primer, and 1 μL P5.i501 (10 μM) primer, and water bringing the total volume to 50 uL. The final PCR product was treated with SPRI purification (1.0×), quantified on the NanoDrop, and sequenced on the MiniSeq using a MiniSeq High Output Reagent Kit (75-cycles) (Illumina). Distribution of gRNAs in the library was analyzed using the MAGeCK algorithm (Li et al., Genome Biol. 15, 554 (2014)). Relevant primers and probes mentioned in these methods are listed in Table 6A-6B.

TABLE 6A
Primers

| Target | | | |
|---|---|---|---|
| (IDT | Ref | | |
| Assay ID) | Seq No. | Exons | Primers 1 and 2 |
| BTN3A1 | NM_194441 | # 4-5 | 5′-AGACAGCCAGCATTTCCA |
| (Hs.PT.58. | | | T-3′ |
| 14608440) | | | (SEQ ID NO: 209) |
| | | | 5′-TTGCCACAGGAAGTAACC |
| | | | G-3′ |
| | | | (SEQ ID NO: 210) |
| BTN3A2 | NM_007047 | # 8-11 | 5′-CCAGTACTTGACTCGTGG |
| (Hs.PT.58. | | | AG-3′ |
| 40346506) | | | (SEQ ID NO: 211) |
| | | | 5′-TTAACAAGGTGGAGCCTC |
| | | | ATC-3′ |
| | | | (SEQ ID NO: 212) |
| BTN2A1 | NM_078476 | # 1b-3 | 5′-GGCAGATTGGAGAGAAGA |
| (Hs.PT.58. | | | GG-3′ |
| 15436751) | | | (SEQ ID NO: 213) |
| | | | 5′-GCCCCACGACAATAAACT |
| | | | G-3′ |
| | | | (SEQ ID NO: 214) |
| ACTB | NM_001101 | # 1-2 | 5′-ACAGAGCCTCGCCTTTG-3′ |
| (Hs.PT.39a. | | | (SEQ ID NO: 215) |
| 22214847 | | | 5′-CCTTGCACATGCCGGAG-3′ |
| | | | (SEQ ID NO: 216) |

TABLE 6B
Probe Sequences

| Target | | | |
|---|---|---|---|
| (IDT | Ref | | |
| Assay ID) | Seq No. | Exons | Probe |
| BTN3A1 | NM_194441 | # 4-5 | 5′-/56-FAM/AGACCCCTT/ |
| (Hs.PT.58. | | | ZEN/CTTCAGGAGCGC/ |
| 14608440) | | | 31ABKFQ/-3′ |
| | | | (SEQ ID NO: 217) |
| BTN3A2 | NM_007047 | # 8-11 | 5′-/56-FAM/TCCGATACC/ |
| (Hs.PT.58. | | | ZEN/AATAAGTCAGCCTGATG |
| 40346506) | | | C/31ABKFQ/-3′ |
| | | | (SEQ ID NO: 218) |
| BTN2A1 | NM_078476 | # 16 - 3 | 5′-/56-FAM/CGTCGAGAA/ |
| (Hs.PT.58. | | | ZEN/CCAGCGGAGAAAAGAA/ |
| 15436751) | | | 31ABKFQ/-3′ |
| | | | (SEQ ID NO: 219) |
| ACTB | NM_001101 | # 1-2 | 5′-/5Cy5/TCATCCATG/ |
| (Hs.PT.39a. | | | TAQ/GTGAGCTGGCGG/ |
| 22214847) | | | 31AbRQSp/-3′ |
| | | | (SEQ ID NO: 220) |

The genome-wide knockout CRISPR library was packaged into lentivirus using HEK293T cells (Takara Bio). In a 15-cm TC-treated dish, about 16 hours before transfection, 12 million cells were seeded in 25 mL of DMEM containing high-glucose and GlutaMAX (Gibco) supplemented with 10% FBS, 100 U/mL Penicillin-Streptomycin (Sigma-Aldrich), 10 mM HEPES (Sigma-Aldrich), 1% MEM Non-essential Amino Acid Solution (Millipore Sigma), and 1 mM sodium pyruvate (Gibco). HEK293T cells were transfected with 17.8 μg gRNA transfer plasmid library, 12 μg pMD2.G (Addgene plasmid #12259), and 22.1 μg psPAX2 (Addgene plasmid #12260) using the FuGENE HD transfection reagent (Promega) following the manufacturer's protocol. Twenty-four hours after transfection, old media was replaced with fresh media supplemented with ViralBoost Reagent (Alstem). Cell supernatant was collected 48 hours after transfection, centrifuged at 300×g (10 min, 4° C.), and transferred into new tubes. Four volumes of the supernatant were mixed with 1 volume of Lentivirus Precipitation Solution (Alstem) and incubated overnight at 4° C. Lentivirus was pelleted at 1500×g (30 min, 4° C.), resuspended in 1/100^{th} of the original volume in cold PBS, and stored at −80° C.

Daudi-Cas9 cells were cultured in supplemented with 10% FBS, 2 mM L-glutamine (Lonza), and 100 U/mL Penicillin-Streptomycin. Cells were confirmed to be negative for mycoplasma with a PCR method. For two weeks prior to lentiviral gRNA delivery, Daudi-Cas9 cells were cultured in complete RPMI supplemented with κ μg/ml blasticidin (Thermo Fisher) (cRPMI+Blast). On the day of lentiviral transduction, 250 million Daudi-Cas9 cells were resuspended in cRPMI+Blast at 3 million cells/mL, supplemented with 4 μg/mL Polybrene (Sigma-Aldrich), and aliquoted into 6-well plates (2.5 mL per well). Each well of cells received 6.25 μL of lentiviral genome-wide KO CRISPR library, and the plates were centrifuged at 300×g for 2 hours at 25° C. After the centrifugation, the cells were rested at 37° C. for 6 hours, the media was replaced with cRPMI+Blast with cells seeded at 0.3 million/mL, and the cells were cultured at 37° C. for 3 days. Three days after transduction, Daudi-Cas9 cells were diluted to 0.3×106 cells/mL and treated with 5 ug/mL puromycin (Thermo Fisher). At this time point, the infection rate was determined to be 21% by staining cells with the 7-AAD viability dye (BioLegend) in FACS buffer (PBS, 0.5% bovine serum albumin [Sigma], 0.02% sodium azide) and assessing levels of BFP+ cells on the Attune NxT flow cytometer (Thermo Fisher). After four days of antibiotic selection, Daudi-Cas9 cells were placed in complete RPMI without blasticidin or puromycin. Puromycin-selected cells were >90% BFP+, as measured by flow cytometry following a viability stain. From this point onwards, Daudi-Cas9 cells were passaged every 2 to 3 days, maintaining at least 45×10⁶ cells at each passage to retain sufficient knockout library diversity (>495× coverage per gRNA in the genome-wide knockout library). For 24 hours prior to the co-culture with expanded γδ T cells cells, genome-wide knockout library Daudi-Cas9 cells were treated with 50 μM of zoledronate (Sigma-Aldrich).

Residual cells in leukoreduction chambers of Trima Apheresis from de-identified donors following informed consent (Vitalant, San Francisco, CA) were used as the source of primary cells for the co-culture screen, under protocols approved by the University of California San Francisco Institutional Review Board (IRB) and the Vitalant IRB. Primary human peripheral blood mononuclear cells (PBMCs) were isolated using Lymphoprep (STEMCELL) and SepMate-50 PBMC Isolation Tubes (STEMCELL). To expand Vγ9Vδ2 T cells, PBMCs were resuspended in cRPMI with 100 U/mL human IL-2 (AmerisourceBergen) and 5 μM zoledronate. PBMC cultures were supplemented with 100 U/mL IL-2 at 2, 4, and 6 days after seeding the cultures. After 8 days of Vγ9Vδ2 T cell expansion, γδ T cells were isolated following the manufacturer's instructions using a custom human γδ T cell negative isolation kit without CD16 and CD25 depletion (STEMCELL). Isolated γδ T cells were confirmed to be >97% Vγ9Vδ2 TCR+ by flow cytometry using APC-conjugated anti-γδ TCR (clone B3) and Pacific Blueconjugatedcanti-Vδ2 TCR (clone B6) antibodies (BioLegend). Both Daudi-Cas9 cells and isolated γδ T cells were resuspended at 2 million cells/mL in cRPMI. For each donor, T cells and Daudi-Cas9 cells were mixed at effector-to-target (E:T) ratios of 1:2 and 1.4. Cultures were supplemented with 5 μM zoledronate and 100 U/mL IL-2. Surviving Daudi-Cas9 cells were harvested after 24 hours of co-culturing with γδ T cells. Using the manufacturer's depletion protocol, the cell mixture was treated with the EasySep Human CD3 Positive Isolation Kit II (STEMCELL). Daudi-Cas9 cells were cultured in cRPMI+Blast for 4 days after isolation from the T cell co-culture and frozen down as cell pellets, which were used to generate sequencing libraries. The final library was sequenced using a NovaSeq 6000 S1 SE100 kit (Illumina).

### BTN3A Expression Screen

Daudi-Cas9 cells were edited with the genome-wide knockout CRISPR library as described above. The screen was performed with 3 replicates of Daudi-Cas9 cell pools, each starting with 250 million cells, that were kept entirely separate starting with the lentiviral transduction step. All the replicates had an infection rate of 23-25%. Per replicate, 180 million Daudi-Cas9 cells were stained with the 7-AAD (Tonbo) viability dye and the Alexa Fluor 647-conjugated anti-BTN3A1 antibody (clone BT3.1, 1:40 dilution) (Novus 630 Biologicals) 14 days after lentiviral transduction. Live BTN3A-high (top ˜25%) and BTN3A-low (bottom ˜25%) Daudi-Cas9 cells were sorted using FACSAria II, FACSAria III, and FACSAria Fusion (BD Biosciences) cell sorters. Each sorted population had between 12 and 23 million cells. Cell pellets were frozen and used to generate sequencing libraries. The final library was sequenced using a NovaSeq 6000 S4 PE150 kit (Illumina).

### Next-Generation Sequencing Library Preparation

Cell pellets were lysed overnight at 66° C. in 400 μL of cell lysis buffer (1% SDS, 50 mM Tris, pH 8, 10 mM EDTA) and 16 μL of sodium chloride (5 M), with 2.5 million cells per 416-μL lysis reaction. 8 μL of RNase A (10 mg/mL, Qiagen) was added to the cell lysis solution and incubated at 37° C. for 1 hour. Eight microliters of Proteinase K (20 mg/mL, Ambion) was then added and incubated at 55° C. for 1 hour. 5PRIME Phase Lock Gel—Light tubes (Quantabio) were prepared by spinning the gel at 17,000×g for 1 minute. Equal volumes of the cell lysis solution and Phenol:Chloroform:Isoamyl alcohol (25:24:1, saturated with 10 mM Tris, pH 8.0, 1 mM EDTA, Sigma) were added to a 5PRIME Phase Lock Gel—Light tube. The tubes were vigorously inverted and centrifuged (17,000×g, 5 min, room temperature). The aqueous layer containing the genomic DNA above the gel was poured into DNA LoBind tubes (Eppendorf). Forty (40) μL of sodium acetate (3 M), 1 μL of GenElute-LPA (Sigma-Aldrich), and 600 μL of isopropanol were added, and the solution was vortexed and frozen at −80° C. Once thawed, the solution was centrifuged at 17,000×g for 30 minutes at 4° C. After discarding the supernatant, the DNA pellet was washed with fresh room temperature ethanol (70/6) and mixed by inverting the tube. The solution was then centrifuged at 17,000×g for 5 minutes at 4° C. The supernatant was removed and the DNA pellet was left to air dry for 15 minutes. The DNA Elution Buffer (Zymo Research) was added to the DNA pellet and incubated for 15 minutes at 65° C. to resuspend the genomic DNA.

A two-step PCR method was used to amplify and index the genomic DNA samples for Next Generation Sequencing (NGS). For the first PCR reaction, 10 μg of genomic DNA was used per 100-μL reaction (0.75 μL of Ex Taq polymerase, 10 μL of 10×ExTaq buffer, 8 μL of dNTPs, 0.5 μL of Read1-Stagger equimolar primer mix (100 μM) (NxTRd1.Stgr0-7 primers), and 0.5 μL of Read2-TRACR primer (100 PM)) to amplify the integrated gRNA. The PCR #1 program was 5 min at 95° C.; 28 cycles of 30 sec at 95° C., 30 sec at 53° C., 20 sec at 72° C.; 10 min at 72° C. The PCR product solution was treated with SPRI purification (1.0×), and the DNA was eluted in 100 μL of water. To index the samples, 2 μL of purified PCR product (1:20 dilution) was used in a 50-μL PCR reaction containing 25 μL of Q5 Ultra II 2× MasterMix (NEB), 1.25 μL of Nextera i5 indexing primer (10 μM) (P5.i501-508 primers), and 1.25 μL of Nextera i7 indexing primer (10 uM) (P7.i701-708 primers). The PCR #2 program was 3 min at 98° C.; 10 cycles of 10 sec at 98° C., 10 sec at 62° C., 25 sec at 72° C.; 2 min at 72° C. The final PCR product was treated with SPRI purification (0.7×), including two washes in 80% ethanol. DNA was eluted in 15 μL of water. The concentration was determined using a Qubit fluorometer (Thermo Fisher), and the library size was confirmed by gel electrophoresis and Bioanalyzer (Agilent). All indexed samples were pooled in equimolar amounts and analyzed by NGS.

### Analysis of Genome-Wide CRISPR Screens

A table of individual guide abundance in each sample was generated using the count command in MAGeCK (version 0.5.8) (Li et al. Genome Biol. 15, 554 (2014)). The MAGeCK test command was used to identify differentially enriched sgRNA targets between the low and high bins or the pre-killing and post-killing conditions. For the co-culture killing screen, all genes with an FDR-adjusted p-value<0.05 were considered significant. For the BTN3A screen, all genes with an FDR-adjusted p-value<0.01 were considered significant. Gene set enrichment analysis (GSEA) for both screens was performed using GSEA (version 4.1.0 [build: 27], UCSD and Broad Institute) (Mootha et al., Nat. Genet. 34, 267-273 (2003); Subramanian et al., Proc. Natl. Acad. Sci. USA 102, 15545-15550 (2005)) using a ranked list of genes with their log-fold change values. The following GSEA settings were used: 1000 permutations, No Collapse, gene sets database C2.CP.KEGG.7.4. Both the web interface and the R package (version 1.0.0) of Correlation AnalyzeR (Millet & Bishop, BMC Bioinformatics 22, 206 (2021)) was used to determine the pairwise and gene set-wide BTN3A1 expression correlations in publicly available samples provided by the ARCH4 Repository (Lachmann et al. Nat. Commun. 9, 1366 (2018)).

sgRNA Plasmids and Lentivirus

To make sgRNA plasmids for arrayed validation studies, individual sgRNAs were cloned into the pKLV2-U6gRNA5(BbsI)-PGKpuro2ABFP-W vector (Addgene plasmid #67974 from Kosuke Yusa), generally following the depositing lab's “Construction of gRNA expression vectors V2015-8-25” protocol. Briefly, the vector was digested with BbsI-HF (New England Biolabs [NEB]), run on a 1% agarose gel, and gel extracted. For each sgRNA, oligo pairs with appropriate overhangs were annealed using T4 Polynucleotide Kinase (NEB) and T4 DNA Ligase Reaction Buffer (NEB). Annealed inserts and the linearized vector were ligated using the T4 DNA Ligase (NEB) and transformed into MultiShot StripWell Stbl3 E. coli competent cells (Invitrogen) that were grown on Lysogeny broth (LB) agar Carbenicillin plates at 37° C. overnight. Single colonies were grown out in ampicillin-containing LB and screened for the correct sgRNA insert by Sanger sequencing PCR amplicons of the insert site. Successful clones were grown and processed with a Plasmid Plus Midi Kit (Qiagen), with the DNA product serving as the transfer plasmid during lentiviral packaging. Collected lentivirus was titrated for optimal transduction in Daudi-Cas9 cells and used to generate single gene Daudi-Cas9 KOs.

Arrayed CRISPR sgRNA KO

To generate single gene Daudi-Cas9 KOs, 3 million cells/mL were resuspended in cRPMI with 4 μg/mL Polybrene. Daudi-Cas9 cells were aliquoted at 150 μL per well into 96-well V-bottom plates. Ten μL of AAVS1 sgRNA virus diluted for optimal transduction was added to the cells, with 3 replicates per sgRNA (6 replicates per AAVS1 sgRNA). The plates were centrifuged at 300×g for 2 hours at 25° C. After the centrifugation, the cells were rested at 37° C. for 6 hours, pelleted, resuspended at 750,000 cells/mL in fresh cRPMI, and cultured at 37° C. for 3 days. Three days after transduction, Daudi-Cas9 cells were diluted to 0.3×106 cells/mL and treated with 5 ug/mL puromycin (Thermo Fisher). After four days of antibiotic selection, Daudi-Cas9 cells were placed in cRPMI without puromycin. From this point onwards, Daudi-Cas9 cells were passaged every 2 to 3 days. Cells were collected at 13 days post-transduction to assess frequency of indels in the CRISPR target site for each of the KOs. At the same time point, the cells were analyzed for BTN3A expression by flow cytometry.

BFP+ (lentivirally induced) Daudi-Cas9 KO cells were blocked with Human TruStain FcX (Fc receptor blocking solution) in FACS buffer for 20 min at 4° C. Blocked cells were stained for 30 min at 4° C. with 7-AAD viability dye (1:150 dilution) and either APC-conjugated anti-CD277 antibody (clone BT3.1, 1:50 dilution) (Miltenyi Biotec) or APC-conjugated IgG1 isotype control antibody (Miltenyi Biotec, 1:50 dilution, anti-KLH, clone IS5-21F5) in FACS buffer. Stained and washed cells were analyzed on the Attune NxT flow cytometer. No appreciable signal was detected in the APC channel when cells were stained with the isotype control antibody.

### CRISPR Genotyping Primers

To determine indel frequency among arrayed Daudi-Cas9 KO cells, an indexed NGS library of amplicons were generated around the CRISPR cute sites of the various knockouts. Primers to generate amplicons around the CRISPR genomic target site were designed with CRISPOR (version 4.8) (Concordet et al., Nucleic Acids Res. 46, W242-W245 (2018)) with the options “--ampLen=250 --ampTm=60”. To analyze the NGS genotyping data, adapter sequences were trimmed from fastq files using cutadapt (version 2.8) (Martin, EMBnet J. 17, 10-12 (2011)) using default settings keeping a minimum read length of 50 bp. Insertions and deletions at each CRISPR target site were then calculated using CRISPResso2 (version 2.0.42) (Clement et al. Nat. Biotechnol. 37, 224-226 (2019)) with the options “--quantification_window_size 3” and “--ignore_substitutions”.

### Pooled CRISPR Genotyping for Arrayed KOs

Approximately 50,000 cells from appropriate samples were pelleted (300×g, 5 min) and resuspended in 50 μL of QuickExtract DNA Extraction Solution (Lucigen). Samples were run on a thermocycler according to the following protocol (QuickExtract PCR): 10 min at 65° C., 5 min 740 at 95° C., hold at 12° C. Samples were stored at −20° C. until further steps. The PCR reaction for each sample consisted of 5 μL of the extracted DNA sample, 1.25 μL of 10 μM pre-mixed forward and reverse primer solution, 12.5 μL of Q5 High-Fidelity 2× Master Mix (NEB), and 6.25 μL of molecular biology grade water. The samples were then run on a thermocycler according to the following PCR #1 program: 3 min at 98° C.; 15 cycles of 20 sec at 94° C., 20 sec at 65° C.-57.5° C. with a 0.5° C. decrease per cycle, 1 min at 72° C.; 20 cycles of 20 sec at 94° C., 20 seconds at 58° C., 1 min at 72° C.; 10 min at 72° C., hold at 4° C. The PCR product was stored at −20° C. until further steps. PCR #1 products were indexed in PCR #2 reaction; 1 μL of PCR #1 product (diluted 1:200), 2.5 μL of 10 μM forward indexing primer, 2.5 μL of 10 μM reverse indexing primer, 12.5 μL of Q5 High-Fidelity 2× Master Mix (NEB), and 6.5 μL molecular biology grade water. PCR reactions were run on a thermocycler according to the following program: 30 sec at 98° C.; 13 cycles of 10 sec at 98° C., 30 sec at 60° C., 30 sec at 72° C.; 2 min at 72° C., hold at 4° C. PCR #2 product was stored at −20° C. until further steps. PCR #2 product was pooled, SPRI purified (1.1×), and eluted in water. The final library was sequenced using a NovaSeq 6000 SP PE150 kit (Illumina).

### Sanger Sequencing Genotyping

Daudi-Cas9 NLRC5 (gRNA #2) KOs were genotyped by Sanger sequencing. Approximately 50,000 cells were pelleted (300×g, 5 min) and resuspended in 50 μL of QuickExtract DNA Extraction Solution. Samples were run on a thermocycler according to the QuickExtract PCR program. Samples were stored at −20° C. until further steps. The PCR reaction for each sample consisted of 1 μL, of the QuickExtract DNA sample, 0.75 μL of 10 μM forward primer, 0.75 μL of 10 μM reverse primer, 12.5 μL of KAPA HiFi HotStart ReadyMix PCR Kit (Roche Diagnostics), and 10 μL molecular biology grade water. The samples were amplified on a thermocycler according to the following protocol: 3 minutes at 95° C.: 35 cycles of 20 seconds at 98° C., 15 seconds at 67° C., 30 seconds at 72° C., 5 minutes at 72° C., hold at 4° C. The amplified products were analyzed using Sanger sequencing and knockout efficiencies were assessed using the TIDE (Tracking of Indels by Decomposition) algorithm (Brinkman et al., Nucleic Acids Res. 42, e168-e168 (2014)).

### RT-qPCR of Daudi KOs and AICAR/991-Treated Cells

For measurement on Daudi-Cas9 KOs, samples were collected at 13 days after lentiviral transduction. For measurements on drug-treated WT Daudi-Cas9 cells, 180 μL of Daudi-Cas9 cells were seeded in a round-bottom 96-well plate at 275,000 cells/mL. All surrounding wells were filled with 200 μL of sterile PBS or water. With four replicates per treatment, cells were treated with 20 μL of AICAR (final concentration 0.5 mM), Compound 991 (final concentration 80 PM), DMSO, or water. The cells were collected for RT-qPCR measurements after 72 hours of incubation. RNA was extracted from approximately 70,000 cells per sample using the Quick-RNA 96 Kit (Zymo Research) or Direct-zol RNA Microprep Kit (Zymo) according to the manufacturer's protocol without the optional on-column DNase I treatment. According to the manufacturer's protocol, 1 μL of RNA was immediately processed using the Maxima First Strand cDNA Synthesis Kit for RT-qPCR with the dsDNase treatment (Thermo Fisher). Two cDNA synthesis reactions, in addition to a reverse transcriptase minus (RT−) negative control reaction, were performed for each biological replicate. RNA template minus (RNA−) negative controls were performed as well. cDNA samples were stored at −20° C. until they were used for RT-qPCR. To perform the RT-qPCR, the two cDNA samples per biological replicate were pooled and diluted 1:1 in molecular biology grade water. Negative controls were diluted the same way. According to the manufacturer's protocol, 3 μL of diluted cDNA and negative controls were used for the RT-qPCR reactions using the PrimeTime Gene Expression Master Mix (Integrated DNA Technologies [IDT]) including a reference dye. RT-qPCR for each biological replicate was performed in triplicate along with the RT-negative control for each biological replicate, the RNA-negative controls, and no cDNA template negative controls. None of the negative controls showed target amplification. Samples were run on the QuantStudio 5 Real-Time PCR System (384-well, Thermo Fisher) according to the following program. 3 minutes at 95° C.; 40 cycles of 5 seconds at 95° C., 30 sec at 60° C. BTN2A1, BTN3A1, BTN3A2, and ACTB loci were amplified using the PrimeTime Standard qPCR Probe Assay (IDT) resuspended with 500 μL IDTE Buffer (IDT). Ct values across the three technical replicates for each sample were assessed for significant outliers resulting from technical failures (any samples in triplicate with a standard deviation above 0.2 were assessed) and subsequently averaged. The following calculations were performed: ΔCt=CtACMB−CtTarget; ΔΔCt=ΔCt(KO or treatment)−average(ΔCt(control)). Individual control ΔCt measurements were used to determine standard deviation of the control ΔΔCt. AAVS1 KO served as the control for qPCR measurements across Daudi KOs, and vehicle controls (DMSO, water) were used for measurements in Daudi cells treated with AICAR and Compound 991.

### Glucose and Pyruvate Dose Response

Daudi-Cas9 KO cells (190 μL) were seeded at 250,000 cells/mL in round-bottom 96-well plates in glucose-free cRPMI (+glutamine, +foetal calf serum, +penicillin/streptomycin, −glucose, −pyruvate) (Fisher Scientific). Ten μL of glucose (Life Tech) or sodium pyruvate (Gibco) at various concentrations were added to the cells. Plate edge wells were filled with 200 μL of sterile water or PBS. The cells were grown at 37° C. for 72 hours, stained with APC-conjugated anti-human CD277 antibody (clone BT3.1, 1:50 dilution) (Miltenyi Biotec) and 7-AAD (1:150 dilution) (Tonbo) in FACS buffer, and analyzed on the Attune NxT flow cytometer.

### Inhibitor Dose Response

Daudi-Cas9 cells (180 μL) were seeded at 275,000 cells/mL in cRPMI in round-bottom 96-well plates. Twenty μL of zoledronate, rotenone (MedChemExpress), oligomycin A (Neta Scientific), FCCP (MedChemExpress), antimycin A (Neta Scientific), AICAR (Sigma), 2-DG (Sigma), Compound 991 (Selleck Chemical), A-769662 (Sigma), ethanol (vehicle), or DMSO (vehicle, at dilutions matching the treatment) at various concentrations were added to the cells. Plate edge wells were filled with 200 μL of sterile water or PBS. The cells were grown at 37° C. for 72 hours, and stained with APC-conjugated anti-human CD277 antibody (clone BT3.1, 1:50 dilution)(Miltenyi Biotec) and 7-AAD (1:150 dilution) (Tonbo). The cells were then analyzed on the Attune NxT flow cytometer.

Daudi-Cas9 AAVS1 and PPAT KO cells (190 μL) were seeded at 250,000 cells/mL in round-bottom 96-well plates. Cells received 10 μL of DMSO (vehicle) or one of the following compounds at a final concentration of 10 μM: sephin1 (APE×BIO), ISRIB (MedChemExpress), guanabenz acetate (MedChemExpress), Sal003 (MedChemExpress), salubrinal (MedChemExpress), raphin1 acetate (MedChemExpress), and rapamycin (MilliporeSigma). Edge wells were filled with 200 μL of sterile PBS or water. After being cultured for 72 hours, the cells were stained with APC-conjugated anti-human CD277 antibody (clone BT3.1, 1:50 dilution) (Miltenyi Biotec) and 7-AAD (1:150 dilution) (Tonbo), and analyzed on the Attune NxT flow cytometer.

Compound C Dose Response in Combination with AICAR or OXPHOS Inhibition

Daudi-Cas9 cells (170 μL) were seeded at 292,000 cells/mL in cRPMI in round-bottom 96-well plates. Ten μL of Compound C (Abcam) were added to all the cells at various concentrations. At indicated concentrations, 20 μL of rotenone, oligomycin A, FCCP, 2-DG, AICAR, or cRPMI (control) were added to the wells that received Compound C. Ten μL of DMSO at dilutions matching Compound C and 20 μL of cRPMI were added to the DMSO-only vehicle control wells. Plate edge wells were filled with 200 μL of sterile water or PBS. The cells were grown at 37° C. for 72 hours, stained with APC-conjugated anti-human CD277 antibody (clone BT3.1, 1:50 dilution) (Miltenyi Biotec) and 7-AAD (1:150 dilution) (Tonbo), and analyzed on the Attune NxT flow cytometer.

### Vg9Vd2 TCR Tetramer Production

The G115 Vγ9Vδ2 TCR clone tetramer was generated using the following methods. The G115 γ-845 chain sequence (Davodeau et al. J. Immunol. 151, 1214-1223 (1993)) was cloned into the pAcGP67A vector with a C-terminal acidic zipper, and the G115 δ-chain sequence (Davodeau et al. (1993)) as cloned into the pAcGP67A vector with a C-terminal AviTag followed by a basic zipper. Zippers stabilized the TCR complex. The TCR was expressed in the High Five baculovirus insect-cell expression system and purified via affinity chromatography over a Ni-NTA column. TCRs were biotinylated and biotinylation was confirmed using a TrapAvidin SDS-PAGE assay. The G115 TCR was then further purified using size-exclusion chromatography (Superdex200 100/300 GL column, GE Healthcare) and purity was confirmed via SDS-PAGE. Tetramers were generated by incubating biotinylated TCR with streptavidin conjugated to the PE fluorophore.

### γδ TCR Tetramer Staining

Daudi-Cas9 KO cells were analyzed 13 and 14 days post-lentiviral transduction. WT Daudi-Cas9 cells were analyzed after being cultured for 72 hours with 0.5 mM AICAR, 80 μM Compound 991, DMSO (vehicle control at the concentration matching Compound 991), or nothing. Cells were washed (300×g, 5 min) in 200 μL FACS buffer containing human serum (PBS, 10% human serum AB [GeminiBio], 3% FBS, 0.03% sodium azide), and stained with 7-AAD (1:150 dilution) on ice in the dark for 20 min. After the first stain, the cells were pelleted (300×g, 5 min) and stained with 160 nM PE-conjugated Vγ9Vδ2 TCR (clone G115) tetramer for 1 hour in the dark at room temperature. Following the tetramer stain, cells were thoroughly washed three times in 200 μL FACS buffer containing human serum (400×g, 5 min). Stained cells were analyzed on the Attune NxT flow cytometer.

### Pathway Visualization

Pathway data visualizations were generated using Cytoscape (version 3.9.0) and the WikiPathways app (version 3.3.7). Glycan glyphs for the N-glycan pathway were generated using GlycanBuilder2 (version 1.12.0) in SNFG format, and were incorporated in the pathway in Cytoscape using the RCy3 package (version 2.14.0) in RStudio (R version 4.0.5). All pathway visualizations were based on WikiPathways models [see webpage at pubmed.ncbi.nlm.nih.gov/33211851/]:
the mevalonate pathway was adapted from WP4718 [see webpage at wikipathways.org/instance/WP4718] and WP197 [see webpage at wikipathways.org/instance/WP197];
the purine biosynthesis pathway was adapted from WP4224 [see webpage at wikipathways.org/instance/WP4224];
the OXPHOS pathway was adapted from WP111 [see webpage at wikipathways.org/instance/WP111];
the iron-sulfur cluster biogenesis pathway corresponds to WP5152 [see webpage at wikipathways.org/instance/WP5152];
the sialylation pathway corresponds to WP5151 [webpage at wikipathways.org/instance/WP5151];
N-glycan biosynthesis pathway was based on WP5153 [webpage at wikipathways.org/instance/WP5153].

### Generation of Co-Culture and BTN3A Regulator Screen Signatures

TCGA bulk RNA-seq and survival data from 11,093 patients were obtained using the R package TCGAbiolinks, and matched normal samples were removed. The signature was generated using genes with significant fold change (FDR<0.01) in the co-culture screen or the BTN3A screen. TCGA samples were scored using the level of the signature adopting a strategy described by Jiang et al. (Nat. Med. 24, 1550-1558 (2018)). A sample's signature level was estimated as the Spearman correlation between normalized gene expression of signature genes and screen score of signature genes: Correlation (Normalized expression, Weighted fold change). The following was used: −log 10(Padj)×sign(Fold Change) as the screen score of each gene. The expression of a signature gene was normalized within the TCGA sample by dividing its average across all 11,093 samples.

### Signature Survival Associations

The Cox proportional hazard model was used to check associations of signature expression with patient survival:

h(t,patient)˜hₒ(t)exp(β″+βl(patient))

where:
h is the hazard function (defined as the risk of death across patients per unit time);
hₒ(t) is the baseline hazard function at time t;
l(patient) is patients' screen signature levels; and
β is the coefficient of survival association.

The significance (Wald's test) of the β is the coefficient of survival association were determined using the R-package “Survival”. To show the association of survival with a signature using a Kaplan-Meier plot, TCGA samples were divided into two groups using the median of the signature levels across samples within a given cancer type and compared the survival between the two groups. The significance of survival difference was estimated using a log-rank test.

To test the dependence of the survival association with the signatures on the presence or absence of γδT cells, the average expression (transcripts per million) of TRGV9 (Vγ9) and TRDV2 (Vδ2) genes in a sample we used as its Vγ9Vδ2 T cell transcript abundance. The likely interaction of a screen signature with TRGV9/TRDV2 transcript abundance was estimated using Cox regression with the following model:

h(t,patient)˜hog(t)exp(β₀+β₁l+β₂g+β₃l*g)

Where l is the signature level and g is the TRGV9/TRDV2 transcript abundance in TCGA samples. The significance of the coefficient of interaction β₃ was estimated by comparing the likelihood of the model with the likelihood of the null model and performing the likelihood ratio test. The null model:

(hₙᵤₗₗ(t,patient)˜ho(t)exp(β₀+β₁l+β₂g+β₃l*g))

To show the interactions using Kaplan-Meier plots, TCGA samples were divided into four groups using the median signature levels and median TRGV9/TRDV2 transcript abundance.

### Software

Plots were generated in ggplot2 in R (version 4.0.2), as well as in Prism 9 (GraphPad). Flow cytometry data were analyzed in FlowJo (version 10.8.0, Beckton Dickinson). Figures were compiled in Illustrator (version 26.0, Adobe). Schematics were created in BioRender.com. The OXPHOS schematic was adapted from “Electron Transport Chain,” by BioRender.com (2021), retrieved from the website app.biorender.com/biorender-templates.

### Data Availability

The sequencing datasets for the two screens will be available in the NCBI Gene Expression Omnibus (GEO) repository (co-culture screen: GSE192828; BTN3A screen: GSE192827).

### REFERENCES

1. Silva-Santos, B., Serre, K. & Norell, H. γδ T cells in cancer. Nat. Rev. Immunol. 15, 683-691 (2015).
2. Silva-Santos, B., Mensurado, S. & Coffelt, S. B. γδ T cells: pleiotropic immune effectors with therapeutic potential in cancer. Nat. Rev. Cancer 19, 392-404 (2019).
3. Sebestyen, Z., Prinz, I., Déchanet-Merville, J., Silva-Santos, B. & Kuball, J. Translating gammadelta (γδ) T cells and their receptors into cancer cell therapies. Nat. Rev. Drug Discov. 19, 169-184 (2020).
4. Raverdeau, M., Cunningham, S. P., Harmon, C. & Lynch, L. γδ T cells in cancer: a small population of lymphocytes with big implications. Clin. Transl. Immunol. 8, e01080 (2019).
5. Groh, V., Steinle, A., Bauer, S. & Spies, T. Recognition of stress-induced MHC molecules by intestinal epithelial γδ T cells. Science 279, 1737-1740 (1998).
6. Strid, J., Sobolev, O., Zafirova, B., Polic, B. & Hayday, A. The Intraepithelial T Cell Response to NKG2D-Ligands Links Lymphoid Stress Surveillance to Atopy. Science 334, 1293-1297 (2011).
7. Girardi, M. et al. Regulation of cutaneous malignancy by γδ T cells. Science 294, 605-609 (2001).
8. Harly, C. et al. Human γδ T cell sensing of AMPK-dependent metabolic tumor reprogramming through TCR recognition of EphA2. Sci. Immunol. 6, eaba9010 (2021).
9. Rigau, M. el al. Butyrophilin 2A1 is essential for phosphoantigen reactivity by γδ T cells. Science 367, eaay5516 (2020).
10. Karunakaran, M. M. et al. Butyrophilin-2A1 Directly Binds Germline-Encoded Regions of the Vγ9Vδ2 TCR and Is Essential for Phosphoantigen Sensing. Immunity 52, 487-498.e6 (2020).
11. Chien, Y., Meyer, C. & Bonneville, M. γδ T cells: first line of defense and beyond. Annu. Rev. Immunol. 32, 121-155 (2014).
12. Brenner, M. B. et al. Identification of a putative second T-cell receptor. Nature 322, 145-149 (1986).
13. Bank, 1. et al. A functional T3 molecule associated with a novel heterodimer on the surface of immature human thymocytes. Nature 322, 179-181 (1986).
14. Lanier, L. L. et al. The gamma T-cell antigen receptor. J. Clin. Immunol. 7, 429-440 (1987).
15. Harly, C. et al. Key implication of CD277/butyrophilin-3 (BTN3A) in cellular stress sensing by a major human γδ T-cell subset. Blood 120, 2269-2279 (2012).
16. Uhlén, M. et al. Tissue-based map of the human proteome. Science 347, 1260419-1260419 (2015).
17. Payne, K. K. et al. BTN3A1 governs antitumor responses by coordinating αβ and γδ T cells. Science 369, 942-949 (2020).
18. Sandstrom, A. et al. The intracellular B30.2 domain of butyrophilin 3A1 binds phosphoantigens to mediate activation of human Vγ9Vδ2 T cells. Immunity 40, 490-500 (2014).
19. Mullen, P. J., Yu, R., Longo, J., Archer, M. C. & Penn, L. Z. The interplay between cell signalling and the mevalonate pathway in cancer. Nat. Rev. Cancer 16, 718-731 (2016).
20. Patel, S. J. et al. Identification of essential genes for cancer immunotherapy. Nature 548, 537-542 (2017).
21. Yu, Z. et al. Identification of a transporter complex responsible for the cytosolic entry of nitrogen-containing bisphosphonates. eLife 7, e36620 (2018).
22. Dang, A. T. et al. NLRC5 promotes transcription of BTN3A1-3 genes and Vγ9Vδ2 T cell-mediated killing. iScience 24, 101900 (2021).
23. Corvaisier, M. et al. Vγ9Vδ2 T cell response to colon carcinoma cells. J. Immunol. 175, 5481-5488 (2005).
24. Sheftel, A., Stehling, O. & Lill, R. Iron-sulfur proteins in health and disease. Trends Endocrinol. Metabolism 21, 302-314 (2010).
25. Voss, M. et al. Shedding of glycan-modifying enzymes by signal peptide peptidase-like 3 (SPPL3) regulates cellular N-glycosylation. EMBO J. 33, 2890-2905 (2014).
26. Jongsma, M. L. M. et al. The SPPL3-Defined Glycosphingolipid Repertoire Orchestrates HLA Class I-Mediated Immune Responses. Immunity 54, 132-150.e9 (2021).
27. Blevins, M. A., Huang, M. & Zhao, R. The Role of CtBP1 in Oncogenic Processes and Its Potential as a Therapeutic Target. Mol Cancer Ther 16, 981-990 (2017).
28. Annaert, W. & Kaether, C. Bring it back, bring it back, don't take it away from me—the sorting receptor RER1. J Cell Sci 133, jcs231423 (2020).
29. Mick, E. et al. Distinct mitochondrial defects trigger the integrated stress response depending on the metabolic state of the cell. eLife 9, e49178 (2020).
30. Herzig, S. & Shaw, R. J. AMPK: guardian of metabolism and mitochondrial homeostasis. Nat Rev Mol Cell Bio 19, 121-135 (2018).
31. Jiang, P. et al. Signatures of T cell dysfunction and exclusion predict cancer immunotherapy response. Nat. Med. 24, 1550-1558 (2018).
32. Park, J. H. et al. Tumor hypoxia represses γδ T cell-mediated antitumor immunity against brain tumors. Nat. Immunol. 22, 336-346 (2021).
33. Meizlish, M. L., Franklin, R. A., Zhou, X. & Medzhitov, R. Tissue Homeostasis and Inflammation. Annu. Rev. Immunol. 39, 1-25 (2021).
34. Warburg, O., Posener, K. & Negelein, E. Ueber den Stoffwechsel der Tumoren. Biochem. Z. 152, 319-344 (1924).
35. Heiden, M. G. V., Cantley, L. C. & Thompson, C. B. Understanding the Warburg Effect: The Metabolic Requirements of Cell Proliferation. Science 324, 1029-1033 (2009).
36. Tzelepis, K. et al. A CRISPR Dropout Screen Identifies Genetic Vulnerabilities and Therapeutic Targets in Acute Myeloid Leukemia. Cell Rep. 17, 1193-1205 (2016).
37. Shifrut, E. et al. Genome-wide CRISPR Screens in Primary Human T Cells Reveal Key Regulators of Immune Function. Cell 175, 1958-1971.e15 (2018).
38. Li, W. et al. MAGeCK enables robust identification of essential genes from genome-scale CRISPR/Cas9 knockout screens. Genome Biol. 15, 554 (2014).
39. Ma, Y. et al. CRISPR/Cas9 Screens Reveal Epstein-Barr Virus-Transformed B Cell Host Dependency Factors. Cell Host Microbe 21, 580-591.e7 (2017).
40. Jiang, S. et al. CRISPR/Cas9-Mediated Genome Editing in Epstein-Barr Virus-Transformed Lymphoblastoid B-Cell Lines. Curr. Protoc. Mol. Biol. 121, 31.12.1-31.12.23 (2018).
41. Mootha, V. K. et al. PGC-1α-responsive genes involved in oxidative phosphorylation are coordinately downregulated in human diabetes. Nat. Genet. 34, 267-273 (2003).
42. Subramanian, A. et al. Gene set enrichment analysis: A knowledge-based approach for interpreting genome-wide expression profiles. Proc. Natl. Acad. Sci. USA 102, 15545-15550 (2005).
43. Miller, H. E. & Bishop, A. J. R. Correlation AnalyzeR: functional predictions from gene coexpression correlations. BMC Bioinformatics 22, 206 (2021).
44. Lachmann, A. et al. Massive mining of publicly available RNA-seq data from human and mouse. Nat. Commun. 9, 1366 (2018).
45. Concordet, J.-P. & Haeussler, M. CRISPOR: intuitive guide selection for CRISPR/Cas9 genome editing experiments and screens. Nucleic Acids Res. 46, W242-W245 (2018).
46. Martin, M. Cutadapt removes adapter sequences from high-throughput sequencing reads. EMBnet J. 17, 10-12 (2011).
47. Clement, K. et al. CRISPResso2 provides accurate and rapid genome editing sequence analysis. Nat. Biotechnol. 37, 224-226 (2019).
48. Brinkman, E. K., Chen, T., Amendola, M. & Steensel, B. van. Easy quantitative assessment of genome editing by sequence trace decomposition. Nucleic Acids Res. 42, e168-e168 (2014).
49. Davodeau, F. el at. Close correlation between Daudi and mycobacterial antigen recognition by human gamma delta T cells and expression of V9JPC1γ/V2DJCδ-encoded T cell receptors. J. Immunol. 151, 1214-1223 (1993).
50. Shannon, P. et al. Cytoscape: A Software Environment for Integrated Models of Biomolecular Interaction Networks. Genome Res. 13, 2498-2504 (2003).
51. Kutmon, M., Lotia, S., Evelo, C. T. & Pico, A. R. WikiPathways App for Cytoscape: Making biological pathways amenable to network analysis and visualization. F1000Res. 3, 152 (2014).
52. Tsuchiya, S. et al. Implementation of GlycanBuilder to draw a wide variety of ambiguous glycans. Carbohyd. Res. 445, 104-116 (2017).
53. Varki, A. et al. Symbol Nomenclature for Graphical Representations of Glycans. Glycobiology 25, 1323-1324 (2015).
54. Gustavsen, J. A., Pai, S., Isserlin, R., Demchak, B. & Pico, A. R. RCy3: Network biology using Cytoscape from within R. F1000Res. 8, 1774 (2019).
55. Jiang, P. et al. Signatures of T cell dysfunction and exclusion predict cancer immunotherapy response. Nat. Med. 24, 1550-1558 (2018).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements:

1. A method comprising: measuring gene expression levels of one or more BTN3A genes, one or more positive or negative BTN3A regulator genes, or a combination thereof in at least one cell sample from one or more subjects; and identifying any subjects whose sample(s) exhibit:
   a. increased BTN3A expression;
   b. increased BTN3A positive regulator expression;
   c. decreased BTN3A negative regulator expression; or
   d. a combination thereof.
2. The method of statement 1, further comprising obtaining T cells from one or more subjects whose sample(s) exhibit:
   a. increased BTN3A expression;
   b. increased BTN3A positive regulator expression;
   c. decreased BTN3A negative regulator expression; or
   d. a combination thereof.
3. The method of statement 2, further comprising expanding the T cells to generate a population of T cells.
4. The method of statement 2 or 3, further comprising administering the T cells or the population of T cells to subjects whose sample(s) exhibit:
   a. increased BTN3A expression;
   b. increased BTN3A positive regulator expression;
   c. decreased BTN3A negative regulator expression; or
   d. a combination thereof.
5. The method of statement 4, wherein the T cells administered are autologous or allogeneic to the subjects.
6. The method of any one of statements 1-5, wherein the T cells comprise gamma-delta T cells.
7. The method of any one of statements 1-6, wherein the T cells comprise Vgamma9Vdelta2 (Vγ9Vδ2) T cells.
8. The method of any one of statements 1-7, wherein one or more BTN3A regulator genes are transcription factor genes, metabolic sensing genes, mevalonate pathway genes, OXPHOS genes, purine biosynthesis (PPAT) genes, or a combination thereof.
9. The method of any one of statements 1-8, wherein one or more positive negative BTN3A regulator genes is listed in Table 1.
10. The method of any one of statements 1-8, wherein one or more positive BTN3A regulator genes is listed in Table 2.
11. The method of any one of statements 1-10, wherein one or more positive BTN3A regulator genes naturally increase BTN3A surface expression.
12. The method of any one of statements 1-10, wherein one or more negative BTN3A regulator genes naturally decrease BTN3A surface expression.
13. The method of any one of statements 1-12, wherein one or more positive BTN3A regulator genes is ECSIT, FBXW7, SPIB, IRF1, NLRC5, IRF8, NDUFA2, NDUFV1, NDUFA13, USP7, C17orf89, RFXAP, UBE2A, SRPK1, NDUFS7, PDS5B, CNOT11, NDUFB7, BTN3A2, FOXRED1, NDUFS8, JMJD6, NDUFS2, NDUFC2, HSF1, ACAD9, NDUFAF5, TIMMDC1, HSD17B10, BRD2, NDUFA6, CNOT4, SPI1, MDH2, DARS2, TMEM261, STIP1, FIBP, FXR1, NFU1, GGNBP2, STAT2, TRUB2, BIRC6, MARS2, NDUFA9, USP19, UBA6, MTG1, AMPK, or KIAA0391.
14. The method of any one of statements 1-13, wherein one or more positive BTN3A regulator genes is Interferon regulatory factor 1 (IRF1), IRF-8, IRF9, NLRC5, SPIB, SPI1, or TIMMDC1.
15. The method of any one of statements 1-14, wherein one or more negative BTN3A regulator genes is CTBP1, UBE2E1, RING1, ZNF217, HDAC8, RUNX1, RBM38, CBFB, RER1, IKZF1, KCTD5, ST6GAL1, ZNF296, NFKBIA, ATIC, TIAL1, CMAS, CSRNP1, GADD45A, EDEM3, AGO2, RNASEH2A, SRD5A3, ZNF281, MAP2K3, SUPT7L, SLC19A1, CCNL1, AUP1, ZRSR2, CDK13, RASA2, ERF, EIF4ENIF1, PRMT7, MOCS3, HSCB, EDC4, CD79A, SLC16A1, RBM10, GALE, MEF2B, FAM96B, ATXN7, COG8, DERL1, TGFBR2, CHTF8, AHCYL1, or a combination thereof.
16. The method of any one of statements 1-15, wherein one or more negative BTN3A regulator genes is ZNF217, CTBP1, RUNX1, GALE, TIMMDC1, NDUFA2, PPAT, CMAS, RER1, FAM96B, or a combination thereof.
17. The method of any one of statements 8-16, wherein one or more of the transcription factor genes is CTBP1, IRF1, IRF8, IRF9, NLRC5, RUNX1, ZNF217, or a combination thereof.
18. The method of any one of statements 8-17, wherein one or more of the mevalonate pathway genes is FDPS, HMGCS1, MVD, FDPS, GGPS1, or a combination thereof.
19. The method of any one of statements 8-18, wherein one or more of the OXPHOS genes is ATP5A1, ATP5B, ATP5C1, ATP5D, ATP5E, ATP5F1, ATP5G1, ATP5G2, ATP5G3, ATP5H, ATP5I, ATP5J, ATP5J2, ATP5L, ATP5O, ATP5S, COX4I1, COX4I2, COX5A, COX5B, COX6A1, COX6A2, COX6B1, COX6B2, COX6C, COX7A1, COX7A2, COX7B, COX7B2, COX7C, COX8A, COX8C, CYC1, NDUFA1, NDUFA10, NDUFA11, NDUFA12, NDUFA13, NDUFA2, NDUFA3, NDUFA4, NDUFA5, NDUFA6, NDUFA7, NDUFA8, NDUFA9, NDUFAB1, NDUFB1, NDUFB10, NDUFB11, NDUFB2, NDUFB3, NDUFB4, NDUFB5, NDUFB6, NDUFB7, NDUFB8, NDUFB9, NDUFC1, NDUFC2, NDUFS1, NDUFS2, NDUFS3, NDUFS4, NDUFS5, NDUFS6, NDUFS7, NDUFS8, NDUFV1, NDUFV2, NDUFV3, SDHA, SDHB, SDHC, SDHD, UQCR10, UQCR11, UQCRC1, UQCRC2, UQCRFS1, UQCRH, UQCRQ, or a combination thereof
20. The method of any one of statements 8-19, wherein one or more of the OXPHOS genes is ATP5, ATP5A1, ATP5B, ATP5D, ATP5J2, COX (e.g., COX4I1, COX5A, COX6B1, COX6C, COX7B, COX8A), GALE, NDUFA (e.g., NDUFA2, NDUFA3, NDUFA6, and/or NDUFB7), NDUFB, NDUFC2, NDUFS, NDUFV1, SDHC, TIMMDC1, UQCRC1, UQCRC2, or a combination thereof.
21. The method of any one of statements 8-20, wherein one or more of the purine biosynthesis (PPAT) genes is PPAT, GART, ADSL, PAICS, PFAS, ATIC, ADSS, GMPS, or a combination thereof.
22. The method of any one of statements 8-21, wherein CtBP1 is a metabolic sensing gene.
23. The method of any one of statements 1-22, further comprising administering an agent that inhibits BTN3A to subjects whose sample(s) exhibit:
   a. increased BTN3A expression;
   b. increased BTN3A positive regulator expression;
   c. decreased BTN3A negative regulator expression; or
   d. a combination thereof.
24. The method of any one of statements 1-23, further comprising administering an agent that inhibits a positive regulator of BTN3A to subjects whose sample(s) exhibit:
   a. increased BTN3A expression;
   b. increased BTN3A positive regulator expression;
   c. decreased BTN3A negative regulator expression; or
   d. a combination thereof.
25. The method of any one of statements 1-24, further comprising administering a chemotherapeutic agent to subjects whose sample(s) exhibit:
   a. increased BTN3A expression;
   b. increased BTN3A positive regulator expression;
   c. decreased BTN3A negative regulator expression; or
   d. a combination thereof.
26. The method of any of statements 1-25, further comprising administering one or more chemotherapeutic agents, anti-viral agents, antibacterial agents, antimicrobial agents, preservatives, or a combination thereof.
27. The method of any of statements 1-26, further comprising administering one or more alkylating agents (e.g., nitrogen mustards, alkyl sulfonates, nitrosoureas, ethylenimines, triazenes); antimetabolites (e.g., folate antagonists, purine analogues, pyrimidine analogues); antibiotics (e.g., anthracyclines, bleomycins, mitomycin, dactinomycin, plicamycin); enzymes (e.g., L-asparaginase); farnesyl-protein transferase inhibitors, hormonal agents (e.g., glucocorticoids, estrogens/antiestrogens, androgens/antiandrogens, progestins, luteinizing hormone-releasing hormone anatagonists, octreotide acetate); microtubule-disruptor agents (e.g., ecteinascidins); microtubule-stabilizing agents (e.g., paclitaxel (Taxol®), docetaxel (Taxotere®), epothilones A-F); vinca alkaloids, epipodophyllotoxins, taxanes; and topoisomerase inhibitors; prenyl-protein transferase inhibitors; hydroxyurea, procarbazine, mitotane, hexamethylmelamine, platinum coordination complexes (e.g., cisplatin, carboplatin).
28. The method of any one of statements 1-27, further comprising administering a composition comprising one or more compounds formulated in an amount sufficient to inhibit or activate at least one BTN3A1 protein regulator.
29. The method of statement 26, wherein one or more of the compounds is Rotenone, Piericidin A, Metformin, α-Keto-γ-(methylthio)butyric acid, 6-Mercaptopurine monohydrate, Mycophenolic Acid, Zoledronate, Risedronate, Alendronate, AICAR, Compound 991, A-769662, 2,4-Dinitrophenol, Berberine, Canagliflozin, Metformin, Methotrexate, Phenformin, PT-1, Quercetin, R419, Resveratrol, 3 (2-(2-(4-(trifluoromethyl)phenylamino)thiazol-4-yl)acetic acid, C2, BPA-CoA, MK-8722, MT 63-78, 0304, PF249, Salicylate, SC4, ZMP, or a combination thereof in an amount that directly or indirectly modulates the activity of BTN3A1 or one or more BTN3A1 protein regulators.
30. The method of any of statements 1-29, used in conjunction with radiation therapy.
31. A method comprising contacting one or more BTN3A1 proteins/nucleic acids or one or more BTN3A1 regulator proteins/nucleic acids with a test agent to provide a test assay mixture, and:
   a. Detecting and/or quantifying the amount of test agent binding to BTN3A1 protein or the amount of test agent binding to one or more BTN3A1 regulator proteins within the test assay mixture;
   b. Detecting and/or quantifying the amount of test agent binding to BTN3A1 nucleic acids or the amount of test agent binding to one or more BTN3A1 regulator nucleic acids within the test assay mixture;
   c. Quantifying BTN3A1 protein or one or more BTN3A1 regulator proteins in the test assay mixture; or
   d. A combination thereof.
32. A method comprising contacting one or more cells that express BTN3A1 or one or BTN3A1 regulators with a test agent to provide a test assay mixture, and:
   Detecting and/or quantifying the amount of BTN3A1 protein on the surface of one or more cells within the test assay mixture;
   Quantifying cell proliferation in the test assay mixture;
   Quantifying the number of cells that express BTN3A1 protein in the population of cells; or
   A combination thereof.
33. The method of statement 31 or 32, wherein the cells express one or more of the following negative BTN3A1 regulators: CTBP1, UBE2E1, RING1, ZNF217, HDAC8, RUNX1, RBM38, CBFB, RER1, IKZF1, KCTD5, ST6GAL1, ZNF296, NFKBIA, ATIC, TIAL1, CMAS, CSRNP1, GADD45A, EDEM3, AGO2, RNASEH2A, SRD5A3, ZNF281, MAP2K3, SUPT7L, SLC19A1, CCNL1, AUP1, ZRSR2, CDK13, RASA2, ERF, EIF4ENIF1, PRMT7, MOCS3, HSCB, EDC4, CD79A, SLC16A1, RBM10, GALE, MEF2B, FAM96B, ATXN7, COG8, DERL1, TGFBR2, CHTF8, or AHCYL1
34. The method of any one of statements 31-33, wherein the cells express one or more of the following positive BTN3A1 regulators ECSIT, FBXW7, SPIB, IRF1, NLRC5, IRF8, NDUFA2, NDUFV1, NDUFA13, USP7, C17orf89, RFXAP, UBE2A, SRPK1, NDUFS7, PDS5B, CNOT11, NDUFB7, BTN3A2, FOXRED1, NDUFS8, JMJD6, NDUFS2, NDUFC2, HSF1, ACAD9, NDUFAF5, TIMMDC1, HSD17B10, BRD2, NDUFA6, CNOT4, SPI1, MDH2, DARS2, TMEM261, STIP1, FIBP, FXR1, NFU1, GGNBP2, STAT2, TRUB2, BIRC6, MARS2, NDUFA9, USP19, UBA6, MTG1, AMPK, or KIAA0391.
35. The method of any one of statements 31-34, wherein the one or more of the cells is a population of cells.
36. The method of any one of statements 31-35, wherein the one or more of the cells are cancer cells, microbially infected cells, T cells, CD4 T cells, CD8 T cells, alpha-beta CD4 T cells, alpha-beta CD8 T cells, gamma-delta (γδ) T cells, Vgamma9Vdelta2 (Vγ9Vδ2) T cells, an immune cells, a leukocyte, a white blood cell, or a combination thereof.
37. The method of any one of statements 31-36, wherein the one or more of the cells has a mutation.
38. The method of statement 37, wherein the mutation is in the BTN3A1 gene, is in any of the BTN3A1 regulator genes, or is a combination thereof.
39. The method of any one of statements 31-38, wherein one or more of the cells is modified to express or over-express one or more of the BTN3A1 regulators.
40. The method of any one of statements 31-39, wherein one or more of the cells is modified to express or over-express BTN3A1.
41. The method of any one of statements 31-40, wherein one or more of the cells naturally express BTN3A1 or a BTN3A1 regulator.
42. The method of any one of statements 31-41, wherein one or more of cells have the potential to express BTN3A1 or one or more BTN3A1 regulators but when initially mixed with a test agent the cells do not express detectable amounts of BTN3A1 or one or more of the BTN3A1 regulators.
43. The method of any one of statements 31-42, wherein one or more of the cells comprise leukemia cells, lymphoma cells, Hodgkin's disease cells, sarcomas of the soft tissue and bone, lung cancer cells, mesothelioma, esophagus cancer cells, stomach cancer cells, pancreatic cancer cells, hepatobiliary cancer cells, small intestinal cancer cells, colon cancer cells, colorectal cancer cells, rectum cancer cells, kidney cancer cells, urethral cancer cells, bladder cancer cells, prostate cancer cells, testis cancer cells, cervical cancer cells, ovarian cancer cells, breast cancer cells, endocrine system cancer cells, skin cancer cells, central nervous system cancer cells, melanoma cells of cutaneous and/or intraocular origin, cancer cells associated with AIDS, or a combination thereof.
44. The method of any one of statements 31-43, wherein one or more of cells comprise metastatic cancer cells, micrometastatic tumor cells, megametastatic tumor cells, recurrent cancer cells, or a combination thereof
45. The method of any one of statements 31-44, wherein one or more of cells are infected with a bacterial, viral, protozoan or other infectious agent.
46. The method of any one of statements 31-45, wherein one or more of cells further comprise an expression cassette encoding a cas nuclease.
47. The method of statement 46, wherein the nuclease is a cas9 nuclease.
48. The method of any one of statements 31-47, wherein proteins and/or cells and the test agents are incubated together for a time and under conditions effective to detect whether the test agent can modulate the expression or activity of BTN3A1, the expression or activity of a BTN3A1 regulator, or the expression or activity of at least one cell in the assay mixture.
49. The method of any one of statements 31-48, wherein the test agent is one or more small molecules, antibodies, nucleic acids, expression cassettes, expression vectors, inhibitory nucleic acids, guide RNAs, nucleases (e.g., one or more cas nucleases), or a combination thereof.
50. The method of any one of statements 31-49, wherein the test agent is one or more of the BTN3A1 regulators described herein, one or more anti-BTN3A1 antibodies, one or more BTN3A1 inhibitory nucleic acids that can modulate the expression of the BTN3A1, one or more guide RNAs that can bind a BTN3A1 nucleic acid, one or more antibodies that can bind any of the BTN3A1 regulators described herein, one or more inhibitory nucleic acid that can modulate the expression of any of the BTN3A1 regulators described herein, one or more guide RNAs that can bind a nucleic acid encoding any of the BTN3A1 regulators described herein, one or more small molecules that can modulate BTN3A1, one or more small molecules that can modulate any of the BTN3A1 regulators, one or more guide RNAs, or a combination thereof.
51. The method of any one of statements 31-50, further comprising antibody staining of BTN3A1, antibody staining of one or more BTN3A1 regulator, cell flow cytometry, cell counting, cell viability, RNA detection, RNA quantification, RNA sequencing, protein detection, SDS-polyacrylamide gel electrophoresis, DNA sequencing, cytokine detection, interferon detection, or a combination thereof.
52. The method of any one of statements 31-51, further comprising quantifying T cell responses in the test assay mixture.
53. A method comprising detecting a mutation in a BTN3A1gene or in one or more BTN3A1 regulator genes within a nucleic acid sample from a mammalian subject; and administering a therapeutic agent to the subject.
54. The method of statement 53, wherein the therapeutic agent is an anti-cancer agent, an anti-bacterial agent, an anti-protozoan agent, an anti-viral agent, or a combination thereof.
55. A composition comprising a test agent identified by the method of any of statements 31-52 that can modulate the expression or activity of BTN3A1.
56. A composition comprising a test agent identified by the method of any of statements 31-55 that can modulate the expression or activity of one or more BTN3A1 regulators.
57. The composition of statement 56, wherein one or more of the BTN3A1 regulators is one or more of the following negative BTN3A1 regulators: CTBP1, UBE2E1, RING1, ZNF217, HDAC8, RUNX1, RBM38, CBFB, RER1, IKZF1, KCTD5, ST6GAL1, ZNF296, NFKBIA, ATIC, TIAL1, CMAS, CSRNP1, GADD45A, EDEM3, AGO2, RNASEH2A, SRD5A3, ZNF281, MAP2K3, SUPT7L, SLC19A1, CCNL1, AUP1, ZRSR2, CDK13, RASA2, ERF, EIF4ENIF1, PRMT7, MOCS3, HSCB, EDC4, CD79A, SLC16A1, RBM10, GALE, MEF2B, FAM96B, ATXN7, COG8, DERL1, TGFBR2, CHTF8, or AHCYL1.
58. The composition of statement 56 or 57, wherein one or more of the BTN3A1 regulators is one or more of the following positive BTN3A1 regulators: ECSIT, FBXW7, SPIB, IRF1, NLRC5, IRF8, NDUFA2, NDUFV1, NDUFA13, USP7, C17orf89, RFXAP, UBE2A, SRPK1, NDUFS7, PDS5B, CNOT11, NDUFB7, BTN3A2, FOXRED1, NDUFS8, JMJD6, NDUFS2, NDUFC2, HSF1, ACAD9, NDUFAF5, TIMMDC1, HSD17B10, BRD2, NDUFA6, CNOT4, SPI1, MDH2, DARS2, TMEM261, STIP1, FIBP, FXR1, NFU1, GGNBP2, STAT2, TRUB2, BIRC6, MARS2, NDUFA9, USP19, UBA6, MTG1, AMPK, or KIAA0391.
59. The composition of any one of statements 55-58, which comprises a small molecule, a peptide, a protein, an antibody, an expression cassette, an expression vector, an inhibitory nucleic acid, a guide RNA, a nuclease, or a combination thereof.
60. A composition comprising one or more BTN3A1 protein regulators.
61. A composition comprising an antibody that specifically binds BTN3A1 or one or more BTN3A1 regulator proteins.
62. A composition comprising an expression cassette or an expression vector comprising a nucleic acid segment comprising one or more coding regions for one or more BTN3A1 regulators.
63. The composition of any one of statements 55-62, further comprising an AMPK inhibitor or AMPK activator.
64. The composition of any one of statements 55-63, wherein one or more of the BTN3A1 regulators is one or more of the following negative BTN3A1 regulators: CTBP1, UBE2E1, RING1, ZNF217, HDAC8, RUNX1, RBM38, CBFB, RER1, IKZF1, KCTD5, ST6GAL1, ZNF296, NFKBIA, ATIC, TIAL1, CMAS, CSRNP1, GADD45A, EDEM3, AGO2, RNASEH2A, SRD5A3, ZNF281, MAP2K3, SUPT7L, SLC19A1, CCNL1, AUP1, ZRSR2, CDK13, RASA2, ERF, EIF4ENIF1, PRMT7, MOCS3, HSCB, EDC4, CD79A, SLC16A1, RBM10, GALE, MEF2B, FAM96B, ATXN7, COG8, DERL1, TGFBR2, CHTF8, or AHCYL1.
65. The composition of any one of statements 55-64, wherein one or more of the BTN3A1 regulators is one or more of the following positive BTN3A1 regulators: ECSIT, FBXW7, SPIB, IRF1, NLRC5, IRF8, NDUFA2, NDUFV1, NDUFA13, USP7, C17orf89, RFXAP, UBE2A, SRPK1, NDUFS7, PDS5B, CNOT11, NDUFB7, BTN3A2, FOXRED1, NDUFS8, JMJD6, NDUFS2, NDUFC2, HSF1, ACAD9, NDUFAF5, TIMMDC1, HSD17B10, BRD2, NDUFA6, CNOT4, SPI1, MDH2, DARS2, TMEM261, STIP1, FIBP, FXR1, NFU1, GGNBP2, STAT2, TRUB2, BIRC6, MARS2, NDUFA9, USP19, UBA6, MTG1, AMPK, or KIAA0391.
66. The composition of any of statements 55-65, further comprising one or more chemotherapeutic agents, anti-viral agents, antibacterial agents, antimicrobial agents, preservatives, or a combination thereof
67. The composition of any of statements 55-66, further comprising one or more alkylating agents (e.g., nitrogen mustards, alkyl sulfonates, nitrosoureas, ethylenimines, triazenes); antimetabolites (e.g., folate antagonists, purine analogues, pyrimidine analogues); antibiotics (e.g., anthracyclines, bleomycins, mitomycin, dactinomycin, plicamycin); enzymes (e.g., L-asparaginase); farnesyl-protein transferase inhibitors, hormonal agents (e.g., glucocorticoids, estrogens/antiestrogens, androgens/antiandrogens, progestins, luteinizing hormone-releasing hormone anatagonists, octreotide acetate); microtubule-disruptor agents (e.g., ecteinascidins); microtubule-stabilizing agents (e.g., paclitaxel (Taxol®), docetaxel (Taxotere®), epothilones A-F); vinca alkaloids, epipodophyllotoxins, taxanes; and topoisomerase inhibitors; prenyl-protein transferase inhibitors; hydroxyurea, procarbazine, mitotane, hexamethylmelamine, platinum coordination complexes (e.g., cisplatin, carboplatin).
68. The composition of any of statements 55-67, used in conjunction with radiation therapy.
69. The composition of any of statements 55-68, formulated in a therapeutically effective amount.
70. A method comprising administering the composition of any of statements 55-69 to a subject.
71. The method or composition of any one of statements 1-70, wherein the subject is a mammal or bird.
72. The method or composition of any one of statements 1-71, wherein the subject is a human, domestic animal, farm animal, zoo animal, experimental animal, pet animal, or a combination thereof.
73. The method or composition of any one of statements 1-72, wherein the subject is one or more mice, rats, guinea pigs, goats, dogs, monkeys, or a combination thereof.
74. The method or composition of any one of statements 1-73, wherein the subject is a human.
75. The method or composition of any one of statements 1-74, comprising administering at least one of the following compounds to the subject: Rotenone, Piericidin A, Metformin, α-Keto-γ-(methylthio)butyric acid, 6-Mercaptopurine monohydrate, Mycophenolic Acid, Zoledronate, Risedronate, Alendronate, or a combination thereof in an amount that directly or indirectly modulates the activity of BTN3A1 or one or more BTN3A1 protein regulators.
76. A composition comprising one or more compounds formulated in an amount sufficient to inhibit or activate at least one BTN3A1 protein regulator.
77. The composition of statement 76, comprising at least one of the following compounds: Rotenone, Piericidin A, Metformin, α-Keto-γ-(methylthio)butyric acid, 6-Mercaptopurine monohydrate, Mycophenolic Acid, Zoledronate, Risedronate, Alendronate, AICAR, Compound 991, A-769662, 2,4-Dinitrophenol, Berberine, Canagliflozin, Metformin, Methotrexate, Phenformin, PT-1, Quercetin, R419, Resveratrol, 3 (2-(2-(4-(trifluoromethyl)phenylamino)thiazol-4-yl)acetic acid, C2, BPA-CoA, MK-8722, MT 63-78, 0304, PF249, Salicylate, SC4, ZMP, or a combination thereof in an amount that directly or indirectly modulates the activity of BTN3A1 or one or more BTN3A1 protein regulators.
78. A method comprising ex vivo modification of any of the genes listed in Table 1 or 2 within at least one lymphoid or myeloid cell, or a combination thereof, to generate at least one modified lymphoid cell, at least one modified myeloid cell, or a mixture of modified lymphoid and modified myeloid cells.
79. The method of statement 78, wherein the modification is one or more deletion, substitution or insertion into one or more genomic sites of any of the genes listed in Table 1 or 2.
80. The method of statement 78 or 79, wherein the modification is transformation of the at least one lymphoid or myeloid cell, or a combination thereof with at least one expression cassette encoding one or more coding regions of the genes listed in Table 1 or 2.
81. The method of statement 78, 79, or 80, wherein the modification is one or more CRISPR-mediated modifications or activations of any of the genes listed in Table 1 or 2.
82. The method of any one of statements 78-81, further comprising administering at least one modified lymphoid cell, at least one modified myeloid cell, or a mixture of modified lymphoid and modified myeloid cells to a subject.
83. The method of any one of statements 78-82, further comprising incubating the at least one modified lymphoid cell, at least one modified myeloid cell, or a mixture of modified lymphoid and modified myeloid cells to form a population of modified cells.
84. The method of statement 83, further comprising administering the population of modified cells to a subject.
85. The method of any one of statements 82 or 84, wherein the subject has a disease or condition.
86. The method of statement 85, wherein the disease or condition is an immune condition or cancer.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising administering T cell therapies, BTN3A inhibitors, or BTN3A negative regulators to a subject whose cell sample(s) exhibit:
a. increased BTN3A expression;
b. increased BTN3A positive regulator expression;
c. decreased BTN3A negative regulator expression; or
d. a combination thereof.

2. The method of claim 1, wherein the T cell therapies comprise gamma-delta (γδ) T cells, Vgamma9Vdelta2 (Vγ9Vδ2) T cells, CD4 T cells, CD8 T cells, alpha-beta CD4 T cells, alpha-beta CD8 T cells, or a combination thereof or combinations thereof.

3. The method of claim 1, wherein one or more of the BTN3A negative regulators is listed in Table 1.

4. The method of claim 1, wherein one or more of the negative BTN3A1 regulators is CTBP1, UBE2E1, RING1, ZNF217, HDAC8, RUNX1, RBM38, CBFB, RER1, IKZF1, KCTD5, ST6GAL1, ZNF296, NFKBIA, ATIC, TIAL1, CMAS, CSRNP1, GADD45A, EDEM3, AGO2, RNASEH2A, SRD5A3, ZNF281, MAP2K3, SUPT7L, SLC19A1, CCNL1, AUP1, ZRSR2, CDK13, RASA2, ERF, EIF4ENIF1, PRMT7, MOCS3, HSCB, EDC4, CD79A, SLC16A1, RBM10, GALE, MEF2B, FAM96B, ATXN7, COG8, DERL1, TGFBR2, CHTF8, AHCYL1, or a combination thereof.

5. The method of claim 1, wherein one or more of the negative BTN3A1 regulators is administered as an expression cassette or expression vector comprising a promoter operably linked to a nucleic acid segment encoding one or more of the negative BTN3A1 regulators.

6. The method of claim 1, wherein one or more of the BTN3A positive regulators is listed in Table 2.

7. The method of claim 1, wherein one or more of the BTN3A positive regulators is ECSIT, FBXW7, SPIB, IRF1, IRF8, IRF9, NLRC5, IRF8, NDUFA2, NDUFV1, NDUFA13, USP7, C17orf89, RFXAP, UBE2A, SRPK1, NDUFS7, PDS5B, CNOT11, NDUFB7, BTN3A2, FOXRED1, NDUFS8, JMJD6, NDUFS2, NDUFC2, HSF1, ACAD9, NDUFAF5, TIMMDC1, HSD17B10, BRD2, NDUFA6, CNOT4, SPI1, MDH2, DARS2, TMEM261, STIP1, FIBP, FXR1, NFU1, GGNBP2, STAT2, TRUB2, BIRC6, MARS2, NDUFA9, USP19, UBA6, MTG1, AMPK, or KIAA0391.

8. The method of claim 1, wherein one or more of the BTN3A positive regulators is one or more of the following OXPHOS genes: ATP5A1, ATP5B, ATP5C1, ATP5D, ATP5E, ATP5F1, ATP5G1, ATP5G2, ATP5G3, ATP5H, ATP5I, ATP5J, ATP5J2, ATP5L, ATP5O, ATP5S, COX4I1, COX4I2, COX5A, COX5B, COX6A1, COX6A2, COX6B1, COX6B2, COX6C, COX7A1, COX7A2, COX7B, COX7B2, COX7C, COX8A, COX8C, CYC1, NDUFA1, NDUFA10, NDUFA11, NDUFA12, NDUFA13, NDUFA2, NDUFA3, NDUFA4, NDUFA5, NDUFA6, NDUFA7, NDUFA8, NDUFA9, NDUFAB1, NDUFB1, NDUFB10, NDUFB11, NDUFB2, NDUFB3, NDUFB4, NDUFB5, NDUFB6, NDUFB7, NDUFB8, NDUFB9, NDUFC1, NDUFC2, NDUFS1, NDUFS2, NDUFS3, NDUFS4, NDUFS5, NDUFS6, NDUFS7, NDUFS8, NDUFV1, NDUFV2, NDUFV3, SDHA, SDHB, SDHC, SDHD, UQCR10, UQCR11, UQCRC1, UQCRC2, UQCRFS1, UQCRH, UQCRQ, or a combination thereof.

9. The method of claim 1, wherein one or more of the BTN3A inhibitors is one or more antibody types, inhibitory nucleic acids, guide RNAs, cas nucleases, expression cassettes, expression vectors, small molecules, or a combination thereof.

10. The method of claim 1, further comprising administering one or more compounds that modulates at least one BTN3A positive regulator or at least one BTN3A negative regulator.

11. The method of claim 1, comprising administering at least one of the following compounds to the subject: Rotenone, Piericidin A, Metformin, α-Keto-γ-(methylthio)butyric acid, 6-Mercaptopurine monohydrate, Mycophenolic Acid, Zoledronate, Risedronate, Alendronate, AICAR, Compound 991, A-769662, 2,4-Dinitrophenol, Berberine, Canagliflozin, Metformin, Methotrexate, Phenformin, PT-1, Quercetin, R419, Resveratrol, 3 (2-(2-(4-(trifluoromethyl)phenylamino)thiazol-4-yl)acetic acid, C2, BPA-CoA, MK-8722, MT 63-78, O304, PF249, Salicylate, SC4, ZMP, or a combination thereof in an amount that directly or indirectly modulates the activity of BTN3A1 or one or more BTN3A1 protein regulators.

12. The method of claim 1, further comprising administering one or more chemotherapeutic agents, anti-viral agents, antibacterial agents, antimicrobial agents, preservatives, or a combination thereof.

13. A method comprising contacting one or more cells that express BTN3A1 or one or BTN3A1 regulators with a test agent to provide a test assay mixture, and:
detecting and/or quantifying the amount of BTN3A1 protein on the surface of one or more cells within the test assay mixture;
quantifying cell proliferation in the test assay mixture;
quantifying the number of cells that express BTN3A1 protein in the population of cells; or
a combination thereof.

14. The method of claim 13, wherein the cells express one or more of the following positive BTN3A regulators: ECSIT, FBXW7, SPIB, IRF1, IRF8, IR9, NLRC5, IRF8, NDUFA2, NDUFV1, NDUFA13, USP7, C17orf89, RFXAP, UBE2A, SRPK1, NDUFS7, PDS5B, CNOT11, NDUFB7, BTN3A2, FOXRED1, NDUFS8, JMJD6, NDUFS2, NDUFC2, HSF1, ACAD9, NDUFAF5, TIMMDC1, HSD17B10, BRD2, NDUFA6, CNOT4, SPI1, MDH2, DARS2, TMEM261, STIP1, FIBP, FXR1, NFU1, GGNBP2, STAT2, TRUB2, BIRC6, MARS2, NDUFA9, USP19, UBA6, MTG1, AMPK, KIAA0391, or a combination thereof.

15. The method of claim 13, wherein the test assay mixture further comprises T cells.

16. The method of claim 15, wherein the T cells are CD4 T cells, CD8 T cells, alpha-beta CD4 T cells, alpha-beta CD8 T cells, gamma-delta (γδ) T cells, Vgamma9Vdelta2 (Vγ9Vδ2) T cells, or a combination thereof.

17. The method of claim 13, wherein one or more of the cells are cancer cells or a cell population comprising cancer cells.

18. The method of claim 17, wherein one or more of cancer cells comprise metastatic cancer cells, micrometastatic tumor cells, megametastatic tumor cells, recurrent cancer cells, or a combination thereof.

19. The method of claim 17, wherein one or more of the cancer cells comprise leukemia cells, lymphoma cells, Hodgkin's disease cells, sarcomas of the soft tissue and bone, lung cancer cells, mesothelioma, esophagus cancer cells, stomach cancer cells, pancreatic cancer cells, hepatobiliary cancer cells, small intestinal cancer cells, colon cancer cells, colorectal cancer cells, rectum cancer cells, kidney cancer cells, urethral cancer cells, bladder cancer cells, prostate cancer cells, testis cancer cells, cervical cancer cells, ovarian cancer cells, breast cancer cells, endocrine system cancer cells, skin cancer cells, central nervous system cancer cells, melanoma cells of cutaneous and/or intraocular origin, cancer cells associated with AIDS, or a combination thereof.

20. The method of claim 13, wherein the test agent is one or more small molecules, antibodies, nucleic acids, expression cassettes, expression vectors, inhibitory nucleic acids, guide RNAs, cas nucleases, or a combination thereof.

21. The method of claim 13, wherein the test agent is one or more of the BTN3A1 regulators, one or more anti-BTN3A1 antibodies, one or more BTN3A1 inhibitory nucleic acids that can modulate the expression of the BTN3A1, one or more guide RNAs that can bind a BTN3A1 nucleic acid, one or more antibodies that can bind one or more of the BTN3A1 regulators, one or more inhibitory nucleic acid that can modulate the expression of one or more of the BTN3A1 regulators, one or more guide RNAs that can bind a nucleic acid encoding one or more of the BTN3A1 regulators, one or more small molecules that can modulate BTN3A1, one or more small molecules that can modulate one or more of the BTN3A1 regulators, one or more guide RNAs, or a combination thereof.

22. The method of claim 13, wherein cells and the test agents are incubated together for a time and under conditions effective to detect whether the test agent can modulate the expression or activity of BTN3A1, the expression or activity of a BTN3A1 regulator, or the growth, viability, or activity of at least one cell in the assay mixture.

23. The method of claim 13, further comprising identifying one or more test agents that
a. reduces the amount of BTN3A1 protein on the surface of one or more cells within the test assay mixture;
b. reduces the number of cells that express BTN3A1 protein in the population of cells;
c. reduces cell proliferation in the test assay mixture; or
d. a combination thereof.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. A method comprising administering Vγ9Vδ2 T cells to a cancer patient whose cancer cells express increased levels of one or more of BTN3A1, NLRC5, IRF1, IRF8, IRF9, SPI1, SPIB, ZNF217, RUNX1, AMPK, FDPS, or a combination thereof, compared to one or more reference values.


SEQUENCE LISTING (220 sequences)

SEQ ID NO: 1 - protein, 513 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 2 - protein, 352 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPGEEMLQMRLHFVK

SEQ ID NO: 3 - protein, 461 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 4 - protein, 378 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPGPPIGQTQQQTRGQGSPVALSQESAQRTDSWGPEEGGES

SEQ ID NO: 5 - protein, 506 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRGWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 6 - protein, 352 aa, Homo sapiens; identical to SEQ ID NO: 2

SEQ ID NO: 7 - protein, 419 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 8 - protein, 440 aa, Homo sapiens
MGSSHLLNKGLPLGVRPPIMNGPLHPRPLVALLDGRDCTVEMPILKDVATVAFCDAQSTQEIHEKVLNEAVGALMYHTITLTREDLEKFKALRIIVRIGSGFDNIDIKSAGDLGIAVCNVPAASVEETADSTLCHILNLYRRATWLHQALREGTRVQSVEQIREVASGAARIRGETLGIIGLGRVGQAVALRAKAFGFNVLFYDPYLSDGVERALGLQRVSTLQDLLFHSDCVTLHCGLNEHNHHLINDFTVKQMRQGAFLVNTARGGLVDEKALAQALKEGRIRGAALDVHESEPFSFSQGPLKDAPNLICTPHAAWYSEQASIEMREEAAREIRRAITGRIPDSLKNCVNKDHLTAATHWASMDPAVVHPELNGAAYRYPPGVVGVAPTGIPAAVEGIVPSAMSLSHGLPPVAHPPHAPSPGQTVKPEADRDHASDQL

SEQ ID NO: 9 - protein, 193 aa, Homo sapiens
MSDDDSRASTSSSSSSSSNQQTEKETNTPKKKESKVSMSKNSKLLSTSAKRIQKELADITLDPPPNCSAGPKGDNIYEWRSTILGPPGSVYEGGVFFLDITFTPEYPFKPPKVTFRTRIYHCNINSQGVICLDILKDNWSPALTISKVLLSICSLLTDCNPADPLVGSIATQYMTNRAEHDRMARQWTKRYAT

SEQ ID NO: 10 - protein, 406 aa, Homo sapiens
MTTPANAQNASKTWELSLYELHRTPQEAIMDGTEIAVSPRSLHSELMCPICLDMLKNTMTTKECLHRFCSDCIVTALRSGNKECPTCRKKLVSKRSLRPDPNFDALISKIYPSREEYEAHQDRVLIRLSRLHNQQALSSSIEEGLRMQAMHRAQRVRRPIPGSDQTTTMSGGEGEPGEGEGDGEDVSSDSAPDSAPGPAPKRPRGGGAGGSSVGTGGGGTGGVGGGAGSEDSGDRGGTLGGGTLGPPSPPGAPSPPEPGGEIELVFRPHPLLVEKGEYCQTRYVKTTGNATVDHLSKYLALRIALERRQQQEAGEPGGPGGGASDTGGPDGCGGEGGGAGGGDGPEEPALPSLEGVSEKQYTIYIAPGGGAFTTLNGSLTLELVNEKFWKVSRPLELCYAPTKDPK

SEQ ID NO: 11 - protein, 1048 aa, Homo sapiens
MQSKVTGNMPTQSLLMYMDGPEVIGSSLGSPMEMEDALSMKGTAVVPFRATQEKNVIQIEGYMPLDCMFCSQTFTHSEDLNKHVLMQHRPTLCEPAVLRVEAEYLSPLDKSQVRTEPPKEKNCKENEFSCEVCGQTFRVAFDVEIHMRTHKDSFTYGCNMCGRRFKEPWFLKNHMRTHNGKSGARSKLQQGLESSPATINEVVQVHAAESISSPYKICMVCGFLFPNKESLIEHRKVHTKKTAFGTSSAQTDSPQGGMPSSREDFLQLFNLRPKSHPETGKKPVRCIPQLDPFTTFQAWQLATKGKVAICQEVKESGQEGSTDNDDSSSEKELGETNKGSCAGLSQEKEKCKHSHGEAPSVDADPKLPSSKEKPTHCSECGKAFRTYHQLVLHSRVHKKDRRAGAESPTMSVDGRQPGTCSPDLAAPLDENGAVDRGEGGSEDGSEDGLPEGIHLDKNDDGGKIKHLTSSRECSYCGKFFRSNYYLNIHLRTHTGEKPYKCEFCEYAAAQKTSLRYHLERHHKEKQTDVAAEVKNDGKNQDTEDALLTADSAQTKNLKRFFDGAKDVTGSPPAKQLKEMPSVFQNVLGSAVLSPAHKDTQDFHKNAADDSADKVNKNPTPAYLDLLKKRSAVETQANNLICRTKADVTPPPDGSTTHNLEVSPKEKQTETAADCRYRPSVDCHEKPLNLSVGALHNCPAISLSKSLIPSITCPFCTFKTFYPEVLMMHQRLEHKYNPDVHKNCRNKSLLRSRRTGCPPALLGKDVPPLSSFCKPKPKSAFPAQSKSLPSAKGKQSPPGPGKAPLTSGIDSSTLAPSNLKSHRPQQNVGVQGAATRQQQSEMFPKTSVSPAPDKTKRPETKLKPLPVAPSQPTLGSSNINGSIDYPAKNDSPWAPPGRDYFCNRSASNTAAEFGEPLPKRLKSSVVALDVDQPGANYRRGYDLPKYHMVRGITSLLPQDCVYPSQALPPKPRFLSSSEVDSPNVLTVQKPYGGSGPLYTCVPAGSPASSSTLEGKRPVSYQHLSNSMAQKRNYENFIGNAHYRPNDKKT

SEQ ID NO: 12 - protein, 377 aa, Homo sapiens
MEEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDHEFFTAYGPDYVLEITPSCRPDRNEPHRIQQILNYIKGNLKHVV

SEQ ID NO: 13 - protein, 453 aa, Homo sapiens
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 14 - protein, 239 aa, Homo sapiens
MLLQPAPCAPSAGFPRPLAAPGAMHGSQKDTTFTKIFVGGLPYHTTDASLRKYFEGFGDIEEAVVITDRQTGKSRGYGFVTMADRAAAERACKDPNPIIDGRKANVNLAYLGAKPRSLQTGFAIGVQQLHPTLIQRTYGLTPHYIYPPAIVQPSVVIPAAPVPSLSSPYIEYTPASPAYAQYPPATYDQYPYAASPATAASFVGYSYPAAVPQALSAAAPAGTTFVQYQAPQLQPDRMQ

SEQ ID NO: 15 - protein, 182 aa, Homo sapiens
MPRVVPDQRSKFENEEFFRKLSRECEIKYTGFRDRPHEERQARFQNACRDGRSEIAFVATGTNLSLQFFPASWQGEQRQTPSREYVDLEREAGKVYLKAPMILNGVCVIWKGWIDLQRLDGMGCLEFDEERAQQEDALAQQAFEEARRRTREFEDRDRSHREEMEVRVSQLLAVTGKKTTRP

SEQ ID NO: 16 - protein, 196 aa, Homo sapiens
MSEGDSVGESVHGKPSVVYRFFTRLGQIYQSWLDKSTPYTAVRWVVTLGLSFVYMIRVYLLQGWYIVTYALGIYHLNLFIAFLSPKVDPSLMEDSDDGPSLPTKQNEEFRPFIRRLPEFKFWHAATKGILVAMVCTFFDAFNVPVFWPILVMYFIMLFCITMKRQIKHMIKYRYIPFTHGKRRYRGKEDAGKAFAS

SEQ ID NO: 17 - protein, 519 aa, Homo sapiens
MDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVGKPHKCGYCGRSYKQRSSLEEHKERCHNYLESMGLPGTLYPVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 18 - protein, 234 aa, Homo sapiens
MAENHCELLSPARGGIGAGLGGGLCRRCSAGLGALAQRPGSVSKWVRLNVGGTYFLTTRQTLCRDPKSFLYRLCQADPDLDSDKDETGAYLIDRDPTYFGPVLNYLRHGKLVINKDLAEEGVLEEAEFYNITSLIKLVKDKIRERDSKTSQVPVKHVYRVLQCQEEELTQMVSTMSDGWKFEQLVSIGSSYNYGNEDQAEFLCVVSKELHNTPYGTASEPSEKAKILQERGSRM

SEQ ID NO: 19 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 20 - protein, 475 aa, Homo sapiens
MSRRKAGSAPRRVEPAPAANPDDEMEMQDLVIELKPEPDAQPQQAPRLGPFSPKEVSSAGRFGGEPHHSPGPMPAGAALLALGPRNPWTLWTPLTPNYPDRQPWTDKHPDLLTCGRCLQTFPLEAITAFMDHKKLGCQLFRGPSRGQGSEREELKALSCLRCGKQFTVAWKLLRHAQWDHGLSIYQTESEAPEAPLLGLAEVAAAVSAVVGPAAEAKSPRASGSGLTRRSPTCPVCKKTLSSFSNLKVHMRSHTGERPYACDQCPYACAQSSKLNRHKKTHRQVPPQSPLMADTSQEQASAAPPEPAVHAAAPTSTLPCSGGEGAGAAATAGVQEPGAPGSGAQAGPGGDTWGAITTEQRTDPANSQKASPKKMPKSGGKSRGPGGSCEFCGKHFTNSSNLTVHRRSHTGERPYTCEFCNYACAQSSKLNRHRRMHGMTPGSTRFECPHCHVPFGLRATLDKHLRQKHPEAAGEA

SEQ ID NO: 21 - protein, 317 aa, Homo sapiens
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 22 - protein, 592 aa, Homo sapiens
MAPGQLALFSVSDKTGLVEFARNLTALGLNLVASGGTAKALRDAGLAVRDVSELTGFPEMLGGRVKTLHPAVHAGILARNIPEDNADMARLDFNLIRVVACNLYPFVKTVASPGVTVEEAVEQIDIGGVTLLRAAAKNHARVTVVCEPEDYVVVSTEMQSSESKDTSLETRRQLALKAFTHTAQYDEAISDYFRKQYSKGVSQMPLRYGMNPHQTPAQLYTLQPKLPITVLNGAPGFINLCDALNAWQLVKELKEALGIPAAASFKHVSPAGAAVGIPLSEDEAKVCMVYDLYKTLTPISAAYARARGADRMSSFGDFVALSDVCDVPTAKIISREVSDGIIAPGYEEEALTILSKKKNGNYCVLQMDQSYKPDENEVRTLFGLHLSQKRNNGVVDKSLFSNVVTKNKDLPESALRDLIVATIAVKYTQSNSVCYAKNGQVIGIGAGQQSRIHCTRLAGDKANYWWLRHHPQVLSMKFKTGVKRAEISNAIDQYVTGTIGEDEDLIKWKALFEEVPELLTEAEKKEWVEKLTEVSISSDAFFPFRDNVDRAKRSGVAYIAAPSGSAADKVVIEACDELGIILAHTNLRLFHH

SEQ ID NO: 23 - protein, 375 aa, Homo sapiens
MMEDDGQPRTLYVGNLSRDVTEVLILQLFSQIGPCKSCKMITEHTSNDPYCFVEFYEHRDAAAALAAMNGRKILGKEVKVNWATTPSSQKKDTSNHFHVFVGDLSPEITTEDIKSAFAPFGKISDARVVKDMATGKSKGYGFVSFYNKLDAENAIVHMGGQWLGGRQIRTNWATRKPPAPKSTQENNTKQLRFEDVVNQSSPKNCTVYCGGIASGLTDQLMRQTFSPFGQIMEIRVFPEKGYSFVRFSTHESAAHAIVSVNGTTIEGHVVKCYWGKESPDMTKNFQQVDYSQWGQWSQVYGNPQQYGQYMANGWQVPPYGVYGQPWNQQGFGVDQSPSAAWMGGFGAQPPQGQAPPPVIPPPNQAGYGMASYQTQ

SEQ ID NO: 24 - protein, 434 aa, Homo sapiens
MDSVEKGAATSVSNPRGRPSRGRPPKLQRNSRGGQGRGVEKPPHLAALILARGGSKGIPLKNIKHLAGVPLIGWVLRAALDSGAFQSVWVSTDHDEIENVAKQFGAQVHRRSSEVSKDSSTSLDAIIEFLNYHNEVDIVGNIQATSPCLHPTDLQKVAEMIREEGYDSVFSVVRRHQFRWSEIQKGVREVTEPLNLNPAKRPRRQDWDGELYENGSFYFAKRHLIEMGYLQGGKMAYYEMRAEHSVDIDVDIDWPIAEQRVLRYGYFGKEKLKEIKLLVCNIDGCLTNGHIYVSGDQKEIISYDVKDAIGISLLKKSGIEVRLISERACSKQTLSSLKLDCKMEVSVSDKLAVVDEWRKEMGLCWKEVAYLGNEVSDEECLKRVGLSGAPADACSTAQKAVGYICKCNGGRGAIREFAEHICLLMEKVNNSCQK

SEQ ID NO: 25 - protein, 589 aa, Homo sapiens
MTGLLKRKFDQLDEDNSSVSSSSSSSGCQSRSCSPSSSVSRAWDSEEEGPWDQMPLPDRDFCGPRSFTPLSILKRARRERPGRVAFDGITVFYFPRCQGFTSVPSRGGCTLGMALRHSACRRFSLAEFAQEQARARHEKLRQRLKEEKLEMLQWKLSAAGVPQAEAGLPPVVDAIDDASVEEDLAVAVAGGRLEEVSFLQPYPARRRRALLRASGVRRIDREEKRELQALRQSREDCGCHCDRICDPETCSCSLAGIKCQMDHTAFPCGCCREGCENPMGRVEFNQARVQTHFIHTLTRLQLEQEAESFRELEAPAQGSPPSPGEEALVPTFPLAKPPMNNELGDNSCSSDMTDSSTASSSASGTSEAPDCPTHPGLPGPGFQPGVDDDSLARILSFSDSDFGGEEEEEEEGSVGNLDNLSCFHPADIFGTSDPGGLASWTHSYSGCSFTSGVLDENANLDASCFLNGGLEGSREGSLPGTSVPPSMDAGRSSSVDLSLSSCDSFELLQALPDYSLGPHYTSQKVSDSLDNIEAPHFPLPGLSPPGDASSCFLESLMGFSEPAAEALDPFIDSQFEDTVPASLMEPVPV

SEQ ID NO: 26 - protein, 165 aa, Homo sapiens
MTLEEFSAGEQKTERMDKVGDALEEVLSKALSQRTITVGVYEAAKLLNVDPDNVVLCLLAADEDDDRDVALQIHFTLIQAFCCENDINILRVSNPGRLAELLLLETDAGPAASEGAEQPPDLHCVLVTNPHSSQWKDPALSQLICFCRESRYMDQWVPVINLPER

SEQ ID NO: 27 - protein, 932 aa, Homo sapiens
MSEAGGRGCGSPVPQRARWRLVAATAAFCLVSATSVWTAGAEPMSREEKQKLGNQVLEMFDHAYGNYMEHAYPADELMPLTCRGRVRGQEPSRGDVDDALGKFSLTLIDSLDTLVVLNKTKEFEDAVRKVLRDVNLDNDVVVSVFETNIRVLGGLLGGHSLAIMLKEKGEYMQWYNDELLQMAKQLGYKLLPAFNTTSGLPYPRINLKFGIRKPEARTGTETDTCTACAGTLILEFAALSRFTGATIFEEYARKALDFLWEKRQRSSNLVGVTINIHTGDWVRKDSGVGAGIDSYYEYLLKAYVLLGDDSFLERFNTHYDAIMRYISQPPLLLDVHIHKPMLNARTWMDALLAFFPGLQVLKGDIRPAIETHEMLYQVIKKHNFLPEAFTTDFRVHWAQHPLRPEFAESTYFLYKATGDPYYLEVGKTLIENLNKYARVPCGFAAMKDVRTGSHEDRMDSFFLAEMFKYLYLLFADKEDIIFDIEDYIFTTEAHLLPLWLSTTNQSISKKNTTSEYTELDDSNFDWTCPNTQILFPNDPLYAQSIREPLKNVVDKSCPRGIIRVEESFRSGAKPPLRARDFMATNPEHLEILKKMGVSLIHLKDGRVQLVQHAIQAASSIDAEDGLRFMQEMIELSSQQQKEQQLPPRAVQIVSHPFFGRVVLTAGPAQFGLDLSKHKETRGFVASSKPSNGCSELTNPEAVMGKIALIQRGQCMFAEKARNIQNAGAIGGIVIDDNEGSSSDTAPLFQMAGDGKDTDDIKIPMLFLFSKEGSIILDAIREYEEVEVLLSDKAKDRDPEMENEEQPSSENDSQNQSGEQISSSSQEVDLVDQESSEENSLNSHPESLSLADMDNAASISPSEQTSNPTENHETTNLNGECTDLDNQLQEQSETEEDSNPNVSWGKKVQPIDSILADWNEDIEAFEMMEKDEL

SEQ ID NO: 28 - protein, 859 aa, Homo sapiens
MYSGAGPALAPPAPPPPIQGYAFKPPPRPDFGTSGRTIKLQANFFEMDIPKIDIYHYELDIKPEKCPRRVNREIVEHMVQHFKTQIFGDRKPVFDGRKNLYTAMPLPIGRDKVELEVTLPGEGKDRIFKVSIKWVSCVSLQALHDALSGRLPSVPFETIQALDVVMRHLPSMRYTPVGRSFFTASEGCSNPLGGGREVWFGFHQSVRPSLWKMMLNIDVSATAFYKAQPVIEFVCEVLDFKSIEEQQKPLTDSQRVKFTKEIKGLKVEITHCGQMKRKYRVCNVTRRPASHQTFPLQQESGQTVECTVAQYFKDRHKLVLRYPHLPCLQVGQEQKHTYLPLEVCNIVAGQRCIKKLTDNQTSTMIRATARSAPDRQEEISKLMRSASFNTDPYVREFGIMVKDEMTDVTGRVLQPPSILYGGRNKAIATPVQGVWDMRNKQFHTGIEIKVWAIACFAPQRQCTEVHLKSFTEQLRKISRDAGMPIQGQPCFCKYAQGADSVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFA

SEQ ID NO: 29 - protein, 299 aa, Homo sapiens
MDLSELERDNTGRCRLSSPVPAVCRKEPCVLGVDEAGRGPVLGPMVYAICYCPLPRLADLEALKVADSKTLLESERERLFAKMEDTDFVGWALDVLSPNLISTSMLGRVKYNLNSLSHDTATGLIQYALDQGVNVTQVFVDTVGMPETYQARLQQSFPGIEVTVKAKADALYPVVSAASICAKVARDQAVKKWQFVEKLQDLDTDYGSGYPNDPKTKAWLKEHVEPVFGFPQFVRFSWRTAQTILEKEAEDVIWEDSASENQEGLRKITSYFLNEGSQARPRSSHRYFLERGLESATSL

SEQ ID NO: 30 - protein, 318 aa, Homo sapiens
MAPWAEAEHSALNPLRAVWLTLTAAFLLTLLLQLLPPGLLPGCAIFQDLIRYGKTKCGEPSRPAACRAFDVPKRYFSHFYIISVLWNGFLLWCLTQSLFLGAPFPSWLHGLLRILGAAQFQGGELALSAFLVLVFLWLHSLRRLFECLYVSVFSNVMIHVVQYCFGLVYYVLVGLTVLSQVPMDGRNAYITGKNLLMQARWFHILGMMMFIWSSAHQYKCHVILGNLRKNKAGVVIHCNHRIPFGDWFEYVSSPNYLAELMIYVSMAVTFGFHNLTWWLVVTNVFFNQALSAFLSHQFYKSKFVSYPKHRKAFLPFLF

SEQ ID NO: 31 - protein, 895 aa, Homo sapiens
MKIGSGFLSGGGGTGSSGGSGSGGGGSGGGGGGGSSGRRAEMEPTFPQGMVMFNHRLPPVTSFTRPAGSAAPPPQCVLSSSTSAAPAAEPPPPPAPDMTFKKEPAASAAAFPSQRTSWGFLQSLVSIKQEKPADPEEQQSHHHHHHHHYGGLFAGAEERSPGLGGGEGGSHGVIQDLSILHQHVQQQPAQHHRDVLLSSSSRTDDHHGTEEPKQDTNVKKAKRPKPESQGIKAKRKPSASSKPSLVGDGEGAILSPSQKPHICDHCSAAFRSSYHLRRHVLIHTGERPFQCSQCSMGFIQKYLLQRHEKIHSREKPFGCDQCSMKFIQKYHMERHKRTHSGEKPYKCDTCQQYFSRTDRLLKHRRTCGEVIVKGATSAEPGSSNHTNMGNLAVLSQGNTSSSRRKTKSKSIAIENKEQKTGKTNESQISNNINMQSYSVEMPTVSSSGGIIGTGIDELQKRVPKLIFKKGSRKNTDKNYLNFVSPLPDIVGQKSLSGKPSGSLGIVSNNSVETIGLLQSTSGKQGQISSNYDDAMQFSKKRRYLPTASSNSAFSINVGHMVSQQSVIQSAGVSVLDNEAPLSLIDSSALNAEIKSCHDKSGIPDEVLQSILDQYSNKSESQKEDPFNIAEPRVDLHTSGEHSELVQEENLSPGTQTPSNDKASMLQEYSKYLQQAFEKSTNASFTLGHGFQFVSLSSPLHNHTLFPEKQIYTTSPLECGFGQSVTSVLPSSLPKPPFGMLFGSQPGLYLSALDATHQQLTPSQELDDLIDSQKNLETSSAFQSSSQKLTSQKEQKNLESSTGFQIPSQELASQIDPQKDIEPRTTYQIENFAQAFGSQFKSGSRVPMTFITNSNGEVDHRVRTSVSDFSGYTNMMSDVSEPCSTRVKTPTSQSYR

SEQ ID NO: 32 - protein, 347 aa, Homo sapiens
MESPASSQPASMPQSKGKSKRKKDLRISCMSKPPAPNPTPPRNLDSRTFITIGDRNFEVEADDLVTISELGRGAYGVVEKVRHAQSGTIMAVKRIRATVNSQEQKRLLMDLDINMRTVDCFYTVTFYGALFREGDVWICMELMDTSLDKFYRKVLDKNMTIPEDILGEIAVSIVRALEHLHSKLSVIHRDVKPSNVLINKEGHVKMCDFGISGYLVDSVAKTMDAGCKPYMAPERINPELNQKGYNVKSDVWSLGITMIEMAILRFPYESWGTPFQQLKQVVEEPSPQLPADRFSPEFVDFTAQCLRKNPAERMSYLELMEHPFFTLHKTKKTDIAAFVKEILGEDS

SEQ ID NO: 33 - protein, 414 aa, Homo sapiens
MNLQRYWGEIPISSSQTNRSSFDLLPREFRLVEVHDPPLHQPSANKPKPPTMLDIPSEPCSLTIHTIQLIQHNRRLRNLIATAQAQNQQQTEGVKTEESEPLPSCPGSPPLPDDLLPLDCKNPNAPFQIRHSDPESDFYRGKGEPVTELSWHSCRQLLYQAVATILAHAGFDCANESVLETLTDVAHEYCLKFTKLLRFAVDREARLGQTPFPDVMEQVFHEVGIGSVLSLQKFWQHRIKDYHSYMLQISKQLSEEYERIVNPEKATEDAKPVKIKEEPVSDITFPVSEELEADLASGDQSLPMGVLGAQSERFPSNLEVEASPQASSAEVNASPLWNLAHVKMEPQESEEGNVSGHGVLGSDVFEEPMSGMSEAGIPQSPDDSDSSYGSHSTDSLMGSSPVFNQRCKKRMRKI

SEQ ID NO: 34 - protein, 591 aa, Homo sapiens
MVPSSPAVEKQVPVEPGPDPELRSWRHLVCYLCFYGFMAQIRPGESFITPYLLGPDKNFTREQVTNEITPVLSYSYLAVLVPVFLLTDYLRYTPVLLLQGLSFVSVWLLLLLGHSVAHMQLMELFYSVTMAARIAYSSYIFSLVRPARYQRVAGYSRAAVLLGVFTSSVLGQLLVTVGRVSFSTLNYISLAFLTFSVVLALFLKRPKRSLFFNRDDRGRCETSASELERMNPGPGGKLGHALRVACGDSVLARMLRELGDSLRRPQLRLWSLWWVFNSAGYYLVVYYVHILWNEVDPTTNSARVYNGAADAASTLLGAITSFAAGFVKIRWARWSKLLIAGVTATQAGLVFLLAHTRHPSSIWLCYAAFVLFRGSYQFLVPIATFQIASSLSKELCALVFGVNTFFATIVKTIITFIVSDVRGLGLPVRKQFQLYSVYFLILSIIYFLGAMLDGLRHCQRGHHPRQPPAQGLRSAAEEKAAQALSVQDKGLGGLQPAQSPPLSPEDSLGAVGPASLEQRQSDPYLAQAPAPQAAEFLSPVTTPSPCTLCSAQASGPEAADETCPQLAVHPPGVSKLGLQCLPSDGVQNVNQ

SEQ ID NO: 35 - protein, 526 aa, Homo sapiens
MASGPHSTATAAAAASSAAPSAGGSSSGTTTTTTTTTGGILIGDRLYSEVSLTIDHSLIPEERLSPTPSMQDGLDLPSETDLRILGCELIQAAGILLRLPQVAMATGQVLFHRFFYSKSFVKHSFEIVAMACINLASKIEEAPRRIRDVINVFHHLRQLRGKRTPSPLILDQNYINTKNQVIKAERRVLKELGFCVHVKHPHKIIVMYLQVLECERNQTLVQTAWNYMNDSLRTNVFVRFQPETIACACIYLAARALQIPLPTRPHWFLLFGTTEEEIQEICIETLRLYTRKKPNYELLEKEVEKRKVALQEAKLKAKGLNPDGTPALSTLGGFSPASKPSSPREVKAEEKSPISINVKTVKKEPEDRQQASKSPYNGVRKDSKRSRNSRSASRSRSRTRSRSRSHTPRRHYNNRRSRSGTYSSRSRSRSRSHSESPRRHHNHGSPHLKAKHTRDDLKSSNRHGHKRKKSRSRSQSKSRDHSDAAKKHRHERGHHRDRRERSRSFERSHKSKHHGGSRSGHGRHRR

SEQ ID NO: 36 - protein, 410 aa, Homo sapiens
MELPSGPGPERLFDSHRLPGDCFLLLVLLLYAPVGFCLLVLRLFLGIHVFLVSCALPDSVLRRFVVRTMCAVLGLVARQEDSGLRDHSVRVLISNHVTPFDHNIVNLLTTCSTPLLNSPPSFVCWSRGFMEMNGRGELVESLKRFCASTRLPPTPLLLFPEEEATNGREGLLRFSSWPFSIQDVVQPLTLQVQRPLVSVTVSDASWVSELLWSLFVPFTVYQVRWLRPVHRQLGEANEEFALRVQQLVAKELGQTGTRLTPADKAEHMKRQRHPRLRPQSAQSSFPPSPGPSPDVQLATLAQRVKEVLPHVPLGVIQRDLAKTGCVDLTITNLLEGAVAFMPEDITKGTQSLPTASASKFPSSGPVTPQPTALTFAKSSWARQESLQERKQALYEYARRRFTERRAQEAD

SEQ ID NO: 37 - protein, 482 aa, Homo sapiens
MAAPEKMTFPEKPSHKKYRAALKKEKRKKRRQELARLRDSGLSQKEEEEDTFIEEQQLEEEKLLERERQRLHEEWLLREQKAQEEFRIKKEKEEAAKKRQEEQERKLKEQWEEQQRKEREEEEQKRQEKKEKEEALQKMLDQAENELENGTTWQNPEPPVDFRVMEKDRANCPFYSKTGACRFGDRCSRKHNFPTSSPTLLIKSMFTTFGMEQCRRDDYDPDASLEYSEEETYQQFLDFYEDVLPEFKNVGKVIQFKVSCNLEPHLRGNVYVQYQSEEECQAALSLFNGRWYAGRQLQCEFCPVTRWKMAICGLFEIQQCPRGKHCNFLHVFRNPNNEFWEANRDIYLSPDRTGSSFGKNSERRERMGHHDDYYSRLRGRRNPSPDHSYKRNGESERKSSRHRGKKSHKRTSKSRERHNSRSRGRNRDRSRDRSRGRGSRSRSRSRSRRSRRSRSQSSSRSRSRGRRRSGNRDRTVQSPKSK

SEQ ID NO: 38 - protein, 1512 aa, Homo sapiens
MPSSSDTALGGGGGLSWAEKKLEERRKRRRFLSPQQPPLLLPLLQPQLLQPPPPPPPLLFLAAPGTAAAAAAAAAASSSCFSPGPPLEVKRLARGKRRAGGRQKRRRGPRAGQEAEKRRVFSLPQPQQDGGGGASSGGGVTPLVEYEDVSSQSEQGLLLGGASAATAATAAGGTGGSGGSPASSSGTQRRGEGSERRPRRDRRSSSGRSKERHREHRRRDGQRGGSEASKSRSRHSHSGEERAEVAKSGSSSSSGGRRKSASATSSSSSSRKDRDSKAHRSRTKSSKEPPSAYKEPPKAYREDKTEPKAYRRRRSLSPLGGRDDSPVSHRASQSLRSRKSPSPAGGGSSPYSRRLPRSPSPYSRRRSPSYSRHSSYERGGDVSPSPYSSSSWRRSRSPYSPVLRRSGKSRSRSPYSSRHSRSRSRHRLSRSRSRHSSISPSTLTLKSSLAAELNKNKKARAAEAARAAEAAKAAEATKAAEAAAKAAKASNTSTPTKGNTETSASASQTNHVKDVKKIKIEHAPSPSSGGTLKNDKAKTKPPLQVTKVENNLIVDKATKKAVIVGKESKSAATKEESVSLKEKTKPLTPSIGAKEKEQHVALVTSTLPPLPLPPMLPEDKEADSLRGNISVKAVKKEVEKKLRCLLADLPLPPELPGGDDLSKSPEEKKTATQLHSKRRPKICGPRYGETKEKDIDWGKRCVDKFDIIGIIGEGTYGQVYKARDKDTGEMVALKKVRLDNEKEGFPITAIREIKILRQLTHQSIINMKEIVTDKEDALDFKKDKGAFYLVFEYMDHDLMGLLESGLVHFNENHIKSFMRQLMEGLDYCHKKNFLHRDIKCSNILLNNRGQIKLADFGLARLYSSEESRPYTNKVITLWYRPPELLLGEERYTPAIDVWSCGCILGELFTKKPIFQANQELAQLELISRICGSPCPAVWPDVIKLPYFNTMKPKKQYRRKLREEFVFIPAAALDLFDYMLALDPSKRCTAEQALQCEFLRDVEPSKMPPPDLPLWQDCHELWSKKRRRQKQMGMTDDVSTIKAPRKDLSLGLDDSRTNTPQGVLPSSQLKSQGSSNVAPVKTGPGQHLNHSELAILLNLLQSKTSVNMADFVQVLNIKVNSETQQQLNKINLPAGILATGEKQTDPSTPQQESSKPLGGIQPSSQTIQPKVETDAAQAAVQSAFAVLLTQLIKAQQSKQKDVLLEERENGSGHEASLQLRPPPEPSTPVSGQDDLIQHQDMRILELTPEPDRPRILPPDQRPPEPPEPPPVTEEDLDYRTENQHVPTTSSSLTDPHAGVKAALLQLLAQHQPQDDPKREGGIDYQAGDTYVSTSDYKDNFGSSSFSSAPYVSNDGLGSSSAPPLERRSFIGNSDIQSLDNYSTASSHSGGPPQPSAFSESFPSSVAGYGDIYLNAGPMLFSGDKDHRFEYSHGPIAVLANSSDPSTGPESTHPLPAKMHNYNYGGNLQENPSGPSLMHGQTWTSPAQGPGYSQGYRGHISTSTGRGRGRGLPY

SEQ ID NO: 39 - protein, 850 aa, Homo sapiens
MAAAAPAAAAASSEAPAASATAEPEAGDQDSREVRVLQSLRGKICEAKNLLPYLGPHKMRDCFCTINLDQEEVYRTQVVEKSLSPFFSEEFYFEIPRTFQYLSFYVYDKNVLQRDLRIGKVAIKKEDLCNHSGKETWFSLQPVDSNSEVQGKVHLELKLNELITENGTVCQQLVVHIKACHGLPLINGQSCDPYATVSLVGPSRNDQKKTKVKKKTSNPQFNEIFYFEVTRSSSYTRKSQFQVEEEDIEKLEIRIDLWNNGNLVQDVFLGEIKVPVNVLRTDSSHQAWYLLQPRDNGNKSSKTDDLGSLRLNICYTEDYVLPSEYYGPLKTLLLKSPDVQPISASAAYILSEICRDKNDAVLPLVRLLLHHDKLVPFATAVAELDLKDTQDANTIFRGNSLATRCLDEMMKIVGGHYLKVTLKPILDEICDSSKSCEIDPIKLKEGDNVENNKENLRYYVDKLFNTIVKSSMSCPTVMCDIFYSLRQMATQRFPNDPHVQYSAVSSFVFLRFFAVAVVSPHTFHLRPHHPDAQTIRTLTLISKTIQTLGSWGSLSKSKSSFKETFMCEFFKMFQEEGYIIAVKKFLDEISSTETKESSGTSEPVHLKEGEMYKRAQGRTRIGKKNFKKRWFCLTSRELTYHKQPGSKDAIYTIPVKNILAVEKLEESSFNKKNMFQVIHTEKPLYVQANNCVEANEWIDVLCRVSRCNQNRLSFYHPSVYLNGNWLCCQETGENTLGCKPCTAGVPADIQIDIDEDRETERIYSLFTLSLLKLQKMEEACGTIAVYQGPQKEPDDYSNFVIEDSVTTFKTIQQIKSIIEKLDEPHEKYRKKRSSSAKYGSKENPIVGKAS

SEQ ID NO: 40 - protein, 548 aa, Homo sapiens
MKTPADTGFAFPDWAYKPESSPGSRQIQLWHFILELLRKEEYQGVIAWQGDYGEFVIKDPDEVARLWGVRKCKPQMNYDKLSRALRYYYNKRILHKTKGKRFTYKFNFNKLVLVNYPFIDVGLAGGAVPQSAPPVPSGGSHFRFPPSTPSEVLSPTEDPRSPPACSSSSSSLFSAVVARRLGRGSVSDCSDGTSELEEPLGEDPRARPPGPPDLGAFRGPPLARLPHDPGVFRVYPRPRGGPEPLSPFPVSPLAGPGSLLPPQLSPALPMTPTHLAYTPSPTLSPMYPSGGGGPSGSGGGSHFSFSPEDMKRYLQAHTQSVYNYHLSPRAFLHYPGLVVPQPQRPDKCPLPPMAPETPPVPSSASSSSSSSSSPFKFKLQPPPLGRRQRAAGEKAVAGADKSGGSAGGLAEGAGALAPPPPPPQIKVEPISEGESEEVEVTDISDEDEEDGEVFKTPRAPPAPPKPEPGEAPGASQCMPLKLRFKRRWSEDCRLEGGGGPAGGFEDEGEDKKVRGEGPGEAGGPLTPRRVSSDLQHATAQLSLEHRDS

SEQ ID NO: 41 - protein, 985 aa, Homo sapiens
MDRRSMGETESGDAFLDLKKPPASKCPHRYTKEELLDIKELPHSKQRPSCLSEKYDSDGVWDPEKWHASLYPASGRSSPVESLKKELDTDRPSLVRRIVDPRERVKEDDLDVVLSPQRRSFGGGCHVTAAVSSRRSGSPLEKDSDGLRLLGGRRIGSGRIISARTFEKDHRLSDKDLRDLRDRDRERDFKDKRFRREFGDSKRVFGERRRNDSYTEEEPEWFSAGPTSQSETIELTGFDDKILEEDHKGRKRTRRRTASVKEGIVECNGGVAEEDEVEVILAQEPAADQEVPRDAVLPEQSPGDFDFNEFFNLDKVPCLASMIEDVLGEGSVSASRFSRWFSNPSRSGSRSSSLGSTPHEELERLAGLEQAILSPGQNSGNYFAPIPLEDHAENKVDILEMLQKAKVDLKPLLSSLSANKEKLKESSHSGVVLSVEEVEAGLKGLKVDQQVKNSTPFMAEHLEETLSAVTNNRQLKKDGDMTAFNKLVSTMKASGTLPSQPKVSRNLESHLMSPAEIPGQPVPKNILQELLGQPVQRPASSNLLSGLMGSLEPTTSLLGQRAPSPPLSQVFQTRAASADYLRPRIPSPIGFTPGPQQLLGDPFQGMRKPMSPITAQMSQLELQQAALEGLALPHDLAVQAANFYQPGFGKPQVDRTRDGFRNRQQRVTKSPAPVHRGNSSSPAPAASITSMLSPSFTPTSVIRKMYESKEKSKEEPASGKAALGDSKEDTQKASEENLLSSSSVPSADRDSSPTTNSKLSALQRSSCSTPLSQANRYTKEQDYRPKATGRKTPTLASPVPTTPFLRPVHQVPLVPHVPMVRPAHQLHPGLVQRMLAQGVHPQHLPSLLQTGVLPPGMDLSHLQGISGPILGQPFYPLPAASHPLLNPRPGTPLHLAMVQQQLQRSVLHPPGSGSHAAAVSVQTTPQNVPSRSGLPHMHSQLEHRPSQRSSSPVGLAKWFGSDVLQQPLPSMPAKVISVDELEYRQ

SEQ ID NO: 42 - protein, 692 aa, Homo sapiens
MKIFCSRANPTTGSVEWLEEDEHYDYHQEIARSSYADMLHDKDRNVKYYQGIRAAVSRVKDRGQKALVLDIGTGTGLLSMMAVTAGADFCYAIEVFKPMADAAVKIVEKNGFSDKIKVINKHSTEVTVGPEGDMPCRANILVTELFDTELIGEGALPSYEHAHRHLVEENCEAVPHRATVYAQLVESGRMWSWNKLFPIHVQTSLGEQVIVPPVDVESCPGAPSVCDIQLNQVSPADFTVLSDVLPMFSIDFSKQVSSSAACHSRRFEPLTSGRAQVVLSWWDIEMDPEGKIKCTMAPFWAHSDPEEMQWRDHWMQCVYFLPQEEPVVQGSALYLVAHHDDYCVWYSLQRTSPEKNERVRQMRPVCDCQAHLLWNRPRFGEINDQDRTDRYVQALRTVLKPDSVCLCVSDGSLLSVLAHHLGVEQVFTVESSAASHKLLRKIFKANHLEDKINIIEKRPELLTNEDLQGRKVSLLLGEPFFTTSLLPWHNLYFWYVRTAVDQHLGPGAMVMPQAASLHAVVVEFRDLWRIRSPCGDCEGFDVHIMDDMIKRALDFRESREAEPHPLWEYPCRSLSEPWQILTFDFQQPVPLQPLCAEGTVELRRPGQSHAAVLWMEYHLTPECTLSTGLLEPADPEGGCCWNPHCKQAVYFFSPAPDPRALLGGPRTVSYAVEFHPDTGDIIMEFRHADTPD

SEQ ID NO: 43 - protein, 692 aa, Homo sapiens; identical to SEQ ID NO: 42

SEQ ID NO: 44 - protein, 235 aa, Homo sapiens
MWRGRAGALLRVWGFWPTGVPRRRPLSCDAASQAGSNYPRCWNCGGPWGPGREDRFFCPQCRALQAPDPTRDYFSLMDCNRSFRVDTAKLQHRYQQLQRLVHPDFFSQRSQTEKDFSEKHSTLVNDAYKTLLAPLSRGLYLLKLHGIEIPERTDYEMDRQFLIEIMEINEKLAEAESEAAMKEIESIVKAKQKEFTDNVSSAFEQDDFEEAKEILTKMRYFSNIEEKIKLKKIPL

SEQ ID NO: 45 - protein, 1401 aa, Homo sapiens
MASCASIDIEDATQHLRDILKLDRPAGGPSAESPRPSSAYNGDLNGLLVPDPLCSGDSTSANKTGLRTMPPINLQEKQVICLSGDDSSTCIGILAKEVEIVASSDSSISSKARGSNKVKIQPVAKYDWEQKYYYGNLIAVSNSFLAYAIRAANNGSAMVRVISVSTSERTLLKGFTGSVADLAFAHLNSPQLACLDEAGNLFVWRLALVNGKIQEEILVHIRQPEGTPLNHFRRIIWCPFIPEESEDCCEESSPTVALLHEDRAEVWDLDMLRSSHSTWPVDVSQIKQGFIVVKGHSTCLSEGALSPDGTVLATASHDGYVKFWQIYIEGQDEPRCLHEWKPHDGRPLSCLLFCDNHKKQDPDVPFWRFLITGADQNRELKMWCTVSWTCLQTIRFSPDIFSSVSVPPSLKVCLDLSAEYLILSDVQRKVLYVMELLQNQEEGHACFSSISEFLLTHPVLSFGIQVVSRCRLRHTEVLPAEEENDSLGADGTHGAGAMESAAGVLIKLFCVHTKALQDVQIRFQPQLNPDVVAPLPTHTAHEDFTFGESRPELGSEGLGSAAHGSQPDLRRIVELPAPADFLSLSSETKPKLMTPDAFMTPSASLQQITASPSSSSSGSSSSSSSSSSSLTAVSAMSSTSAVDPSLTRPPEELTLSPKLQLDGSLTMSSSGSLQASPRGLLPGLLPAPADKLTPKGPGQVPTATSALSLELQEVEPLGLPQASPSRTRSPDVISSASTALSQDIPEIASEALSRGFGSSAPEGLEPDSMASAASALHLLSPRPRPGPELGPQLGLDGGPGDGDRHNTPSLLEAALTQEASTPDSQVWPTAPDITRETCSTLAESPRNGLQEKHKSLAFHRPPYHLLQQRDSQDASAEQSDHDDEVASLASASGGFGTKVPAPRLPAKDWKTKGSPRTSPKLKRKSKKDDGDAAMGSRLTEHQVAEPPEDWPALIWQQQRELAELRHSQEELLQRLCTQLEGLQSTVTGHVERALETRHEQEQRRLERALAEGQQRGGQLQEQLTQQLSQALSSAVAGRLERSIRDEIKKTVPPCVSRSLEPMAGQLSNSVATKLTAVEGSMKENISKLLKSKNLTDAIARAAADTLQGPMQAAYREAFQSVVLPAFEKSCQAMFQQINDSFRLGTQEYLQQLESHMKSRKAREQEAREPVLAQLRGLVSTLQSATEQMAATVAGSVRAEVQHQLHVAVGSLQESILAQVQRIVKGEVSVALKEQQAAVTSSIMQAMRSAAGTPVPSAHLDCQAQQAHILQLLQQGHLNQAFQQALTAADLNLVLYVCETVDPAQVFGQPPCPLSQPVLLSLIQQLASDLGTRTDLKLSYLEEAVMHLDHSDPITRDHMGSVMAQVRQKLFQFLQAEPHNSLGKAARRLSLMLHGLVTPSLP

SEQ ID NO: 46 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 47 - protein, 500 aa, Homo sapiens
MPPAVGGPVGYTPPDGGWGWAVVIGAFISIGFSYAFPKSITVFFKEIEGIFHATTSEVSWISSIMLAVMYGGGPISSILVNKYGSRIVMIVGGCLSGCGLIAASFCNTVQQLYVCIGVIGGLGLAFNLNPALTMIGKYFYKRRPLANGLAMAGSPVFLCTLAPLNQVFFGIFGWRGSFLILGGLLLNCCVAGALMRPIGPKPTKAGKDKSKASLEKAGKSGVKKDLHDANTDLIGRHPKQEKRSVFQTINQFLDLTLFTHRGFLLYLSGNVIMFFGLFAPLVFLSSYGKSQHYSSEKSAFLLSILAFVDMVARPSMGLVANTKPIRPRIQYFFAASVVANGVCHMLAPLSTTYVGFCVYAGFFGFAFGWLSSVLFETLMDLVGPQRFSSAVGLVTIVECCPVLLGPPLLGRLNDMYGDYKYTYWACGVVLIISGIYLFIGMGINYRLLAKEQKANEQKKESKEEETSIDVAGKPNEVTKAAESPDQKDTDGGPKEEESPV

SEQ ID NO: 48 - protein, 930 aa, Homo sapiens
MEYERRGGRGDRTGRYGATDRSQDDGGENRSRDHDYRDMDYRSYPREYGSQEGKHDYDDSSEEQSAEDSYEASPGSETQRRRRRRHRHSPTGPPGFPRDGDYRDQDYRTEQGEEEEEEEDEEEEEKASNIVMLRMLPQAATEDDIRGQLQSHGVQAREVRLMRNKSSGQSRGFAFVEFSHLQDATRWMEANQHSLNILGQKVSMHYSDPKPKINEDWLCNKCGVQNFKRREKCFKCGVPKSEAEQKLPLGTRLDQQTLPLGGRELSQGLLPLPQPYQAQGVLASQALSQGSEPSSENANDTIILRNLNPHSTMDSILGALAPYAVLSSSNVRVIKDKQTQLNRGFAFIQLSTIVEAAQLLQILQALHPPLTIDGKTINVEFAKGSKRDMASNEGSRISAASVASTAIAAAQWAISQASQGGEGTWATSEEPPVDYSYYQQDEGYGNSQGTESSLYAHGYLKGTKGPGITGTKGDPTGAGPEASLEPGADSVSMQAFSRAQPGAAPGIYQQSAEASSSQGTAANSQSYTIMSPAVLKSELQSPTHPSSALPPATSPTAQESYSQYPVPDVSTYQYDETSGYYYDPQTGLYYDPNSQYYYNAQSQQYLYWDGERRTYVPALEQSADGHKETGAPSKEGKEKKEKHKTKTAQQIAKDMERWARSLNKQKENFKNSFQPISSLRDDERRESATADAGYAILEKKGALAERQHTSMDLPKLASDDRPSPPRGLVAAYSGESDSEEEQERGGPEREEKLTDWQKLACLLCRRQFPSKEALIRHQQLSGLHKQNLEIHRRAHLSENELEALEKNDMEQMKYRDRAAERREKYGIPEPPEPKRRKYGGISTASVDFEQPTRDGLGSDNIGSRMLQAMGWKEGSGLGRKKQGIVTPIEAQTRVRGSGLGARGSSYGVTSTESYKETLHKTMVTRFNEAQ

SEQ ID NO: 49 - protein, 348 aa, Homo sapiens
MAEKVLVTGGAGYIGSHTVLELLEAGYLPVVIDNFHNAFRGGGSLPESLRRVQELTGRSVEFEEMDILDQGALQRLFKKYSFMAVIHFAGLKAVGESVQKPLDYYRVNLTGTIQLLEIMKAHGVKNLVFSSSATVYGNPQYLPLDEAHPTGGCTNPYGKSKFFIEEMIRDLCQADKTWNAVLLRYFNPTGAHASGCIGEDPQGIPNNLMPYVSQVAIGRREALNVFGNDYDTEDGTGVRDYIHVVDLAKGHIAALRKLKEQCGCRIYNLGTGTGYSVLQMVQAMEKASGKKIPYKVVARREGDVAACYANPSLAQEELGWTAALGLDRMCEDLWRWQKQNPSGFGTQA

SEQ ID NO: 50 - protein, 365 aa, Homo sapiens
MGRKKIQISRILDQRNRQVTFTKRKFGLMKKAYELSVLCDCEIALIIFNSANRLFQYASTDMDRVLLKYTEYSEPHESRTNTDILETLKRRGIGLDGPELEPDEGPEEPGEKFRRLAGEGGDPALPRPRLYPAAPAMPSPDVVYGALPPPGCDPSGLGEALPAQSRPSPFRPAAPKAGPPGLVHPLFSPSHLTSKTPPPLYLPTEGRRSDLPGGLAGPRGGLNTSRSLYSGLQNPCSTATPGPPLGSFPFLPGGPPVGAEAWARRVPQPAAPPRRPPQSASSLSASLRPPGAPATFLRPSPIPCSSPGPWQSLCGLGPPCAGCPWPTAGPGRRSPGGTSPERSPGTARARGDPTSLQASSEKTQQ

SEQ ID NO: 51 - protein, 163 aa, Homo sapiens
MVGGGGVGGGLLENANPLIYQRSGERPVTAGEEDEQVPDSIDAREIFDLIRSINDPEHPLTLEELNVVEQVRVQVSDPESTVAVAFTPTIPHCSMATLIGLSIKVKLLRSLPQRFKMDVHITPGTHASEHAVNKQLADKERVAAALENTHLLEVVNQCLSARS

SEQ ID NO: 52 - protein, 892 aa, Homo sapiens
MSERAADDVRGEPRRAAAAAGGAAAAAARQQQQQQQQQQPPPPQPQRQQHPPPPPRRTRPEDGGPGAASTSAAAMATVGERRPLPSPEVMLGQSWNLWVEASKLPGKDGTELDESFKEFGKNREVMGLCREDMPIFGFCPAHDDFYLVVCNDCNQVVKPQAFQSHYERRHSSSSKPPLAVPPTSVFSFFPSLSKSKGGSASGSNRSSSGGVLSASSSSSKLLKSPKEKLQLRGNTRPMHPIQQSRVPHGRIMTPSVKVEKIHPKMDGTLLKSAVGPTCPATVSSLVKPGLNCPSIPKPTLPSPGQILNGKGLPAPPTLEKKPEDNSNNRKFLNKRLSEREFDPDIHCGVIDLDTKKPCTRSLTCKTHSLTQRRAVQGRRKRFDVLLAEHKNKTREKELIRHPDSQQPPQPLRDPHPAPPRTSQEPHQNPHGVIPSESKPFVASKPKPHTPSLPRPPGCPAQQGGSAPIDPPPVHESPHPPLPATEPASRLSSEEGEGDDKEESVEKLDCHYSGHHPQPASFCTFGSRQIGRGYYVFDSRWNRLRCALNLMVEKHLNAQLWKKIPPVPSTTSPISTRIPHRTNSVPTSQCGVSYLAAATVSTSPVLLSSTCISPNSKSVPAHGTTLNAQPAASGAMDPVCSMQSRQVSSSSSSPSTPSGLSSVPSSPMSRKPQKLKSSKSLRPKESSGNSTNCQNASSSTSGGSGKKRKNSSPLLVHSSSSSSSSSSSSHSMESFRKNCVAHSGPPYPSTVTSSHSIGLNCVTNKANAVNVRHDQSGRGPPTGSPAESIKRMSVMVNSSDSTLSLGPFIHQSNELPVNSHGSFSHSHTPLDKLIGKKRKCSPSSSSINNSSSKPTKVAKVPAVNNVHMKHTGTIPGAQGLMNSSLLHQPKARP

SEQ ID NO: 53 - protein, 612 aa, Homo sapiens
MATAATIPSVATATAAALGEVEDEGLLASLFRDRFPEAQWRERPDVGRYLRELSGSGLERLRREPERLAEERAQLLQQTRDLAFANYKTFIRGAECTERIHRLFGDVEASLGRLLDRLPSFQQSCRNFVKEAEEISSNRRMNSLTLNRHTEILEILEIPQLMDTCVRNSYYEEALELAAYVRRLERKYSSIPVIQGIVNEVRQSMQLMLSQLIQQLRTNIQLPACLRVIGYLRRMDVFTEAELRVKFLQARDAWLRSILTAIPNDDPYFHITKTIEASRVHLFDIITQYRAIFSDEDPLLPPAMGEHTVNESAIFHGWVLQKVSQFLQVLETDLYRGIGGHLDSLLGQCMYFGLSFSRVGADFRGQLAPVFQRVAISTFQKAIQETVEKFQEEMNSYMLISAPAILGTSNMPAAVPATQPGTLQPPMVLLDFPPLACFLNNILVAFNDLRLCCPVALAQDVTGALEDALAKVTKIILAFHRAEEAAFSSGEQELFVQFCTVFLEDLVPYLNRCLQVLFPPAQIAQTLGIPPTQLSKYGNLGHVNIGAIQEPLAFILPKRETLFTLDDQALGPELTAPAPEPPAEEPRLEPAGPACPEGGRAETQAEPPSVGP

SEQ ID NO: 54 - protein, 251 aa, Homo sapiens
MSDIGDWFRSIPAITRYWFAATVAVPLVGKLGLISPAYLFLWPEAFLYRFQIWRPITATFYFPVGPGTGFLYLVNLYFLYQYSTRLETGAFDGRPADYLFMLLFNWICIVITGLAMDMQLLMIPLIMSVLYVWAQLNRDMIVSFWFGTRFKACYLPWVILGFNYIIGGSVINELIGNLVGHLYFFLMFRYPMDLGGRNFLSTPQFLYRWLPSRRGGVSGFGVPPASMRRAADQNGGGGRHNWGQGFRLGDQ

SEQ ID NO: 55 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 56 - protein, 121 aa, Homo sapiens
MVQIVISSARAGGLAEWVLMELQGEIEARYSTGLAGNLLGDLHYTTEGIPVLIVGHHILYGKIIHLEKPFAVLVKHTPGDQDCDELGRETGTRYLVTALIKDKILFKTRPKPIITSVPKKV

SEQ ID NO: 57 - protein, 530 aa, Homo sapiens
MSMPDAMPLPGVGEELKQAKEIEDAEKYSFMATVTKAPKKQIQFADDMQEFTKFPTKTGRRSLSRSISQSSTDSYSSAASYTDSSDDEVSPREKQQTNSKGSSNFCVKNIKQAEFGRREIEIAEQDMSALISLRKRAQGEKPLAGAKIVGCTHITAQTAVLIETLCALGAQCRWSACNIYSTQNEVAAALAEAGVAVFAWKGESEDDFWWCIDRCVNMDGWQANMILDDGGDLTHWVYKKYPNVFKKIRGIVEESVTGVHRLYQLSKAGKLCVPAMNVNDSVTKQKFDNLYCCRESILDGLKRTTDVMFGGKQVVVCGYGEVGKGCCAALKALGAIVYITEIDPICALQACMDGFRVVKLNEVIRQVDVVITCTGNKNVVTREHLDRMKNSCIVCNMGHSNTEIDVTSLRTPELTWERVRSQVDHVIWPDGKRVVLLAEGRLLNLSCSTVPTFVLSITATTQALALIELYNAPEGRYKQDVYLLPKKMDEYVASLHLPSFDAHLTELTDDQAKYLGLNKNGPFKPNYYRY

SEQ ID NO: 58 - protein, 431 aa, Homo sapiens
MSWVQATLLARGLCRAWGGTCGAALTGTSISQVPRRLPRGLHCSAAAHSSEQSLVPSPPEPRQRPTKALVPFEDLFGQAPGGERDKASFLQTVQKFAEHSVRKRGHIDFIYLALRKMREYGVERDLAVYNQLLNIFPKEVFRPRNIIQRIFVHYPRQQECGIAVLEQMENHGVMPNKETEFLLIQIFGRKSYPMLKLVRLKLWFPRFMNVNPFPVPRDLPQDPVELAMFGLRHMEPDLSARVTIYQVPLPKDSTGAADPPQPHIVGIQSPDQQAALARHNPARPVFVEGPFSLWLRNKCVYYHILRADLLPPEEREVEETPEEWNLYYPMQLDLEYVRSGWDNYEFDINEVEEGPVFAMCMAGAHDQATMAKWIQGLQETNPTLAQIPVVFRLAGSTRELQTSSAGLEEPPLPEDHQEEDDNLQRQQQGQS

SEQ ID NO: 59 - protein, 707 aa, Homo sapiens
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 60 - protein, 262 aa, Homo sapiens
MLALEAAQLDGPHFSCLYPDGVFYDLDSCKHSSYPDSEGAPDSLWDWTVAPPVPATPYEAFDPAAAAFSHPQAAQLCYEPPTYSPAGNLELAPSLEAPGPGLPAYPTENFASQTLVPPAYAPYPSPVLSEEEDLPLDSPALEVSDSESDEALVAGPEGKGSEAGTRKKLRLYQFLLGLLTRGDMRECVWWVEPGAGVFQFSSKHKELLARRWGQQKGNRKRMTYQKLARALRNYAKTGEIRKVKRKLTYQFDSALLPAVRRA

SEQ ID NO: 61 - protein, 325 aa, Homo sapiens
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKNQRKERKSKSSRDAKSKAKRKSCGDSSPDTFSDGLSSSTLPDDHSSYTVPGYMQDLEVEQALTPALSPCAVSSTLPDWHIPVEVVPDSTSDLYNFQVSPMPSTSEATTDEDEEGKLPEDIMKLLEQSEWQPTNVDGKGYLLNEPGVQPTSVYGDFSCKEEPEIDSPGGDIGLSLQRVFTDLKNMDATWLDSLLTPVRLPSIQAIPCAP

SEQ ID NO: 62 - protein, 1866 aa, Homo sapiens
MDPVGLQLGNKNLWSCLVRLLTKDPEWLNAKMKFFLPNTDLDSRNETLDPEQRVILQLNKLHVQGSDTWQSFIHCVCMQLEVPLDLEVLLLSTFGYDDGFTSQLGAEGKSQPESQLHHGLKRPHQSCGSSPRRKQCKKQQLELAKKYLQLLRTSAQQRYRSQIPGSGQPHAFHQVYVPPILRRATASLDTPEGAIMGDVKVEDGADVSISDLFNTRVNKGPRVTVLLGKAGMGKTTLAHRLCQKWAEGHLNCFQALFLFEFRQLNLITRFLTPSELLFDLYLSPESDHDTVFQYLEKNADQVLLIFDGLDEALQPMGPDGPGPVLTLFSHLCNGTLLPGCRVMATSRPGKLPACLPAEAAMVHMLGFDGPRVEEYVNHFFSAQPSREGALVELQTNGRLRSLCAVPALCQVACLCLHHLLPDHAPGQSVALLPNMTQLYMQMVLALSPPGHLPTSSLLDLGEVALRGLETGKVIFYAKDIAPPLIAFGATHSLLTSFCVCTGPGHQQTGYAFTHLSLQEFLAALHLMASPKVNKDTLTQYVTLHSRWVQRTKARLGLSDHLPTFLAGLASCTCRPFLSHLAQGNEDCVGAKQAAVVQVLKKLATRKLTGPKVVELCHCVDETQEPELASLTAQSLPYQLPFHNFPLTCTDLATLTNILEHREAPIHLDFDGCPLEPHCPEALVGCGQIENLSFKSRKCGDAFAEALSRSLPTMGRLQMLGLAGSKITARGISHLVKALPLCPQLKEVSFRDNQLSDQVVLNIVEVLPHLPRLRKLDLSSNSICVSTLLCLARVAVTCPTVRMLQAREADLIFLLSPPTETTAELQRAPDLQESDGQRKGAQSRSLTLRLQKCQLQVHDAEALIALLQEGPHLEEVDLSGNQLEDEGCRLMAEAASQLHIARKLDLSNNGLSVAGVHCVLRAVSACWTLAELHISLQHKTVIFMFAQEPEEQKGPQERAAFLDSLMLQMPSELPLSSRRMRLTHCGLQEKHLEQLCKALGGSCHLGHLHLDFSGNALGDEGAARLAQLLPGLGALQSLNLSENGLSLDAVLGLVRCFSTLQWLFRLDISFESQHILLRGDKTSRDMWATGSLPDFPAAAKFLGFRQRCIPRSLCLSECPLEPPSLTRLCATLKDCPGPLELQLSCEFLSDQSLETLLDCLPQLPQLSLLQLSQTGLSPKSPFLLANTLSLCPRVKKVDLRSLHHATLHFRSNEEEEGVCCGRFTGCSLSQEHVESLCWLLSKCKDLSQVDLSANLLGDSGLRCLLECLPQVPISGLLDLSHNSISQESALYLLETLPSCPRVREASVNLGSEQSFRIHFSREDQAGKTLRLSECSFRPEHVSRLATGLSKSLQLTELTLTQCCLGQKQLAILLSLVGRPAGLFSLRVQEPWADRARVLSLLEVCAQASGSVTEISISETQQQLCVQLEFPRQEENPEAVALRLAHCDLGAHHSLLVGQLMETCARLQQLSLSQVNLCEDDDASSLLLQSLLLSLSELKTFRLTSSCVSTEGLAHLASGLGHCHHLEELDLSNNQFDEEGTKALMRALEGKWMLKRLDLSHLLLNSSTLALLTHRLSQMTCLQSLRLNRNSIGDVGCCHLSEALRAATSLEELDLSHNQIGDAGVQHLATILPGLPELRKIDLSGNSISSAGGVQLAESLVLCRRLEELMLGCNALGDPTALGLAQELPQHLRVLHLPFSHLGPGGALSLAQALDGSPHLEEISLAENNLAGGVLRFCMELPLLRQIDLVSCKIDNQTAKLLTSSFTSCPALEVILLSWNLLGDEAAAELAQVLPQMGRLKRVDLEKNQITALGAWLLAEGLAQGSSIQVIRLWNNPIPCDMAQHLKSQEPRLDFAFFDNQPQAPWGT

SEQ ID NO: 63 - protein, 426 aa, Homo sapiens
MCDRNGGRRLRQWLIEQIDSSMYPGLIWENEEKSMFRIPWKHAGKQDYNQEVDASIFKAWAVFKGKFKEGDKAEPATWKTRLRCALNKSPDFEEVTDRSQLDISEPYKVYRIVPEEEQKCKLGVATAGCVNEVTEMECGRSEIDELIKEPSVDDYMGMIKRSPSPPEACRSQLLPDWWAQQPSTGVPLVTGYTTYDAHHSAFSQMVISFYYGGKLVGQATTTCPEGCRLSLSQPGLPGTKLYGPEGLELVRFPPADAIPSERQRQVTRKLFGHLERGVLLHSSRQGVFVKRLCQGRVFCSGNAVVCKGRPNKLERDEVVQVFDTSQFFRELQQFYNSQGRLPDGRVVLCFGEEFPDMAPLRSKLILVQIEQLYVRQLAEEAGKSCGAGSVMQAPEEPPPDQVFRMFPDICASHQRSFFRENQQITV

SEQ ID NO: 64 - protein, 99 aa, Homo sapiens
MAAAAASRGVGAKLGLREIRIHLCQRSPGSQGVRDFIEKRYVELKKANPDLPILIRECSDVQPKLWARYAFGQETNVPLNNFSADQVTRALENVLSGKA

SEQ ID NO: 65 - protein, 464 aa, Homo sapiens
MLATRRLLGWSLPARVSVRFSGDTTAPKKTSFGSLKDEDRIFTNLYGRHDWRLKGSLSRGDWYKTKEILLKGPDWILGEIKTSGLRGRGGAGFPTGLKWSFMNKPSDGRPKYLVVNADEGEPGTCKDREILRHDPHKLLEGCLVGGRAMGARAAYIYIRGEFYNEASNLQVAIREAYEAGLIGKNACGSGYDFDVFVVRGAGAYICGEETALIESIEGKQGKPRLKPPFPADVGVFGCPTTVANVETVAVSPTICRRGGTWFAGFGRERNSGTKLFNISGHVNHPCTVEEEMSVPLKELIEKHAGGVTGGWDNLLAVIPGGSSTPLIPKSVCETVLMDFDALVQAQTGLGTAAVIVMDRSTDIVKAIARLIEFYKHESCGQCTPCREGVDWMNKVMARFVRGDARPAEIDSLWEISKQIEGHTICALGDGAAWPVQGLIRHFRPELEERMQRFAQQHQARQAAS

SEQ ID NO: 66 - protein, 144 aa, Homo sapiens
MAASKVKQDMPPPGGYGPIDYKRNLPRRGLSGYSMLAIGIGTLIYGHWSIMKWNRERRRLQIEDFEARIALLPLLQAETDRRTLQMLRENLEEEAIIMKDVPDWKVGESVFHTTRWVPPLIGELYGLRTTEEALHASHGFMWYT

SEQ ID NO: 67 - protein, 1102 aa, Homo sapiens
MNHQQQQQQQKAGEQQLSEPEDMEMEAGDTDDPPRITQNPVINGNVALSDGHNTAEEDMEDDTSWRSEATFQFTVERFSRLSESVLSPPCFVRNLPWKIMVMPRFYPDRPHQKSVGFFLQCNAESDSTSWSCHAQAVLKIINYRDDEKSFSRRISHLFFHKENDWGFSNFMAWSEVTDPEKGFIDDDKVTFEVFVQADAPHGVAWDSKKHTGYVGLKNQGATCYMNSLLQTLFFTNQLRKAVYMMPTEGDDSSKSVPLALQRVFYELQHSDKPVGTKKLTKSFGWETLDSFMQHDVQELCRVLLDNVENKMKGTCVEGTIPKLFRGKMVSYIQCKEVDYRSDRREDYYDIQLSIKGKKNIFESFVDYVAVEQLDGDNKYDAGEHGLQEAEKGVKFLTLPPVLHLQLMRFMYDPQTDQNIKINDRFEFPEQLPLDEFLQKTDPKDPANYILHAVLVHSGDNHGGHYVVYLNPKGDGKWCKFDDDVVSRCTKEEAIEHNYGGHDDDLSVRHCTNAYMLVYIRESKLSEVLQAVTDHDIPQQLVERLQEEKRIEAQKRKERQEAHLYMQVQIVAEDQFCGHQGNDMYDEEKVKYTVFKVLKNSSLAEFVQSLSQTMGFPQDQIRLWPMQARSNGTKRPAMLDNEADGNKTMIELSDNENPWTIFLETVDPELAASGATLPKFDKDHDVMLFLKMYDPKTRSLNYCGHIYTPISCKIRDLLPVMCDRAGFIQDTSLILYEEVKPNLTERIQDYDVSLDKALDELMDGDIIVFQKDDPENDNSELPTAKEYFRDLYHRVDVIFCDKTIPNDPGFVVTLSNRMNYFQVAKTVAQRLNTDPMLLQFFKSQGYRDGPGNPLRHNYEGTLRDLLQFFKPRQPKKLYYQQLKMKITDFENRRSFKCIWLNSQFREEEITLYPDKHGCVRDLLEECKKAVELGEKASGKLRLLEIVSYKIIGVHQEDELLECLSPATSRTFRIEEIPLDQVDIDKENEMLVTVAHFHKEVFGTFGIPFLLRIHQGEHFREVMKRIQSLLDIQEKEFEKFKFAIVMMGRHQYINEDEYEVNLKDFEPQPGNMSHPRPWLGLDHFNKAPKRSRYTYLEKAIKIHN

SEQ ID NO: 68 - protein, 74 aa, Homo sapiens
MSANGAVWGRVRSRLRAFPERLAACGAEAAAYGRCVQASTAPGGRLSKDFCAREFEALRSCFAAAAKKTLEGGC

SEQ ID NO: 69 - protein, 272 aa, Homo sapiens
MEAQGVAEGAGPGAASGVPHPAALAPAAAPTLAPASVAAAASQFTLLVMQPCAGQDEAAAPGGSVGAGKPVRYLCEGAGDGEEEAGEDEADLLDTSDPPGGGESAASLEDLEDEETHSGGEGSSGGARRRGSGGGSMSKTCTYEGCSETTSQVAKQRKPWMCKKHRNKMYKDKYKKKKSDQALNCGGTASTGSAGNVKLEESADNILSIVKQRTGSFGDRPARPTLLEQVLNQKRLSLLRSPEVVQFLQKQQQLLNQQVLEQRQQQFPGTSM

SEQ ID NO: 70 - protein, 152 aa, Homo sapiens
MSTPARRRLMRDFKRLQEDPPAGVSGAPSENNIMVWNAVIFGPEGTPFEDGTFKLTIEFTEEYPNKPPTVRFVSKMFHPNVYADGSICLDILQNRWSPTYDVSSILTSIQSLLDEPNPNSPANSQAAQLYQENKREYEKRVSAIVEQSWRDC

SEQ ID NO: 71 - protein, 655 aa, Homo sapiens
MERKVLALQARKKRTKAKKDKAQRKSETQHRGSAPHSESDLPEQEEEILGSDDDEQEDPNDYCKGGYHLVKIGDLFNGRYHVIRKLGWGHFSTVWLSWDIQGKKFVAMKVVKSAEHYTETALDEIRLLKSVRNSDPNDPNREMVVQLLDDFKISGVNGTHICMVFEVLGHHLLKWIIKSNYQGLPLPCVKKIIQQVLQGLDYLHTKCRIIHTDIKPENILLSVNEQYIRRLAAEATEWQRSGAPPPSGSAVSTAPQPKPADKMSKNKKKKLKKKQKRQAELLEKRMQEIEEMEKESGPGQKRPNKQEESESPVERPLKENPPNKMTQEKLEESSTIGQDQTLMERDTEGGAAEINCNGVIEVINYTQNSNNETLRHKEDLHNANDCDVQNLNQESSFLSSQNGDSSTSQETDSCTPITSEVSDTMVCQSSSTVGQSFSEQHISQLQESIRAEIPCEDEQEQEHNGPLDNKGKSTAGNFLVNPLEPKNAEKLKVKIADLGNACWVHKHFTEDIQTRQYRSLEVLIGSGYNTPADIWSTACMAFELATGDYLFEPHSGEEYTRDEDHIALIIELLGKVPRKLIVAGKYSKEFFTKKGDLKHITKLKPWGLFEVLVEKYEWSQEEAAGFTDFLLPMLELIPEKRATAAECLRHPWLNS

SEQ ID NO: 72 - protein, 213 aa, Homo sapiens
MAVLSAPGLRGFRILGLRSSVGPAVQARGVHQSVATDGPSSTQPALPKARAVAPKPSSRGEYVVAKLDDLVNWARRSSLWPMTFGLACCAVEMMHMAAPRYDMDRFGVVFRASPRQSDVMIVAGTLTNKMAPALRKVYDQMPEPRYVVSMGSCANGGGYYHYSYSVVRGCDRIVPVDIYIPGCPPTAEALLYGILQLQRKIKRERRLQIWYRR

SEQ ID NO: 73 - protein, 1447 aa, Homo sapiens
MAHSKTRTNDGKITYPPGVKEISDKISKEEMVRRLKMVVKTFMDMDQDSEEEKELYLNLALHLASDFFLKHPDKDVRLLVACCLADIFRIYAPEAPYTSPDKLKDIFMFITRQLKGLEDTKSPQFNRYFYLLENIAWVKSYNICFELEDSNEIFTQLYRTLFSVINNGHNQKVHMHMVDLMSSIICEGDTVSQELLDTVLVNLVPAHKNLNKQAYDLAKALLKRTAQAIEPYITNFFNQVLMLGKTSISDLSEHVFDLILELYNIDSHLLLSVLPQLEFKLKSNDNEERLQVVKLLAKMFGAKDSELASQNKPLWQCYLGRFNDIHVPIRLECVKFASHCLMNHPDLAKDLTEYLKVRSHDPEEAIRHDVIVSIVTAAKKDILLVNDHLLNFVRERTLDKRWRVRKEAMMGLAQIYKKYALQSAAGKDAAKQIAWIKDKLLHIYYQNSIDDRLLVERIFAQYMVPHNLETTERMKCLYYLYATLDLNAVKALNEMWKCQNLLRHQVKDLLDLIKQPKTDASVKAIFSKVMVITRNLPDPGKAQDFMKKFTQVLEDDEKIRKQLEVLVSPTCSCKQAEGCVREITKKLGNPKQPTNPFLEMIKFLLERIAPVHIDTESISALIKQVNKSIDGTADDEDEGVPTDQAIRAGLELLKVLSFTHPISFHSAETFESLLACLKMDDEKVAEAALQIFKNTGSKIEEDFPHIRSALLPVLHHKSKKGPPRQAKYAIHCIHAIFSSKETQFAQIFEPLHKSLDPSNLEHLITPLVTIGHIALLAPDQFAAPLKSLVATFIVKDLLMNDRLPGKKTTKLWVPDEEVSPETMVKIQAIKMMVRWLLGMKNNHSKSGTSTLRLLTTILHSDGDLTEQGKISKPDMSRLRLAAGSAIVKLAQEPCYHEIITLEQYQLCALAINDECYQVRQVFAQKLHKGLSRLRLPLEYMAICALCAKDPVKERRAHARQCLVKNINVRREYLKQHAAVSEKLLSLLPEYVVPYTIHLLAHDPDYVKVQDIEQLKDVKECLWFVLEILMAKNENNSHAFIRKMVENIKQTKDAQGPDDAKMNEKLYTVCDVAMNIIMSKSTTYSLESPKDPVLPARFFTQPDKNFSNTKNYLPPEMKSFFTPGKPKTTNVLGAVNKPLSSAGKQSQTKSSRMETVSNASSSSNPSSPGRIKGRLDSSEMDHSENEDYTMSSPLPGKKSDKRDDSDLVRSELEKPRGRKKTPVTEQEEKLGMDDLTKLVQEQKPKGSQRSRKRGHTASESDEQQWPEEKRLKEDILENEDEQNSPPKKGKRGRPPKPLGGGTPKEEPTMKTSKKGSKKKSGPPAPEEEEEEERQSGNTEQKSKSKQHRVSRRAQQRAESPESSAIESTQSTPQKGRGRPSKTPSPSQPKKNVRVGRSKQAATKENDSSEEVDVFQGSSPVDDIPQEETEEEEVSTVNVRRRSAKRERR

SEQ ID NO: 74 - protein, 510 aa, Homo sapiens
MPGGGASAASGRLLTAAEQRGSREAAGSASRSGFGGSGGGRGGASGPGSGSGGPGGPAGRMSLTPKELSSLLSIISEEAGGGSTFEGLSTAFHHYFSKADHFRLGSVLVMLLQQPDLLPSAAQRLTALYLLWEMYRTEPLAANPFAASFAHLLNPAPPARGGQEPDRPPLSGFLPPITPPEKFFLSQLMLAPPRELFKKTPRQIALMDVGNMGQSVDISGLQLALAERQSELPTQSKASFPSILSDPDPDSSNSGFDSSVASQITEALVSGPKPPIESHFRPEFIRPPPPLHICEDELAWLNPTEPDHAIQWDKSMCVKNSTGVEIKRIMAKAFKSPLSSPQQTQLLGELEKDPKLVYHIGLTPAKLPDLVENNPLVAIEMLLKLMQSSQITEYFSVLVNMDMSLHSMEVVNRLTTAVDLPPEFIHLYISNCISTCEQIKDKYMQNRLVRLVCVFLQSLIRNKIINVQDLFIEVQAFCIEFSRIREAAGLFRLLKTLDTGETPSETKMSK

SEQ ID NO: 75 - protein, 137 aa, Homo sapiens
MGAHLVRRYLGDASVEPDPLQMPTFPPDYGFPERKEREMVATQQEMMDAQLRLQLRDYCAHHLIRLLKCKRDSFPNFLACKQERHDWDYCEHRDYVMRMKEFERERRLLQRKKRREKKAAELAKGQGPGEVDPKVAL

SEQ ID NO: 76 - protein, 334 aa, Homo sapiens
MKMASSLAFLLLNFHVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPWIAALAGTLPILLLLLAGASYFLWRQQKEITALSSEIESEQEMKEMGYAATEREISLRESLQEELKRKKIQYLTRGEESSSDTNKSA

SEQ ID NO: 77 - protein, 486 aa, Homo sapiens
MIRRVLPHGMGRGLLTRRPGTRRGGFSLDWDGKVSEIKKKIKSILPGRSCDLLQDTSHLPPEHSDVVIVGGGVLGLSVAYWLKKLESRRGAIRVLVVERDHTYSQASTGLSVGGICQQFSLPENIQLSLFSASFLRNINEYLAVVDAPPLDLRFNPSGYLLLASEKDAAAMESNVKVQRQEGAKVSLMSPDQLRNKFPWINTEGVALASYGMEDEGWFDPWCLLQGLRRKVQSLGVLFCQGEVTRFVSSSQRMLTTDDKAVVLKRIHEVHVKMDRSLEYQPVECAIVINAAGAWSAQIAALAGVGEGPPGTLQGTKLPVEPRKRYVYVWHCPQGPGLETPLVADTSGAYFRREGLGSNYLGGRSPTEQEEPDPANLEVDHDFFQDKVWPHLALRVPAFETLKVQSAWAGYYDYNTFDQNGVVGPHPLVVNMYFATGFSGHGLQQAPGIGRAVAEMVLKGRFQTIDLSPFLFTRFYLGEKIQENNII

SEQ ID NO: 78 - protein, 210 aa, Homo sapiens
MRCLTTPMLLRALAQAARAGPPGGRSLHSSAVAATYKYVNMQDPEMDMKSVTDRAARTLLWTELFRGLGMTLSYLFREPATINYPFEKGPLSPRFRGEHALRRYPSGEERCIACKLCEAICPAQAITIEAEPRADGSRRTTRYDIDMTKCIYCGFCQEACPVDAIVEGPNFEFSTETHEELLYNKEKLLNNGDKWEAEIAANIQADYLYR

SEQ ID NO: 79 - protein, 403 aa, Homo sapiens
MNHKSKKRIREAKRSARPELKDSLDWTRHNYYESFSLSPAAVADNVERADALQLSVEEFVERYERPYKPVVLLNAQEGWSAQEKWTLERLKRKYRNQKFKCGEDNDGYSVKMKMKYYIEYMESTRDDSPLYIFDSSYGEHPKRRKLLEDYKVPKFFTDDLFQYAGEKRRPPYRWFVMGPPRSGTGIHIDPLGTSAWNALVQGHKRWCLFPTSTPRELIKVTRDEGGNQQDEAITWFNVIYPRTQLPTWPPEFKPLEILQKPGETVFVPGGWWHVVLNLDTTIAITQNFASSTNFPVVWHKTVRGRPKLSRKWYRILKQEHPELAVLADSVDLQESTGIASDSSSDSSSSSSSSSSDSDSECESGSEGDGTVHRRKKRRTCSMVGNGDTTSQDDCVSKERSSSR

SEQ ID NO: 80 - protein, 463 aa, Homo sapiens
MAALRALCGFRGVAAQVLRPGAGVRLPIQPSRGVRQWQPDVEWAQQFGGAVMYPSKETAHWKPPPWNDVDPPKDTIVKNITLNFGPQHPAAHGVLRLVMELSGEMVRKCDPHIGLLHRGTEKLIEYKTYLQALPYFDRLDYVSMMCNEQAYSLAVEKLLNIRPPPRAQWIRVLFGEITRLLNHIMAVTTHALDLGAMTPFFWLFEEREKMFEFYERVSGARMHAAYIRPGGVHQDLPLGLMDDIYQFSKNFSLRLDELEELLTNNRIWRNRTIDIGVVTAEEALNYGFSGVMLRGSGIQWDLRKTQPYDVYDQVEFDVPVGSRGDCYDRYLCRVEEMRQSLRIIAQCLNKMPPGEIKVDDAKVSPPKRAEMKTSMESLIHHFKLYTEGYQVPPGATYTAIEAPKGEFGVYLVSDGSSRPYRCKIKAPGFAHLAGLDKMSKGHMLADVVAIIGTQDIVFGEVDR

SEQ ID NO: 81 - protein, 119 aa, Homo sapiens
MIARRNPEPLRFLPDEARSLPPPKLTDPRLLYIGFLGYCSGLIDNLIRRRPIATAGLHRQLLYITAFFFAGYYLVKREDYLYAVRDREMFGYMKLHPEDFPEEDKKTYGEIFEKFHPIR

SEQ ID NO: 82 - protein, 529 aa, Homo sapiens
MDLPVGPGAAGPSNVPAFLTKLWTLVSDPDTDALICWSPSGNSFHVFDQGQFAKEVLPKYFKHNNMASFVRQLNMYGFRKVVHIEQGGLVKPERDDTEFQHPCFLRGQEQLLENIKRKVTSVSTLKSEDIKIRQDSVTKLLTDVQLMKGKQECMDSKLLAMKHENEALWREVASLRQKHAQQQKVVNKLIQFLISLVQSNRILGVKRKIPLMLNDSGSAHSMPKYSRQFSLEHVHGSGPYSAPSPAYSSSSLYAPDAVASSGPIISDITELAPASPMASPGGSIDERPLSSSPLVRVKEEPPSPPQSPRVEEASPGRPSSVDTLLSPTALIDSILRESEPAPASVTALTDARGHTDTEGRPPSPPPTSTPEKCLSVACLDKNELSDHLDAMDSNLDNLQTMLSSHGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 83 - protein, 621 aa, Homo sapiens
MSGCGLFLRTTAAARACRGLVVSTANRRLLRTSPPVRAFAKELFLGKIKKKEVFPFPEVSQDELNEINQFLGPVEKFFTEEVDSRKIDQEGKIPDETLEKLKSLGLFGLQVPEEYGGLGFSNTMYSRLGEIISMDGSITVTLAAHQAIGLKGIILAGTEEQKAKYLPKLASGEHIAAFCLTEPASGSDAASIRSRATLSEDKKHYILNGSKVWITNGGLANIFTVFAKTEVVDSDGSVKDKITAFIVERDFGGVTNGKPEDKLGIRGSNTCEVHFENTKIPVENILGEVGDGFKVAMNILNSGRFSMGSVVAGLLKRLIEMTAEYACTRKQFNKRLSEFGLIQEKFALMAQKAYVMESMTYLTAGMLDQPGFPDCSIEAAMVKVFSSEAAWQCVSEALQILGGLGYTRDYPYERILRDTRILLIFEGTNEILRMYIALTGLQHAGRILTTRIHELKQAKVSTVMDTVGRRLRDSLGRTVDLGLTGNHGVVHPSLADSANKFEENTYCFGRTVETLLLRFGKTIMEEQLVLKRVANILINLYGMTAVLSRASRSIRIGLRNHDHEVLLANTFCVEAYLQNLFSLSQLDKYAPENLDEQIKKVSQQILEKRAYICAHPLDRTC

SEQ ID NO: 84 - protein, 345 aa, Homo sapiens
MLRPAGLWRLCRRPWAARVPAENLGRREVTSGVSPRGSTSPRTLNIFDRDLKRKQKNWAARQPEPTKFDYLKEEVGSRIADRVYDIPRNFPLALDLGCGRGYIAQYLNKETIGKFFQADIAENALKNSSETEIPTVSVLADEEFLPFKENTFDLVVSSLSLHWVNDLPRALEQIHYILKPDGVFIGAMFGGDTLYELRCSLQLAETEREGGFSPHISPFTAVNDLGHLLGRAGFNTLTVDTDEIQVNYPGMFELMEDLQGMGESNCAWNRKALLHRDTMLAAAAVYREMYRNEDGSVPATYQIYYMIGWKYHESQARPAERGSATVSFGELGKINNLMPPGKKSQ

SEQ ID NO: 85 - protein, 285 aa, Homo sapiens
MEVPPPAPRSFLCRALCLFPRVFAAEAVTADSEVLEERQKRLPYVPEPYYPESGWDRLRELFGKDEQQRISKDLANICKTAATAGIIGWVYGGIPAFIHAKQQYIEQSQAEIYHNRFDAVQSAHRAATRGFIRYGWRWGWRTAVFVTIFNTVNTSLNVYRNKDALSHFVIAGAVTGSLFRINVGLRGLVAGGIIGALLGTPVGGLLMAFQKYSGETVQERKQKDRKALHELKLEEWKGRLQVTEHLPEKIESSLQEDEPENDAKKIEALLNLPRNPSVIDKQDKD

SEQ ID NO: 86 - protein, 261 aa, Homo sapiens
MAAACRSVKGLVAVITGGASGLGLATAERLVGQGASAVLLDLPNSGGEAQAKKLGNNCVFAPADVTSEKDVQTALALAKGKFGRVDVAVNCAGIAVASKTYNLKKGQTHTLEDFQRVLDVNLMGTFNVIRLVAGEMGQNEPDQGGQRGVIINTASVAAFEGQVGQAAYSASKGGIVGMTLPIARDLAPIGIRVMTIAPGLFGTPLLTSLPEKVCNFLASQVPFPSRLGDPAEYAHLVQAIIENPFLNGEVIRLDGAIRMQP

SEQ ID NO: 87 - protein, 801 aa, Homo sapiens
MLQNVTPHNKLPGEGNAGLLGLGPEAAAPGKRIRKPSLLYEGFESPTMASVPALQLTPANPPPPEVSNPKKPGRVTNQLQYLHKVVMKALWKHQFAWPFRQPVDAVKLGLPDYHKIIKQPMDMGTIKRRLENNYYWAASECMQDFNTMFTNCYIYNKPTDDIVLMAQTLEKIFLQKVASMPQEEQELVVTIPKNSHKKGAKLAALQGSVTSAHQVPAVSSVSHTALYTPPPEIPTTVLNIPHPSVISSPLLKSLHSAGPPLLAVTAAPPAQPLAKKKGVKRKADTTTPTPTAILAPGSPASPPGSLEPKAARLPPMRRESGRPIKPPRKDLPDSQQQHQSSKKGKLSEQLKHCNGILKELLSKKHAAYAWPFYKPVDASALGLHDYHDIIKHPMDLSTVKRKMENRDYRDAQEFAADVRLMFSNCYKYNPPDHDVVAMARKLQDVFEFRYAKMPDEPLEPGPLPVSTAMPPGLAKSSSESSSEESSSESSSEEEEEEDEEDEEEEESESSDSEEERAHRLAELQEQLRAVHEQLAALSQGPISKPKRKREKKEKKKKRKAEKHRGRAGADEDDKGPRAPRPPQPKKSKKASGSGGGSAALGPSGFGPSGGSGTKLPKKATKTAPPALPTGYDSEEEEESRPMSYDEKRQLSLDINKLPGEKLGRVVHIIQAREPSLRDSNPEEIEIDFETLKPSTLRELERYVLSCLRKKPRKPYTIKKPVGKTKEELALEKKRELEKRLQDVSGQLNSTKKPPKKANEKTESSSAQQVAVSRLSASSSSSDSSSSSSSSSSSDTSDSDSG

SEQ ID NO: 88 - protein, 128 aa, Homo sapiens
MAGSGVRQATSTASTFVKPIFSRDMNEAKRRVRELYRAWYREVPNTVHQFQLDITVKMGRDKVREMFMKNAHVTDPRVVDLLVIKGKIELEETIKVWKQRTHVMRFFHETEAPRPKDFLSKFYVGHDP

SEQ ID NO: 89 - protein, 575 aa, Homo sapiens
MSRSPDAKEDPVECPLCMEPLEIDDINFFPCTCGYQICRFCWHRIRTDENGLCPACRKPYPEDPAVYKPLSQEELQRIKNEKKQKQNERKQKISENRKHLASVRVVQKNLVFVVGLSQRLADPEVLKRPEYFGKFGKIHKVVINNSTSYAGSQGPSASAYVTYIRSEDALRAIQCVNNVVVDGRTLKASLGTTKYCSYFLKNMQCPKPDCMYLHELGDEAASFTKEEMQAGKHQEYEQKLLQELYKLNPNFLQLSTGSVDKNKNKVTPLQRYDTPIDKPSDSLSIGNGDNSQQISNSDTPSPPPGLSKSNPVIPISSSNHSARSPFEGAVTESQSLFSDNFRHPNPIPSGLPPFPSSPQTSSDWPTAPEPQSLFTSETIPVSSSTDWQAAFGFGSSKQPEDDLGFDPFDVTRKALADLIEKELSVQDQPSLSPTSLQNSSSHTTTAKGPGSGFLHPAAATNANSLNSTFSVLPQRFPQFQQHRAVYNSFSFPGQAARYPWMAFPRNSIMHLNHTANPTSNSNFLDLNLPPQHNTGLGGIPVAGEEEVKVSTMPLSTSSHSLQQGQQPTSLHTTVA

SEQ ID NO: 90 - protein, 270 aa, Homo sapiens
MLQACKMEGFPLVPPPSEDLVPYDTDLYQRQTHEYYPYLSSDGESHSDHYWDFHPHHVHSEFESFAENNFTELQSVQPPQLQQLYRHMELEQMHVLDTPMVPPHPSLGHQVSYLPRMCLQYPSLSPAQPSSDEEEGERQSPPLEVSDGEADGLEPGPGLLPGETGSKKKIRLYQFLLDLLRSGDMKDSIWWVDKDKGTFQFSSKHKEALAHRWGIQKGNRKKMTYQKMARALRNYGKTGEVKKVKKKLTYQFSGEVLGRGGLAERRHPPH

SEQ ID NO: 91 - protein, 338 aa, Homo sapiens
MLSALARPASAALRRSFSTSAQNNAKVAVLGASGGIGQPLSLLLKNSPLVSRLTLYDIAHTPGVAADLSHIETKAAVKGYLGPEQLPDCLKGCDVVVIPAGVPRKPGMTRDDLFNTNATIVATLTAACAQHCPEAMICVIANPVNSTIPITAEVFKKHGVYNPNKIFGVTTLDIVRANTFVAELKGLDPARVNVPVIGGHAGKTIIPLISQCTPKVDFPQDQLTALTGRIQEAGTEVVKAKAGAGSATLSMAYAGARFVFSLVDAMNGKEGVVECSFVKSQETECTYFSTPLLLGKKGIEKNLGIGKVSSFEEKMISDAIPELKASIKKGEDFVKTLK

SEQ ID NO: 92 - protein, 645 aa, Homo sapiens
MYFPSWLSQLYRGLSRPIRRTTQPIWGSLYRSLLQSSQRRIPEFSSFVVRTNTCGELRSSHLGQEVTLCGWIQYRRQNTFLVLRDFDGLVQVIIPQDESAASVKKILCEAPVESVVQVSGTVISRPAGQENPKMPTGEIEIKVKTAELLNACKKLPFEIKNFVKKTEALRLQYRYLDLRSFQMQYNLRLRSQMVMKMREYLCNLHGFVDIETPTLFKRTPGGAKEFLVPSREPGKFYSLPQSPQQFKQLLMVGGLDRYFQVARCYRDEGSRPDRQPEFTQIDIEMSFVDQTGIQSLIEGLLQYSWPNDKDPVVVPFPTMTFAEVLATYGTDKPDTRFGMKIIDISDVFRNTEIGFLQDALSKPHGTVKAICIPEGAKYLKRKDIESIRNFAADHFNQEILPVFLNANRNWNSPVANFIMESQRLELIRLMETQEEDVVLLTAGEHNKACSLLGKLRLECADLLETRGVVLRDPTLFSFLWVVDFPLFLPKEENPRELESAHHPFTAPHPSDIHLLYTEPKKARSQHYDLVLNGNEIGGGSIRIHNAELQRYILATLLKEDVKMLSHLLQALDYGAPPHGGIALGLDRLICLVTGSPSIRDVIAFPKSFRGHDLMSNTPDSVPPEELKPYHIRVSKPTDSKAERAH

SEQ ID NO: 93 - protein, 116 aa, Homo sapiens
MGSRLSQPFESYITAPPGTAAAPAKPAPPATPGAPTSPAEHRLLKTCWSCRVLSGLGLMGAGGYVYWVARKPMKMGYPPSPWTITQMVIGLSENQGIATWGIVVMADPKGKAYRVV

SEQ ID NO: 94 - protein, 543 aa, Homo sapiens
MEQVNELKEKGNKALSVGNIDDALQCYSEAIKLDPHNHVLYSNRSAAYAKKGDYQKAYEDGCKTVDLKPDWGKGYSRKAAALEFLNRFEEAKRTYEEGLKHEANNPQLKEGLQNMEARLAERKFMNPFNMPNLYQKLESDPRTRTLLSDPTYRELIEQLRNKPSDLGTKLQDPRIMTTLSVLLGVDLGSMDEEEEIATPPPPPPPKKETKPEPMEEDLPENKKQALKEKELGNDAYKKKDFDTALKHYDKAKELDPTNMTYITNQAAVYFEKGDYNKCRELCEKAIEVGRENREDYRQIAKAYARIGNSYFKEEKYKDAIHFYNKSLAEHRTPDVLKKCQQAEKILKEQERLAYINPDLALEEKNKGNECFQKGDYPQAMKHYTEAIKRNPKDAKLYSNRAACYTKLLEFQLALKDCEECIQLEPTFIKGYTRKAAALEAMKDYTKAMDVYQKALDLDSSCKEAADGYQRCMMAQYNRHDSPEDVKRRAMADPEVQQIMSDPAMRLILEQMQKDPQALSEHLKNPVIAQKIQKLMDVGLIAIR

SEQ ID NO: 95 - protein, 364 aa, Homo sapiens
MTSELDIFVGNTTLIDEDVYRLWLDGYSVTDAVALRVRSGILEQTGATAAVLQSDTMDHYRTFHMLERLLHAPPKLLHQLIFQIPPSRQALLIERYYAFDEAFVREVLGKKLSKGTKKDLDDISTKTGITLKSCRRQFDNFKRVFKVVEEMRGSLVDNIQQHFLLSDRLARDYAAIVFFANNRFETGKKKLQYLSFGDFAFCAELMIQNWTLGAVGEAPTDPDSQMDDMDMDLDKEFLQDLKELKVLVADKDLLDLHKSLVCTALRGKLGVFSEMEANFKNLSRGLVNVAAKLTHNKDVRDLFVDLVEKFVEPCRSDHWPLSDVRFFLNQYSASVHSLDGFRHQALWDRYMGTLRGCLLRLYHD

SEQ ID NO: 96 - protein, 621 aa, Homo sapiens
MAELTVEVRGSNGAFYKGFIKDVHEDSLTVVFENNWQPERQVPFNEVRLPPPPDIKKEISEGDEVEVYSRANDQEPCGWWLAKVRMMKGEFYVIEYAACDATYNEIVTFERLRPVNQNKTVKKNTFFKCTVDVPEDLREACANENAHKDFKKAVGACRIFYHPETTQLMILSASEATVKRVNILSDMHLRSIRTKLMLMSRNEEATKHLECTKQLAAAFHEEFVVREDLMGLAIGTHGSNIQQARKVPGVTAIELDEDTGTFRIYGESADAVKKARGFLEFVEDFIQVPRNLVGKVIGKNGKVIQEIVDKSGVVRVRIEGDNENKLPREDGMVPFVFVGTKESIGNVQVLLEYHIAYLKEVEQLRMERLQIDEQLRQIGSRSYSGRGRGRRGPNYTSGYGTNSELSNPSETESERKDELSDWSLAGEDDRDSRHQRDSRRRPGGRGRSVSGGRGRGGPRGGKSSISSVLKDPDSNPYSLLDNTESDQTADTDASESHHSTNRRRRSRRRRTDEDAVLMDGMTESDTASVNENGLVTVADYISRAESQSRQRNLPRETLAKNKKEMAKDVIEEHGPSEKAINGPTSASGDDISKLQRTPGEEKINTLKEENTQEAAVLNGVS

SEQ ID NO: 97 - protein, 254 aa, Homo sapiens
MAATARRGWGAAAVAAGLRRRFCHMLKNPYTIKKQPLHQFVQRPLFPLPAAFYHPVRYMFIQTQDTPNPNSLKFIPGKPVLETRTMDFPTPAAAFRSPLARQLFRIEGVKSVFFGPDFITVTKENEELDWNLLKPDIYATIMDFFASGLPLVTEETPSGEAGSEEDDEVVAMIKELLDTRIRPTVQEDGGDVIYKGFEDGIVQLKLQGSCTSCPSSIITLKNGIQNMLQFYIPEVEGVEQVMDDESDEKEANSP

SEQ ID NO: 98 - protein, 697 aa, Homo sapiens
MARLVAVCRDGEEEFPFERRQIPLYIDDTLTMVMEFPDNVLNLDGHQNNGAQLKQFIQRHGMLKQQDLSIAMVVTSREVLSALSQLVPCVGCRRSVERLFSQLVESGNPALEPLTVGPKGVLSVTRSCMTDAKKLYTLFYVHGSKLNDMIDAIPKSKKNKRCQLHSLDTHKPKPLGGCWMDVWELMSQECRDEVVLIDSSCLLETLETYLRKHRFCTDCKNKVLRAYNILIGELDCSKEKGYCAALYEGLRCCPHERHIHVCCETDFIAHLLGRAEPEFAGGRRERHAKTIDIAQEEVLTCLGIHLYERLHRIWQKLRAEEQTWQMLFYLGVDALRKSFEMTVEKVQGISRLEQLCEEFSEEERVRELKQEKKRQKRKNRRKNKCVCDIPTPLQTADEKEVSQEKETDFIENSSCKACGSTEDGNTCVEVIVTNENTSCTCPSSGNLLGSPKIKKGLSPHCNGSDCGYSSSMEGSETGSREGSDVACTEGICNHDEHGDDSCVHHCEDKEDDGDSCVECWANSEENDTKGKNKKKKKKSKILKCDEHIQKLGSCITDPGNRETSGNTMHTVFHRDKTKDTHPESCCSSEKGGQPLPWFEHRKNVPQFAEPTETLFGPDSGKGAKSLVELLDESECTSDEEIFISQDEIQSFMANNQSFYSNREQYRQHLKEKFNKYCRLNDHKRPICSGWLTTAGAN

SEQ ID NO: 99 - protein, 851 aa, Homo sapiens
MAQWEMLQNLDSPFQDQLHQLYSHSLLPVDIRQYLAVWIEDQNWQEAALGSDDSKATMLFFHFLDQLNYECGRCSQDPESLLLQHNLRKFCRDIQPFSQDPTQLAEMIFNLLLEEKRILIQAQRAQLEQGEPVLETPVESQQHEIESRILDLRAMMEKLVKSISQLKDQQDVFCFRYKIQAKGKTPSLDPHQTKEQKILQETLNELDKRRKEVLDASKALLGRLTTLIELLLPKLEEWKAQQQKACIRAPIDHGLEQLETWFTAGAKLLFHLRQLLKELKGLSCLVSYQDDPLTKGVDLRNAQVTELLQRLLHRAFVVETQPCMPQTPHRPLILKTGSKFTVRTRLLVRLQEGNESLTVEVSIDRNPPQLQGFRKFNILTSNQKTLTPEKGQSQGLIWDFGYLTLVEQRSGGSGKGSNKGPLGVTEELHIISFTVKYTYQGLKQELKTDTLPVVIISNMNQLSIAWASVLWFNLLSPNLQNQQFFSNPPKAPWSLLGPALSWQFSSYVGRGLNSDQLSMLRNKLFGQNCRTEDPLLSWADFTKRESPPGKLPFWTWLDKILELVHDHLKDLWNDGRIMGFVSRSQERRLLKKTMSGTFLLRFSESSEGGITCSWVEHQDDDKVLIYSVQPYTKEVLQSLPLTEIIRHYQLLTEENIPENPLRFLYPRIPRDEAFGCYYQEKVNLQERRKYLKHRLIVVSNRQVDELQQPLELKPEPELESLELELGLVPEPELSLDLEPLLKAGLDLGPELESVLESTLEPVIEPTLCMVSQTVPEPDQGPVSQPVPEPDLPCDLRHLNTEPMEIFRNCVKIEEIMPNGDPLLAGQNTVDEVYVSRPSHFYTDGPLMPSDF

SEQ ID NO: 100 - protein, 331 aa, Homo sapiens
MGSAGLSRLHGLFAVYKPPGLKWKHLRDTVELQLLKGLNARKPPAPKQRVRFLLGPMEGSEEKELTLTATSVPSFINHPLVCGPAFAHLKVGVGHRLDAQASGVLVLGVGHGCRLLTDMYNAHLTKDYTVRGLLGKATDDFREDGRLVEKTTYDHVTREKLDRILAVIQGSHQKALVMYSNLDLKTQEAYEMAVRGLIRPMNKSPMLITGIRCLYFAPPEFLLEVQCMHETQKELRKLVHEIGLELKTTAVCTQVRRTRDGFFTLDSALLRTQWDLTNIQDAIRAATPQVAAELEKSLSPGLDTKQLPSPGWSWDSQGPSSTLGLERGAGQ

SEQ ID NO: 101 - protein, 4857 aa, Homo sapiens
MVTGGGAAPPGTVTEPLPSVIVLSAGRKMAAAAAAASGPGCSSAAGAGAAGVSEWLVLRDGCMHCDADGLHSLSYHPALNAILAVTSRGTIKVIDGTSGATLQASALSAKPGGQVKCQYISAVDKVIFVDDYAVGCRKDLNGILLLDTALQTPVSKQDDVVQLELPVTEAQQLLSACLEKVDISSTEGYDLFITQLKDGLKNTSHETAANHKVAKWATVTFHLPHHVLKSIASAIVNELKKINQNVAALPVASSVMDRLSYLLPSARPELGVGPGRSVDRSLMYSEANRRETFTSWPHVGYRWAQPDPMAQAGFYHQPASSGDDRAMCFTCSVCLVCWEPTDEPWSEHERHSPNCPFVKGEHTQNVPLSVTLATSPAQFPCTDGTDRISCFGSGSCPHFLAAATKRGKICIWDVSKLMKVHLKFEINAYDPAIVQQLILSGDPSSGVDSRRPTLAWLEDSSSCSDIPKLEGDSDDLLEDSDSEEHSRSDSVTGHTSQKEAMEVSLDITALSILQQPEKLQWEIVANVLEDTVKDLEELGANPCLTNSKSEKTKEKHQEQHNIPFPCLLAGGLLTYKSPATSPISSNSHRSLDGLSRTQGESISEQGSTDNESCTNSELNSPLVRRTLPVLLLYSIKESDEKAGKIFSQMNNIMSKSLHDDGFTVPQIIEMELDSQEQLLLQDPPVTYIQQFADAAANLTSPDSEKWNSVFPKPGTLVQCLRLPKFAEEENLCIDSITPCADGIHLLVGLRTCPVESLSAINQVEALNNLNKLNSALCNRRKGELESNLAVVNGANISVIQHESPADVQTPLIIQPEQRNVSGGYLVLYKMNYATRIVTLEEEPIKIQHIKDPQDTITSLILLPPDILDNREDDCEEPIEDMQLTSKNGFEREKTSDISTLGHLVITTQGGYVKILDLSNFEILAKVEPPKKEGTEEQDTFVSVIYCSGTDRLCACTKGGELHFLQIGGTCDDIDEADILVDGSLSKGIEPSSEGSKPLSNPSSPGISGVDLLVDQPFTLEILTSLVELTRFETLTPRFSATVPPCWVEVQQEQQQRRHPQHLHQQHHGDAAQHTRTWKLQTDSNSWDEHVFELVLPKACMVGHVDFKFVLNSNITNIPQIQVTLLKNKAPGLGKVNALNIEVEQNGKPSLVDLNEEMQHMDVEESQCLRLCPFLEDHKEDILCGPVWLASGLDLSGHAGMLTLTSPKLVKGMAGGKYRSFLIHVKAVNERGTEEICNGGMRPVVRLPSLKHQSNKGYSLASLLAKVAAGKEKSSNVKNENTSGTRKSENLRGCDLLQEVSVTIRRFKKTSISKERVQRCAMLQFSEFHEKLVNTLCRKTDDGQITEHAQSLVLDTLCWLAGVHSNGPGSSKEGNENLLSKTRKFLSDIVRVCFFEAGRSIAHKCARFLALCISNGKCDPCQPAFGPVLLKALLDNMSFLPAATTGGSVYWYFVLLNYVKDEDLAGCSTACASLLTAVSRQLQDRLTPMEALLQTRYGLYSSPFDPVLFDLEMSGSSCKNVYNSSIGVQSDEIDLSDVLSGNGKVSSCTAAEGSFTSLTGLLEVEPLHFTCVSTSDGTRIERDDAMSSFGVTPAVGGLSSGTVGEASTALSSAAQVALQSLSHAMASAEQQLQVLQEKQQQLLKLQQQKAKLEAKLHQTTAAAAAAASAVGPVHNSVPSNPVAAPGFFIHPSDVIPPTPKTTPLFMTPPLTPPNEAVSVVINAELAQLFPGSVIDPPAVNLAAHNKNSNKSRMNPLGSGLALAISHASHFLQPPPHQSIIIERMHSGARRFVTLDFGRPILLTDVLIPTCGDLASLSIDIWTLGEEVDGRRLVVATDISTHSLILHDLIPPPVCRFMKITVIGRYGSTNARAKIPLGFYYGHTYILPWESELKLMHDPLKGEGESANQPEIDQHLAMMVALQEDIQCRYNLACHRLETLLQSIDLPPLNSANNAQYFLRKPDKAVEEDSRVFSAYQDCIQLQLQLNLAHNAVQRLKVALGASRKMLSETSNPEDLIQTSSTEQLRTIIRYLLDTLLSLLHASNGHSVPAVLQSTFHAQACEELFKHLCISGTPKIRLHTGLLLVQLCGGERWWGQFLSNVLQELYNSEQLLIFPQDRVFMLLSCIGQRSLSNSGVLESLLNLLDNLLSPLQPQLPMHRRTEGVLDIPMISWVVMLVSRLLDYVATVEDEAAAAKKPLNGNQWSFINNNLHTQSLNRSSKGSSSLDRLYSRKIRKQLVHHKQQLNLLKAKQKALVEQMEKEKIQSNKGSSYKLLVEQAKLKQATSKHFKDLIRLRRTAEWSRSNLDTEVTTAKESPEIEPLPFTLAHERCISVVQKLVLFLLSMDFTCHADLLLFVCKVLARIANATRPTIHLCEIVNEPQLERLLLLLVGTDFNRGDISWGGAWAQYSLTCMLQDILAGELLAPVAAEAMEEGTVGDDVGATAGDSDDSLQQSSVQLLETIDEPLTHDITGAPPLSSLEKDKEIDLELLQDLMEVDIDPLDIDLEKDPLAAKVFKPISSTWYDYWGADYGTYNYNPYIGGLGIPVAKPPANTEKNGSQTVSVSVSQALDARLEVGLEQQAELMLKMMSTLEADSILQALTNTSPTLSQSPTGTDDSLLGGLQAANQTSQLIIQLSSVPMLNVCFNKLFSMLQVHHVQLESLLQLWLTLSLNSSSTGNKENGADIFLYNANRIPVISLNQASITSFLTVLAWYPNTLLRTWCLVLHSLTLMTNMQLNSGSSSAIGTQESTAHLLVSDPNLIHVLVKFLSGTSPHGTNQHSPQVGPTATQAMQEFLTRLQVHLSSTCPQIFSEFLLKLIHILSTERGAFQTGQGPLDAQVKLLEFTLEQNFEVVSVSTISAVIESVTFLVHHYITCSDKVMSRSGSDSSVGARACFGGLFANLIRPGDAKAVCGEMTRDQLMFDLLKLVNILVQLPLSGNREYSARVSVTTNTTDSVSDEEKVSGGKDGNGSSTSVQGSPAYVADLVLANQQIMSQILSALGLCNSSAMAMIIGASGLHLTKHENFHGGLDAISVGDGLFTILTTLSKKASTVHMMLQPILTYMACGYMGRQGSLATCQLSEPLLWFILRVLDTSDALKAFHDMGGVQLICNNMVTSTRAIVNTARSMVSTIMKFLDSGPNKAVDSTLKTRILASEPDNAEGIHNFAPLGTITSSSPTAQPAEVLLQATPPHRRARSAAWSYIFLPEEAWCDLTIHLPAAVLLKEIHIQPHLASLATCPSSVSVEVSADGVNMLPLSTPVVTSGLTYIKIQLVKAEVASAVCLRLHRPRDASTLGLSQIKLLGLTAFGTTSSATVNNPFLPSEDQVSKTSIGWLRLLHHCLTHISDLEGMMASAAAPTANLLQTCAALLMSPYCGMHSPNIEVVLVKIGLQSTRIGLKLIDILLRNCAASGSDPTDLNSPLLFGRLNGLSSDSTIDILYQLGTTQDPGTKDRIQALLKWVSDSARVAAMKRSGRMNYMCPNSSTVEYGLLMPSPSHLHCVAAILWHSYELLVEYDLPALLDQELFELLFNWSMSLPCNMVLKKAVDSLLCSMCHVHPNYFSLLMGWMGITPPPVQCHHRLSMTDDSKKQDLSSSLTDDSKNAQAPLALTESHLATLASSSQSPEAIKQLLDSGLPSLLVRSLASFCFSHISSSESIAQSIDISQDKLRRHHVPQQCNKMPITADLVAPILRFLTEVGNSHIMKDWLGGSEVNPLWTALLFLLCHSGSTSGSHNLGAQQTSARSASLSSAATTGLTTQQRTAIENATVAFFLQCISCHPNNQKLMAQVLCELFQTSPQRGNLPTSGNISGFIRRLFLQLMLEDEKVTMFLQSPCPLYKGRINATSHVIQHPMYGAGHKFRTLHLPVSTTLSDVLDRVSDTPSITAKLISEQKDDKEKKNHEEKEKVKAENGFQDNYSVVVASGLKSQSKRAVSATPPRPPSRRGRTIPDKIGSTSGAEAANKIITVPVFHLFHKLLAGQPLPAEMTLAQLLTLLYDRKLPQGYRSIDLTVKLGSRVITDPSLSKTDSYKRLHPEKDHGDLLASCPEDEALTPGDECMDGILDESLLETCPIQSPLQVFAGMGGLALIAERLPMLYPEVIQQVSAPVVTSTTQEKPKDSDQFEWVTIEQSGELVYEAPETVAAEPPPIKSAVQTMSPIPAHSLAAFGLFLRLPGYAEVLLKERKHAQCLLRLVLGVTDDGEGSHILQSPSANVLPTLPFHVLRSLFSTTPLTTDDGVLLRRMALEIGALHLILVCLSALSHHSPRVPNSSVNQTEPQVSSSHNPTSTEEQQLYWAKGTGFGTGSTASGWDVEQALTKQRLEEEHVTCLLQVLASYINPVSSAVNGEAQSSHETRGQNSNALPSVLLELLSQSCLIPAMSSYLRNDSVLDMARHVPLYRALLELLRAIASCAAMVPLLLPLSTENGEEEEEQSECQTSVGTLLAKMKTCVDTYTNRLRSKRENVKTGVKPDASDQEPEGLTLLVPDIQKTAEIVYAATTSLRQANQEKKLGEYSKKAAMKPKPLSVLKSLEEKYVAVMKKLQFDTFEMVSEDEDGKLGFKVNYHYMSQVKNANDANSAARARRLAQEAVTLSTSLPLSSSSSVFVRCDEERLDIMKVLITGPADTPYANGCFEFDVYFPQDYPSSPPLVNLETTGGHSVRFNPNLYNDGKVCLSILNTWHGRPEEKWNPQTSSFLQVLVSVQSLILVAEPYFNEPGYERSRGTPSGTQSSREYDGNIRQATVKWAMLEQIRNPSPCFKEVIHKHFYLKRVEIMAQCEEWIADIQQYSSDKRVGRTMSHHAAALKRHTAQLREELLKLPCPEGLDPDTDDAPEVCRATTGAEETLMHDQVKPSSSKELPSDFQL

SEQ ID NO: 102 - protein, 593 aa, Homo sapiens
MLRTSVLRLLGRTGASRLSLLEDFGPRYYSSGSLSAGDDACDVRAYFTTPIFYVNAAPHIGHLYSALLADALCRHRRLRGPSTAATRFSTGTDEHGLKIQQAAATAGLAPTELCDRVSEQFQQLFQEAGISCTDFIRTTEARHRVAVQHFWGVLKSRGLLYKGVYEGWYCASDECFLPEAKVTQQPGPSGDSFPVSLESGHPVSWTKEENYIFRLSQFRKPLQRWLRGNPQAITPEPFHHVVLQWLDEELPDLSVSRRSSHLHWGIPVPGDDSQTIYVWLDALVNYLTVIGYPNAEFKSWWPATSHIIGKDILKFHAIYWPAFLLGAGMSPPQRICVHSHWTVCGQKMSKSLGNVVDPRTCLNRYTVDGFRYFLLRQGVPNWDCDYYDEKVVKLLNSELADALGGLLNRCTAKRINPSETYPAFCTTCFPSEPGLVGPSVRAQAEDYALVSAVATLPKQVADHYDNFRIYKALEAVSSCVRQTNGFVQRHAPWKLNWESPVDAPWLGTVLHVALECLRVFGTLLQPVTPSLADKLLSRLGVSASERSLGELYFLPRFYGHPCPFEGRRLGPETGLLFPRLDQSRTWLVKAHRT

SEQ ID NO: 103 - protein, 377 aa, Homo sapiens
MAAAAQSRVVRVLSMSRSAITAIATSVCHGPPCRQLHHALMPHGKGGRSSVSGIVATVFGATGFLGRYVVNHLGRMGSQVIIPYRCDKYDIMHLRPMGDLGQLLFLEWDARDKDSIRRVVQHSNVVINLIGRDWETKNFDFEDVFVKIPQAIAQLSKEAGVEKFIHVSHLNANIKSSSRYLRNKAVGEKVVRDAFPEAIIVKPSDIFGREDRFLNSFASMHRFGPIPLGSLGWKTVKQPVYVVDVSKGIVNAVKDPDANGKSFAFVGPSRYLLFHLVKYIFAVAHRLFLPFPLPLFAYRWVARVFEISPFEPWITRDKVERMHITDMKLPHLPGLEDLGIQATPLELKAIEVLRRHRTYRWLSAEIEDVKPAKTVNI

SEQ ID NO: 104 - protein, 1318 aa, Homo sapiens
MSGGASATGPRRGPPGLEDTTSKKKQKDRANQESKDGDPRKETGSRYVAQAGLEPLASGDPSASASHAAGITGSRHRTRLFFPSSSGSASTPQEEQTKEGACEDPHDLLATPTPELLLDWRQSAEEVIVKLRVGVGPLQLEDVDAAFTDTDCVVRFAGGQQWGGVFYAEIKSSCAKVQTRKGSLLHLTLPKKVPMLTWPSLLVEADEQLCIPPLNSQTCLLGSEENLAPLAGEKAVPPGNDPVSPAMVRSRNPGKDDCAKEEMAVAADAATLVDEPESMVNLAFVKNDSYEKGPDSVVVHVYVKEICRDTSRVLFREQDFTLIFQTRDGNFLRLHPGCGPHTTFRWQVKLRNLIEPEQCTFCFTASRIDICLRKRQSQRWGGLEAPAARVGGAKVAVPTGPTPLDSTPPGGAPHPLTGQEEARAVEKDKSKARSEDTGLDSVATRTPMEHVTPKPETHLASPKPTCMVPPMPHSPVSGDSVEEEEEEEKKVCLPGFTGLVNLGNTCFMNSVIQSLSNTRELRDFFHDRSFEAEINYNNPLGTGGRLAIGFAVLLRALWKGTHHAFQPSKLKAIVASKASQFTGYAQHDAQEFMAFLLDGLHEDLNRIQNKPYTETVDSDGRPDEVVAEEAWQRHKMRNDSFIVDLFQGQYKSKLVCPVCAKVSITFDPFLYLPVPLPQKQKVLPVFYFAREPHSKPIKFLVSVSKENSTASEVLDSLSQSVHVKPENLRLAEVIKNRFHRVFLPSHSLDTVSPSDTLLCFELLSSELAKERVVVLEVQQRPQVPSVPISKCAACQRKQQSEDEKLKRCTRCYRVGYCNQLCQKTHWPDHKGLCRPENIGYPFLVSVPASRLTYARLAQLLEGYARYSVSVFQPPFQPGRMALESQSPGCTTLLSTGSLEAGDSERDPIQPPELQLVTPMAEGDTGLPRVWAAPDRGPVPSTSGISSEMLASGPIEVGSLPAGERVSRPEAAVPGYQHPSEAMNAHTPQFFIYKIDSSNREQRLEDKGDTPLELGDDCSLALVWRNNERLQEFVLVASKELECAEDPGSAGEAARAGHFTLDQCLNLFTRPEVLAPEEAWYCPQCKQHREASKQLLLWRLPNVLIVQLKRFSFRSFIWRDKINDLVEFPVRNLDLSKFCIGQKEEQLPSYDLYAVINHYGGMIGGHYTACARLPNDRSSQRSDVGWRLFDDSTVTTVDESQVVTRYAYVLFYRRRNSPVERPPRAGHSEHHPDLGPAAEAAASQASRIWQELEAEEEPVPEGSGPLGPWGPQDWVGPLPRGPTTPDEGCLRYFVLGTVAALVALVLNVFYPLVSQSRWR

SEQ ID NO: 105 - protein, 1052 aa, Homo sapiens
MEGSEPVAAHQGEEASCSSWGTGSTNKNLPIMSTASVEIDDALYSRQRYVLGDTAMQKMAKSHVFLSGMGGLGLEIAKNLVLAGIKAVTIHDTEKCQAWDLGTNFFLSEDDVVNKRNRAEAVLKHIAELNPYVHVTSSSVPFNETTDLSFLDKYQCVVLTEMKLPLQKKINDFCRSQCPPIKFISADVHGIWSRLFCDFGDEFEVLDTTGEEPKEIFISNITQANPGIVTCLENHPHKLETGQFLTFREINGMTGLNGSIQQITVISPFSFSIGDTTELEPYLHGGIAVQVKTPKTVFFESLERQLKHPKCLIVDFSNPEAPLEIHTAMLALDQFQEKYSRKPNVGCQQDSEELLKLATSISETLEEKPDVNADIVHWLSWTAQGFLSPLAAAVGGVASQEVLKAVTGKFSPLCQWLYLEAADIVESLGKPECEEFLPRGDRYDALRACIGDTLCQKLQNLNIFLVGCGAIGCEMLKNFALLGVGTSKEKGMITVTDPDLIEKSNLNRQFLFRPHHIQKPKSYTAADATLKINSQIKIDAHLNKVCPTTETIYNDEFYTKQDVIITALDNVEARRYVDSRCLANLRPLLDSGTMGTKGHTEVIVPHLTESYNSHRDPPEEEIPFCTLKSFPAAIEHTIQWARDKFESSFSHKPSLFNKFWQTYSSAEEVLQKIQSGHSLEGCFQVIKLLSRRPRNWSQCVELARLKFEKYFNHKALQLLHCFPLDIRLKDGSLFWQSPKRPPSPIKFDLNEPLHLSFLQNAAKLYATVYCIPFAEEDLSADALLNILSEVKIQEFKPSNKVVQTDETARKPDHVPISSEDERNAIFQLEKAILSNEATKSDLQMAVLSFEKDDDHNGHIDFITAASNLRAKMYSIEPADRFKTKRIAGKIIPAIATTTATVSGLVALEMIKVTGGYPFEAYKNCFLNLAIPIVVFTETTEVRKTKIRNGISFTIWDRWTVHGKEDFTLLDFINAVKEKYGIEPTMVVQGVKMLYVPVMPGHAKRLKLTMHKLVKPTTEKKYVDLTVSFAPDIDGDEDLPGPPVRYYFSHDTD

SEQ ID NO: 106 - protein, 334 aa, Homo sapiens
MRLTPRALCSAAQAAWRENFPLCGRDVARWFPGHMAKGLKKMQSSLKLVDCIIEVHDARIPLSGRNPLFQETLGLKPHLLVLNKMDLADLTEQQKIMQHLEGEGLKNVIFTNCVKDENVKQIIPMVTELIGRSHRYHRKENLEYCIMVIGVPNVGKSSLINSLRRQHLRKGKATRVGGEPGITRAVMSKIQVSERPLMFLLDTPGVLAPRIESVETGLKLALCGTVLDHLVGEETMADYLLYTLNKHQRFGYVQHYGLGSACDNVERVLKSVAVKLGKTQKVKVLTGTGNVNIIQPNYPAAARDFLQTFRRGLLGSVMLDLDVLRGHPPAETLP

SEQ ID NO: 107 - protein, 583 aa, Homo sapiens
MTFYLFGIRSFPKLWKSPYLGLGPGHSYVSLFLADRCGIRNQQRLFSLKTMSPQNTKATNLIAKARYLRKDEGSNKQVYSVPHFFLAGAAKERSQMNSQTEDHALAPVRNTIQLPTQPLNSEEWDKLKEDLKENTGKTSFESWIISQMAGCHSSIDVAKSLLAWVAAKNNGIVSYDLLVKYLYLCVFHMQTSEVIDVFEIMKARYKTLEPRGYSLLIRGLIHSDRWREALLLLEDIKKVITPSKKNYNDCIQGALLHQDVNTAWNLYQELLGHDIVPMLETLKAFFDFGKDIKDDNYSNKLLDILSYLRNNQLYPGESFAHSIKTWFESVPGKQWKGQFTTVRKSGQCSGCGKTIESIQLSPEEYECLKGKIMRDVIDGGDQYRKTTPQELKRFENFIKSRPPFDVVIDGLNVAKMFPKVRESQLLLNVVSQLAKRNLRLLVLGRKHMLRRSSQWSRDEMEEVQKQASCFFADDISEDDPFLLYATLHSGNHCRFITRDLMRDHKACLPDAKTQRLFFKWQQGHQLAIVNRFPGSKLTFQRILSYDTVVQTTGDSWHIPYDEDLVERCSCEVPTKWLCLHQKT

SEQ ID NO: 108 - protein, 393 aa, Homo sapiens
MASGRARCTRKLRNWVVEQVESGQFPGVCWDDTAKTMFRIPWKHAGKQDFREDQDAAFFKAWAIFKGKYKEGDTGGPAVWKTRLRCALNKSSEFKEVPERGRMDVAEPYKVYQLLPPGIVSGQPGTQKVPSKRQHSSVSSERKEEEDAMQNCTLSPSVLQDSLNNEEEGASGGAVHSDIGSSSSSSSPEPQEVTDTTEAPFQGDQRSLEFLLPPEPDYSLLLTFIYNGRVVGEAQVQSLDCRLVAEPSGSESSMEQVLFPKPGPLEPTQRLLSQLERGILVASNPRGLFVQRLCPIPISWNAPQAPPGPGPHLLPSNECVELFRTAYFCRDLVRYFQGLGPPPKFQVTLNFWEESHGSSHTPQNLITVKMEQAFARYLLEQTPEQQAAILSLV

SEQ ID NO: 109 - RNA, 9 nt, Artificial Sequence
  A synthetic sequence
uucaagaga

SEQ ID NO: 110 - DNA, 19 bp, Artificial Sequence
  A synthetic sequence
gggagccggttacttcctg

SEQ ID NO: 111 - DNA, 19 bp, Artificial Sequence
  A synthetic sequence
cttcttcaggagcgcccag

SEQ ID NO: 112 - DNA, 19 bp, Artificial Sequence
  A synthetic sequence
tcttggaagtaacagcggt

SEQ ID NO: 113 - DNA, 19 bp, Artificial Sequence
  A synthetic sequence
agagttgagagaaatggca

SEQ ID NO: 114 - DNA, 19 bp, Artificial Sequence
  A synthetic sequence
acgtcacagcctctgacag

SEQ ID NO: 115 - DNA, 19 bp, Artificial Sequence
  A synthetic sequence
tgctgcttcttgggggagc

SEQ ID NO: 116 - DNA, 19 bp, Artificial Sequence
  A synthetic sequence
gcgggatggcatcactgca

SEQ ID NO: 117 - DNA, 19 bp, Artificial Sequence
  A synthetic sequence
tgtgcactggtctcaggta

SEQ ID NO: 118 - DNA, 19 bp, Artificial Sequence
  A synthetic sequence
cagtccagtcaatagggat

SEQ ID NO: 119 - DNA, 19 bp, Artificial Sequence
  A synthetic sequence
ctggggctactgacgcgcg

SEQ ID NO: 120 - DNA, 19 bp, Artificial Sequence
  A synthetic sequence
tgcctgtttgttccggagc

SEQ ID NO: 121 - DNA, 19 bp, Artificial Sequence
  A synthetic sequence
cagggcggcgatccacctc

SEQ ID NO: 122 - DNA, 19 bp, Artificial Sequence
  A synthetic sequence
tctccatgcctgatgcaga

SEQ ID NO: 123 - DNA, 19 bp, Artificial Sequence
  A synthetic sequence
agacacttcggaccctccg

SEQ ID NO: 124 - DNA, 19 bp, Artificial Sequence
  A synthetic sequence
gggtacggggcatatgccg

SEQ ID NO: 125 - DNA, 19 bp, Artificial Sequence
  A synthetic sequence
cacccccgtggtcgcagaa

SEQ ID NO: 126 - DNA, 19 bp, Artificial Sequence
  A synthetic sequence
acagggttgcatcactagc

SEQ ID NO: 127 - DNA, 19 bp, Artificial Sequence
  A synthetic sequence
gttgatgtgaagggttaca

SEQ ID NO: 128 - DNA, 19 bp, Artificial Sequence
  A synthetic sequence
ctaggccgatacaaagcag

SEQ ID NO: 129 - DNA, 19 bp, Artificial Sequence
  A synthetic sequence
cacgtcctcgataccccca

SEQ ID NO: 130 - DNA, 19 bp, Artificial Sequence
  A synthetic sequence
cacctcctcgatatctggc

SEQ ID NO: 131 - DNA, 19 bp, Artificial Sequence
  A synthetic sequence
gctagcgaagttctccgtg

SEQ ID NO: 132 - DNA, 19 bp, Artificial Sequence
  A synthetic sequence
agggaacttctgatggtac

SEQ ID NO: 133 - DNA, 19 bp, Artificial Sequence
  A synthetic sequence
tagaaaactccaagataaa

SEQ ID NO: 134 - DNA, 19 bp, Artificial Sequence
  A synthetic sequence
ggaagcatgctgccaagca

SEQ ID NO: 135 - DNA, 19 bp, Artificial Sequence
  A synthetic sequence
ttcgcaatcttgggatcaa

SEQ ID NO: 136 - DNA, 19 bp, Artificial Sequence
  A synthetic sequence
agccgagatgctaagagca

SEQ ID NO: 137 - DNA, 19 bp, Artificial Sequence
  A synthetic sequence
atactcgtgcgtttggcgt

SEQ ID NO: 138 - DNA, 19 bp, Artificial Sequence
  A synthetic sequence
cctcgtggctggccccgag

SEQ ID NO: 139 - DNA, 19 bp, Artificial Sequence
  A synthetic sequence
tatccgcacatcgccgtca

SEQ ID NO: 140 - DNA, 19 bp, Artificial Sequence
  A synthetic sequence
cgattctctcactgagccg

SEQ ID NO: 141 - DNA, 19 bp, Artificial Sequence
  A synthetic sequence
tttgactgatgagtccact

SEQ ID NO: 142 - DNA, 19 bp, Artificial Sequence
  A synthetic sequence
aggtttcatacacagtcca

SEQ ID NO: 143 - DNA, 19 bp, Artificial Sequence
  A synthetic sequence
ttcttccaactgtccttgc

SEQ ID NO: 144 - DNA, 19 bp, Artificial Sequence
  A synthetic sequence
gttagagacgctattccgc

SEQ ID NO: 145 - DNA, 19 bp, Artificial Sequence
  A synthetic sequence
cccccggccgcacacctga

SEQ ID NO: 146 - DNA, 19 bp, Artificial Sequence
  A synthetic sequence
aagagcttgatagtaacaa

SEQ ID NO: 147 - DNA, 19 bp, Artificial Sequence
  A synthetic sequence
atgaggggccatgaagacg

SEQ ID NO: 148 - DNA, 19 bp, Artificial Sequence
  A synthetic sequence
ttccccccgaatcccagcg

SEQ ID NO: 149 - DNA, 19 bp, Artificial Sequence
  A synthetic sequence
ttaatagcaacaaatagag

SEQ ID NO: 150 - DNA, 19 bp, Artificial Sequence
  A synthetic sequence
tgcttcttgtcggcttgaa

SEQ ID NO: 151 - DNA, 19 bp, Artificial Sequence
  A synthetic sequence
gaggtctcggacagcgagt

SEQ ID NO: 152 - DNA, 19 bp, Artificial Sequence
  A synthetic sequence
tccatcagatgcttgtacg

SEQ ID NO: 153 - DNA, 19 bp, Artificial Sequence
  A synthetic sequence
cgttaggtacctgtgcgaa

SEQ ID NO: 154 - DNA, 19 bp, Artificial Sequence
  A synthetic sequence
agcccctcatttcaatgag

SEQ ID NO: 155 - DNA, 19 bp, Artificial Sequence
  A synthetic sequence
tgtatcagttgctgccacc

SEQ ID NO: 156 - DNA, 19 bp, Artificial Sequence
  A synthetic sequence
gcccctgaaaattctcccc

SEQ ID NO: 157 - DNA, 19 bp, Artificial Sequence
  A synthetic sequence
acgaggagactcactcggg

SEQ ID NO: 158 - DNA, 19 bp, Artificial Sequence
  A synthetic sequence
cgggtcgaaggcttcatag

SEQ ID NO: 159 - DNA, 19 bp, Artificial Sequence
  A synthetic sequence
gtgtgcatgctagtaactg

SEQ ID NO: 160 - DNA, 19 bp, Artificial Sequence
  A synthetic sequence
tgaagtctcgttgaaactg

SEQ ID NO: 161 - DNA, 19 bp, Artificial Sequence
  A synthetic sequence
tggtgctggacgtcggctc

SEQ ID NO: 162 - DNA, 19 bp, Artificial Sequence
  A synthetic sequence
atccgcctaccccgctcca

SEQ ID NO: 163 - DNA, 19 bp, Artificial Sequence
  A synthetic sequence
ggacttgtagggatgcacc

SEQ ID NO: 164 - DNA, 19 bp, Artificial Sequence
  A synthetic sequence
agcactggctcagagggtc

SEQ ID NO: 165 - DNA, 19 bp, Artificial Sequence
  A synthetic sequence
ctccacgatgaccgtgccc

SEQ ID NO: 166 - DNA, 19 bp, Artificial Sequence
  A synthetic sequence
tcacgctggtcgactgccc

SEQ ID NO: 167 - DNA, 19 bp, Artificial Sequence
  A synthetic sequence
atgagtacattgccgcgct

SEQ ID NO: 168 - DNA, 19 bp, Artificial Sequence
  A synthetic sequence
tcaccacgtgcttgggtac

SEQ ID NO: 169 - DNA, 19 bp, Artificial Sequence
  A synthetic sequence
ccgtgacctccaaggcgag

SEQ ID NO: 170 - DNA, 19 bp, Artificial Sequence
  A synthetic sequence
gccgtgagctacatgtcgc

SEQ ID NO: 171 - DNA, 19 bp, Artificial Sequence
  A synthetic sequence
accattggcgtggatccgc

SEQ ID NO: 172 - DNA, 19 bp, Artificial Sequence
  A synthetic sequence
agtcacgtgtcctaccgtc

SEQ ID NO: 173 - DNA, 19 bp, Artificial Sequence
  A synthetic sequence
gtatgggaactaatgtcgc

SEQ ID NO: 174 - DNA, 19 bp, Artificial Sequence
  A synthetic sequence
tattctacccatgctgccc

SEQ ID NO: 175 - DNA, 19 bp, Artificial Sequence
  A synthetic sequence
gcaaaagatgcctgcctga

SEQ ID NO: 176 - DNA, 19 bp, Artificial Sequence
  A synthetic sequence
cacgtcaccttggagccga

SEQ ID NO: 177 - DNA, 19 bp, Artificial Sequence
  A synthetic sequence
aatctcttactgaagctgc

SEQ ID NO: 178 - DNA, 19 bp, Artificial Sequence
  A synthetic sequence
cgactctaccccgtgctgc

SEQ ID NO: 179 - DNA, 19 bp, Artificial Sequence
  A synthetic sequence
cgactcggtgcacctcgcc

SEQ ID NO: 180 - DNA, 19 bp, Artificial Sequence
  A synthetic sequence
ggggggttcgtagcagagc

SEQ ID NO: 181 - DNA, 19 bp, Artificial Sequence
  A synthetic sequence
cagctcgtctacacctgca

SEQ ID NO: 182 - DNA, 19 bp, Artificial Sequence
  A synthetic sequence
gcactcacgtctctggcgc

SEQ ID NO: 183 - DNA, 19 bp, Artificial Sequence
  A synthetic sequence
cggttccagtctctcggtg

SEQ ID NO: 184 - DNA, 19 bp, Artificial Sequence
  A synthetic sequence
gcacggtgctcaccacgtc

SEQ ID NO: 185 - DNA, 19 bp, Artificial Sequence
  A synthetic sequence
attccatctatggctccac

SEQ ID NO: 186 - DNA, 19 bp, Artificial Sequence
  A synthetic sequence
tcatccggaccatcatcgg

SEQ ID NO: 187 - DNA, 19 bp, Artificial Sequence
  A synthetic sequence
accccagctgtgcaccggt

SEQ ID NO: 188 - DNA, 19 bp, Artificial Sequence
  A synthetic sequence
actatttctgtagtaccaa

SEQ ID NO: 189 - DNA, 19 bp, Artificial Sequence
  A synthetic sequence
tgctcgacttttccggcca

SEQ ID NO: 190 - DNA, 19 bp, Artificial Sequence
  A synthetic sequence
cacctgcgcaacctcgccg

SEQ ID NO: 191 - DNA, 19 bp, Artificial Sequence
  A synthetic sequence
caagtgagtagagaagatc

SEQ ID NO: 192 - DNA, 19 bp, Artificial Sequence
  A synthetic sequence
agcagaaacgtgtcctccg

SEQ ID NO: 193 - DNA, 19 bp, Artificial Sequence
  A synthetic sequence
cgctagtcccacccgcgga

SEQ ID NO: 194 - DNA, 19 bp, Artificial Sequence
  A synthetic sequence
tgcgatcaccactggcccg

SEQ ID NO: 195 - DNA, 19 bp, Artificial Sequence
  A synthetic sequence
acttctgtcaccgccttgc

SEQ ID NO: 196 - DNA, 19 bp, Artificial Sequence
  A synthetic sequence
cacgcaaggccacggtgcc

SEQ ID NO: 197 - DNA, 19 bp, Artificial Sequence
  A synthetic sequence
cagtacgtcatgctctcgg

SEQ ID NO: 198 - DNA, 19 bp, Artificial Sequence
  A synthetic sequence
agcatctcatacacacgcg

SEQ ID NO: 199 - DNA, 19 bp, Artificial Sequence
  A synthetic sequence
cctcggaactcacggcatg

SEQ ID NO: 200 - DNA, 19 bp, Artificial Sequence
  A synthetic sequence
tggctctgggtcggttgga

SEQ ID NO: 201 - DNA, 19 bp, Artificial Sequence
  A synthetic sequence
accaaagcttctgtcaggc

SEQ ID NO: 202 - DNA, 19 bp, Artificial Sequence
  A synthetic sequence
gccttcctcttcgtcggcg

SEQ ID NO: 203 - DNA, 19 bp, Artificial Sequence
  A synthetic sequence
caggtctgaagcgcctgac

SEQ ID NO: 204 - DNA, 19 bp, Artificial Sequence
  A synthetic sequence
gaccgattccgtggagtgc

SEQ ID NO: 205 - DNA, 19 bp, Artificial Sequence
  A synthetic sequence
caacctcacgaccggatca

SEQ ID NO: 206 - DNA, 19 bp, Artificial Sequence
  A synthetic sequence
agcatctccgctcatggca

SEQ ID NO: 207 - DNA, 19 bp, Artificial Sequence
  A synthetic sequence
ggcctgggtcgtcgcgacc

SEQ ID NO: 208 - DNA, 19 bp, Artificial Sequence
  A synthetic sequence
gccagcacacactcatgcc

SEQ ID NO: 209 - DNA, 19 bp, Artificial Sequence
  A synthetic sequence
agacagccagcatttccat

SEQ ID NO: 210 - DNA, 19 bp, Artificial Sequence
  A synthetic sequence
ttgccacaggaagtaaccg

SEQ ID NO: 211 - DNA, 20 bp, Artificial Sequence
  A synthetic sequence
ccagtacttgactcgtggag

SEQ ID NO: 212 - DNA, 21 bp, Artificial Sequence
  A synthetic sequence
ttaacaaggtggagcctcatc

SEQ ID NO: 213 - DNA, 20 bp, Artificial Sequence
  A synthetic sequence
ggcagattggagagaagagg

SEQ ID NO: 214 - DNA, 19 bp, Artificial Sequence
  A synthetic sequence
gccccacgacaataaactg

SEQ ID NO: 215 - DNA, 17 bp, Artificial Sequence
  A synthetic sequence
acagagcctcgcctttg

SEQ ID NO: 216 - DNA, 17 bp, Artificial Sequence
  A synthetic sequence
ccttgcacatgccggag

SEQ ID NO: 217 - DNA, 21 bp, Artificial Sequence
  A synthetic sequence
agaccccttcttcaggagcgc

SEQ ID NO: 218 - DNA, 27 bp, Artificial Sequence
  A synthetic sequence
tccgataccaataagtcagcctgatgc

SEQ ID NO: 219 - DNA, 25 bp, Artificial Sequence
  A synthetic sequence
cgtcgagaaccagcggagaaaagaa

SEQ ID NO: 220 - DNA, 21 bp, Artificial Sequence
  A synthetic sequence
tcatccatggtgagctggcgg